SEQUENCE LISTING (19 sequences)

SEQ ID NO: 1 - protein, 447 aa, synthetic construct
  source 1..447: note = XmAb20717 anti-CTLA4 VH-CH1-hinge-CH2-CH3; XmAb20717 anti-CTLA4 x anti-PD1 Fab-scFv-Fc Heavy Chain 1
EVQLVESGGGLVKPGGSLRLSCAASGFTFSSYTMHWVRQAPGKGLEWVSFISYDGNNKYYADSVKGRFTISRDNAKNSLYLQMNSLRAEDTAVYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 2 - protein, 480 aa, synthetic construct
  source 1..480: note = XmAb20717 anti-PD1 scFv-linker-CH2-CH3; XmAb20717 anti-CTLA4 x anti-PD1 Fab-scFv-Fc Heavy Chain 2
EIVLTQSPATLSASPGERVTLTCRASQSVGNDVAWYQQKPGQAPRLLINYASHRYTGVPDRFTGSGYGTEFTLTISSVQSEDFGVYYCQQDFSSPRTFGGGTKVEIKGKPGSGKPGSGKPGSGKPGSEVQLVESGGGLVKPGGSLRLSCVASGFTFSNYWMNWVRQAPGKGLEWVAEIRLYSNNYATHYAESVKGRFTISRDDSKSTLYLQMNNLKTEDTGVYYCTRYYGNYGGYFDVWGRGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - protein, 215 aa, synthetic construct
  source 1..215: note = XmAb20717 anti-CTLA4 VL-CL; XmAb20717 anti-CTLA4 x anti-PD1 Fab-scFv-Fc Light Chain
EIVLTQSPGTLSLSPGERATLSCRASQSVSSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, synthetic construct
  source 1..5: note = XmAb20717 Anti-PD1 VH CDR1
NYWMN

SEQ ID NO: 5 - protein, 19 aa, synthetic construct
  source 1..19: note = XmAb20717 Anti-PD1 VH CDR2
EIRLYSNNYATHYAESVKG

SEQ ID NO: 6 - protein, 11 aa, synthetic construct
  source 1..11: note = XmAb20717 Anti-PD1 VH CDR3
YYGNYGGYFDV

SEQ ID NO: 7 - protein, 11 aa, synthetic construct
  source 1..11: note = XmAb20717 Anti-PD1 VL CDR1
RASQSVGNDVA

SEQ ID NO: 8 - protein, 7 aa, synthetic construct
  source 1..7: note = XmAb20717 Anti-PD1 VL CDR2
YASHRYT

SEQ ID NO: 9 - protein, 9 aa, synthetic construct
  source 1..9: note = XmAb20717 Anti-PD1 VL CDR3
QQDFSSPRT

SEQ ID NO: 10 - protein, 5 aa, synthetic construct
  source 1..5: note = XmAb20717 Anti-CTLA4 VH CDR1
SYTMH

SEQ ID NO: 11 - protein, 17 aa, synthetic construct
  source 1..17: note = XmAb20717 Anti- CTLA4 VH CDR2
FISYDGNNKYYADSVKG

SEQ ID NO: 12 - protein, 9 aa, synthetic construct
  source 1..9: note = XmAb20717 Anti- CTLA4 VH CDR3
TGWLGPFDY

SEQ ID NO: 13 - protein, 12 aa, synthetic construct
  source 1..12: note = XmAb20717 Anti- CTLA4 VL CDR1
RASQSVSSSYLA

SEQ ID NO: 14 - protein, 7 aa, synthetic construct
  source 1..7: note = XmAb20717 Anti- CTLA4 VL CDR2
GAFSRAT

SEQ ID NO: 15 - protein, 9 aa, synthetic construct
  source 1..9: note = XmAb20717 Anti- CTLA4 VL CDR3
QQYGSSPWT

SEQ ID NO: 16 - protein, 122 aa, synthetic construct
  source 1..122: note = XmAb20717 Anti-PD1 VH
EVQLVESGGGLVKPGGSLRLSCVASGFTFSNYWMNWVRQAPGKGLEWVAEIRLYSNNYATHYAESVKGRFTISRDDSKSTLYLQMNNLKTEDTGVYYCTRYYGNYGGYFDVWGRGTLVTVSS

SEQ ID NO: 17 - protein, 107 aa, synthetic construct
  source 1..107: note = XmAb20717 Anti-PD1 VL
EIVLTQSPATLSASPGERVTLTCRASQSVGNDVAWYQQKPGQAPRLLINYASHRYTGVPDRFTGSGYGTEFTLTISSVQSEDFGVYYCQQDFSSPRTFGGGTKVEIK

SEQ ID NO: 18 - protein, 118 aa, synthetic construct
  source 1..118: note = XmAb20717 Anti-CTLA4 VH
EVQLVESGGGLVKPGGSLRLSCAASGFTFSSYTMHWVRQAPGKGLEWVSFISYDGNNKYYADSVKGRFTISRDNAKNSLYLQMNSLRAEDTAVYYCARTGWLGPFDYWGQGTLVTVSS

SEQ ID NO: 19 - protein, 108 aa, synthetic construct
  source 1..108: note = XmAb20717 Anti-CTLA4 VL
EIVLTQSPGTLSLSPGERATLSCRASQSVSSSYLAWYQQKPGQAPRLLIYGAFSRATGIPDRFSGSGSGTDFTLTISRLEPEDFAVYYCQQYGSSPWTFGQGTKVEIK

(19) United States
(12) Patent Application Publication
(10) Pub. No.: US 2023/0074793 A1
(43) Pub. Date: Mar. 9, 2023

(54) TREATMENT WITH A BISPECIFIC ANTIBODY THAT BINDS CTLA4 AND PD1

(71) Applicant: XENCOR, INC., Monrovia, CA (US)

(72) Inventors: Barbara HICKINGBOTTOM, Solana Beach, CA (US); Paul FOSTER, Rancho Santa Fe, CA (US); Raphael CLYNES, West Nyack, NY (US); Ying DING, San Diego, CA (US); Lei BAO, San Diego, CA (US); Catherine AVERSA-FLEENER, Escondido, CA (US)

(73) Assignee: XENCOR, INC., Monrovia, CA (US)

(21) Appl. No.: 17/817,845

(22) Filed: Aug. 5, 2022

Related U.S. Application Data
(60) Provisional application No. 63/346,194, filed on May 26, 2022.
(60) Provisional application No. 63/238,616, filed on Aug. 30, 2021.
(60) Provisional application No. 63/230,605, filed on Aug. 6, 2021.
(60) Provisional application No. 63/230,583, filed on Aug. 6, 2021.
(60) Provisional application No. 63/230,531, filed on Aug. 6, 2021.

(51) Int. Cl. C07K 16/28; A61P 35/00

(52) U.S. Cl. CPC C07K 16/2827; C07K 16/2896; A61P 35/00; C07K 2317/24; C07K 2317/55; C07K 2317/622; A61K 2039/505

(57) ABSTRACT

Provided herein, in certain aspects, are methods of treating a cancer in a subject, comprising administering a bispecific anti-CTLA4×anti-PD1 antibody to the subject.

### CROSS-REFERENCE TO RELATED APPLICATIONS

The present application claims the benefit of priority to U.S. Ser. No. 63/230,531 filed Aug. 6, 2021, U.S. Ser. No. 63/230,583 filed Aug. 6, 2021, U.S. Ser. No. 63/230,605 filed Aug. 6, 2021, U.S. Ser. No. 63/238,616 filed Aug. 30, 2021, and U.S. Ser. No. 63/346,194 filed May 26, 2022, the disclosure of each of which is incorporated herein by reference in its entirety.

### 1. FIELD

Provided herein, in certain aspects, are methods of treating a cancer in a subject, comprising administering a bispecific anti-CTLA4×anti-PD1 antibody to the subject.

### 2. SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14718-047-999_SEQ_LISTING.xml”, was created on Aug. 1, 2022 and is 22,608 bytes in size.

### 3. SUMMARY

Provided herein, in certain aspects, are methods of treating a cancer in a subject, comprising administering a bispecific anti-CTLA4×anti-PD1 antibody to the subject. Also provided herein, in certain aspects, are methods of achieving a positive therapeutic response against a cancer through administering a bispecific anti-CTLA4×anti-PD1 antibody to a human subject. In certain embodiments, the cancer is a solid cancerous tumor. In one aspect, provided herein is a method of treating a solid cancerous tumor in a subject comprising administering to the subject a bispecific anti-PD1×CTLA4 antibody. In another aspect, provided herein is a method of treating a prostate cancer in a subject comprising administering to the subject a bispecific anti-PD1×CTLA4 antibody. In another aspect, provided herein is a method of treating a gynecologic cancer in a subject comprising administering to the subject a bispecific anti-PD1×CTLA4 antibody. In another aspect, provided herein is a method of treating a genitourinary cancer in a subject comprising administering to the subject a bispecific anti-PD1×CTLA4 antibody.

In various aspects and embodiments of the methods provided herein, the bispecific anti-PD1×CTLA4 antibody is XmAb®20717 (also referred to herein as XmAb®717 or vudalimab). In other aspects and embodiments of the methods provided herein, the bispecific anti-PD1×CTLA4 antibody is a biosimilar, biobetter, or bioequivalent of XmAb®20717. In yet other aspects and embodiments of the methods provided herein, bispecific anti-PD1×CTLA4 antibody comprises a first monomer comprising SEQ ID NO: 1, a second monomer comprising SEQ ID NO: 2, and a light chain comprising SEQ ID NO: 3. In yet other aspects and embodiments of the methods provided herein, bispecific anti-PD1×CTLA4 antibody consists of a first monomer consisting of SEQ ID NO: 1, a second monomer consisting of SEQ ID NO: 2, and a light chain consisting of SEQ ID NO: 3. In certain embodiments, the bispecific anti-PD1×CTLA4 antibody is administered intravenously. In a specific embodiment, the subject is a male human subject. In other specific embodiments, the solid cancerous tumor is a prostate cancer.

In one aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the prostate cancer is microsatellite instability-high (MSI-H) prostate cancer. In one embodiment, the prostate cancer is mismatch repair deficient (MMRD) prostate cancer. In one embodiment, the subject receives treatment about every 2 weeks (Q2W) for about two years.

In another aspect, provided herein is a method of treating an aggressive variant (anaplastic) adenocarcinoma of the prostate (AVPCa) in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the method further comprising orally administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating an aggressive variant (anaplastic) adenocarcinoma of the prostate (AVPCa) in a male human subject in need thereof, wherein the subject has not previously been administered docetaxel, and wherein the method comprises: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the method further comprises administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter.

In another aspect, provided herein is a method of treating a prostate cancer in a male human subject in need thereof, wherein the subject has not previously been administered docetaxel, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, wherein the subject has not previously been administered a PARP inhibitor, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In some embodiments, the method further comprising administering olaparib at a dose of about 300 mg. In some embodiments, the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

In another aspect, provided herein is a method of treating an advanced gynecologic or genitourinary malignancy in a human subject in need thereof, the method comprising administering to the subject a dose of a bispecific antibody according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle, wherein the bispecific antibody comprises a first monomer comprising SEQ ID NO:1, a second monomer comprising SEQ ID NO:2, and a light chain comprising SEQ ID NO:3. In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments of the methods provided herein, if the weight of the subject changes by more than 10% from baseline, the subject is optionally reassigned to a new dosing level and one or more subsequent doses are administered to the subject at the new dosing level. In some embodiments of the methods provided herein, if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

### 4. BRIEF DESCRIPTION OF THE DRAWINGS

FIG. 1 depicts the structure of the antibody described herein. The XmAb®20717 has a “bottle opener” format (also referred to as the “triple F” format). Bottle opener format antibodies include a) a first monomer that includes a first Fc domain and an scFv region, wherein the scFv includes a first variable heavy chain and a first variable light chain (also referred herein as a “scFv-Fc heavy chain;” b) a second monomer that includes a VH-CH1-hinge-CH2-CH3, wherein VH is a second variable heavy chain and CH2 and CH3 is a second Fc domain (also referred herein as a “Fab-Fc heavy chain;” and c) a light chain that includes a second variable light chain. As shown in FIG. 1, the scFv is the PD1 binding domain and the second variable heavy chain and second variable light chain for the CTLA4 binding domain. It should be noted that the scFv and Fab domains can be switched (e.g., anti-PD1 as a Fab and anti-CTLA4 as a scFv).

FIG. 2 depicts the amino acid sequences of the XmAb®20717 antibody. The antibody is named using the Fab variable region first and the scFv variable region second, separated by a dash, followed by the chain designation (Fab-Fc heavy chain, scFv-Fc heavy chain or light chain). CDRs are underlined and slashes indicate the border(s) of the variable regions.

FIG. 3 shows potent antitumor immune response in a mCRPC patient following XmAb®20717 treatment, as shown by multiplex immunofluorescence staining of paited pre-treatment and post-treatment biopsies.

FIG. 4 depicts a study design showing the study population, cohorts, and their respective treatments with the XmAb®20717 antibody.

FIG. 5 depicts the study schema for subjects treated with the XmAb®20717 antibody.

FIG. 6 depicts a graph showing study cohorts and treatment.

FIG. 7 depicts the dosing schedules for XmAb®20717 and combination chemotherapy infusions using carboplatin and cabazitaxel (or docetaxel) (Cohorts A, B, & E).

FIG. 8 depicts the study flowchart using a flat dose of XmAb®20717 based on the body weight of a subject.

### 5. DETAILED DESCRIPTION

### 5.1 Definitions

In order that the application may be more completely understood, several definitions are set forth below. Such definitions are meant to encompass grammatical equivalents.

As used herein, and unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “about” in relation to a reference numerical value can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

Unless otherwise indicated, the term “at least” preceding a series of elements is to be understood to refer to every element in the series.

The terms “a,” “an,” “the” and similar referents used in the context of describing the disclosure (especially in the context of the following claims) are to be construed to cover both the singular and the plural, unless otherwise indicated herein or clearly contradicted by context.

By “CTLA4,” “CTLA4,” “cytotoxic T-lymphocyte-associated protein 4,” “CD152,” or “cluster of differentiation 152” (e.g., GenBank Accession Number NP 001032720 (human isoform without transmembrane) and NP 005205 (human isoform with transmembrane)) as used herein is meant a coinhibitory receptor that is present on the surface of CD4+ and CD8+ T cells that is upregulated in inflammatory environments in which activated T cells are present. Unless otherwise stated herein, the bispecific antibodies provided herein bind to the extracellular domain of human CTLA4. CTLA4 is a member of the immunoglobulin superfamily. CTLA4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. CTLA4 is capable of providing a physiological counterbalance to immune cell activation and thereby to control the intensity of the immune response. It exerts this effect by outcompeting CD28, a costimulatory molecule necessary for T-cell activation, for binding to CD80 and CD86 on antigen-presenting cells and tumor cells. The net effect of CTLA4 up-regulation is down-modulation of T-cell activation (Postow et al., J Clin Oncol. 2015; 33(17):1974-1982). CTLA4 can also inhibit T cell responses directly via SHP-2 and PP2A dephosphorylation of TCR-proximal signaling proteins such as CD3 and LAT. CTLA4 is also known to bind PI3K.

By “PD1,” “PD1,” “Programmed cell death protein 1,” “CD279,” and “cluster of differentiation 279” (e.g., GenBank Accession Number NP_0015009 (human)) as used herein is meant a type I membrane protein that is a member of the extended CD28/CTLA4 family of T cell regulators. Unless otherwise stated herein, the bispecific antibodies provided herein bind to the extracellular domain of human PD1. PD1 includes an extracellular IgV domain followed by a transmembrane region and an intracellular tail PD1 is expressed on the surface of activated T cells, B cells and macrophage and is upregulated in the context of chronic and persistent antigen stimulation. In human subjects with cancer, PD1 is upregulated on the surface of activated tumor-infiltrating CD8+ T cells, as well as activated B cells and myeloid cells. Its primary ligands, PDL1 and PDL2, may be expressed on a wide range of cell types including antigen-presenting cells and tumor cells, and the overall effect of engagement of the ligands is to limit, terminate, or attenuate the cytotoxic and cytokine-producing capacity of cytotoxic T cells. This, in turn, results in an ineffective antitumor immune response and the persistence of tumors (Postow et al., J Clin Oncol. 2015; 33(17):1974-1982).

By “bispecific” or “bispecific antibody” herein is meant any non-native or alternate antibody formats, including those described herein, that bind to two different antigens (e.g., PD1×CTLA4 bispecific antibodies, such as XmAb®20717).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In this case, any amino acid substitution is always to an amino acid coded for by DNA, e.g., the 20 amino acids that have codons in DNA and RNA. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234. In this case, any amino acid insertion is to an amino acid coded for by DNA, e.g., the 20 amino acids that have codons in DNA and RNA.

By “amino acid deletion” or “deletion” as used herein is meant the removal of an amino acid sequence at a particular position in a parent polypeptide sequence. For example, K447-, K4476_, K447del or K447# designates a deletion a deletion of lysine at position 446. As is known in the art and more fully disclosed below, N- or C-terminal clipping can occur during production, including the lysine at position 447, the glycine at position 446, or additional amino acids. In some cases, the nucleic acids encoding the heavy chains herein can terminate at position 447, 446, 445, 444, etc.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about twenty amino acid modifications compared to the parent. For example, the variant Fc domains of XmAb®717 can have 14 amino acid variants as compared to a human IgG1. As described below, in some embodiments the parent polypeptide, for example an Fc parent polypeptide, is a human wild type sequence, such as the Fc region from IgG1, IgG2, IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants provided herein are defined according to the amino acid modifications that compose them. Thus, for example, N434S or 434S is an Fc variant with the substitution serine at position 434 relative to the parent Fc polypeptide, where the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, N434S/M428L is the same Fc variant as M428L/N434S, and so on. For all positions discussed herein that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. For clarity, variant proteins within the scope of the present disclosure retain the biological activity of the parent protein, although it can differ in scale or scope. Thus, for example, amino acid substitutions can be made in the anti-CTLA4 antigen binding domains provided herein as long as they retain the ability to bind to human CTLA4 as measured by a BIOCORE or Octet assay.

As used herein, “protein” herein is meant at least two covalently attached amino acids, which includes proteins, polypeptides, oligopeptides and peptides.

By “residue” as used herein is meant a position in a protein and its associated amino acid identity. For example, Asparagine 297 (also referred to as Asn297 or N297) is a residue at position 297 in the human antibody IgG1.

As used herein, the term “antibody” is used in a broad sense and includes immunoglobulin or antibody molecules. In general, antibodies are proteins or peptide chains that exhibit binding specificity to a specific antigen. In specific embodiments, the antibodies provided herein are based on IgG1, IgG2, or IgG4. In some embodiments, the antibodies provided herein are based on IgG1, IgG2, or IgG4 and contain amino acid variants. In many preferred embodiments, the antibodies provided herein are based on human IgG1, and contain amino acid variants as outlined herein. Antibody light chains of vertebrate species can be assigned to one of two clearly distinct types, namely kappa and lambda, based on the amino acid sequences of their constant domains. In one embodiment, an antibody provided herein comprises a kappa light chain. In other embodiments, an antibody provided herein comprises a lambda light chain. In certain embodiments, an antibody provided herein comprises a kappa light chain and a lambda light chain. Accordingly, the antibodies provided herein can, in certain embodiments, contain a kappa light chain constant domain. In other embodiments, antibodies provided herein can, in certain embodiments, contain lambda light chain constant domains. In some embodiments, the antibodies provided herein can contain kappa light chain and lambda light chain constant domains.

In addition to the heavy and light constant domains, antibodies contain an antigen-binding region that is made up of a light chain variable (VL) domain (or region) and a heavy chain variable (VH) domain (or region). The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A “CDR” refers to one of three hypervariable regions (VH CDR1, VH CDR2 or VH CDR3) within the non-framework region of the immunoglobulin (Ig or antibody) VH β-sheet framework, or one of three hypervariable regions (VL CDR1, VL CDR2 or VL CDR3) within the non-framework region of the antibody VL β-sheet framework. Accordingly, CDRs are variable region sequences interspersed within the framework region sequences. The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

A number of hypervariable region delineations are in use and are encompassed herein, for example, in the tables and/or Examples provided below. The “Kabat” CDRs are based on sequence variability and are the most commonly used (see, e.g., Kabat et al., Sequences of Proteins of Immunological Interest, 5th Ed. Public Health Service, National Institutes of Health, Bethesda, Md. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures. An additional a universal numbering system can also be used, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lafranc et al., supra). An Exemplary system may also be used that combines Kabat and Chothia.

| | Exem- | | | | | | |
|---|---|---|---|---|---|---|---|
| | plary | | | | | | |
| | (Kabat + | | | | | | |
| | Chothia) | IMGT | Kabat | AbM | Chothia | Contact | Xencor |
| V_{H} | 26-35 | 27-38 | 31-35 | 26-35 | 26-32 | 30-35 | 27-35 |
| CDR1 | | | | | | | |
| V_{H} | 50-65 | 56-65 | 50-65 | 50-58 | 53-55 | 47-58 | 54-61 |
| CDR2 | | | | | | | |
| V_{H} | 95-102 | 105-117 | 95-102 | 95-102 | 96-101 | 93-101 | 103-116 |
| CDR3 | | | | | | | |
| V_{L} | 24-34 | 27-38 | 24-34 | 24-34 | 26-32 | 30-36 | 27-38 |
| CDR1 | | | | | | | |
| V_{L} | 50-56 | 56-65 | 50-56 | 50-56 | 50-52 | 46-55 | 56-62 |
| CDR2 | | | | | | | |
| V_{L} | 89-97 | 105-117 | 89-97 | 89-97 | 91-96 | 89-96 | 97-105 |
| CDR3 | | | | | | | |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, hinge, CH2 and CH3 regions of the heavy chain and the CL region of the light chain.

The term “framework” or “FR” residues are those variable region residues flanking the CDRs. FR residues are those variable domain residues other than the hypervariable region residues or CDR residues.

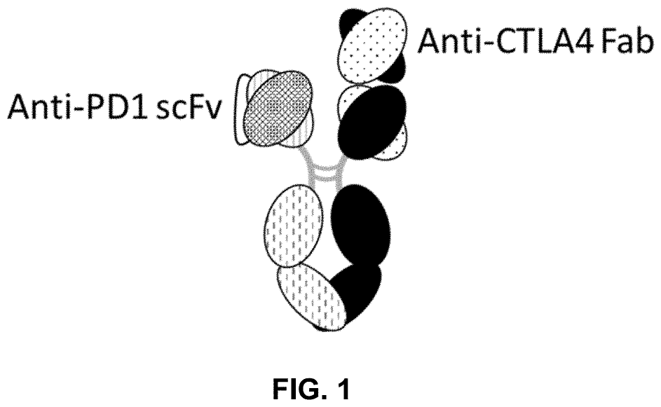

By “antigen binding domain” or “ABD” herein is meant a set of six Complementary Determining Regions (CDRs) that, when present as part of a polypeptide sequence, specifically binds a target antigen as discussed herein. Thus, a “CTLA4 antigen binding domain” binds human CTLA4. A “checkpoint antigen binding domain” binds a target checkpoint antigen as outlined herein. As is known in the art, these CDRs are generally present as a first set of variable heavy CDRs (vhCDRs or VHCDRs) and a second set of variable light CDRs (v1CDRs or VLCDRs), each comprising three CDRs: vhCDR1, vhCDR2, vhCDR3 for the heavy chain and v1CDR1, v1CDR2 and v1CDR3 for the light. The CDRs are present in the variable heavy and variable light domains, respectively, and together form an Fv region. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the v1CDR1, v1CDR2 and v1CDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh (including optional domain linkers on each side, depending on the format used (e.g., from FIG. 1). In general, the C-terminus of the scFv domain is attached to the N-terminus of the hinge in the second monomer.

By “Fab” or “Fab region” as used herein is meant the polypeptide that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody provided herein. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the VL and VH domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

By “amino acid” and “amino acid identity” as used herein is meant one of the 20 naturally occurring amino acids that are coded for by DNA and RNA.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIe; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

By “FcRn” or “neonatal Fc Receptor” as used herein is meant a protein that binds the IgG antibody Fc region and is encoded at least in part by an FcRn gene. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin. polypeptide that is modified to generate a variant, and by “parent antibody” as used herein is meant an unmodified antibody that is modified to generate a variant antibody.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus, the definition of “Fe domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.) Human IgG Fc domains are of particular use in the methods provided herein, and can be the Fc domain from human IgG1, IgG2 or IgG4.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

By “position” as used herein is meant a location in the sequence of a protein. Positions may be numbered sequentially, or according to an established format, for example the EU index for antibody numbering.

By “target antigen” as used herein is meant the molecule that is bound specifically by the antigen binding domain comprising the variable regions of a given antibody. As discussed below, in the present case the target antigens are CTLA4 and PD1.

By “strandedness” in the context of the monomers of the heterodimeric antibodies provided herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses a target antigen.

By “host cell” in the context of producing a bispecific antibody provided herein is meant a cell that contains the exogeneous nucleic acids encoding the components of the bispecific antibody and is capable of expressing the bispecific antibody under suitable conditions. Suitable host cells are discussed herein.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and v1CDR1, v1CDR2 and v1CDR3 for the variable light domain) and four framework (FR) regions, arranged from amino-terminus to carboxy-terminus in the following order: FR1-CDR1-FR2-CDR2-FR3-CDR3-FR4.

Also provided is a number of antibody domains that have sequence identity to human antibody domains, or to the anti-CTLA4 domains, PD1 domains, or Fc domains of XmAb®717. Sequence identity between two similar sequences (e.g., antibody variable domains) can be measured by algorithms such as that of Smith, T. F. & Waterman, M. S. (1981) “Comparison of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters. Accordingly, suitable Fv or Fc domains can be, e.g., 90%, 95%, 98%, 99%, or 100% identical to the parental domains.

By “wild type or WT” herein is meant an amino acid sequence or a nucleotide sequence that is found in nature, including allelic variations. A WT protein has an amino acid sequence or a nucleotide sequence that has not been intentionally modified.

The antibodies provided herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10′ M, at least about 10⁻⁵ M, at least about 10⁻⁶M, at least about 10⁻⁷M, at least about 10⁻⁸M, at least about 10⁻⁹M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

As used herein, the terms “positive therapeutic response,” “treat,” “treatment,” and/or “treating” refer to the reduction or amelioration or elimination of the progression, severity and/or effect associated with a solid cancerous tumor (e.g., prostate cancer, or an advanced gynecologic or genitourinary malignancy) described herein, or the improvement in the solid cancerous tumor (e.g., prostate cancer, or an advanced gynecologic or genitourinary malignancy) condition, or the improvement in the disease associated with the solid cancerous tumor (e.g., prostate cancer, or an advanced gynecologic or genitourinary malignancy), or the increase in the immune system response of the human subject (e.g., male subject for prostate cancer, or an advanced gynecologic or genitourinary malignancy), or the amelioration of one or more symptoms (preferably, one or more discernible symptoms) of a solid cancerous tumor (e.g., prostate cancer, or an advanced gynecologic or genitourinary malignancy) described herein resulting from the administration of one or more therapies. In specific embodiments, the terms “positive therapeutic response,” “treat,” “treatment,” and/or “treating” refer to the amelioration of at least one measurable physical parameter of a solid cancerous tumor (e.g., prostate cancer, or an advanced gynecologic or genitourinary malignancy) described herein, such as tumor size, rate of tumor growth, number of tumor cells, tumor invasiveness, presence of metastasis, or extent of metastasis. In other embodiments the terms “positive therapeutic response,” “treat,” “treatment,” and/or “treating” refer to the inhibition of the progression of a solid cancerous tumor (e.g., prostate cancer, or an advanced gynecologic or genitourinary malignancy) described herein, either physically by, e.g., stabilization of a discernible symptom, physiologically by, e.g., stabilization of a physical parameter, or both. In an exemplary embodiment, achieving a positive therapeutic response against a solid cancerous tumor (e.g., prostate cancer, or an advanced gynecologic or genitourinary malignancy) provides an improvement, or a lack of progression, in the disease associated with the tumor or the tumor condition, and/or an improvement, or a lack of progression, in the symptoms associated with the disease or condition.

For example, achieving a positive therapeutic response against a solid cancerous tumor (e.g., prostate cancer) refers to one or more of the following: (1) a reduction in the number of cancer cells (e.g., prostate cancer cells); (2) an increase in cell death (e.g., prostate cancer cell death); (3) inhibition of cell survival (e.g., prostate cancer cell survival); (5) inhibition (i.e., slowing to some extent, preferably lack of progression) of cancer growth (e.g., prostate cancer growth), such as stable disease; (6) inhibition of cancer cell metastasis (e.g., prostate cancer cell metastasis); (7) an increase in progression-free survival; (8) an increase in overall survival rate; and (9) some relief from one or more symptoms associated with the disease or condition.

Additional descriptions regarding positive therapeutic responses can be found in the RECIST criteria (Eisenhauer et al. Eur J Can. 2009; 45:228-47; Chalian et al. Onc. 2011; 31:2093-105); the imRECIST criteria (Hodi et al. J Clin Oncol. 2018: JCO2017751644. doi: 10.1200/JCO.2017.75.1644); the modified IrRC criteria (Wolchok et al. Clin Cancer Res. 2009; 15(23):7412-20); and the PCWG3 criteria (Scher et al. Clin Oncol. 2016; 34(12):1402-18). Each of the aforementioned references is herein incorporated by reference in its entirety for disclosures relating to the respective criteria. Exemplary embodiments for the assessment of solid cancerous tumor treatment using the RECIST guidelines are also provided elsewhere herein.

In some embodiments, achieving a positive therapeutic response against a solid cancerous tumor (e.g., prostate cancer, or an advanced gynecologic or genitourinary malignancy) involves administering the compositions described herein for a pre-specified period of time, discontinuing administration for another specific period of time, and resuming administration of the compositions described herein for yet another specific period of time. In some embodiments, achieving a positive therapeutic response against a solid cancerous tumor (e.g., prostate cancer, or an advanced gynecologic or genitourinary malignancy) involves administering the compositions described herein until one of the responses described herein is achieved, pausing administration of the compositions described herein while this positive therapeutic response continues to be observed, and resuming administration of the compositions described herein if this positive therapeutic response ceases to be observed.

Positive therapeutic responses against a solid cancerous tumor (e.g., prostate cancer, or an advanced gynecologic or genitourinary malignancy) can be determined by standardized response criteria specific to the disease associated with the solid cancerous tumor (e.g., prostate cancer, or an advanced gynecologic or genitourinary malignancy). Solid cancerous tumor (e.g., prostate cancer, or an advanced gynecologic or genitourinary malignancy) response can be assessed for changes in tumor morphology (i.e., with neo-adjuvant use of a therapy, such as assessment of pathological response) or tumor metrics (i.e., overall tumor burden, tumor size, and the like) using screening techniques such as magnetic resonance imaging (MM) scan, positron emission tomography (PET) scan, x-radiographic imaging, radionuclide scan, computed tomographic (CT) scan, bone scan imaging, endoscopy, tumor sampling including bone marrow aspiration (BMA), and counting of tumor marker levels and/or tumor cells in the circulation.

A positive therapeutic response or treating according to the present disclosure can include an improvement in one or more symptoms associated with a solid cancerous tumor or cancer of the disclosure. In some embodiments, a symptom includes feeling less tired, feeling less weak, feeling less dizzy or lightheaded, reduction in shortness of breath, reduction in fever, fewer infections, quicker recovery from infections, reduction in ease of bruising, reduction in bleeding episodes, weight gain, reduction in night sweats, gain of appetite, reduction in abdominal swelling, reduction in lymph node swelling, reduction in bone or joint pain, and reduction in thymus swelling.

In certain embodiments, the positive therapeutic response is as compared to the response in the subject prior to treatment with the bispecific anti-CTLA4×anti-PD1 antibody.

A positive therapeutic response or treating according to the present disclosure includes a “therapeutically effective amount” of the medicaments used. A “therapeutically effective amount” refers to an amount effective, at dosages and for periods of time necessary, to achieve a desired therapeutic result. In some embodiments, the desired therapeutic result is a complete response, partial response or stable disease. In specific embodiments, the desired therapeutic result is an improvement in response according to the RECIST guidelines (v. 1.1) as outlined in Eisenhauer et al. Eur. J. Cancer, 2009, 45:228-247, which is incorporated herein by reference in its entirety regarding, e.g., tumor response evaluation criteria. For example, in the case of evaluation of target lesions, for example, a “Complete Response” (CR) refers to the disappearance of all target lesions, where any pathological lymph nodes (whether target or non-target) must have reduction in short axis to <10 mm. A “Partial Response” (PR) refers to at least a 30% decrease in the sum of diameters of target lesions, taking as reference the baseline sum diameters. “Progressive Disease” (PD) refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). “Stable Disease” (SD) refers to neither sufficient shrinkage to qualify for PR nor sufficient increase to qualify for PD, taking as reference the smallest sum diameters while on study. In the case of evaluation of non-target lesions, a “Complete Response” (CR) refers to the disappearance of all non-target lesions and normalization of tumor marker level. All lymph nodes must be non-pathological in size (<10 mm short axis). A “Non-CR/Non-PD” refers to persistence of one or more non-target lesion(s) and/or maintenance of tumor marker level above the normal limits. A “Progressive Disease” (PD) refers to the unequivocal progression of existing non-target lesions. (Note: the appearance of one or more new lesions is also considered progression).

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of the compositions described herein to achieve a positive therapeutic response against a solid cancerous tumor (e.g., prostate cancer) may be evaluated in an animal model system predictive of efficacy in human tumors.

Alternatively, this property of a composition may be evaluated by examining the ability of its components to inhibit cell growth or to induce apoptosis by in vitro assays known to the skilled practitioner. A therapeutically effective amount of a composition described herein may decrease tumor size, or otherwise ameliorate symptoms in a subject. One of ordinary skill in the art would be able to determine such amounts based on such factors as the human subject's size, the severity of the human subject's symptoms, and the particular composition or route of administration selected.

The terms “patient,” “subject,” and “human subject” can be used interchangeably herein.

### 5.2 Overview

Disclosed herein are methods of treating a cancer that include cells expressing CTLA4 (“CTLA4-expressing cancer”), for example, a solid cancerous tumor of the disclosure, through the administration of certain bispecific anti-PD1×CTLA4 antibodies at particular dosages. The term “CTLA4-expressing cancer” can refer to a cancer that expresses CTLA4 or a cancer that overexpresses CTLA4 as compared to normal tissue. The term “PD1-expressing cancer” can refer to a cancer that expresses PD1 or a cancer that overexpresses PD1. Thus, the methods provided herein include treating a cancer that expresses both CTLA4 and PD1, for example, a solid cancerous tumor, through the administration of certain bispecific anti-PD1×CTLA4 antibodies at particular dosages. The term “PD1-expressing cancer” can refer to a cancer that expresses PD1 or a cancer that overexpresses PD1. Also provided are methods of combination therapies, for example, methods of treating a cancer that include cells expressing CTLA4 (“CTLA4-expressing cancer”), through the administration of certain bispecific anti-PD1×CTLA4 antibodies (e.g., XmAb®20717) in combination with one or more therapies that can ameliorate side effects of an anti-CTLA4×anti-PD1 bispecific antibody.

In some aspects, the methods provided herein are directed to the administration of the bispecific anti-PD1×CTLA4 antibodies (e.g., XmAb®20717) for the treatment of particular solid cancerous tumors (e.g., prostate cancer) as outlined herein. The disclosure provides methods of achieving a positive therapeutic response against a solid cancerous tumor (e.g., prostate cancer) through the administration of XmAb®20717 according to a dosage regimen described herein. The present disclosure also provides methods of treating solid cancerous tumors through the administration of XmAb®20717 according to a dosage regimen described herein. Also provided herein are methods of achieving a positive therapeutic response against prostate cancer through the administration of XmAb®20717, carboplatin, and cabazitaxel, according to a dosage regimen described herein. Also provided herein are methods of achieving a positive therapeutic response against prostate cancer through the administration of XmAb®20717, carboplatin, and docetaxel, according to a dosage regimen described herein. The disclosure also provides methods of achieving a positive therapeutic response against prostate cancer through the administration of XmAb®20717 and olaparib according to a dosage regimen described herein.

In other aspects, the methods provided herein are directed to the administration of the bispecific anti-PD1×CTLA4 antibodies (e.g., XmAb®20717) for the treatment of an advanced gynecologic or genitourinary malignancy as outlined herein. The disclosure provides methods of achieving a positive therapeutic response against the advanced gynecologic or genitourinary malignancy through the administration of XmAb®20717 according to a dosage regimen described herein. The present disclosure also provides methods of treating an advanced gynecologic or genitourinary malignancy through the administration of XmAb®20717 according to a dosage regimen described herein. In certain embodiments, the malignancy is an advanced gynecologic malignancy. In other embodiments, the malignancy is an advanced genitourinary malignancy. In an embodiment, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In an embodiment, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In an embodiment, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In an embodiment, the malignancy is a high-risk metastatic castration-resistant prostate cancer (mCRPC). In an embodiment, the malignancy is an advanced endometrial carcinoma that is not microsatellite instability-high (MSI-H) or deficient mismatch repair (dMMR). In certain embodiments, the subject is intravenously administered the bispecific anti-CTLA4×anti-PD1 antibody at a dose of about 1200 mg if the subject weighs 80 kg or more, wherein the dose is administered on day 1 of each 21 day treatment cycle. In certain embodiments, the subject is intravenously administered the bispecific anti-CTLA4×anti-PD1 antibody at a dose of about 1000 mg if the subject weighs less than 80 kg, wherein the dose is administered on day 1 of each 21 day treatment cycle.

It will be understood that XmAb®20717 can be used as the bispecific anti-CTLA4×anti-PD1 antibody in all methods of the invention provided herein.

It will also be understood that that a biosimilar of XmAb®20717 can be used as the bispecific anti-CTLA4×anti-PD1 antibody in all methods of the invention provided herein.

It will further be understood that a bioequivalent of XmAb®20717 can be used as the bispecific anti-CTLA4×anti-PD1 antibody in all methods of the invention provided herein.

### 5.3 Antibodies

Provided herein are methods directed to the administration of XmAb®20717 to achieve a positive therapeutic response against a solid cancerous tumor (e.g., prostate cancer, or an advanced gynecologic or genitourinary malignancy). The present disclosure is directed to the administration of XmAb®20717 for the treatment of a solid cancerous tumor (e.g., prostate cancer, or an advanced gynecologic or genitourinary malignancy), as described herein and in U.S. patent application Ser. No. 15/623,314, US Publication No. 2018/0118836, U.S. Prov. Pat. App. Nos. 62/350,145, 62/355,511, and 62/420,500, all of which are expressly incorporated herein by reference, particularly for the bispecific formats of the figures, as well as all sequences, Figures and accompanying Legends therein.

Amino acid sequences of exemplary bispecific anti-CTL4×anti-PD1 antibodies useful in the methods provided herein are shown in Table 1 below.

TABLE 1
Amino Acid Sequences of Exemplary Bispecific anti-CTLA4 × anti-PD1
Antibodies

| | | SEQ ID |
|---|---|---|
| Description | Sequence | NO: |
| XmAb®20717 | EVQLVESGGGLVKPGGSLRLSCAASGFTFSSYTMHWVRQAPGKGLEW | 1 |
| Anti-CTLA4 | VSFISYDGNNKYYADSVKGRFTISRDNAKNSLYLQMNSLRAEDTAVY | |
| VH-CH1-hinge-CH2- | YCARTGWLGPFDYWGQGTLVTVSSASTKGPSVFPLAPSSKSTSGGTA | |
| CH3 | ALGCLVKDYFPEPVTVSWNSGALTSGVHTFPAVLQSSGLYSLSSVVT | |
| XmAb®20717 anti- | VPSSSLGTQTYICNVNHKPSDTKVDKKVEPKSCDKTHTCPPCPAPPV | |
| CTLA4 × anti-PD1 | AGPSVFLFPPKPKDTLMISRTPEVTCVVVDVKHEDPEVKFNWYVDGV | |
| Fab-scFv-Fc Heavy | EVHNAKTKPREEEYNSTYRVVSVLTVLHQDWLNGKEYKCKVSNKALP | |
| Chain 1 | APIEKTISKAKGQPREPQVYTLPPSREEMTKNQVSLTCDVSGFYPSD | |
| | IAVEWESDGQPENNYKTTPPVLDSDGSFFLYSKLTVDKSRWEQGDVF | |
| | SCSVLHEALHSHYTQKSLSLSPGK | |
| XmAb®20717 | EIVLTQSPATLSASPGERVTLTCRASQSVGNDVAWYQQKPGQAPRLL | 2 |
| Anti-PD1 | INYASHRYTGVPDRFTGSGYGTEFTLTISSVQSEDFGVYYCQQDFSS | |
| scFv-linker-CH2- | PRTFGGGTKVEIKGKPGSGKPGSGKPGSGKPGSEVQLVESGGGLVKP | |
| CH3 | GGSLRLSCVASGFTFSNYWMNWVRQAPGKGLEWVAEIRLYSNNYATH | |
| XmAb®20717 anti- | YAESVKGRFTISRDDSKSTLYLQMNNLKTEDTGVYYCTRYYGNYGGY | |
| CTLA4 × anti-PD1 | FDVWGRGTLVTVSSEPKSSDKTHTCPPCPAPPVAGPSVFLFPPKPKD | |
| Fab-scFv-Fc Heavy | TLMISRTPEVTCVVVDVKHEDPEVKFNWYVDGVEVHNAKTKPREEQY | |
| Chain 2 | NSTYRVVSVLTVLHQDWLNGKEYKCKVSNKALPAPIEKTISKAKGQP | |
| | REPQVYTLPPSREQMTKNQVKLTCLVKGFYPSDIAVEWESNGQPENN | |
| | YKTTPPVLDSDGSFFLYSKLTVDKSRWQQGNVFSCSVLHEALHSHYT | |
| | QKSLSLSPGK | |
| XmAb®20717 | EIVLTQSPGTLSLSPGERATLSCRASQSVSSSYLAWYQQKPGQAPRL | 3 |
| Anti-CTLA4 | LIYGAFSRATGIPDRFSGSGSGTDFTLTISRLEPEDFAVYYCQQYGS | |
| VL-CL | SPWTFGQGTKVEIKRTVAAPSVFIFPPSDEQLKSGTASVVCLLNNFY | |
| XmAb®20717 | PREAKVQWKVDNALQSGNSQESVTEQDSKDSTYSLSSTLTLSKADYE | |
| anti-CTLA4 × anti- | KHKVYACEVTHQGLSSPVTKSFNRGEC | |
| PD1 | | |
| Fab-scFv-Fc Light | | |
| Chain | | |
| XmAb®20717 | NYWMN | 4 |
| Anti-PD1 VH CDR1 | | |
| XmAb®20717 | EIRLYSNNYATHYAESVKG | 5 |
| Anti-PD1 VH CDR2 | | |
| XmAb®20717 | YYGNYGGYFDV | 6 |
| Anti-PD1 VH CDR3 | | |
| XmAb®20717 | RASQSVGNDVA | 7 |
| Anti-PD1 VL CDR1 | | |
| XmAb®20717 | YASHRYT | 8 |
| Anti-PD1 VL CDR2 | | |
| XmAb®20717 | QQDFSSPRT | 9 |
| Anti-PD1 VL CDR3 | | |
| XmAb®20717 | SYTMH | 10 |
| Anti-CTLA4 VH | | |
| CDR1 | | |
| XmAb®20717 | FISYDGNNKYYADSVKG | 11 |
| Anti- CTLA4 VH | | |
| CDR2 | | |
| XmAb®20717 | TGWLGPFDY | 12 |
| Anti- CTLA4 VH | | |
| CDR3 | | |
| XmAb®20717 | RASQSVSSSYLA | 13 |
| Anti- CTLA4 VL | | |
| CDR1 | | |
| XmAb®20717 | GAFSRAT | 14 |
| Anti- CTLA4 VL | | |
| CDR2 | | |
| XmAb®20717 | QQYGSSPWT | 15 |
| Anti- CTLA4 VL | | |
| CDR3 | | |
| XmAb®20717 | EVQLVESGGGLVKPGGSLRLSCVASGFTFSNYWMNWVRQAPGKGLEW | 16 |
| Anti-PD1 VH | VAEIRLYSNNYATHYAESVKGRFTISRDDSKSTLYLQMNNLKTEDTG | |
| | VYYCTRYYGNYGGYFDVWGRGTLVTVSS | |
| XmAb®20717 | EIVLTQSPATLSASPGERVTLTCRASQSVGNDVAWYQQKPGQAPRLL | 17 |
| Anti-PD1 VL | INYASHRYTGVPDRFTGSGYGTEFTLTISSVQSEDFGVYYCQQDFSS | |
| | PRTFGGGTKVEIK | |
| XmAb®20717 | EVQLVESGGGLVKPGGSLRLSCAASGFTFSSYTMHWVRQAPGKGLEW | 18 |
| Anti-CTLA4 VH | VSFISYDGNNKYYADSVKGRFTISRDNAKNSLYLQMNSLRAEDTAVY | |
| | YCARTGWLGPFDYWGQGTLVTVSS | |
| XmAb®20717 | EIVLTQSPGTLSLSPGERATLSCRASQSVSSSYLAWYQQKPGQAPRL | 19 |
| Anti-CTLA4 VL | LIYGAFSRATGIPDRFSGSGSGTDFTLTISRLEPEDFAVYYCQQYGS | |
| | SPWTFGQGTKVEIK | |

In some embodiments, the bispecific anti-CTLA4×anti-PD1 antibodies (e.g., XmAb®20717) have a “bottle opener” format (also referred to as the “triple F” format) as is generally depicted in FIG. 1. In this embodiment, the PD1 antigen binding domain is the scFv in the bottle opener format and the CTLA4 antigen binding domain is the Fab in the bottle opener format (terms as used in US Publication No. 20180118836 A1, all of which are expressly incorporated by reference in their entirety and specifically for all the definitions, sequences of CTLA4 antigen binding domains and sequences of PD1 antigen binding domains).

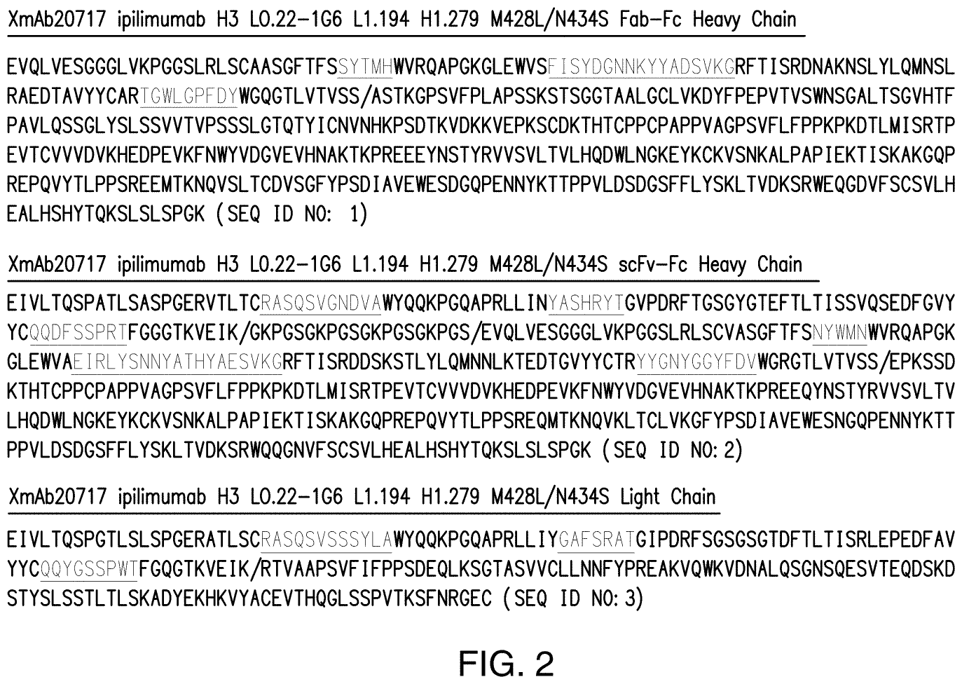

The anti-PD1 scFv antigen binding domain can have the sequence depicted in FIG. 2, Table 1. The anti-CTLA4 Fab binding domain can have the sequence depicted in FIG. 2, Table 1.

One bispecific antibody of particular use in the methods provided herein, XmAb®20717, is shown in FIG. 2 and Table 1. In certain embodiments, XmAb®20717 includes a first monomer comprising SEQ ID NO: 1, a second monomer comprising SEQ ID NO: 2, and a light chain comprising SEQ ID NO: 3. XmAb®20717 can be made as known in the art. In certain embodiments, XmAb®20717 is made by expressing a nucleic acid composition that includes a) a first nucleic that encodes a first amino acid monomer comprising “Fab-Fc Heavy Chain;” b) a second nucleic that encodes a second amino acid monomer comprising “scFv-Fc Heavy Chain;” and c) a third nucleic that encodes a “light chain,” as depicted in FIG. 2.

Additionally, as is known in the art, N- and/or C-terminal clipping can occur during protein synthesis, whereby the heavy chains depicted herein may have the C-terminal lysine (K447) removed, as well as the penultimate glycine (G446), and optionally additional amino acids residues (e.g., from 1, 2, 3, 4, 5, 6, 7, 8, 9, 10 or more C-terminal amino acids can be removed). Alternatively, the nucleic acids encoding the heavy chains of the bispecific antibodies can be engineered such that these terminal residues are eliminated entirely to facilitate additional homogeneity. Thus, included within the definition of XmAb717 are C-terminally truncated versions. As is known in the art, this C-terminal clipping can also occur in vivo in the patient.

In some embodiments, the nucleic acids that encode for each of these three amino acid sequences can be incorporated into one or more expression vectors for expression. In certain embodiments, XmAb®20717 consists of a first monomer comprising SEQ ID NO: 1, a second monomer comprising SEQ ID NO: 2, and a light chain comprising SEQ ID NO: 3. In certain embodiments, XmAb®20717 includes a first monomer consisting of SEQ ID NO: 1, a second monomer comprising SEQ ID NO: 2, and a light chain comprising SEQ ID NO: 3. In certain embodiments, XmAb®20717 includes a first monomer comprising SEQ ID NO: 1, a second monomer consisting of SEQ ID NO: 2, and a light chain comprising SEQ ID NO: 3. In certain embodiments, XmAb®20717 includes a first monomer comprising SEQ ID NO: 1, a second monomer comprising SEQ ID NO: 2, and a light chain consisting of SEQ ID NO: 3. In certain embodiments, XmAb®20717 includes a first monomer consisting of SEQ ID NO: 1, a second monomer consisting of SEQ ID NO: 2, and a light chain comprising SEQ ID NO: 3. In certain embodiments, XmAb®20717 includes a first monomer consisting of SEQ ID NO: 1, a second monomer comprising SEQ ID NO: 2, and a light chain consisting of SEQ ID NO: 3. In certain embodiments, XmAb®20717 includes a first monomer comprising SEQ ID NO: 1, a second monomer consisting of SEQ ID NO: 2, and a light chain consisting of SEQ ID NO: 3. In certain embodiments, XmAb®20717 includes a first monomer consisting of SEQ ID NO: 1, a second monomer consisting of SEQ ID NO: 2, and a light chain consisting of SEQ ID NO: 3.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1.

In one embodiment, the bispecific anti-CTLA4×anti-PD1 antibody comprises a first antigen binding domain that binds to PD1, wherein the first antigen binding domain comprises a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the bispecific anti-CTLA4×anti-PD1 antibody comprises a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1.

In one embodiment, the bispecific anti-CTLA4×anti-PD1 antibody comprises a second antigen binding domain that binds to CTLA4, wherein the second antigen binding domain comprises a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the bispecific anti-CTLA4×anti-PD1 antibody comprises a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1, and a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence provided in Table 1, and a VL comprising a VL CDR1, VL CDR2 and VL CDR3 having an amino acid sequence of a VL CDR1, VL CDR2 and VL CDR3, respectively, of a VL having an amino acid sequence provided in Table 1.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 16.

In one embodiment, the bispecific anti-CTLA4×anti-PD1 antibody comprises a first antigen binding domain that binds to PD1, wherein the first antigen binding domain comprises a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 17.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 16, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 17.

In one embodiment, the bispecific anti-CTLA4×anti-PD1 antibody comprises a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 18.

In one embodiment, the bispecific anti-CTLA4×anti-PD1 antibody comprises a second antigen binding domain that binds to CTLA4, wherein the first antigen binding domain comprises a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 19.

In one embodiment, the bispecific anti-CTLA4×anti-PD1 antibody comprises a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 18, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 19.

In one embodiment, the bispecific anti-CTLA4×anti-PD1 antibody comprises a first antigen binding domain that binds to PD1, wherein the first antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 16, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 17, and a second antigen binding domain that binds to CTLA4, wherein the second antigen binding domain comprises a VH comprising a VH CDR1, VH CDR2 and VH CDR3 having an amino acid sequence of a VH CDR1, VH CDR2 and VH CDR3, respectively, of a VH having an amino acid sequence of SEQ ID NO: 18, and a VL comprising a VL CDR1, VL CDR2 and VL CDR3 having an amino acid sequence of a VL CDR1, VL CDR2 and VL CDR3, respectively, of a VL having an amino acid sequence of SEQ ID NO: 19.

In one embodiment, the bispecific anti-CTLA4×anti-PD1 antibody comprises a first antigen binding domain that binds to PD1, wherein the first antigen binding domain comprises a VH domain comprising a VH CDR1, a VH CDR2, and a VH CDR3 having an amino acid sequence of SEQ ID NO:4, SEQ ID NO:5, and SEQ ID NO:6, respectively.

In one embodiment, the bispecific anti-CTLA4×anti-PD1 antibody comprises a first antigen binding domain that binds to PD1, wherein the first antigen binding domain comprises a VL domain comprising a VL CDR1, a VL CDR2, and a VL CDR3 having an amino acid sequence of SEQ ID NO:7, SEQ ID NO:8, and SEQ ID NO:9, respectively.

In one embodiment, the bispecific anti-CTLA4×anti-PD1 antibody comprises a first antigen binding domain that binds to PD1, wherein the first antigen binding domain comprises a VH domain comprising a VH CDR1, a VH CDR2, and a VH CDR3 having the amino acid sequence of SEQ ID NO:4, SEQ ID NO:5, and SEQ ID NO:6, respectively, and a VL domain comprising a VL CDR1, a VL CDR2, and a VL CDR3 having an amino acid sequence of SEQ ID NO:7, SEQ ID NO:8, and SEQ ID NO:9, respectively.

In one embodiment, the bispecific anti-CTLA4×anti-PD1 antibody comprises a second antigen binding domain that binds to CTLA4, wherein the second antigen binding domain comprises a VH domain comprising a VH CDR1, a VH CDR2, and a VH CDR3 having an amino acid sequence of SEQ ID NO:10, SEQ ID NO:11, and SEQ ID NO:12, respectively.

In one embodiment, the bispecific anti-CTLA4×anti-PD1 antibody comprises a second antigen binding domain that binds to CTLA4, wherein the second antigen binding domain comprises a VL domain comprising a VL CDR1, a VL CDR2, and a VL CDR3 having an amino acid sequence of SEQ ID NO:13, SEQ ID NO:14, and SEQ ID NO:15, respectively.

In one embodiment, the bispecific anti-CTLA4×anti-PD1 antibody comprises a second antigen binding domain that binds to CTLA4, wherein the second antigen binding domain comprises a VH domain comprising a VH CDR1, a VH CDR2, and a VH CDR3 having an amino acid sequence of SEQ ID NO:10, SEQ ID NO:11, and SEQ ID NO:12, respectively, and a VL domain comprising a VL CDR1, a VL CDR2, and a VL CDR3 having an amino acid sequence of SEQ ID NO:13, SEQ ID NO:14, and SEQ ID NO:15, respectively.

In one embodiment, the bispecific anti-CTLA4×anti-PD1 antibody comprises a first antigen binding domain that binds to PD1 and a second antigen binding domain that binds to CTLA4, wherein the first antigen binding domain comprises a VH domain comprising a VH CDR1, a VH CDR2, and a VH CDR3 having an amino acid sequence of SEQ ID NO:4, SEQ ID NO:5, and SEQ ID NO:6, respectively, and a VL domain comprising a VL CDR1, a VL CDR2, and a VL CDR3 having an amino acid sequence of SEQ ID NO:7, SEQ ID NO:8, and SEQ ID NO:9, respectively, and the second antigen binding domain comprises a VH domain comprising a VH CDR1, a VH CDR2, and a VH CDR3 having an amino acid sequence of SEQ ID NO:10, SEQ ID NO:11, and SEQ ID NO:12, respectively, and a VL domain comprising a VL CDR1, a VL CDR2, and a VL CDR3 having an amino acid sequence of SEQ ID NO:13, SEQ ID NO:14, and SEQ ID NO:15, respectively.

In one embodiment, the bispecific anti-CTLA4×anti-PD1 antibody comprises a first antigen binding domain that binds to PD1, wherein the first antigen binding domain comprises a VH domain having an amino acid sequence that is about 90%, 95%, 98%, 99%, or 100% identical to the amino acid sequence of SEQ ID NO:16. In one embodiment, the first antigen binding domain comprises a VH domain having an amino acid sequence that is about 90% identical to the amino acid sequence of SEQ ID NO:16. In another embodiment, the first antigen binding domain comprises a VH domain having an amino acid sequence that is about 95% identical to the amino acid sequence of SEQ ID NO:16. In another embodiment, the first antigen binding domain comprises a VH domain having an amino acid sequence that is about 98% identical to the amino acid sequence of SEQ ID NO:16. In another embodiment, the first antigen binding domain comprises a VH domain having an amino acid sequence that is about 99% identical to the amino acid sequence of SEQ ID NO:16. In one embodiment, the first antigen binding domain comprises a VH domain having an amino acid sequence that is about 100% identical to the amino acid sequence of SEQ ID NO:16.

In one embodiment, the bispecific anti-CTLA4×anti-PD1 antibody comprises a first antigen binding domain that binds to PD1, wherein the first antigen binding domain comprises a VL domain having an amino acid sequence that is about 90%, 95%, 98%, 99%, or 100% identical to the amino acid sequence of SEQ ID NO:17. In one embodiment, the first antigen binding domain comprises a VL domain having an amino acid sequence that is about 90% identical to the amino acid sequence of SEQ ID NO:17. In another embodiment, the first antigen binding domain comprises a VL domain having an amino acid sequence that is about 95% identical to the amino acid sequence of SEQ ID NO:17. In another embodiment, the first antigen binding domain comprises a VL domain having an amino acid sequence that is about 98% identical to the amino acid sequence of SEQ ID NO:17. In another embodiment, the first antigen binding domain comprises a VL domain having an amino acid sequence that is about 99% identical to the amino acid sequence of SEQ ID NO:17. In another embodiment, the first antigen binding domain comprises a VL domain having an amino acid sequence that is about 100% identical to the amino acid sequence of SEQ ID NO:17.

In one embodiment, the bispecific anti-CTLA4×anti-PD1 antibody comprises a first antigen binding domain that binds to PD1, wherein the first antigen binding domain comprises a VH domain having an amino acid sequence that is about 90%, 95%, 98%, 99%, or 100% identical to the amino acid sequence of SEQ ID NO:16 and a VL domain having an amino acid sequence that is about 90%, 95%, 98%, 99%, or 100% identical to the amino acid sequence of SEQ ID NO:17. In one embodiment, the first antigen binding domain comprises a VH domain having an amino acid sequence that is about 90%, identical to the amino acid sequence of SEQ ID NO:16 and a VL domain having an amino acid sequence that is about 90% identical to the amino acid sequence of SEQ ID NO:17. In another embodiment, the first antigen binding domain comprises a VH domain having an amino acid sequence that is about 95%, identical to the amino acid sequence of SEQ ID NO:16 and a VL domain having an amino acid sequence that is about 95% identical to the amino acid sequence of SEQ ID NO:17. In another embodiment, the first antigen binding domain comprises a VH domain having an amino acid sequence that is about 98%, identical to the amino acid sequence of SEQ ID NO:16 and a VL domain having an amino acid sequence that is about 98% identical to the amino acid sequence of SEQ ID NO:17. In another embodiment, the first antigen binding domain comprises a VH domain having an amino acid sequence that is about 99%, identical to the amino acid sequence of SEQ ID NO:16 and a VL domain having an amino acid sequence that is about 99% identical to the amino acid sequence of SEQ ID NO:17. In another embodiment, the first antigen binding domain comprises a VH domain having an amino acid sequence that is about 100%, identical to the amino acid sequence of SEQ ID NO:16 and a VL domain having an amino acid sequence that is about 100% identical to the amino acid sequence of SEQ ID NO:17.

In one embodiment, the bispecific anti-CTLA4×anti-PD1 antibody comprises a second antigen binding domain that binds to CTLA4, wherein the second antigen binding domain comprises a VH domain having an amino acid sequence that is about 90%, 95%, 98%, 99%, or 100% identical to the amino acid sequence of SEQ ID NO:18. In one embodiment, the second antigen binding domain comprises a VH domain having an amino acid sequence that is about 90% identical to the amino acid sequence of SEQ ID NO:18. In another embodiment, the second antigen binding domain comprises a VH domain having an amino acid sequence that is about 95% identical to the amino acid sequence of SEQ ID NO:18. In another embodiment, the second antigen binding domain comprises a VH domain having an amino acid sequence that is about 98% identical to the amino acid sequence of SEQ ID NO:18. In another embodiment, the second antigen binding domain comprises a VH domain having an amino acid sequence that is about 99% identical to the amino acid sequence of SEQ ID NO:18. In another embodiment, the second antigen binding domain comprises a VH domain having an amino acid sequence that is about 100% identical to the amino acid sequence of SEQ ID NO:18.

In one embodiment, the bispecific anti-CTLA4×anti-PD1 antibody comprises a second antigen binding domain that binds to CTLA4, wherein the second antigen binding domain comprises a VL domain having an amino acid sequence that is about 90%, 95%, 98%, 99%, or 100% identical to the amino acid sequence of SEQ ID NO:19. In one embodiment, the second antigen binding domain comprises a VL domain having an amino acid sequence that is about 90% identical to the amino acid sequence of SEQ ID NO:19. In another embodiment, the second antigen binding domain comprises a VL domain having an amino acid sequence that is about 95% identical to the amino acid sequence of SEQ ID NO:19. In another embodiment, the second antigen binding domain comprises a VL domain having an amino acid sequence that is about 98% identical to the amino acid sequence of SEQ ID NO:19. In another embodiment, the second antigen binding domain comprises a VL domain having an amino acid sequence that is about 99% identical to the amino acid sequence of SEQ ID NO:19. In another embodiment, the second antigen binding domain comprises a VL domain having an amino acid sequence that is about 100% identical to the amino acid sequence of SEQ ID NO:19.

In one embodiment, the bispecific anti-CTLA4×anti-PD1 antibody comprises a second antigen binding domain that binds to CTLA4, wherein the second antigen binding domain comprises a VH domain having an amino acid sequence that is about 90%, 95%, 98%, 99%, or 100% identical to the amino acid sequence of SEQ ID NO:18 and a VL domain having an amino acid sequence that is about 90%, 95%, 98%, 99%, or 100% identical to the amino acid sequence of SEQ ID NO:19. In one embodiment, the second antigen binding domain comprises a VH domain having an amino acid sequence that is about 90%, identical to the amino acid sequence of SEQ ID NO:18 and a VL domain having an amino acid sequence that is about 90% identical to the amino acid sequence of SEQ ID NO:19. In another embodiment, the second antigen binding domain comprises a VH domain having an amino acid sequence that is about 95%, identical to the amino acid sequence of SEQ ID NO:18 and a VL domain having an amino acid sequence that is about 95% identical to the amino acid sequence of SEQ ID NO:19. In another embodiment, the second antigen binding domain comprises a VH domain having an amino acid sequence that is about 98%, identical to the amino acid sequence of SEQ ID NO:18 and a VL domain having an amino acid sequence that is about 98% identical to the amino acid sequence of SEQ ID NO:19. In another embodiment, the second antigen binding domain comprises a VH domain having an amino acid sequence that is about 99%, identical to the amino acid sequence of SEQ ID NO:18 and a VL domain having an amino acid sequence that is about 99% identical to the amino acid sequence of SEQ ID NO:19. In another embodiment, the second antigen binding domain comprises a VH domain having an amino acid sequence that is about 100%, identical to the amino acid sequence of SEQ ID NO:18 and a VL domain having an amino acid sequence that is about 100% identical to the amino acid sequence of SEQ ID NO:19.

In one embodiment, the bispecific anti-CTLA4×anti-PD1 antibody comprises a first antigen binding domain that binds to PD1 and a second antigen binding domain that binds to CTLA4, wherein the first antigen binding domain comprises a VH domain having an amino acid sequence that is about 90%, 95%, 98%, 99%, or 100% identical to the amino acid sequence of SEQ ID NO:16 and a VL domain having an amino acid sequence that is about 90%, 95%, 98%, 99%, or 100% identical to the amino acid sequence of SEQ ID NO:17, and the second antigen binding domain comprises a VH domain having an amino acid sequence that is about 90%, 95%, 98%, 99%, or 100% identical to the amino acid sequence of SEQ ID NO:18 and a VL domain having an amino acid sequence that is about 90%, 95%, 98%, 99%, or 100% identical to the amino acid sequence of SEQ ID NO:19. In one embodiment, the first antigen binding domain comprises a VH domain having an amino acid sequence that is about 90%, identical to the amino acid sequence of SEQ ID NO:16 and a VL domain having an amino acid sequence that is about 90% identical to the amino acid sequence of SEQ ID NO:17, and the second antigen binding domain comprises a VH domain having an amino acid sequence that is about 90%, identical to the amino acid sequence of SEQ ID NO:18 and a VL domain having an amino acid sequence that is about 90% identical to the amino acid sequence of SEQ ID NO:19. In another embodiment, the first antigen binding domain comprises a VH domain having an amino acid sequence that is about 95%, identical to the amino acid sequence of SEQ ID NO:16 and a VL domain having an amino acid sequence that is about 95% identical to the amino acid sequence of SEQ ID NO:17, and the second antigen binding domain comprises a VH domain having an amino acid sequence that is about 95%, identical to the amino acid sequence of SEQ ID NO:18 and a VL domain having an amino acid sequence that is about 95% identical to the amino acid sequence of SEQ ID NO:19. In another embodiment, the first antigen binding domain comprises a VH domain having an amino acid sequence that is about 98%, identical to the amino acid sequence of SEQ ID NO:16 and a VL domain having an amino acid sequence that is about 98% identical to the amino acid sequence of SEQ ID NO:17, and the second antigen binding domain comprises a VH domain having an amino acid sequence that is about 98%, identical to the amino acid sequence of SEQ ID NO:18 and a VL domain having an amino acid sequence that is about 98% identical to the amino acid sequence of SEQ ID NO:19. In another embodiment, the first antigen binding domain comprises a VH domain having an amino acid sequence that is about 99%, identical to the amino acid sequence of SEQ ID NO:16 and a VL domain having an amino acid sequence that is about 99% identical to the amino acid sequence of SEQ ID NO:17, and the second antigen binding domain comprises a VH domain having an amino acid sequence that is about 99%, identical to the amino acid sequence of SEQ ID NO:18 and a VL domain having an amino acid sequence that is about 99% identical to the amino acid sequence of SEQ ID NO:19. In another embodiment, the first antigen binding domain comprises a VH domain having an amino acid sequence that is about 100% identical to the amino acid sequence of SEQ ID NO:16 and a VL domain having an amino acid sequence that is about 100% identical to the amino acid sequence of SEQ ID NO:17, and the second antigen binding domain comprises a VH domain having an amino acid sequence that is about 100% identical to the amino acid sequence of SEQ ID NO:18 and a VL domain having an amino acid sequence that is about 100% identical to the amino acid sequence of SEQ ID NO:19.

In some embodiments, the first monomer is about 90% identical to the amino acid sequence of SEQ ID NO:1. In some embodiments, the first monomer is about 95% identical to the amino acid sequence of SEQ ID NO:1. In some embodiments, the first monomer is about 97% identical to the amino acid sequence of SEQ ID NO:1. In some embodiments, the first monomer is about 98% identical to the amino acid sequence of SEQ ID NO:1. In some embodiments, the first monomer is about 99% identical to the amino acid sequence of SEQ ID NO:1. In some embodiments, the first monomer is about 100% identical to the amino acid sequence of SEQ ID NO:1.

In some embodiments, the second monomer is about 90% identical to the amino acid sequence of SEQ ID NO:2. In some embodiments, the second monomer is about 95% identical to the amino acid sequence of SEQ ID NO:2. In some embodiments, the second monomer is about 97% identical to the amino acid sequence of SEQ ID NO:2. In some embodiments, the second monomer is about 98% identical to the amino acid sequence of SEQ ID NO:2. In some embodiments, the second monomer is about 99% identical to the amino acid sequence of SEQ ID NO:2. In some embodiments, the second monomer is about 100% identical to the amino acid sequence of SEQ ID NO:2.

In some embodiments, the third monomer is about 90% identical to the amino acid sequence of SEQ ID NO:3. In some embodiments, the third monomer is about 95% identical to the amino acid sequence of SEQ ID NO:3. In some embodiments, the third monomer is about 97% identical to the amino acid sequence of SEQ ID NO:3. In some embodiments, the third monomer is about 98% identical to the amino acid sequence of SEQ ID NO:3. In some embodiments, the third monomer is about 99% identical to the amino acid sequence of SEQ ID NO:3. In some embodiments, the third monomer is about 100% identical to the amino acid sequence of SEQ ID NO:3.

In some embodiments, the first monomer is about 90% identical to the amino acid sequence of SEQ ID NO:1, and the second monomer is about 90% identical to the amino acid sequence of SEQ ID NO:2. In some embodiments, the first monomer is about 95% identical to the amino acid sequence of SEQ ID NO:1, and the second monomer is about 95% identical to the amino acid sequence of SEQ ID NO:2. In some embodiments, the first monomer is about 97% identical to the amino acid sequence of SEQ ID NO:1, and the second monomer is about 97% identical to the amino acid sequence of SEQ ID NO:2. In some embodiments, the first monomer is about 98% identical to the amino acid sequence of SEQ ID NO:1, and the second monomer is about 98% identical to the amino acid sequence of SEQ ID NO:2. In some embodiments, the first monomer is about 99% identical to the amino acid sequence of SEQ ID NO:1, and the second monomer is about 99% identical to the amino acid sequence of SEQ ID NO:2. In some embodiments, the first monomer is about 100% identical to the amino acid sequence of SEQ ID NO:1, and the second monomer is about 100% identical to the amino acid sequence of SEQ ID NO:2.

In some embodiments, the first monomer is about 90% identical to the amino acid sequence of SEQ ID NO:1, and the third monomer is about 90% identical to the amino acid sequence of SEQ ID NO:3. In some embodiments, the first monomer is about 95% identical to the amino acid sequence of SEQ ID NO:1, and the third monomer is about 95% identical to the amino acid sequence of SEQ ID NO:3. In some embodiments, the first monomer is about 97% identical to the amino acid sequence of SEQ ID NO:1, and the third monomer is about 97% identical to the amino acid sequence of SEQ ID NO:3. In some embodiments, the first monomer is about 98% identical to the amino acid sequence of SEQ ID NO:1, and the third monomer is about 98% identical to the amino acid sequence of SEQ ID NO:3. In some embodiments, the first monomer is about 99% identical to the amino acid sequence of SEQ ID NO:1, and the third monomer is about 99% identical to the amino acid sequence of SEQ ID NO:3. In some embodiments, the first monomer is about 100% identical to the amino acid sequence of SEQ ID NO:1, and the third monomer is about 100% identical to the amino acid sequence of SEQ ID NO:3.

In some embodiments, the second monomer is about 90% identical to the amino acid sequence of SEQ ID NO:2, and the third monomer is about 90% identical to the amino acid sequence of SEQ ID NO:3. In some embodiments, the second monomer is about 95% identical to the amino acid sequence of SEQ ID NO:2, and the third monomer is about 95% identical to the amino acid sequence of SEQ ID NO:3. In some embodiments, the second monomer is about 97% identical to the amino acid sequence of SEQ ID NO:2, and the third monomer is about 97% identical to the amino acid sequence of SEQ ID NO:3. In some embodiments, the second monomer is about 98% identical to the amino acid sequence of SEQ ID NO:2, and the third monomer is about 98% identical to the amino acid sequence of SEQ ID NO:3. In some embodiments, the second monomer is about 99% identical to the amino acid sequence of SEQ ID NO:2, and the third monomer is about 99% identical to the amino acid sequence of SEQ ID NO:3. In some embodiments, the second monomer is about 100% identical to the amino acid sequence of SEQ ID NO:2, and the third monomer is about 100% identical to the amino acid sequence of SEQ ID NO:3.

In some embodiments, the first monomer is about 90% identical to the amino acid sequence of SEQ ID NO:1, the second monomer is about 90% identical to the amino acid sequence of SEQ ID NO:2, and the third monomer is about 90% identical to the amino acid sequence of SEQ ID NO:3. In some embodiments, the first monomer is about 95% identical to the amino acid sequence of SEQ ID NO:1, the second monomer is about 95% identical to the amino acid sequence of SEQ ID NO:2, and the third monomer is about 95% identical to the amino acid sequence of SEQ ID NO:3. In some embodiments, the first monomer is about 97% identical to the amino acid sequence of SEQ ID NO:1, the second monomer is about 97% identical to the amino acid sequence of SEQ ID NO:2, and the third monomer is about 97% identical to the amino acid sequence of SEQ ID NO:3. In some embodiments, the first monomer is about 98% identical to the amino acid sequence of SEQ ID NO:1, the second monomer is about 98% identical to the amino acid sequence of SEQ ID NO:2, and the third monomer is about 98% identical to the amino acid sequence of SEQ ID NO:3. In some embodiments, the first monomer is about 99% identical to the amino acid sequence of SEQ ID NO:1, the second monomer is about 99% identical to the amino acid sequence of SEQ ID NO:2, and the third monomer is about 99% identical to the amino acid sequence of SEQ ID NO:3. In some embodiments, the first monomer is about 100% identical to the amino acid sequence of SEQ ID NO:1, the second monomer is about 100% identical to the amino acid sequence of SEQ ID NO:2, and the third monomer is about 100% identical to the amino acid sequence of SEQ ID NO:3.

As is known in the art, the nucleic acids encoding the components provided herein can be incorporated into expression vectors as is known in the art, and depending on the host cells used to produce an antibody of the disclosure (e.g., XmAb®20717). Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors provided herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer and the nucleic acid encoding a light chain, as applicable depending on the format, are each contained within a single expression vector, generally under different or the same promoter controls. In embodiments of particular use in the methods provided herein, each of these two or three nucleic acids are contained on a different expression vector. That is, in certain embodiments, a first expression vector comprises the nucleic acid encoding the first monomer, a second expression vector comprises the nucleic acid encoding the second monomer, and a third expression vector comprises the nucleic acid encoding the light chain.

XmAb®20717 can be made by culturing host cells comprising the expression vector(s) as is well known in the art. Once produced, traditional antibody purification steps are done, including an ion exchange chromatography step. As discussed in U.S. patent application Ser. No. 15/623,314 and US Publication No. 2018/0118836, hereby incorporated by reference in their entirety and particularly for the discussions concerning purification, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, XmAb®20717 can be administered to human subjects according to a dosage regimen described herein.

### 5.4 Pharmaceutical Compositions and Pharmaceutical Administration

Bispecific anti-CTLA4×anti-PD1 antibody (e.g., XmAb®20717) provided herein can be incorporated into pharmaceutical compositions suitable for administration to a human subject according to a dosage regimen described herein. In some embodiments, XmAb®20717 is incorporated into pharmaceutical compositions suitable for administration to a human subject according to a dosage regimen described herein. As used herein, “dosage regimen” refers to a systematic plan of drug administration regarding formulation, route of administration, drug dose, dosing interval and/or treatment duration. Typically, the pharmaceutical composition comprises a anti-CTLA4×anti-PD1 antibody (e.g., XmAb®20717) and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents (such as a polysorbate), preservatives or buffers (such as an organic acid, which as a citrate or an acetate), which enhance the shelf life or effectiveness of the bispecific anti-CTLA4×anti-PD1 antibodies (e.g., XmAb®20717). Examples of pharmaceutically acceptable carriers include polysorbates (polysorbate-80).

In an exemplary embodiment, the pharmaceutical composition comprises a bispecific anti-CTLA4×anti-PD1 antibody (e.g., XmAb®20717), and a preservative or buffer. In an exemplary embodiment, the pharmaceutical composition comprises a bispecific anti-CTLA4×anti-PD1 antibody (e.g., XmAb®20717), and histidine. In an exemplary embodiment, the pharmaceutical composition comprises a bispecific anti-CTLA4×anti-PD1 antibody (e.g., XmAb®20717), and an acetate. In an exemplary embodiment, the pharmaceutical composition comprises a bispecific anti-CTLA4×anti-PD1 antibody (e.g., XmAb®20717), and sodium acetate. In an exemplary embodiment, the pharmaceutical composition comprises a bispecific anti-CTLA4×anti-PD1 antibody (e.g., XmAb®20717) and a citrate. In an exemplary embodiment, the pharmaceutical composition comprises a bispecific anti-CTLA4×anti-PD1 antibody (e.g., XmAb®20717) and sodium citrate. In one embodiment, the pharmaceutical composition comprises a bispecific anti-CTLA4×anti-PD1 antibody (e.g., XmAb®20717) and a succinate. In one embodiment, the pharmaceutical composition comprises a bispecific anti-CTLA4×anti-PD1 antibody (e.g., XmAb®20717) and sodium succinate.

In an exemplary embodiment, the pharmaceutical composition comprises a bispecific anti-CTLA4×anti-PD1 antibody (e.g., XmAb®20717) and an isotonic agent. In an exemplary embodiment, the pharmaceutical composition comprises a bispecific anti-CTLA4×anti-PD1 antibody (e.g., XmAb®20717) and a polyalcohol. In an exemplary embodiment, the pharmaceutical composition comprises a bispecific anti-CTLA4×anti-PD1 antibody (e.g., XmAb®20717) and mannitol. In an exemplary embodiment, the pharmaceutical composition comprises a bispecific anti-CTLA4×anti-PD1 antibody (e.g., XmAb®20717) and sorbitol. In an exemplary embodiment, the pharmaceutical composition comprises a bispecific anti-CTLA4×anti-PD1 antibody (e.g., XmAb®20717) and sodium chloride. In an exemplary embodiment, the pharmaceutical composition comprises a bispecific anti-CTLA4×anti-PD1 antibody (e.g., XmAb®20717) and potassium chloride.

In an exemplary embodiment, the pharmaceutical composition comprises a bispecific anti-CTLA4×anti-PD1 antibody (e.g., XmAb®20717) and a wetting or emulsifying agent. In an exemplary embodiment, the pharmaceutical composition comprises a bispecific anti-CTLA4×anti-PD1 antibody (e.g., XmAb®20717) and a polysorbate. In an exemplary embodiment, the pharmaceutical composition comprises a bispecific anti-CTLA4×anti-PD1 antibody (e.g., XmAb®20717) and polysorbate-80.

In an exemplary embodiment, the pharmaceutical composition comprises a bispecific anti-CTLA4×anti-PD1 antibody (e.g., XmAb®20717) and an intravenous solution stabilizer. In an exemplary embodiment, the intravenous solution stabilizer comprises a polysorbate and a citrate. In an exemplary embodiment, the pharmaceutical composition comprises a bispecific anti-CTLA4×anti-PD1 antibody (e.g., XmAb®20717) and sodium citrate and polysorbate-80. In one embodiment, the intravenous solution stabilizer comprises a polysorbate and a succinate. In one embodiment, the pharmaceutical composition comprises a bispecific anti-CTLA4×anti-PD1 antibody (e.g., XmAb®20717) and sodium succinate and polysorbate-80.

In an exemplary embodiment, the pharmaceutical composition comprises a bispecific anti-CTLA4×anti-PD1 antibody (e.g., XmAb®20717) and a buffer and an isotonic agent. In an exemplary embodiment, the pharmaceutical composition comprises a bispecific anti-CTLA4×anti-PD1 antibody (e.g., XmAb®20717) and a buffer and sorbitol. In an exemplary embodiment, the pharmaceutical composition comprises a bispecific anti-CTLA4×anti-PD1 antibody (e.g., XmAb®20717) and an acetate and an isotonic agent. In an exemplary embodiment, the pharmaceutical composition comprises a bispecific anti-CTLA4×anti-PD1 antibody (e.g., XmAb®20717) and histidine and an isotonic agent. In an exemplary embodiment, the pharmaceutical composition comprises a bispecific anti-CTLA4×anti-PD1 antibody (e.g., XmAb®20717) and an acetate and sorbitol. In an exemplary embodiment, the pharmaceutical composition comprises a bispecific anti-CTLA4×anti-PD1 antibody (e.g., XmAb®20717) and sodium acetate and sorbitol. In an exemplary embodiment, the pharmaceutical composition comprises a bispecific anti-CTLA4×anti-PD1 antibody (e.g., XmAb®20717) and histidine and sorbitol. In one embodiment, the pharmaceutical composition comprises a bispecific anti-CTLA4×anti-PD1 antibody (e.g., XmAb®20717) and a buffer and sodium chloride. In one embodiment, the pharmaceutical composition comprises a bispecific anti-CTLA4×anti-PD1 antibody (e.g., XmAb®20717) and a succinate and an isotonic agent. In one embodiment, the pharmaceutical composition comprises a bispecific anti-CTLA4×anti-PD1 antibody (e.g., XmAb®20717) and a succinate and sodium chloride. In one embodiment, the pharmaceutical composition comprises a bispecific anti-CTLA4×anti-PD1 antibody (e.g., XmAb®20717) and sodium succinate and sodium chloride.

In an exemplary embodiment, the pharmaceutical composition comprises a bispecific anti-CTLA4×anti-PD1 antibody (e.g., XmAb®20717) and a buffer and an isotonic agent and an intravenous solution stabilizer. In an exemplary embodiment, the pharmaceutical composition comprises a bispecific anti-CTLA4×anti-PD1 antibody (e.g., XmAb®20717) and a buffer and sorbitol and an intravenous solution stabilizer. In an exemplary embodiment, the pharmaceutical composition comprises a bispecific anti-CTLA4×anti-PD1 antibody (e.g., XmAb®20717) and an acetate and an isotonic agent and an intravenous solution stabilizer. In an exemplary embodiment, the pharmaceutical composition comprises a bispecific anti-CTLA4×anti-PD1 antibody (e.g., XmAb®20717) and histidine and an isotonic agent and an intravenous solution stabilizer. In an exemplary embodiment, the pharmaceutical composition comprises a bispecific anti-CTLA4×anti-PD1 antibody (e.g., XmAb®20717) and an acetate and sorbitol and an intravenous solution stabilizer. In an exemplary embodiment, the pharmaceutical composition comprises a bispecific anti-CTLA4×anti-PD1 antibody (e.g., XmAb®20717) and sodium acetate and sorbitol and an intravenous solution stabilizer. In an exemplary embodiment, the pharmaceutical composition comprises a bispecific anti-CTLA4×anti-PD1 antibody (e.g., XmAb®20717) and histidine and sorbitol and an intravenous solution stabilizer.

In an exemplary embodiment, the pharmaceutical composition comprises a bispecific anti-CTLA4×anti-PD1 antibody (e.g., XmAb®20717) and sodium chloride. In an exemplary embodiment, the pharmaceutical composition comprises a bispecific anti-CTLA4×anti-PD1 antibody (e.g., XmAb®20717) and sodium chloride and polysorbate-80. In an exemplary embodiment, the pharmaceutical composition comprises a bispecific anti-CTLA4×anti-PD1 antibody (e.g., XmAb®20717) and sodium citrate and sodium chloride. In an exemplary embodiment, the pharmaceutical composition comprises a bispecific anti-CTLA4×anti-PD1 antibody (e.g., XmAb®20717) and sodium citrate, sodium chloride, and polysorbate-80. In an exemplary embodiment, the pharmaceutical composition comprises a bispecific anti-CTLA4×anti-PD1 antibody (e.g., XmAb®20717) and sodium citrate, sodium chloride, sodium acetate, sorbitol and polysorbate-80. In an exemplary embodiment, the pharmaceutical composition comprises a bispecific anti-CTLA4×anti-PD1 antibody (e.g., XmAb®20717) and sodium citrate, sodium chloride, histidine, sorbitol and polysorbate-80.

In one embodiment, the pharmaceutical composition comprises a bispecific anti-CTLA4×anti-PD1 antibody (e.g., XmAb®20717) and sodium succinate and sucrose. In one embodiment, the pharmaceutical composition comprises a bispecific anti-CTLA4×anti-PD1 antibody (e.g., XmAb®20717) and sodium succinate and polysorbate-80. In one embodiment, the pharmaceutical composition comprises a bispecific anti-CTLA4×anti-PD1 antibody (e.g., XmAb®20717) and sodium succinate and sodium chloride. In one embodiment, the pharmaceutical composition comprises a bispecific anti-CTLA4×anti-PD1 antibody (e.g., XmAb®20717) and sucrose and polysorbate-80. In one embodiment, the pharmaceutical composition comprises a bispecific anti-CTLA4×anti-PD1 antibody (e.g., XmAb®20717) and sucrose and sodium chloride. In one embodiment, the pharmaceutical composition comprises a bispecific anti-CTLA4×anti-PD1 antibody (e.g., XmAb®20717) and sucrose and polysorbate-80. In one embodiment, the pharmaceutical composition comprises a bispecific anti-CTLA4×anti-PD1 antibody (e.g., XmAb®20717) and polysorbate-80 and sodium chloride. In one embodiment, the pharmaceutical composition comprises a bispecific anti-CTLA4×anti-PD1 antibody (e.g., XmAb®20717) and sodium succinate and sucrose and polysorbate-80. In one embodiment, the pharmaceutical composition comprises a bispecific anti-CTLA4×anti-PD1 antibody (e.g., XmAb®20717) and sodium succinate and sucrose and sodium chloride. In one embodiment, the pharmaceutical composition comprises a bispecific anti-CTLA4×anti-PD1 antibody (e.g., XmAb®20717) and sucrose and polysorbate-80 and sodium chloride. In one embodiment, the pharmaceutical composition comprises a bispecific anti-CTLA4×anti-PD1 antibody (e.g., XmAb®20717) and sucrose and polysorbate-80 and sodium chloride and sodium succinate.

The pharmaceutical compositions provided herein may be in a variety of forms.

These include, for example, liquid, semi-solid and solid dosage forms, such as liquid solutions (e.g., injectable and infusible solutions), dispersions or suspensions. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibody is administered by intravenous infusion or injection.

Pharmaceutical compositions typically must be sterile and stable under the conditions of manufacture and storage. Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle that contains a basic dispersion medium and the required other ingredients from those enumerated herein.

In some embodiments, prophylactic premedication can be administered to a subject to reduce infusion related reactions. In some embodiments, acetaminophen (e.g., 650 mg orally), diphenhydramine hydrochloride (e.g., 25-50 mg IV) and/or dexamethasone (e.g., 10 mg IV) is administered to a subject to reduce infusion related reactions. In some embodiments, diphenhydramine hydrochloride (e.g., 50 mg IV) and/or dexamethasone (e.g., 10-20 mg IV) is administered to a subject to reduce infusion related reactions. In some embodiments, epinephrine and/or bronchodilators is administered to a subject to reduce infusion related reactions. In some embodiments, epinephrine, antihistamines, corticosteroids, IV fluids, vasopressors, oxygen, bronchodilators, and/or acetaminophen is administered to a subject to reduce infusion related reactions. In some embodiments, one or more medications to reduce infusion related reactions is administered to a subject prior to the administration of a bispecific anti-CTLA4×anti-PD1 antibody (e.g., XmAb®20717) to the subject. In some embodiments, one or more medications to reduce infusion related reactions is administered to a subject prior to but on the same day as the administration of a bispecific anti-CTLA4×anti-PD1 antibody (e.g., XmAb®20717) to the subject. In some embodiments, one or more medications to reduce infusion related reactions is administered to a subject after the administration of a bispecific anti-CTLA4×anti-PD1 antibody (e.g., XmAb®20717) to the subject.

The anti-CTLA4×anti-PD1 antibody (e.g., XmAb®20717) can be administered by a variety of methods known in the art. In an exemplary embodiment, the route/mode of administration is intravenous injection. As will be appreciated by the skilled artisan, the route and/or mode of administration will vary depending upon the desired results.

In some embodiments, the bispecific anti-CTLA4×anti-PD1 antibody (e.g., XmAb®20717) is a front line therapy, second line therapy, third line therapy, fourth line therapy, fifth line therapy, or sixth line therapy.

A medical professional can readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

### 5.5 Combination Therapy

In some embodiments, the bispecific anti-CTLA4×anti-PD1 antibody (e.g., XmAb®20717) is administered with Carboplatin (e.g., prior to, after, or at the same time or same day). In some embodiments, the methods of the present disclosure comprises administration of Carboplatin to a subject. In some embodiments, a pharmaceutical composition of the disclosure comprises Carboplatin. Carboplatin can be obtained through purchase from companies such as Sagent Pharma, Meitheal Pharmaceuticals, AdooQ Bioscience or Accord BioPharma (as Paraplatin®). Carboplatin is available in 10 mg/mL solutions. Calvert's formula can be utilized to calculate the dose of carboplatin.

In some embodiments, the bispecific anti-CTLA4×anti-PD1 antibody (e.g., XmAb®20717) is administered with Cabazitaxel (e.g., prior to, after, or at the same time or same day). In some embodiments, the methods of the present disclosure comprises administration of Cabazitaxel to a subject. In some embodiments, a pharmaceutical composition of the disclosure comprises Cabazitaxel. Cabazitaxel (Jevtana®, Sanofi-Aventis) can be obtained through purchase in 60 mg/1.5 mL solutions.

In some embodiments, the bispecific anti-CTLA4×anti-PD1 antibody (e.g., XmAb®20717) is administered with Docetaxel (e.g., prior to, after, or at the same time or same day). In some embodiments, the methods of the present disclosure comprises administration of Docetaxel to a subject. In some embodiments, a pharmaceutical composition of the disclosure comprises Docetaxel. Docetaxel (Taxotere®, Sanofi-Aventis) can be obtained through purchase in 20 mg/mL and 80 mg/4 mL solutions.

In some embodiments, the bispecific anti-CTLA4×anti-PD1 antibody (e.g., XmAb®20717) is administered with Prednisone (e.g., prior to, after, or at the same time or same day). In some embodiments, the methods of the present disclosure comprises administration of Prednisone to a subject. In some embodiments, a pharmaceutical composition of the disclosure comprises Prednisone. Prednisone can be obtained through purchase from companies such as Roxane Laboratories in 5 mg/mL solutions.

In some embodiments, the bispecific anti-CTLA4×anti-PD1 antibody (e.g., XmAb®20717) is administered with Olaparib (e.g., prior to, after, or at the same time or same day). In some embodiments, the methods of the present disclosure comprises administration of Olaparib to a subject. In some embodiments, a pharmaceutical composition of the disclosure comprises Olaparib. Olaparib (LynparzaR, AstraZeneca) can be obtained through purchase in 100 mg or 150 mg tablets. In an exemplary embodiment, a 300 mg oral dosage of olaparib is provided by the administration of two 150 mg tablets to the male human subject. In an exemplary embodiment, a 300 mg oral dosage of olaparib is provided by the administration of three 100 mg tablets to the male human subject. In an exemplary embodiment, a twice daily 300 mg oral dosage of olaparib is provided by the administration of two administrations per day of two 150 mg tablets to the male human subject, or two administrations per day of three 100 mg tablets to the male human subject or a combination thereof.

In specific embodiments the bispecific anti-CTLA4×anti-PD1 antibody (e.g., XmAb®20717) is the only therapeutic antibody administered to the subject during the treatment methods provided herein.

In some embodiments, a method of the disclosure comprises administering the bispecific anti-CTLA4×anti-PD1 antibody (e.g., XmAb®20717) to a subject as a monotherapy. As used herein, the term “monotherapy” refers to the use of a single agent (e.g., the bispecific anti-CTLA4×anti-PD1 antibody (e.g., XmAb®20717)), without a second active agent, to treat the same indication, e.g., the same solid cancerous tumor. In some embodiments, the term “monotherapy” does not exclude one or more additional agent(s) from being administered to a subject if the one or more additional agent(s) is/are not administered for treating the same solid cancerous tumor in the subject. For example, in some embodiments, the term “monotherapy” does not exclude one or more additional agent(s) used to prevent or ameliorate injection site side effects, fever, and/or any other side effect associated with the bispecific anti-CTLA4×anti-PD1 antibody (e.g., XmAb®20717) of the disclosure from being administered to a subject. In some embodiments, the term “monotherapy” does not exclude treatment with one or more additional agent(s) used for treating or ameliorating another disease or disorder in the subject (e.g., a disease or disorder that is not the solid cancerous tumor that the bispecific anti-CTLA4×anti-PD1 antibody (e.g., XmAb®20717) of the disclosure is being used to treat). In some embodiments, the another disease or disorder in the subject is another cancer that is not a solid cancerous tumor of the disclosure. In some embodiments, the another disease or disorder in the subject is another cancer that is not the solid cancerous tumor that the bispecific anti-CTLA4×anti-PD1 antibody (e.g., XmAb®20717) of the disclosure is being used to treat. In some embodiments, the term “monotherapy” does not exclude previous treatment with another agent that was used for treating or attempting to treat the solid cancerous tumor in a subject (e.g., a subject undergoing monotherapy treatment with the bispecific anti-CTLA4×anti-PD1 antibody (e.g., XmAb®20717) of the disclosure can be a subject that was previously treated with another agent for the solid cancerous tumor; e.g., the XmAb®20717 treatment is a second line of therapy). In some embodiments, a subject receiving monotherapy treatment with the bispecific anti-CTLA4×anti-PD1 antibody (e.g., XmAb®20717) of the disclosure is a subject with a solid cancerous tumor that is resistant to agents that were previously used for treating the solid cancerous tumor. In some embodiments, the term “monotherapy” does not exclude previous treatment with another agent prior to the first dose of the bispecific anti-CTLA4×anti-PD1 antibody (e.g., XmAb®20717) of the disclosure. In some embodiments, the term “monotherapy” does not exclude premedication (e.g., premedication with antihistamine, acetaminophen, hypertension agents, steroids, and the like).

However, in certain instances, a bispecific anti-CTLA4×anti-PD1 antibody (e.g., XmAb®20717) described herein can be used in combination with another therapeutic agent (e.g., Carboplatin, Cabazitaxel, Docetaxel, Prednisone, Olaparib, or any other appropriate therapeutic agent). Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In certain embodiments, the two or more treatments are delivered during the maintenance period following administration of the bispecific anti-CTLA4×anti-PD1 antibody and after the disorder has been cured or eliminated. In some embodiments, combination with another therapeutic agent refers to another therapeutic agent that is known to treat or is used to treat the same disease or disorder that the bispecific anti-CTLA4×anti-PD1 antibody (e.g., XmAb®20717) of the disclosure is being used to treat in the subject. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces one or more symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The bispecific anti-CTLA4×anti-PD1 antibody (e.g., XmAb®20717) and at least one additional therapeutic agent can be administered simultaneously, in the same or in separate compositions, or sequentially. For sequential administration, the bispecific anti-CTLA4×anti-PD1 antibody (e.g., XmAb®20717) described herein can be administered first, and the additional agent can be administered second, or vice versa.

The bispecific anti-CTLA4×anti-PD1 antibody (e.g., XmAb®20717) and/or one or more additional therapeutic agents, procedures or modalities can be administered during periods of active disorder, or during a period of positive therapeutic response or less active disease. The bispecific anti-CTLA4×anti-PD1 antibody (e.g., XmAb®20717) can be administered before the other treatment, concurrently with the treatment, post-treatment, or during a positive therapeutic response to the disorder.

When administered in combination, the bispecific anti-CTLA4×anti-PD1 antibody (e.g., XmAb®20717) and the one or more additional agents (e.g., second or third agent) can be administered in an amount or dose that is higher, lower or the same than the amount or dosage of each agent used individually, e.g., as a monotherapy. In some embodiments, the administered amount or dosage of the bispecific anti-CTLA4×anti-PD1 antibody (e.g., XmAb®20717) and the one or more additional agents (e.g., second or third agent), is lower (e.g., at least about 10%, at least about 20%, at least about 30%, at least about 40%, or at least about 50%) than the amount or dosage of each agent used individually, e.g., as a monotherapy. In other embodiments, the amount or dosage of the bispecific anti-CTLA4×anti-PD1 antibody (e.g., XmAb®20717) and the one or more additional agents (e.g., second or third agent, that results in a desired effect (e.g., treatment of cancer) is lower (e.g., at least about 10%, at least about 20%, at least about 30%, at least about 40%, or at least about 50%) than the amount or dosage of each agent used individually, e.g., as a monotherapy, required to achieve the same therapeutic effect.

5.5.1. Combination Therapy: Side-Effect Ameliorating Agent

In some embodiments, a bispecific antibody is administered to a human subject in combination with one or more side-effect ameliorating agent(s). In an exemplary embodiment, the one or more side-effect ameliorating agent(s) is administered prior to the first administration of the bispecific antibody. In an exemplary embodiment, the one or more side-effect ameliorating agent(s) is administered prior to each administration of the bispecific antibody.

Possible side effects include, but are not limited to, increased amounts of alanine transaminase (ALT) in the blood, increased amounts of aspartate transaminase (AST) in the blood, fever, vomiting, nausea, diarrhea, hypotension, hypoxia, rash, dysphagia, gastroparesis, capillary leak syndrome, hypophosphatemia, anemia, fatigue, and increased lipase in the blood. Symptoms of treatment with the bispecific anti-CTLA4×anti-PD1 antibody (e.g., XmAb®20717) of the disclosure can include high fevers, nausea, transient hypotension, hypoxia, and the like. Symptoms can also include clinical constitutional signs and symptoms such as fever, fatigue, anorexia, myalgias, arthralgias, nausea, vomiting, headache, clinical skin signs, and rash. Symptoms can include clinical gastrointestinal signs and symptoms such as nausea, vomiting and diarrhea. Symptoms can include clinical respiratory signs and symptoms such as tachypnea and hypoxemia. Symptoms can include clinical cardiovascular signs and symptoms such as tachycardia, widened pulse pressure, hypotension, increased cardiac output (early) and potentially diminished cardiac output. Symptoms can include clinical coagulation signs and symptoms such as elevated d-dimer, hypofibrinogenemia with or without bleeding. Symptoms can include clinical renal signs and symptoms such as azotemia. Symptoms can include clinical hepatic signs and symptoms such as transaminitis and hyperbilirubinemia. Symptoms can include clinical neurologic signs and symptoms such as headache, mental status changes, confusion, delirium, word finding difficulty or frank aphasia, hallucinations, tremor, dysmetria, altered gait, and seizures.

In an exemplary embodiment, the administration of XmAb®20717 described herein to the human subject produces a low rate of one or more symptoms described herein. In an exemplary embodiment, the administration of XmAb®20717 described herein to the human subject produces a low level of one or more symptoms described herein. In an exemplary embodiment, the administration of XmAb®20717 described herein to the human subject produces a low Grade (such as Grade 1 or Grade 2) of one or more symptoms described herein. In an exemplary embodiment, the administration of XmAb®20717 described herein to the human subject produces a low Grade (such as Grade 1 or Grade 2) of at least one symptom.

In one embodiment, the one or more side-effect ameliorating agent(s) include steroids, antihistamines, anti-allergic agents, antinausea agents (or anti-emetics), analgesic agents, antipyretic agents, cytoprotective agents, vasopressor agents, anticonvulsant agents, anti-inflammatories, or any combination thereof. In some embodiments, epinephrine, antihistamines, corticosteroids, IV fluids, vasopressors, oxygen, bronchodilators, and/or acetaminophen is administered to a subject. In some embodiments, at least one of epinephrine, antihistamines, corticosteroids, IV fluids, vasopressors, oxygen, bronchodilators, and/or acetaminophen is administered to a subject prior to administration of the bispecific anti-CTLA4×anti-PD1 antibody (e.g., XmAb®20717) to the subject. In some embodiments, at least one of epinephrine, antihistamines, corticosteroids, IV fluids, vasopressors, oxygen, bronchodilators, and/or acetaminophen is administered to a subject after administration of the bispecific anti-CTLA4×anti-PD1 antibody (e.g., XmAb®20717) to the subject. In some embodiments, at least one of epinephrine, antihistamines, corticosteroids, IV fluids, vasopressors, oxygen, bronchodilators, and/or acetaminophen is administered to a subject on the same day as the administration of the bispecific anti-CTLA4×anti-PD1 antibody (e.g., XmAb®20717) to the subject.

5.5.2. Combination Therapy: Side-Effect Ameliorating Agent, Steroid

In one embodiment, the side-effect ameliorating agent is a steroid. In one embodiment, the steroid is a corticosteroid. In one embodiment, the corticosteroid is a glucocorticoid. In one embodiment, the corticosteroid is betamethasone, dexamethasone, prednisone, prednisolone, methylprednisolone (e.g., 0.5-1 mg/kg/day or 1-2 mg/kg/day), triamcinolone, or any combination thereof. In one embodiment, the corticosteroid is hydrocortisone, cortisone, betamethasone, or any combination thereof. In one embodiment, the steroid is fludrocortisone. In one embodiment, the steroid is dexamethasone.

5.5.3. Combination Therapy: Side-Effect Ameliorating Agent, Antihistamine

In one embodiment, the side-effect ameliorating agent is an antihistamine. In one embodiment, the antihistamine is an H₁ antagonist. In one embodiment, the H₁ antagonist is acrivastine, azelastine, bilastine, bromodiphenhydramine, brompheniramine, buclizine, carbinoxamine, cetirizine (Zyrtec®), chlorodiphenhydramine, chlorphenamine, clemastine, cyclizine, cyproheptadine, dexbrompheniramine, dexchlorpheniramine, dimenhydrinate, dimetindene, diphenhydramine, doxylamine, ebastine, embramine, fexofenadine (Allegra®), hydroxyzine (Vistarin, loratadine (Claritin®), meclizine, mirtazapine, olopatadine, orphenadrine, phenindamine, pheniramine, phenyltoloxamine, promethazine, quetiapine (Seroquel®), rupatadine (Alergoliber®), tripelennamine, triprolidine, or any combination thereof.

In one embodiment, the antihistamine is acrivastine. In one embodiment, the antihistamine is cetirizine. In one embodiment, the antihistamine is diphenhydramine. In one embodiment, the antihistamine is Benadryl®.

In one embodiment, the antihistamine is an H₁ inverse agonist. In one embodiment, the H₁ inverse agonist is acrivastine, cetirizine, levocetirizine, desloratadine, pyrilamine, or any combination thereof.

In one embodiment, the antihistamine is an H₂ antihistamine. In one embodiment, the H₂ antihistamine is an H₂ antagonist. In one embodiment, the H₂ antihistamine is an H₂ inverse agonist. In one embodiment, the H₂ antihistamine is cimetidine, famotidine, lafutidine, nizatidine, ranitidine, roxatidine, tiotidine, or any combination thereof.

5.5.4. Combination Therapy: Side-Effect Ameliorating Agent, Anti-Allergy Agent

In one embodiment, the side-effect ameliorating agent is an antiallergy agent. In one embodiment, the side-effect ameliorating agent is antihistamines, glucocorticoids, epinephrine (adrenaline), mast cell stabilizers, antileukotriene agents, anti-cholinergics, decongestants, or any combination thereof. In one embodiment, the side-effect ameliorating agent is a decongestant. In one embodiment, the side-effect ameliorating agent is an adrenaline releasing agent. In one embodiment, the side-effect ameliorating agent is levomethamphetamine, phenylpropanolamine, propylhexedrine (Benzedrex®), loratadine, or any combination thereof. In one embodiment, the side-effect ameliorating agent is an α-adrenergic receptor agonist. In one embodiment, the side-effect ameliorating agent is naphazoline, oxymetazoline, phenylephrine, synephrine, tetryzoline, tramazoline, xylometazoline, or any combination thereof.

5.5.5. Combination Therapy: Side-Effect Ameliorating Agent, Antinausea Agents (or Anti-Emetic)

In one embodiment, the side-effect ameliorating agent is an antinausea agent. In one embodiment, the side-effect ameliorating agent is an antiemetic agent. In one embodiment, the side-effect ameliorating agent is a 5-HT₃ receptor antagonist. In one embodiment, the side-effect ameliorating agent is a dolasetron (Anzeme®), granisetron (Kytril®, Sancus®), ondansetron (Zofran), tropisetron (Setrovel®, Navoba®), palonosetron (Aloxi®), mirtazapine (Remeron®), or any combination thereof. In one embodiment, the side-effect ameliorating agent is a dopamine antagonist. In one embodiment, the side-effect ameliorating agent is a 5-HT₃ receptor antagonist. In one embodiment, the side-effect ameliorating agent is domperidone (Motilium®), olanzapine (Zyprexa®), droperidol, haloperidol, chlorpromazine, prochlorperazine, alizapride, prochlorperazine (Compazine®, Stemzine®, Buccastem®, Stemetil®, Phenotil®), metoclopramide (Reglan), or any combination thereof. In one embodiment, the side-effect ameliorating agent is a NK1 receptor antagonist. In one embodiment, the side-effect ameliorating agent is aprepitant or fosaprepitant (Emend®), casopitant, rolapitant (Varubi®), or any combination thereof. In one embodiment, the side-effect ameliorating agent is an anticholinergic. In one embodiment, the side-effect ameliorating agent is scopolamine.

5.5.6. Combination Therapy: Side-Effect Ameliorating Agent, Analgesic and/or Antipyretic Agent

In one embodiment, the side-effect ameliorating agent is an analgesic agent. In one embodiment, the side-effect ameliorating agent is an antipyretic agent. In one embodiment, the side-effect ameliorating agent is a salicylate, any derivative thereof, or any combination thereof. In one embodiment, the salicylate is selected from the group consisting of aspirin, diflunisal, salsalate, salicylic acid, any derivative thereof, or any combination thereof. In one embodiment, the salicylate is choline salicylate, magnesium salicylate, sodium salicylate, or any combination thereof. In one embodiment, the side-effect ameliorating agent is aspirin. In one embodiment, the side-effect ameliorating agent is acetaminophen, any derivative thereof. In one embodiment, the side-effect ameliorating agent is an NSAID, any derivative thereof. In one embodiment, the NSAID is a propionic acid derivative. In one embodiment, the NSAID is ibuprofen, dexibuprofen, naproxen, fenoprofen, ketoprofen, dexketoprofen, flurbiprofen, oxaprozin, loxoprofen, any derivative thereof, or any combination thereof. In one embodiment, the NSAID is ibuprofen. In one embodiment, the NSAID is naproxen. In one embodiment, the NSAID is an acetic acid derivative. In one embodiment, the NSAID is indomethacin, tolmetin, sulindac, etodolac, ketorolac, diclofenac, aceclofenac, nabumetone, any derivative thereof, or any combination thereof. In one embodiment, the NSAID is an enolic acid derivative. In one embodiment, the NSAID is piroxicam, meloxicam, tenoxicam, droxicam, lornoxicam, phenylbutazone, any derivative thereof, or any combination thereof. In one embodiment, the NSAID is an anthranilic acid derivative. In one embodiment, the NSAID is mefenamic acid, meclofenamic acid, flufenamic acid, tolfenamic acid, any derivative thereof, or any combination thereof. In one embodiment, the side-effect ameliorating agent is phenazone, metamizole, nabumetone, any derivative thereof, or any combination thereof. In one embodiment, the side-effect ameliorating agent is an opiate. In one embodiment, the side-effect ameliorating agent is codeine, morphine, thebaine, fentanyl, or any combination thereof. In one embodiment, the side-effect ameliorating agent is dihydrocodeine, oxymorphol, oxycodone, oxymorphone, metopon, or any combination thereof.

5.5.7. Combination Therapy: Side-Effect Ameliorating Agent, Vasopressor Agent

In one embodiment, the side-effect ameliorating agent is a vasopressor agent. In one embodiment, the vasopressor agent is norepinephrine, phenylephrine, epinephrine, ephedrine, dopamine, vasopressin, or any combination thereof. In one embodiment, the vasopressor agent is dobutamine, midodrine, amezinium, or any combination thereof.

5.5.8. Combination Therapy: Side-Effect Ameliorating Agent, Anticonvulsant agent

In one embodiment, the side-effect ameliorating agent is an anticonvulsant agent. In one embodiment, the anticonvulsant is an aldehyde. In one embodiment, the aldehyde is paraldehyde. In one embodiment, the anticonvulsant is an aromatic allylic alcohol. In one embodiment, the aromatic allylic alcohol is stiripentol. In one embodiment, the anticonvulsant is a barbiturate. In one embodiment, the barbiturate is phenobarbital, primidone, methylphenobarbital, barbexaclone, or any combination thereof. In one embodiment, the anticonvulsant is a benzodiazepine. In one embodiment, the benzodiazepine is clobazam, clonazepam, clorazepate, diazepam, midazolam, lorazepam, nitrazepam, temazepam, nimetazepam, or any combination thereof. In one embodiment, the anticonvulsant is a carboxamide. In one embodiment, the carboxamide is carbamazepine, oxcarbazepine, eslicarbazepine acetate or any combination thereof. In one embodiment, the anticonvulsant is a fatty acid. In one embodiment, the fatty acid is a valproate. In one embodiment, the valproate is valproic acid, sodium valproate, divalproex sodium, or any combination thereof. In one embodiment, the valproate is vigabatrin, progabide, and tiagabine. In one embodiment, the anticonvulsant is a fructose derivative. In one embodiment, the fructose derivative is topiramate. In one embodiment, the anticonvulsant is a GABA analog. In one embodiment, the GABA analog is gabapentin, pregabalin, or any combination thereof. In one embodiment, the anticonvulsant is a hydantoin. In one embodiment, the hydantoin is ethotoin, phenytoin, mephenytoin, fosphenytoin, or any combination thereof. In one embodiment, the anticonvulsant is an oxazolidinedione. In one embodiment, the oxazolidinedione is paramethadione, trimethadione, ethadione, or any combination thereof. In one embodiment, the anticonvulsant is a propionate. In one embodiment, the anticonvulsant is a pyrimidinedione. In one embodiment, the anticonvulsant is a pyrrolidine. In one embodiment, the pyrrolidine is brivaracetam, etiracetam, levetiracetam, seletracetam, or any combination thereof. In one embodiment, the anticonvulsant is levetiracetam. In one embodiment, the anticonvulsant is a succinimide. In one embodiment, the succinimide is ethosuximide, phensuximide, mesuximide, or any combination thereof. In one embodiment, the anticonvulsant is a sulfonamide. In one embodiment, the succinimide is acetazolamide, sultiame, methazolamide, zonisamide, or any combination thereof. In one embodiment, the anticonvulsant is a triazine. In one embodiment, the triazine is lamotrigine. In one embodiment, the anticonvulsant is a urea. In one embodiment, the urea is pheneturide, phenacemide, or any combination thereof. In one embodiment, the anticonvulsant is a valproylamide. In one embodiment, the anticonvulsant is a valproylamide. In one embodiment, the valproylamide is valpromide, valnoctamide, or any combination thereof. In one embodiment, the anticonvulsant is perampanel, stiripentol, pyridoxine, or any combination thereof.

5.5.9. Combination Therapy: Side-Effect Ameliorating Agent, TNFα Inhibitor

In one embodiment, the side-effect ameliorating agent is an anti-inflammatory agent. In one embodiment, the side-effect ameliorating agent is a TNF-α inhibitor. In one embodiment, the TNF-α inhibitor is an antibody. Examples of an anti-TNFα antibody molecule such as, infliximab (Remicade®) (e.g., 5 mg/kg), adalimumab (Humira®), certolizumab pegol (Cimzia), golimumab (Simponi®), or any combination thereof. Another example of a TNFα inhibitor is a fusion protein such as entanercept (Enbrel®). In one embodiment, the TNF-α inhibitor is a small molecule. Small molecule inhibitor of TNFα include, but are not limited to, xanthine derivatives (e.g., pentoxifylline), bupropion, or any combination thereof.

5.5.10. Combination Therapy: Side-Effect Ameliorating Agent, IL6 Inhibitor

In one embodiment, the side-effect ameliorating agent is an anti-inflammatory agent. In one embodiment, the side-effect ameliorating agent is a IL-6 inhibitor. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, FM101, or any combination thereof. In one embodiment, the anti-IL-6 antibody molecule is tocilizumab.

The methods described herein can comprise administering a bispecific antibody described herein to a human subject and further administering one or more agents to manage elevated levels of a soluble factor resulting from treatment with a bispecific antibody. In one embodiment, the soluble factor elevated in the human subject is one or more of IFN-γ, TNFα, IL-2 and IL-6. In an embodiment, the factor elevated in the human subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. Examples include anakinra or rilonacept or canakinumab.

In one embodiment, the side-effect ameliorating agent is one that reduces an immune-mediated side effect. Exemplary immune-mediated side effects include, but are not limited to pneumonitis, colitis, hepatitis, nephritis and renal dysfunction, hypothyroidism, hyperthyroidism, and endocrinopathies (e.g., hypophysitis, Type 1 diabetes mellitus and thyroid disorders such as hypothyroidism and hyperthyroidism). In one embodiment, the side-effect ameliorating agent reduces embryofetal toxicity.

In an embodiment, the human subject can be administered an antipyretic agent. In an embodiment, the human subject can be administered an analgesic agent.

5.5.11. Side-Effect Combinations and Amounts

In one embodiment, a steroid is administered prior to the bispecific antibody. In one embodiment, the steroid is administered in an amount between about 5 mg and about 30 mg. In one embodiment, the steroid described herein is administered in an amount between about 5 mg and about 25 mg. In one embodiment, the steroid is administered in an amount between about 5 mg and about 15 mg. In one embodiment, the steroid is administered in an amount between about 8 mg and about 12 mg. In one embodiment, the steroid is administered in an amount between about 10 mg and about 20 mg. In one embodiment, the steroid is administered in an amount of about 10 mg. In one embodiment, the steroid is administered in an amount of 10 mg. In one embodiment, the steroid is administered in an amount between about 18 mg and about 22 mg. In one embodiment, the steroid is administered in an amount of about 20 mg. In one embodiment, the steroid is administered in an amount of 20 mg. In one embodiment, the steroid is dexamethasone. In one embodiment, the steroid is dexamethasone and is administered in an amount between about 10 mg and about 20 mg. In one embodiment, the steroid is dexamethasone and is administered in an amount of about 10 mg. In one embodiment, the steroid is dexamethasone. In one embodiment, the steroid is dexamethasone and is administered in an amount of about 20 mg. In one embodiment, the steroid is dexamethasone and is administered between about 45 minutes and 75 minutes before each administration of a bispecific anti-CTLA4×anti-PD1 antibody (e.g., XmAb®20717). In one embodiment, the steroid is dexamethasone and is administered about 60 minutes before each administration of a bispecific anti-CTLA4×anti-PD1 antibody (e.g., XmAb®20717). In one embodiment, the steroid is dexamethasone and is administered about 60 minutes before an administration of bispecific anti-CTLA4×anti-PD1 antibody (e.g., XmAb®20717). In one embodiment, about 20 mg of dexamethasone is administered about 60 minutes before each administration of a bispecific anti-CTLA4×anti-PD1 antibody (e.g., XmAb®20717). In one embodiment, about 20 mg of dexamethasone is administered about 60 minutes before an administration of a bispecific anti-CTLA4×anti-PD1 antibody (e.g., XmAb®20717).

In one embodiment, an antihistamine is administered prior to the bispecific antibody. In one embodiment, the antihistamine is an H₁ antagonist. In one embodiment, the H₁ antagonist is a first generation H₁ antagonist. In one embodiment, the antihistamine is an ethanolamine. In one embodiment, the ethanolamine is diphenhydramine, carbinoxamine, doxylamine, orphenadrine, bromazine, clemastine, dimenhydrinate, or any combination thereof. In one embodiment, the antihistamine is diphenhydramine. In one embodiment, the antihistamine is administered in an amount between about 20 mg and 60 mg. In one embodiment, the antihistamine is administered in an amount between about 20 mg and 30 mg. In one embodiment, the antihistamine is administered in an amount of about 25 mg. In one embodiment, the antihistamine is administered in an amount of 25 mg. In one embodiment, the antihistamine is administered in an amount between about 40 mg and 60 mg. In one embodiment, the antihistamine is administered in an amount between about 45 mg and 55 mg. In one embodiment, the antihistamine is administered in an amount of about 50 mg. In one embodiment, the antihistamine is administered in an amount of 50 mg. In one embodiment, the antihistamine is diphenhydramine and the amount between about 20 mg and about 30 mg. In one embodiment, the antihistamine is diphenhydramine and the amount is about 25 mg. In one embodiment, the antihistamine is diphenhydramine and is administered between about 20 minutes and 70 minutes before each administration of a bispecific anti-CTLA4×anti-PD1 antibody (e.g., XmAb®20717). In one embodiment, the antihistamine is diphenhydramine and is administered between about 30 minutes and 60 minutes before each administration of a bispecific anti-CTLA4×anti-PD1 antibody (e.g., XmAb®20717). In one embodiment, the antihistamine is diphenhydramine and is administered between about 30 minutes and 60 minutes before an administration of a bispecific anti-CTLA4×anti-PD1 antibody (e.g., XmAb®20717). In one embodiment, about 25 mg of diphenhydramine is administered between about 30 minutes and 60 minutes before each administration of a bispecific anti-CTLA4×anti-PD1 antibody (e.g., XmAb®20717). In one embodiment, about 25 mg of diphenhydramine is administered between about 30 minutes and 60 minutes before an administration of a bispecific anti-CTLA4×anti-PD1 antibody (e.g., XmAb®20717).

In one embodiment, acetaminophen is administered prior to the bispecific antibody. In one embodiment, acetaminophen is administered in an amount between about 100 mg and 1000 mg. In one embodiment, acetaminophen is administered in an amount between about 400 mg and 600 mg. In one embodiment, acetaminophen is administered in an amount of about 500 mg. In one embodiment, acetaminophen is administered in an amount of 500 mg. In one embodiment, acetaminophen is administered in an amount between about 500 mg and 800 mg. In one embodiment, acetaminophen is administered in an amount between about 550 mg and 750 mg. In one embodiment, acetaminophen is administered in an amount between about 600 mg and 700 mg. In one embodiment, acetaminophen is administered in an amount of about 650 mg. In one embodiment, acetaminophen is administered in an amount of 650 mg. In one embodiment, the acetaminophen is administered between about 15 minutes and about 45 minutes before each administration of a bispecific anti-CTLA4×anti-PD1 antibody (e.g., XmAb®20717). In one embodiment, the acetaminophen is administered about 30 minutes before each administration of a bispecific anti-CTLA4×anti-PD1 antibody (e.g., XmAb®20717). In one embodiment, the acetaminophen is administered between about 60 minutes and about 30 minutes before an administration of a bispecific anti-CTLA4×anti-PD1 antibody (e.g., XmAb®20717). In one embodiment, the acetaminophen is administered between about 60 minutes and about 30 minutes before each administration of a bispecific anti-CTLA4×anti-PD1 antibody (e.g., XmAb®20717). In one embodiment, about 650 mg of acetaminophen is administered about 30 minutes before each administration of a bispecific anti-CTLA4×anti-PD1 antibody (e.g., XmAb®20717).

In one embodiment, a steroid, an H₁ antagonist, and acetaminophen are administered prior to the bispecific anti-CTLA4×anti-PD1 antibody (e.g., XmAb®20717). In one embodiment, dexamethasone, an H₁ antagonist, and acetaminophen are administered prior to the bispecific anti-CTLA4×anti-PD1 antibody (e.g., XmAb®20717). In one embodiment, a steroid, diphenhydramine, and acetaminophen are administered prior to the bispecific anti-CTLA4×anti-PD1 antibody (e.g., XmAb®20717). In one embodiment, dexamethasone, diphenhydramine, and acetaminophen are administered prior to the bispecific anti-CTLA4×anti-PD1 antibody (e.g., XmAb®20717). In one embodiment, dexamethasone is administered in an amount of about 10 mg or about 20 mg, diphenhydramine is administered in an amount of about 25 mg, and acetaminophen is administered in an amount of about 650 mg prior to the bispecific anti-CTLA4×anti-PD1 antibody (e.g., XmAb®20717). In an exemplary embodiment, if the human subject is not experiencing a symptom after 1 or 2 or 3 or 4 consecutive doses of the bispecific anti-CTLA4×anti-PD1 antibody (e.g., XmAb®20717), then administration of a side-effect ameliorating agent described herein does not occur.

In one embodiment, an antinausea agent is administered prior to the bispecific antibody. In one embodiment, the antinausea agent is a 5-HT₃ receptor antagonist. In one embodiment, the 5-HT₃ receptor antagonist is administered in an amount between about 5 mg and 30 mg. In one embodiment, the 5-HT₃ receptor antagonist is administered in an amount between about 5 mg and 15 mg. In one embodiment, the 5-HT₃ receptor antagonist is administered in an amount between about 5 mg and 10 mg. In one embodiment, the 5-HT₃ receptor antagonist is administered in an amount of about 8 mg. In one embodiment, the 5-HT₃ receptor antagonist is administered in an amount of 8 mg. In one embodiment, the 5-HT₃ receptor antagonist is ondansetron.

In one embodiment, an NK1 receptor antagonist is administered prior to the bispecific antibody. In one embodiment, the NK1 receptor antagonist is administered in an amount between about 100 mg and 300 mg. In one embodiment, the NK1 receptor antagonist is administered in an amount between about 125 mg and 200 mg. In one embodiment, the NK1 receptor antagonist is administered in an amount between about 125 mg and 175 mg. In one embodiment, the NK1 receptor antagonist is administered in an amount of about 150 mg. In one embodiment, the NK1 receptor antagonist is administered in an amount of 150 mg. In one embodiment, the NK1 receptor antagonist is aprepitant, fosaprepitant, or combination thereof. In one embodiment, the NK1 receptor antagonist is fosaprepitant dimeglumine.

### 5.6 Methods of Treatment

In an exemplary embodiment, provided are methods for treating CTLA4 expressing cancers. In an exemplary embodiment, provided are methods for treating PD1 expressing cancers. In an exemplary embodiment, provided are methods for treating CTLA4 and/or PD1 expressing cancers. In an exemplary embodiment, provided are methods for treating solid cancerous tumors. The compositions of the disclosure can be used to treat certain solid cancerous tumors, for example, as described herein. In some embodiments, a composition of the disclosure is administered according to a method of the disclosure to treat a solid cancerous tumor. In some embodiments, the solid cancerous tumor is receptive to treatment by an antibody which binds to PD1. In some embodiments, the solid cancerous tumor is receptive to treatment by an antibody which binds to CTLA4. In some embodiments, the solid cancerous tumor is receptive to treatment by an antibody which binds to PD1 and CTLA4. In some embodiments, the cancer or the solid cancerous tumor is refractory. In some embodiments, the cancer or the solid cancerous tumor is relapsed. In some embodiments, the cancer or the solid cancerous tumor is refractory and relapsed.

In an exemplary embodiment, the compositions provided herein are used to treat a gynecologic or genitourinary cancer. In some embodiments, the cancer or cancerous solid tumor is an ovarian cancer, a clear cell cancer, an endometrial cancer, a cervical cancer, or a prostate cancer. In some embodiments, the composition provided herein is used to treat any suitable patient, for example, the patients described in the examples.

In one aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the prostate cancer is microsatellite instability-high (MSI-H) prostate cancer. In one embodiment, the prostate cancer is mismatch repair deficient (MMRD) prostate cancer. In one embodiment, the subject receives treatment about every 2 weeks (Q2W) for about two years.

In another aspect, provided herein is a method of treating an aggressive variant (anaplastic) adenocarcinoma of the prostate (AVPCa) in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the method further comprising orally administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating an aggressive variant (anaplastic) adenocarcinoma of the prostate (AVPCa) in a male human subject in need thereof, wherein the subject has not previously been administered docetaxel, and wherein the method comprises: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the method further comprises administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter.

In another aspect, provided herein is a method of treating a prostate cancer in a male human subject in need thereof, wherein the subject has not previously been administered docetaxel, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, wherein the subject has not previously been administered a PARP inhibitor, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In some embodiments, the method further comprising administering olaparib at a dose of about 300 mg. In some embodiments, the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

In another aspect, provided herein is a method of treating an advanced gynecologic or genitourinary malignancy in a human subject in need thereof, the method comprising administering to the subject a dose of a bispecific antibody according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle, wherein the bispecific antibody comprises a first monomer comprising SEQ ID NO:1, a second monomer comprising SEQ ID NO:2, and a light chain comprising SEQ ID NO:3. In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments of the methods provided herein, if the weight of the subject changes by more than 10% from baseline, the subject is optionally reassigned to a new dosing level and one or more subsequent doses are administered to the subject at the new dosing level. In some embodiments of the methods provided herein, if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

In an exemplary embodiment, the prostate cancer is mCRPC. In an exemplary embodiment, the prostate cancer is microsatellite instability-high [MSI-H]. In an exemplary embodiment, the prostate cancer is mismatch repair deficient [MMRD]. In an exemplary embodiment, the mCRPC is microsatellite instability-high [MSI-H]. In an exemplary embodiment, the mCRPC is mismatch repair deficient [MMRD].

In an exemplary embodiment, the prostate cancer is MSI-H positive. In an exemplary embodiment, the prostate cancer is MMRD positive. In an exemplary embodiment, the mCRPC is MSI-H positive. In an exemplary embodiment, the mCRPC is MMRD positive.

In an exemplary embodiment, the prostate cancer is aggressive variant (anaplastic) adenocarcinoma of the prostate (AVPCa). In an exemplary embodiment, the mCRPC is aggressive variant (anaplastic) adenocarcinoma of the prostate (AVPCa). In an exemplary embodiment, the prostate cancer is neuroendocrine prostate cancer. In an exemplary embodiment, the mCRPC is neuroendocrine prostate cancer.

In an exemplary embodiment, the prostate cancer is positive for Rb1. In an exemplary embodiment, the prostate cancer is positive for TP53. In an exemplary embodiment, the prostate cancer is positive for PTENb. In an exemplary embodiment, the mCRPC is positive for aberrancy for at least two of Rb1, TP53, and PTENb. In an exemplary embodiment, the mCRPC is positive for Rb1. In an exemplary embodiment, the mCRPC is positive for TP53. In an exemplary embodiment, the mCRPC is positive for PTENb. In an exemplary embodiment, the mCRPC is positive for aberrancy for at least two of Rb1, TP53, and PTENb.

In an exemplary embodiment, the prostate cancer has a homologous recombination deficiency (HRD). In an exemplary embodiment, the prostate cancer is mCRPC with a homologous recombination deficiency. In an exemplary embodiment, the prostate cancer has a cyclin-dependent kinase 12 (CDK12) mutation. In an exemplary embodiment, the prostate cancer is mCRPC with a CDK12 mutation.

In an exemplary embodiment, the prostate cancer is HRD positive in at least one gene selected from the group consisting of BRCA1, BRCA2, ATM, PALB2, CHEK2, and FANCA. In an exemplary embodiment, the prostate cancer is mCRPC and is HRD positive in at least one gene selected from the group consisting of BRCA1, BRCA2, ATM, PALB2, CHEK2, and FANCA. In an exemplary embodiment, the prostate cancer is positive for biallelic loss of CDK12. In an exemplary embodiment, the prostate cancer is mCRPC and is positive for biallelic loss of CDK12.

In an exemplary embodiment, the prostate cancer is positive for HRD in one or more of the following genes: BRCA1, BRCA2, ATM, PALB2, CHEK2, FANCA; and/or positive for biallelic loss of CDK12. In an exemplary embodiment, the prostate cancer is mCRPC which is a HRD/CDK12 mutation positive cancer which is one of the following: Positive for HRD in one or more of the following genes: BRCA1, BRCA2, ATM, PALB2, CHEK2, FANCA; and/or Positive for biallelic loss of CDK12.

In an exemplary embodiment, the prostate cancer is not positive for aberrancy for at least two of Rb1, TP53, and PTENb; not positive for HRD in one or more of the following genes: BRCA1, BRCA2, ATM, PALB2, CHEK2, FANCA; not positive for biallelic loss of CDK12; and not positive for microsatellite instability-high [MSI-H] or mismatch repair deficient [MMRD]. In an exemplary embodiment, the mCRPC is not positive for aberrancy for at least two of Rb1, TP53, and PTENb; not positive for HRD in one or more of the following genes: BRCA1, BRCA2, ATM, PALB2, CHEK2, FANCA; not positive for biallelic loss of CDK12; and not positive for microsatellite instability-high [MSI-H] or mismatch repair deficient [MMRD].

In an exemplary embodiment, the human male subject with prostate cancer, prior to the dosing regimen, or the first occurrence of the dosing regimen, had not been administered a PARP inhibitor. In an exemplary embodiment, the human male subject with mCRPC, prior to the dosing regimen, or the first occurrence of the dosing regimen, had not been administered a PARP inhibitor.

In an exemplary embodiment, the prostate cancer is a HRD/CDK12 mutation positive cancer, and the human male subject with prostate cancer, prior to the dosing regimen, or the first occurrence of the dosing regimen, had not been administered a PARP inhibitor. In an exemplary embodiment, the prostate cancer is mCRPC and is a HRD/CDK12 mutation positive cancer, and the human male subject with prostate cancer, prior to the dosing regimen, or the first occurrence of the dosing regimen, had not been administered a PARP inhibitor. In an exemplary embodiment, the prostate cancer is a HRD/CDK12 mutation positive cancer which is one of the following: positive for HRD in one or more of the following genes: BRCA1, BRCA2, ATM, PALB2, CHEK2, FANCA; and/or positive for biallelic loss of CDK12, and the human male subject with prostate cancer, prior to the dosing regimen, or the first occurrence of the dosing regimen, had not been administered a PARP inhibitor. In an exemplary embodiment, the prostate cancer is mCRPC which is a HRD/CDK12 mutation positive cancer which is one of the following: Positive for HRD in one or more of the following genes: BRCA1, BRCA2, ATM, PALB2, CHEK2, FANCA; and/or Positive for biallelic loss of CDK12, and the human male subject with prostate cancer, prior to the dosing regimen, or the first occurrence of the dosing regimen, had not been administered a PARP inhibitor.

In an exemplary embodiment, the prostate cancer described herein is a primary tumor. In an exemplary embodiment, the prostate cancer described herein is a locally advanced primary tumor. In an exemplary embodiment, the prostate cancer described herein is a locally advanced primary tumor. In an exemplary embodiment, the prostate cancer described herein is a metastatic tumor.

In some embodiments, the cancer is a clear cell carcinoma. In some embodiments, the clear cell carcinoma is a clear cell ovarian, endometrial, or peritoneal carcinoma. In some embodiments, the clear cell carcinoma is a persistent or recurrent clear cell carcinoma of the ovary, peritoneum, or endometrium. In exemplary embodiments, the clear cell carcinoma was previously treated with a platinum-based systemic chemotherapy. In some embodiments, the cancer is a platinum-resistant high-grade serous ovarian cancer (HGSOC). In some embodiments, the patient with clear cell carcinoma has not been diagnosed with carcinosarcoma.

In some embodiments, the cancer is an endometrial cancer. In some embodiments, the endometrial carcinoma is an advance endometrial carcinoma. In particular embodiments, the advanced endometrial cancer is not MSI-H or deficient mismatch repair (dMMR). In exemplary embodiments, the endometrial cancer is an immune-checkpoint-inhibitor-refractory microsatellite stable (MSS) endometrial cancer (EC).

In some embodiments, the cancer is a cervical cancer. In exemplary embodiments, the cervical cancer is a previous treated recurrent or metastatic cervical cancer. In some embodiments, the previously treated cancer was treated with chemotherapy or an immunotherapy. In some embodiments the chemotherapy is a standard-of-care systemic chemotherapy. In some embodiments, the immunotherapy is an FDA-approved immunotherapy.

In an exemplary embodiment, the solid cancerous tumor is melanoma. In an exemplary embodiment, the solid cancerous tumor is melanoma, excluding uveal melanoma. In an exemplary embodiment, the solid cancerous tumor is cervical cancer. In an exemplary embodiment, the solid cancerous tumor is cervical carcinoma. In an exemplary embodiment, the solid cancerous tumor is breast carcinoma. In an exemplary embodiment, the solid cancerous tumor is breast carcinoma that is estrogen receptor, progesterone receptor, and human epidermal growth factor receptor 2 (HER2) negative (triple negative breast cancer [TNBC]). In an exemplary embodiment, the solid cancerous tumor is hepatocellular cancer. In an exemplary embodiment, the solid cancerous tumor is hepatocellular carcinoma. In an exemplary embodiment, the solid cancerous tumor is urothelial cancer. In an exemplary embodiment, the solid cancerous tumor is urothelial carcinoma. In an exemplary embodiment, the solid cancerous tumor is bladder cancer. In an exemplary embodiment, the solid cancerous tumor is head and neck cancer. In an exemplary embodiment, the solid cancerous tumor is squamous cell carcinoma of the head and neck. In an exemplary embodiment, the solid cancerous tumor is renal cell cancer. In an exemplary embodiment, the solid cancerous tumor is renal cell carcinoma. In an exemplary embodiment, the solid cancerous tumor is clear cell predominant type renal cell carcinoma.

In an exemplary embodiment, the solid cancerous tumor is colorectal cancer. In an exemplary embodiment, the solid cancerous tumor is MSI-high colorectal cancer. In an exemplary embodiment, the solid cancerous tumor is colorectal carcinoma. In an exemplary embodiment, the solid cancerous tumor is high microsatellite instability colorectal carcinoma. In an exemplary embodiment, the solid cancerous tumor is mismatch repair deficient colorectal carcinoma. In an exemplary embodiment, the solid cancerous tumor is endometrial cancer. In an exemplary embodiment, the solid cancerous tumor is MSI-high endometrial cancer. In an exemplary embodiment, the solid cancerous tumor is endometrial carcinoma. In an exemplary embodiment, the solid cancerous tumor is high microsatellite instability endometrial carcinoma. In an exemplary embodiment, the solid cancerous tumor is mismatch repair deficient endometrial carcinoma. In an exemplary embodiment, the solid cancerous tumor is small cell lung cancer. In an exemplary embodiment, the solid cancerous tumor is small cell lung carcinoma. In an exemplary embodiment, the solid cancerous tumor is non-small cell lung carcinoma. In an exemplary embodiment, the solid cancerous tumor is non-small cell lung cancer. In an exemplary embodiment, the solid cancerous tumor is gastric cancer. In an exemplary embodiment, the solid cancerous tumor is gastric adenocarcinoma. In an exemplary embodiment, the solid cancerous tumor is astroesophageal junction cancer. In an exemplary embodiment, the solid cancerous tumor is gastroesophageal junction adenocarcinoma.

In an exemplary embodiment, the solid cancerous tumor is a microsatellite instability-high cancer. In an exemplary embodiment, the solid cancerous tumor is a mismatch repair deficient cancer.

In an exemplary embodiment, the solid cancerous tumor is mesothelioma. In an exemplary embodiment, the solid cancerous tumor is neuroendocrine cancer. In an exemplary embodiment, the solid cancerous tumor is high-grade neuroendocrine cancer. In an exemplary embodiment, the solid cancerous tumor is neuroendocrine carcinoma. In an exemplary embodiment, the solid cancerous tumor is anal cancer. In an exemplary embodiment, the solid cancerous tumor is anal carcinoma. In an exemplary embodiment, the solid cancerous tumor is squamous cell carcinoma of the anus.

In an exemplary embodiment, the solid cancerous tumor is prostate cancer. In an exemplary embodiment, the solid cancerous tumor is castration-resistant prostate carcinoma. In an exemplary embodiment, the solid cancerous tumor is nasopharyngeal cancer. In an exemplary embodiment, the solid cancerous tumor is nasopharyngeal carcinoma. In an exemplary embodiment, the solid cancerous tumor is Cholangiocarcinoma. In an exemplary embodiment, the solid cancerous tumor is basal cell cancer. In an exemplary embodiment, the solid cancerous tumor is basal cell skin cancer. In an exemplary embodiment, the solid cancerous tumor is basal cell carcinoma. In an exemplary embodiment, the solid cancerous tumor is ovarian cancer. In an exemplary embodiment, the solid cancerous tumor is ovarian carcinoma. In an exemplary embodiment, the solid cancerous tumor is fallopian tube cancer. In an exemplary embodiment, the solid cancerous tumor is fallopian tube carcinoma.

In an exemplary embodiment, the solid cancerous tumor is thymus cancer. In an exemplary embodiment, the solid cancerous tumor is thymoma. In an exemplary embodiment, the solid cancerous tumor is thymic carcinoma. In an exemplary embodiment, the solid cancerous tumor is penile cancer. In an exemplary embodiment, the solid cancerous tumor is Squamous Cell Carcinoma of the Penis. In an exemplary embodiment, the solid cancerous tumor is vulvar cancer. In an exemplary embodiment, the solid cancerous tumor is vulvar carcinoma. In an exemplary embodiment, the solid cancerous tumor is solid tumors with published evidence of anti-tumor activity with anti-PD1/PDL1 and/or anti-CTLA4- directed therapy. In an exemplary embodiment, the solid cancerous tumor is malignant adnexal tumor. In an exemplary embodiment, the solid cancerous tumor is malignant adnexal neoplasm. In an exemplary embodiment, the solid cancerous tumor is salivary gland cancer. In an exemplary embodiment, the solid cancerous tumor is non-squamous cell salivary gland carcinoma. In an exemplary embodiment, the solid cancerous tumor is bile duct cancer. In an exemplary embodiment, the solid cancerous tumor is bile duct carcinoma. In an exemplary embodiment, the solid cancerous tumor described herein in a primary tumor. In an exemplary embodiment, the solid cancerous tumor described herein is a metastatic tumor.

In some embodiments, the cancer is treated according to a method described herein. In one embodiment, the cancer is treated (such as by achieving a positive therapeutic response) by administering a bispecific anti-CTLA4×anti-PD1 antibody (e.g., XmAb®20717) to the human subject in one or more cycles. Each cycle comprises dose amount(s) of a bispecific anti-CTLA4×anti-PD1 antibody (e.g., XmAb®20717) provided according to a dosage regimen described herein. Each cycle can last for one or more weeks or months, or until a positive therapeutic response is achieved, or so long as there is a positive therapeutic response. In one embodiment, administration of the bispecific anti-CTLA4×anti-PD1 antibody (e.g., XmAb®20717) stops after the positive therapeutic response is achieved, and the administration of the bispecific anti-CTLA4×anti-PD1 antibody (e.g., XmAb®20717) begins again once the positive therapeutic response diminishes or disappears. In one embodiment, the bispecific anti-CTLA4×anti-PD1 antibody (e.g., XmAb®20717) is administered until partial remission. In one embodiment, the bispecific anti-CTLA4×anti-PD1 antibody (e.g., XmAb®20717) is administered until complete remission.

In some embodiments, each cycle is 21 days. In some embodiments, each cycle is from 1-10 days, 1-15 days, 1-20 days, 1-21 days, 1-25 days, 1-28 days, 1-30 days, 1-45 days. In some embodiments, the method of treatment comprises a bispecific anti-CTLA4×anti-PD1 antibody (e.g., XmAb®20717) being administered in one to twenty cycles. In one embodiment, the method of treatment comprises a bispecific anti-CTLA4×anti-PD1 antibody (e.g., XmAb®20717) being administered in one, or two, or three, or four, or five, or six, or seven, or eight, or nine, or ten, or more than ten cycles. In one embodiment, the dosage regimen has a dose amount (quantity of an antibody) and an administration time (the length of time in which the dose amount is administered).

In one embodiment, the method comprises a first cycle. In one embodiment, the method comprises a first cycle and a second cycle. In one embodiment, the method comprises a first cycle and a second cycle, where each cycle is different or wherein each cycle is the same. In one embodiment, the method comprises a first cycle and a second cycle and a third cycle. In one embodiment, the method comprises a first cycle and a second cycle and a third cycle, where each cycle is different or wherein each cycle is the same. In one embodiment, the method comprises a first cycle and a second cycle and a third cycle and a fourth cycle. In one embodiment, the method comprises a first cycle and a second cycle and a third cycle and a fourth cycle, where each cycle is different or wherein each cycle is the same. In one embodiment, the method comprises a first cycle and a second cycle and a third cycle and a fourth cycle and a fifth cycle. In one embodiment, the method comprises a first cycle and a second cycle and a third cycle and a fourth cycle and a fifth cycle, where each cycle is different or wherein each cycle is the same. In one embodiment, the method comprises a first cycle and a second cycle and a third cycle and a fourth cycle and a fifth cycle and a sixth cycle. In one embodiment, the method comprises a first cycle and a second cycle and a third cycle and a fourth cycle and a fifth cycle and a sixth cycle, where each cycle is different or wherein each cycle is the same. In one embodiment, the method comprises a first cycle and a second cycle and a third cycle and a fourth cycle and a fifth cycle and a sixth cycle and a seventh cycle. In one embodiment, the method comprises a first cycle and a second cycle and a third cycle and a fourth cycle and a fifth cycle and a sixth cycle and a seventh cycle, where each cycle is different or wherein each cycle is the same. In one embodiment, the method comprises a first cycle and a second cycle and a third cycle and a fourth cycle and a fifth cycle and a sixth cycle and a seventh cycle and an eighth cycle. In one embodiment, the method comprises a first cycle and a second cycle and a third cycle and a fourth cycle and a fifth cycle and a sixth cycle and a seventh cycle and an eighth cycle, where each cycle is different or wherein each cycle is the same. In one embodiment, the method comprises a first cycle and a second cycle and a third cycle and a fourth cycle and a fifth cycle and a sixth cycle and a seventh cycle and an eighth cycle and a ninth cycle. In one embodiment, the method comprises a first cycle and a second cycle and a third cycle and a fourth cycle and a fifth cycle and a sixth cycle and a seventh cycle and an eighth cycle and a ninth cycle, where each cycle is different or wherein each cycle is the same. In some embodiments, different dosages (e.g., of the bispecific anti-CTLA4×anti-PD1 antibody) are used in each cycle or in each administration. In some embodiments, the same dosage (e.g., of the bispecific anti-CTLA4×anti-PD1 antibody) is used between administrations in the same cycle. In some embodiments, the same dosage (e.g., of the bispecific anti-CTLA4×anti-PD1 antibody) is used between any two cycles, any three cycles, any four cycles, or more than any four cycles. In some embodiments, different dosage (e.g., of the bispecific anti-CTLA4×anti-PD1 antibody) is used between administrations in the same cycle. In some embodiments, different dosage (e.g., of the bispecific anti-CTLA4×anti-PD1 antibody) is used between any two cycles, any three cycles, any four cycles, or more than any four cycles.

It will be understood that XmAb®20717 can be used as the bispecific anti-CTLA4×anti-PD1 antibody in all methods of the invention provided herein. It will be understood that a biosimilar or XmAb®20717 can be used as the bispecific anti-CTLA4×anti-PD1 antibody in all methods of the invention provided herein. It will be understood that a bioequivalent or XmAb®20717 can be used as the bispecific anti-CTLA4×anti-PD1 antibody in all methods of the invention provided herein.

Methods of Achieving a Positive Therapeutic Response Against Prostate Cancer

The compositions of the disclosure can be used to achieve a positive therapeutic response against prostate cancer. In an exemplary embodiment, a bispecific anti-CTLA4×anti-PD1 antibody (e.g., XmAb®20717) composition is administered according to a method described herein to achieve a positive therapeutic response against prostate cancer. In an exemplary embodiment, a bispecific anti-CTLA4×anti-PD1 antibody (e.g., XmAb®20717) composition and a carboplatin composition and a cabazitaxel composition are administered according to a method described herein to achieve a positive therapeutic response against prostate cancer. In an exemplary embodiment, a bispecific anti-CTLA4×anti-PD1 antibody (e.g., XmAb®20717) composition and a carboplatin composition and a docetaxel composition are administered according to a method described herein to achieve a positive therapeutic response against prostate cancer. In an exemplary embodiment, a bispecific anti-CTLA4×anti-PD1 antibody (e.g., XmAb®20717) composition and an olaparib composition are administered according to a method described herein to achieve a positive therapeutic response against prostate cancer. In an exemplary embodiment, the prostate cancer is receptive to treatment by an antibody which binds to CTLA4. In an exemplary embodiment, the prostate cancer is receptive to treatment by an antibody which binds to PD1. In an exemplary embodiment, the prostate cancer is receptive to treatment by an antibody which binds to CTLA4 and PD1. In an exemplary embodiment, the prostate cancer is receptive to treatment by carboplatin. In an exemplary embodiment, the prostate cancer is receptive to treatment by cabazitaxel. In an exemplary embodiment, the prostate cancer is receptive to treatment by docetaxel. In an exemplary embodiment, the prostate cancer is receptive to treatment by olaparib.

### 5.7 Assessment of Positive Therapeutic Response

In an exemplary embodiment, the positive therapeutic response can be assessed by assays or tools described herein. In an exemplary embodiment, the positive therapeutic response is a reduction in cancer tumor size, a reduction in the rate of cancer tumor growth, a reduction in the number of cancer-associated cells, an increase in cancer-associated cell death, an inhibition of cancer-associated cell survival, and/or an inhibition (i.e., slowing to some extent, preferably halting) of cancer-associated proliferation. In an exemplary embodiment, the positive therapeutic response is a reduction in prostate cancer tumor size, a reduction in the rate of prostate cancer tumor growth, a reduction in the number of prostate cancer-associated cells, an increase in prostate cancer-associated cell death, an inhibition of prostate cancer-associated cell survival, an inhibition (i.e., slowing to some extent, preferably halting) of prostate cancer-associated proliferation, and/or a lowering of prostate-specific antigen (PSA) levels in the male human subject.

In some embodiments, the desired therapeutic result is a complete response, partial response or stable disease. In specific embodiments, the desired therapeutic result is an improvement in response according to the RECIST guidelines (v. 1.1) as outlined in Eisenhauer et al. Eur. J. Cancer, 2009, 45:228-247, which is incorporated herein by reference in its entirety regarding, e.g., tumor response evaluation criteria.

In certain embodiments, the positive therapeutic response is assessed by the size of the target lesion(s). In some embodiments, the positive therapeutic response is a complete response as compared to baseline. In other embodiments, the positive therapeutic response is a partial response as compared to baseline. In some embodiments, the subject does not have progressive disease as compared to baseline. In other embodiments, the subject has stable disease as compared to baseline.

In certain embodiments, the positive therapeutic response is assessed by the size of the non-target lesion(s). In some embodiments, the positive therapeutic response is a complete response as compared to baseline. In other embodiments, the positive therapeutic response is a partial response as compared to baseline. In some embodiments, the subject does not have progressive disease as compared to baseline. In other embodiments, the subject has stable disease as compared to baseline.

In an exemplary embodiment, measuring of visceral lesions are used to assess the positive therapeutic response. Visceral lesions can be measured by computed tomography, or by caliper measurement by clinical examination, or by chest X-ray. The measurement methods are standardized and known to one of skill in the art. In an exemplary embodiment, a visceral lesion baseline assessment is taken prior to the first administration of the bispecific anti-CTLA4×anti-PD1 antibody (e.g., XmAb®20717). In an exemplary embodiment, a visceral lesion baseline assessment is taken prior to the first administration of the bispecific anti-CTLA4×anti-PD1 antibody (e.g., XmAb®20717), and involves: i) assessing the visceral lesions which have a minimum diameter along the longest plane of measurement of at least 10 mm by CT, 10 mm by caliper measurement by clinical examination, and 20 mm by chest X-ray, thereby assessing the target visceral lesions; ii) adding the sum of the diameters together of up to 5 of the target visceral lesions, thereby obtaining the visceral lesion baseline assessment. In an exemplary embodiment, a visceral lesion baseline assessment is taken between 28 days and 2 days prior to the first administration of the bispecific anti-CTLA4×anti-PD1 antibody (e.g., XmAb®20717). In an exemplary embodiment, a visceral lesion response assessment is taken after the first administration of the bispecific anti-CTLA4×anti-PD1 antibody (e.g., XmAb®20717). In an exemplary embodiment, a visceral lesion response assessment is taken between about 52 days and about 56 days after the first administration of the bispecific anti-CTLA4×anti-PD1 antibody (e.g., XmAb®20717). In an exemplary embodiment, a visceral lesion response assessment is taken after the first administration of the bispecific anti-CTLA4×anti-PD1 antibody (e.g., XmAb®20717) and involves: i) assessing the diameter of the up to 5 target visceral lesions; ii) adding the sum of the diameters together of up to 5 of the target visceral lesions, thereby obtaining the visceral lesion response assessment. In an exemplary embodiment, a visceral lesion baseline assessment is taken prior to the first administration of the bispecific anti-CTLA4×anti-PD1 antibody (e.g., XmAb®20717) or carboplatin or cabazitaxel or docetaxel. In an exemplary embodiment, a visceral lesion baseline assessment is taken prior to the first administration of the bispecific anti-CTLA4×anti-PD1 antibody (e.g., XmAb®20717) or carboplatin or cabazitaxel or docetaxel, and involves: i) assessing the visceral lesions which have a minimum diameter along the longest plane of measurement of at least 10 mm by CT, 10 mm by caliper measurement by clinical examination, and 20 mm by chest X-ray, thereby assessing the target visceral lesions; ii) adding the sum of the diameters together of up to 5 of the target visceral lesions, thereby obtaining the visceral lesion baseline assessment. In an exemplary embodiment, a visceral lesion baseline assessment is taken between 28 days and 2 days prior to the first administration of the bispecific anti-CTLA4×anti-PD1 antibody (e.g., XmAb®20717) or carboplatin or cabazitaxel or docetaxel. In an exemplary embodiment, a visceral lesion response assessment is taken after the first administration of the bispecific anti-CTLA4×anti-PD1 antibody (e.g., XmAb®20717) or carboplatin or cabazitaxel or docetaxel. In an exemplary embodiment, a visceral lesion response assessment is taken between about 52 days and about 56 days (about 8 weeks) after the first administration of the bispecific anti-CTLA4×anti-PD1 antibody (e.g., XmAb®20717) or carboplatin or cabazitaxel or docetaxel. In an exemplary embodiment, a visceral lesion response assessment is taken after the first administration of the bispecific anti-CTLA4×anti-PD1 antibody (e.g., XmAb®20717) or carboplatin or cabazitaxel or docetaxel and involves: i) assessing the diameter of the up to 5 target visceral lesions; ii) adding the sum of the diameters together of up to 5 of the target visceral lesions, thereby obtaining the visceral lesion response assessment. In an exemplary embodiment, a visceral lesion baseline assessment is taken prior to the first administration of the bispecific anti-CTLA4×anti-PD1 antibody (e.g., XmAb®20717) or olaparib. In an exemplary embodiment, a visceral lesion baseline assessment is taken prior to the first administration of the bispecific anti-CTLA4×anti-PD1 antibody (e.g., XmAb®20717) or olaparib, and involves: i) assessing the visceral lesions which have a minimum diameter along the longest plane of measurement of at least 10 mm by CT, 10 mm by caliper measurement by clinical examination, and 20 mm by chest X-ray, thereby assessing the target visceral lesions; ii) adding the sum of the diameters together of up to 5 of the target visceral lesions, thereby obtaining the visceral lesion baseline assessment. In an exemplary embodiment, a visceral lesion baseline assessment is taken between 28 days and 2 days prior to the first administration of the bispecific anti-CTLA4×anti-PD1 antibody (e.g., XmAb®20717) or olaparib. In an exemplary embodiment, a visceral lesion response assessment is taken after the first administration of the bispecific anti-CTLA4×anti-PD1 antibody (e.g., XmAb®20717) or olaparib. In an exemplary embodiment, a visceral lesion response assessment is taken between about 52 days and about 56 days (about 8 weeks) after the first administration of the bispecific anti-CTLA4×anti-PD1 antibody (e.g., XmAb®20717) or olaparib. In an exemplary embodiment, a visceral lesion response assessment is taken after the first administration of the bispecific anti-CTLA4×anti-PD1 antibody (e.g., XmAb®20717) or olaparib and involves: i) assessing the diameter of the up to 5 target visceral lesions; ii) adding the sum of the diameters together of up to 5 of the target visceral lesions, thereby obtaining the visceral lesion response assessment.

In an exemplary embodiment, the positive therapeutic response is achieved against the prostate cancer when there are fewer visceral lesions in the response assessment than in the baseline assessment. In an exemplary embodiment, the positive therapeutic response is achieved against the prostate cancer when the visceral lesions are smaller in the response assessment than in the baseline assessment. In an exemplary embodiment, the positive therapeutic response is achieved when the visceral lesion response assessment is at least 10%, or at least 20%, or at least 30%, or at least 40%, or at least 50%, or at least 60%, or last 70%, or at least 80%, or at least 90%, or 100% below the visceral lesion baseline assessment. In an exemplary embodiment, the positive therapeutic response is achieved when the visceral lesion response assessment is at least 30%, below the visceral lesion baseline assessment.

In an exemplary embodiment, the solid cancerous tumor can be assessed for positive therapeutic purposes by assessing one or more properties of the solid cancerous tumor in the patient prior to the administration of the compositions described herein, thereby obtaining a baseline assessment or evaluation. After the first administration of at least one of the compositions described herein, the solid cancerous tumor property is assessed or evaluated again, thereby obtaining a response assessment. By comparing the baseline assessment and the response assessment, a determination can be made if the positive therapeutic response has been achieved. If the response assessment is an improvement over the baseline assessment, then the positive therapeutic response against prostate cancer is achieved. In an exemplary embodiment, more than one response assessment is made at different times during the course of treatment (during one or more of the times in which the dosing regimen is implemented). In an exemplary embodiment, assessing the solid cancerous tumor is done by assessing one or more oncologic markers of the human subject. In an exemplary embodiment, the oncologic marker is selected from the group consisting of prostate-specific antigen (PSA) levels (for prostate cancer), bone lesions, visceral lesions, and malignant lymph nodes. For clarity, a positive therapeutic response can be in one oncologic marker category or more than one. That is, for example, a combination of lowered PSA level and/or reduced number of bone lesions and/or reduced diameter sum of target visceral lesions and/or reduced diameter sum of target malignant lymph nodes marks the achievement of the positive therapeutic response.

In an exemplary embodiment, PSA levels are used to assess a positive therapeutic response. PSA levels are obtained by PSA tests, and PSA tests are standardized and known to one of skill in the art. In an exemplary embodiment, a PSA level baseline assessment is taken prior to the first administration of the bispecific anti-CTLA4×anti-PD1 antibody (e.g., XmAb®20717). In an exemplary embodiment, a PSA level baseline assessment is taken between 28 days and 2 days prior to the first administration of the bispecific anti-CTLA4×anti-PD1 antibody (e.g., XmAb®20717). In an exemplary embodiment, a PSA level baseline assessment is taken the day prior to the first administration of bispecific anti-CTLA4×anti-PD1 antibody (e.g., XmAb®20717). In an exemplary embodiment, a PSA level response assessment is taken after the first administration of the bispecific anti-CTLA4×anti-PD1 antibody (e.g., XmAb®20717). In an exemplary embodiment, a PSA level response assessment is taken between about 21 days and about 28 days after the intravenous dose of the bispecific anti-CTLA4×anti-PD1 antibody (e.g., XmAb®20717). In an exemplary embodiment, a PSA level baseline assessment is taken prior to the first administration of the bispecific anti-CTLA4×anti-PD1 antibody (e.g., XmAb®20717) or carboplatin or cabazitaxel or docetaxel. In an exemplary embodiment, a PSA level baseline assessment is taken between 28 days and 2 days prior to the first administration of the bispecific anti-CTLA4×anti-PD1 antibody (e.g., XmAb®20717) or carboplatin or cabazitaxel or docetaxel. In an exemplary embodiment, a PSA level baseline assessment is taken the day prior to the first administration of the bispecific anti-CTLA4×anti-PD1 antibody (e.g., XmAb®20717) or carboplatin or cabazitaxel or docetaxel. In an exemplary embodiment, a PSA level response assessment is taken after the first administration of the bispecific anti-CTLA4×anti-PD1 antibody (e.g., XmAb®20717) or carboplatin or cabazitaxel or docetaxel. In an exemplary embodiment, a PSA level response assessment is taken between about 21 days and about 28 days after the intravenous dose of the bispecific anti-CTLA4×anti-PD1 antibody (e.g., XmAb®20717). In an exemplary embodiment, a PSA level response assessment is taken between about 26 days and about 30 days, or between about 54 days and about 58 days, or between about 82 days and about 86 days, or between about 110 days and about 114 days, after the first administration of the bispecific anti-CTLA4×anti-PD1 antibody (e.g., XmAb®20717) or carboplatin or cabazitaxel or docetaxel. In an exemplary embodiment, a PSA level response assessment is taken between about 82 days and about 86 days after the first administration of the bispecific anti-CTLA4×anti-PD1 antibody (e.g., XmAb®20717) or carboplatin or cabazitaxel or docetaxel. In an exemplary embodiment, a PSA level baseline assessment is taken prior to the first administration of the bispecific anti-CTLA4×anti-PD1 antibody (e.g., XmAb®20717) or olaparib. In an exemplary embodiment, a PSA level baseline assessment is taken between 28 days and 2 days prior to the first administration of the bispecific anti-CTLA4×anti-PD1 antibody (e.g., XmAb®20717) or olaparib. In an exemplary embodiment, a PSA level baseline assessment is taken the day prior to the first administration of the bispecific anti-CTLA4×anti-PD1 antibody (e.g., XmAb®20717) or olaparib. In an exemplary embodiment, a PSA level response assessment is taken after the first administration of the bispecific anti-CTLA4×anti-PD1 antibody (e.g., XmAb®20717) or olaparib. In an exemplary embodiment, a PSA level response assessment is taken between about 21 days and about 28 days after the intravenous dose of the bispecific anti-CTLA4×anti-PD1 antibody (e.g., XmAb®20717). In an exemplary embodiment, a PSA level response assessment is taken between about 26 days and about 30 days (about 4 weeks), or between about 54 days and about 58 days (about 7 weeks), or between about 82 days and about 86 days (about 12 weeks), or between about 110 days and about 114 days, after the first administration of the bispecific anti-CTLA4×anti-PD1 antibody (e.g., XmAb®20717) or olaparib. In an exemplary embodiment, a PSA level response assessment is taken between about 82 days and about 86 days (about 12 weeks) after the first administration of the bispecific anti-CTLA4×anti-PD1 antibody (e.g., XmAb®20717) or olaparib.

In an exemplary embodiment, the positive therapeutic response is achieved when the PSA level in the response assessment is below the PSA level in the baseline assessment. In an exemplary embodiment, the positive therapeutic response is achieved when the PSA level in the response assessment is at least 10%, or at least 20%, or at least 30%, or at least 40%, or at least 50%, or at least 60%, or last 70%, or at least 80%, or at least 90% below the PSA level in the baseline assessment. In an exemplary embodiment, the positive therapeutic response is achieved when the PSA level in the response assessment is at least 50% below the PSA level in the baseline assessment.

In an exemplary embodiment, measuring of bone lesions are used to assess the positive therapeutic response. Bone lesions can be measured by a bone scan, and these are standardized and known to one of skill in the art. In an exemplary embodiment, a bone lesion baseline assessment is taken prior to the first administration of the bispecific anti-CTLA4×anti-PD1 antibody (e.g., XmAb®20717). In an exemplary embodiment, a bone lesion baseline assessment is taken between 28 days and 2 days prior to the first administration of the bispecific anti-CTLA4×anti-PD1 antibody (e.g., XmAb®20717). In an exemplary embodiment, a bone lesion response assessment is taken after the first administration of the bispecific anti-CTLA4×anti-PD1 antibody (e.g., XmAb®20717). In an exemplary embodiment, a bone lesion response assessment is taken between about 52 days and about 56 days (about 8 weeks) after the first administration of the bispecific anti-CTLA4×anti-PD1 antibody (e.g., XmAb®20717). In an exemplary embodiment, a bone lesion response assessment involves counting the number of bone lesions in a bone scan taken between about 52 days and about 56 days (about 8 weeks) after the first administration of the bispecific anti-CTLA4×anti-PD1 antibody (e.g., XmAb®20717). In an exemplary embodiment, a bone lesion baseline assessment is taken prior to the first administration of the bispecific anti-CTLA4×anti-PD1 antibody (e.g., XmAb®20717) or carboplatin or cabazitaxel or docetaxel. In an exemplary embodiment, a bone lesion baseline assessment involves counting the number of bone lesions in a bone scan taken prior to the first administration of the bispecific anti-CTLA4×anti-PD1 antibody (e.g., XmAb®20717) or carboplatin or cabazitaxel or docetaxel. In an exemplary embodiment, a bone lesion baseline assessment is taken between 28 days and 2 days prior to the first administration of the bispecific anti-CTLA4×anti-PD1 antibody (e.g., XmAb®20717) or carboplatin or cabazitaxel or docetaxel. In an exemplary embodiment, a bone lesion response assessment is taken after the first administration of the bispecific anti-CTLA4×anti-PD1 antibody (e.g., XmAb®20717) or carboplatin or cabazitaxel or docetaxel. In an exemplary embodiment, a bone lesion response assessment is taken between about 52 days and about 56 days (about 8 weeks) after the first administration of the bispecific anti-CTLA4×anti-PD1 antibody (e.g., XmAb®20717) or carboplatin or cabazitaxel or docetaxel. In an exemplary embodiment, a bone lesion response assessment involves counting the number of bone lesions in a bone scan taken between about 52 days and about 56 days (about 8 weeks) after the first administration of the bispecific anti-CTLA4×anti-PD1 antibody (e.g., XmAb®20717) or carboplatin or cabazitaxel or docetaxel. In an exemplary embodiment, a bone lesion baseline assessment is taken prior to the first administration of the bispecific anti-CTLA4×anti-PD1 antibody (e.g., XmAb®20717) or olaparib. In an exemplary embodiment, a bone lesion baseline assessment involves counting the number of bone lesions in a bone scan taken prior to the first administration of the bispecific anti-CTLA4×anti-PD1 antibody (e.g., XmAb®20717) or olaparib. In an exemplary embodiment, a bone lesion baseline assessment is taken between 28 days and 2 days prior to the first administration of the bispecific anti-CTLA4×anti-PD1 antibody (e.g., XmAb®20717) or olaparib. In an exemplary embodiment, a bone lesion response assessment is taken after the first administration of the bispecific anti-CTLA4×anti-PD1 antibody (e.g., XmAb®20717) or olaparib. In an exemplary embodiment, a bone lesion response assessment is taken between about 52 days and about 56 days (about 8 weeks) after the first administration of the bispecific anti-CTLA4×anti-PD1 antibody (e.g., XmAb®20717) or olaparib. In an exemplary embodiment, a bone lesion response assessment involves counting the number of bone lesions in a bone scan taken between about 52 days and about 56 days (about 8 weeks) after the first administration of the bispecific anti-CTLA4×anti-PD1 antibody (e.g., XmAb®20717) or olaparib.

In an exemplary embodiment, the positive therapeutic response is achieved against the solid cancerous tumor (e.g., prostate cancer) when there are fewer bone lesions in the response assessment than in the baseline assessment. In an exemplary embodiment, the positive therapeutic response is achieved against the solid cancerous tumor (e.g., prostate cancer) when the number of bone lesions in the response assessment is lower than the number of bone lesions in the baseline assessment. In an exemplary embodiment, the positive therapeutic response is achieved when the number of bone lesions in the bone scan in the response assessment is at least 1 fewer, or at least 2 fewer, or at least 3 fewer, or at least 4 fewer, or at least 5 fewer than the number of bone lesions in the bone scan in the baseline assessment. In an exemplary embodiment, the positive therapeutic response is achieved against the solid cancerous tumor (e.g., prostate cancer) when the bone lesions are smaller in the response assessment than in the baseline assessment.

In an exemplary embodiment, measuring of malignant lymph nodes are used to assess the positive therapeutic response. Malignant lymph nodes can be measured by computed tomography. The measurement method is standardized and known to one of skill in the art. In an exemplary embodiment, a malignant lymph node baseline assessment is taken prior to the first administration of the bispecific anti-CTLA4×anti-PD1 antibody (e.g., XmAb®20717). In an exemplary embodiment, a malignant lymph node baseline assessment is taken prior to the first administration of the bispecific anti-CTLA4×anti-PD1 antibody (e.g., XmAb®20717), and involves: assessing the malignant lymph nodes which have a minimum diameter of at least 15 mm in the short axis by CT, thereby assessing the target malignant lymph nodes; ii) adding the sum of the diameters together of up to 5 of the target malignant lymph nodes, thereby obtaining the target malignant lymph node baseline assessment. In an exemplary embodiment, a malignant lymph node baseline assessment is taken between 28 days and 2 days prior to the first administration of the bispecific anti-CTLA4×anti-PD1 antibody (e.g., XmAb®20717). In an exemplary embodiment, a malignant lymph node response assessment is taken after the first administration of the bispecific anti-CTLA4×anti-PD1 antibody (e.g., XmAb®20717). In an exemplary embodiment, a malignant lymph node response assessment is taken between about 52 days and about 56 days after the first administration of the bispecific anti-CTLA4×anti-PD1 antibody (e.g., XmAb®20717). In an exemplary embodiment, a visceral lesion response assessment is taken after the first administration of the bispecific anti-CTLA4×anti-PD1 antibody (e.g., XmAb®20717) and involves: i) assessing the diameter of the up to 5 target malignant lymph nodes; ii) adding the sum of the diameters together of up to 5 of the target malignant lymph nodes, thereby obtaining the target malignant lymph node response assessment. In an exemplary embodiment, a malignant lymph node baseline assessment is taken prior to the first administration of the bispecific anti-CTLA4×anti-PD1 antibody (e.g., XmAb®20717) or carboplatin or cabazitaxel or docetaxel. In an exemplary embodiment, a malignant lymph node baseline assessment is taken prior to the first administration of the bispecific anti-CTLA4×anti-PD1 antibody (e.g., XmAb®20717) or carboplatin or cabazitaxel or docetaxel, and involves: assessing the malignant lymph nodes which have a minimum diameter of at least 15 mm in the short axis by CT, thereby assessing the target malignant lymph nodes; ii) adding the sum of the diameters together of up to 5 of the target malignant lymph nodes, thereby obtaining the target malignant lymph node baseline assessment. In an exemplary embodiment, a malignant lymph node baseline assessment is taken between 28 days and 2 days prior to the first administration of the bispecific anti-CTLA4×anti-PD1 antibody (e.g., XmAb®20717) or carboplatin or cabazitaxel or docetaxel. In an exemplary embodiment, a malignant lymph node response assessment is taken after the first administration of the bispecific anti-CTLA4×anti-PD1 antibody (e.g., XmAb®20717) or carboplatin or cabazitaxel or docetaxel. In an exemplary embodiment, a malignant lymph node response assessment is taken between about 52 days and about 56 days after the first administration of the bispecific anti-CTLA4×anti-PD1 antibody (e.g., XmAb®20717) or carboplatin or cabazitaxel or docetaxel. In an exemplary embodiment, a visceral lesion response assessment is taken after the first administration of the bispecific anti-CTLA4×anti-PD1 antibody (e.g., XmAb®20717) or carboplatin or cabazitaxel or docetaxel and involves: i) assessing the diameter of the up to 5 target malignant lymph nodes; ii) adding the sum of the diameters together of up to 5 of the target malignant lymph nodes, thereby obtaining the target malignant lymph node response assessment. In an exemplary embodiment, a malignant lymph node baseline assessment is taken prior to the first administration of the bispecific anti-CTLA4×anti-PD1 antibody (e.g., XmAb®20717) or olaparib. In an exemplary embodiment, a malignant lymph node baseline assessment is taken prior to the first administration of the bispecific anti-CTLA4×anti-PD1 antibody (e.g., XmAb®20717) or olaparib, and involves: assessing the malignant lymph nodes which have a minimum diameter of at least 15 mm in the short axis by CT, thereby assessing the target malignant lymph nodes; ii) adding the sum of the diameters together of up to 5 of the target malignant lymph nodes, thereby obtaining the target malignant lymph node baseline assessment. In an exemplary embodiment, a malignant lymph node baseline assessment is taken between 28 days and 2 days prior to the first administration of the bispecific anti-CTLA4×anti-PD1 antibody (e.g., XmAb®20717) or olaparib. In an exemplary embodiment, a malignant lymph node response assessment is taken after the first administration of the bispecific anti-CTLA4×anti-PD1 antibody (e.g., XmAb®20717) or olaparib. In an exemplary embodiment, a malignant lymph node response assessment is taken between about 52 days and about 56 days after the first administration of the bispecific anti-CTLA4×anti-PD1 antibody (e.g., XmAb®20717) or olaparib. In an exemplary embodiment, a visceral lesion response assessment is taken after the first administration of the bispecific anti-CTLA4×anti-PD1 antibody (e.g., XmAb®20717) or olaparib and involves: i) assessing the diameter of the up to 5 target malignant lymph nodes; ii) adding the sum of the diameters together of up to 5 of the target malignant lymph nodes, thereby obtaining the target malignant lymph node response assessment.

In an exemplary embodiment, the positive therapeutic response is achieved against the prostate cancer when there are fewer malignant lymph nodes in the response assessment than in the baseline assessment. In an exemplary embodiment, the positive therapeutic response is achieved against the prostate cancer when the malignant lymph nodes are smaller in the response assessment than in the baseline assessment. In an exemplary embodiment, the positive therapeutic response is achieved when the malignant lymph node response assessment is at least 10%, or at least 20%, or at least 30%, or at least 40%, or at least 50%, or at least 60%, or last 70%, or at least 80%, or at least 90%, or 100% below the malignant lymph node baseline assessment. In an exemplary embodiment, the positive therapeutic response is achieved when the malignant lymph node response assessment is at least 30%, below the malignant lymph node baseline assessment.

5.7.1. RECIST Guidelines

In some embodiments, disease or disease progression following treatment using a methods provided herein is determined based on the Response Evaluation Criteria in Solid Tumors (RECIST 1.1). In some embodiments, the objective response rate (ORR) or best observed response (BOR) is determined using RECIST 1.1. In some embodiments, progression of a disease of the disclosure is determined based on the RECIST guidelines. The following are selected definitions and tables from Revised RECIST Guidelines (Version 1.1; Eisenhauer et al, Eur J Cancer. 2009; 45:228-47; Chalian et al., Rad Onc. 2011; 31:2093-2105, each of which is herein incorporated by reference in its entirety).

5.7.1.1 Measurability of Visceral Lesion at Baseline

In an exemplary embodiment, at baseline, visceral lesions can be categorized as measurable or non-measurable as follows:

5.7.1.1.1 Measurable Visceral Lesions

In an exemplary embodiment, visceral lesions can be accurately measured in at least 1 dimension (longest diameter in the plane of measurement is to be recorded) with a minimum size of:
10 mm by computed tomography (CT) scan (CT scan slice thickness no greater than 5 mm)
10 mm caliper measurement by clinical exam (lesions which cannot be accurately measured with calipers should be recorded as non-measurable)
20 mm by chest X-ray

5.7.1.2 Measurability of Lymph Nodes at Baseline

In an exemplary embodiment, for malignant lymph nodes to be considered pathologically enlarged and measurable, a lymph node can be ≥15 mm in short axis when assessed by CT scan (CT scan slice thickness recommended to be no greater than 5 mm). At baseline and in follow-up, the short axis can be measured and followed.

5.7.1.3 Non-Measurable Lesions

In an exemplary embodiment, Non-measurable lesions are all other lesions, including
Small lesions (longest diameter<10 mm or pathological lymph nodes with P10 to <15 mm short axis) as well as truly non-measurable lesions
Lesions considered truly non-measurable include leptomeningeal disease, ascites, pleural or pericardial effusion, inflammatory breast disease, lymphangitic involvement of skin or lung, and abdominal masses/abdominal organomegaly identified by physical exam that is not measurable by reproducible imaging techniques.

5.7.1.4 Response Criteria

5.7.1.4.1 Target Lesions

In an exemplary embodiment, up to 5 target lesions may be identified and assessed for response. The following definitions can be used to determine objective tumor response for target lesions:
Complete Response (CR): Disappearance of all target lesions.—Any pathological lymph nodes (whether target or nontarget) should have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum should also demonstrate an absolute increase of at least 5 mm. (Note: the appearance of one or more new lesions is also considered progression.)
Stable Disease (SD): Neither sufficient shrinkage to qualify for PR nor sufficient increase to qualify for PD, taking as reference the smallest sum of diameters while on study.

5.7.1.4.2 Nontarget Lesions

In an exemplary embodiment, while some nontarget lesions may actually be measurable, they need not be measured and instead should be assessed only qualitatively at the time points specified in the protocol. In an exemplary embodiment, the following tumor response definitions apply to the group of nontarget lesions:
Complete Response (CR): Disappearance of all nontarget lesions and normalization of tumor marker level. All lymph nodes should be nonpathological in size (<10 mm short axis).
Non-CR/Non-PD: Persistence of one or more nontarget lesion(s) and/or maintenance of tumor marker level above the normal limit.
Progressive Disease (PD): Unequivocal progression of existing nontarget lesions. (Note: the appearance of one or more new lesions is also considered progression.)

5.7.1.4.3 New Lesions

In an exemplary embodiment, the appearance of new malignant lesions denotes disease progression. There are no specific criteria for the identification of new radiographic lesions; however, the finding of a new lesion should be unequivocal: i.e., not attributable to differences in scanning technique, change in imaging modality or findings thought to represent something other than tumor (for example, some ‘new’ bone lesions may be simply healing or flare of preexisting lesions). This is particularly important when the subject's baseline lesions show partial or complete response. For example, necrosis of a liver lesion may be reported on a CT scan report as a ‘new’ cystic lesion, which it is not.

In an exemplary embodiment, a lesion identified on a follow-up study in an anatomical location that was not scanned at baseline is considered a new lesion and will indicate disease progression. An example of this is the subject who has visceral disease at baseline and while on study has a CT or magnetic resonance imaging (MRI) brain ordered which reveals metastases. The subject's brain metastases are considered to be evidence of PD even if he/she did not have brain imaging at baseline.

In an exemplary embodiment, if a new lesion is equivocal, for example, because of its small size, continued therapy and follow-up evaluation will clarify if it represents truly new disease. If repeat scans confirm there is definitely a new lesion, then progression should be declared using the date of the initial scan.

5.7.1.5 Evaluation of Overall Response

The best overall response is the best response recorded from the start of the study treatment until the end of treatment, taking into account any requirement for confirmation. Responses after the termination of study drug may be included in the evaluation as long as the subject has not initiated new anticancer therapy.

The subject's overall response assignment will depend on the findings of both target and nontarget disease and will also take into consideration the appearance of new lesions. This is captured in Table 2.

TABLE 2
Assessment of Overall Response in Subjects
with Target (±Nontarget Disease)

| Target | Nontarget | New | Overall |
|---|---|---|---|
| Lesions | Lesions | Lesions | Response |
| CR | CR | No | CR |
| CR | Non-CR/non-PD | No | PR |
| CR | Not evaluated | No | PR |
| PR | Non-PD or not all evaluated | No | PR |
| SD | Non-PD or not all evaluated | No | SD |
| Not all | Non-PD | No | Not |
| evaluated | | | evaluable |
| PD | Any | Yes or No | PD |
| Any | PD | Yes or No | PD |
| Any | Any | Yes | PD |

CR = complete response, PD = progressive disease; PR = partial response, SD = stable disease.

5.7.2. Prostate Cancer Working Group Guidelines (PCWG3)

In some embodiments, disease or disease progression is determined based on the Prostate Cancer Working Group Guidelines (PCWG3). In some embodiments, progression of a disease of the disclosure is determined based on the PCWG3 guidelines. The following are selected definitions and tables from PCWG3 Guidelines (Scher et al. J Clin Oncol. 2016; 34(12):1402-18).

5.7.2.1 Baseline Assessments

In an exemplary embodiment, baseline PSA, bone scan, visceral lesions and lymph nodes can be measured as follows:

5.7.2.1.1 PSA

In an exemplary embodiment, PSA can be measured during screening and at baseline. In some embodiments, PSA is monitored through early rises for a minimum of 12 weeks (unless there is other evidence of progression). In some embodiments, progression in subjects without PSA reduction from baseline is indicated by a rise in PSA≥25% and >2 ng/mL above baseline value after 12 weeks of treatment. In some embodiments, progression in subjects with PSA reduction from baseline is indicated by a rise in PSA of ≥25% and ≥2 ng/mL above nadir and confirmed by a second value obtained at least 3 weeks later (i.e., a confirmed rising trend).

5.7.2.1.2 Visceral Lesions at Baseline

In an exemplary embodiment, visceral lesions can be accurately measured in at least one dimension (longest diameter in the plane of measurement is to be recorded) with a minimum size of:
10 mm by computed tomography (CT) scan (CT scan slice thickness no greater than 5 mm)
10 mm caliper measurement by clinical exam (lesions which cannot be accurately measured with calipers should be recorded as non-measurable)
20 mm by chest X-ray

In an exemplary embodiment, up to 5 lesions per site of disease (lungs, liver, adrenal, and central nervous system) are measured and recorded separately as measurable disease.

In an exemplary embodiment, visceral lesions considered truly non-measurable include leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

In an exemplary embodiment, visceral lesions which do not meet the above size criteria are non-measurable.

5.7.2.1.3 Lymph Nodes at Baseline

In an exemplary embodiment, lymph nodes can be measured in the short axis and recorded as follows:
Malignant lymph nodes: To be considered pathologically enlarged and measurable, a lymph node should be ≥15 mm in short axis when assessed by CT scan (CT scan slice thickness recommended to be no greater than 5 mm). At baseline and in follow-up, only the short axis will be measured and followed.
Nodes≥10 mm to <15 mm in short axis are pathologic, subject to clinical discretion, and non-measurable.
Nodes<10 mm are normal.

In an exemplary embodiment, pelvic and extrapelvic lymph nodes are recorded separately, and up to five nodal lesions total are recorded.

5.7.2.1.4 Bone Scan

In an exemplary embodiment, positivity on bone scan defines metastatic disease to bone. In an exemplary embodiment, the total number of lesions related to metastatic disease can be recorded.

5.7.2.2 Response Assessments

5.7.2.2.1 PSA

In an exemplary embodiment, serial PSA measurement can be performed and recorded through the study. In an exemplary embodiment, because a favorable effect on PSA may be delayed, treatment should be continued through early rises for a minimum of 12 weeks unless there is other evidence of progression.

In an exemplary embodiment, early rises in PSA (before 12 weeks) will not be considered progression unless there is other evidence of disease progression.

5.7.2.2.2 Visceral and Nodal Lesions

In an exemplary embodiment, changes in lymph nodes (pelvic and extrapelvic), lung, liver, adrenal, and central nervous system sites will be recorded separately.
Up to 5 target lesions per site of disease can be measured
Changes in visceral (lung, liver, adrenal, central nervous system) lesions≥10 mm in longest dimension can be recorded. The type of progression (growth of existing lesions versus development of new lesions) should be recorded by site.
Previously normal lymph nodes (<10 mm) should have grown by ≥5 mm in the short axis and be 10 mm in the short axis to be considered to have progressed
Nodes that have progressed to ≥10 mm to <15 mm are pathologic, subject to clinical discretion, and nonmeasurable
For existing pathologic adenopathy (≥15 mm), progression is defined per RECIST 1.1

5.7.2.2.3 Bone Scan

In an exemplary embodiment, exclude pseudoprogression in the absence of symptoms or other signs of progression.
First post-treatment scan: Progression is defined as at least two new lesions with at least 2 additional lesions on the next scan (2+2 rule)
All scans after the first post-treatment scan: At least 2 new lesions relative to the first post-treatment scan, confirmed on a subsequent scan

5.7.2.3 Evaluation of Overall Response

In an exemplary embodiment, PSA response and measurable disease are being evaluated in this study. Both measurable and non-measurable lesions can be evaluated at each restaging to determine whether they have completely resolved or persisted, or whether new lesions have appeared.

5.7.2.3.1 Response Criteria:

In an exemplary embodiment, subjects can be assessed using the following criteria:

5.7.2.3.2 Evaluation of Target Lesions
Complete Response (CR): Disappearance of all target lesions. Any pathological lymph nodes (whether target or nontarget) should have reduction in short axis to <10 mm
Partial Response (PR): At least a 30% decrease in the sum of diameter of target lesions taking as reference the baseline sum diameter
Progressive Disease (PD): At least a 20% increase in the sum diameter of target lesions taking as references the smallest sum diameter on the study (including baseline sum if that is the smallest sum on study). The sum should also demonstrate an absolute increase of at least 5 mm.
Stable Disease (SD): Neither sufficient shrinkage to qualify for PR nor sufficient increase to qualify for PD

5.7.2.3.3 Evaluation of Nontarget Lesions
Complete Response (CR): Disappearance of all nontarget lesions. All lymph nodes should be nonpathological in nature (<10 mm short axis).
Non-CR/Non-PD: Persistence of 1 or more nontarget lesions
Progressive Disease (PD): Unequivocal progression. Note that the designation of overall progression solely on the basis of a change in nontarget disease in the face of stable or responding target disease will be extremely rare. Examples of progression when the subject has only non-measurable disease include an increase in pleural effusion from “trace” to “large” or an increase in lymphangitic disease from localized to widespread. Refer to Section 4.3.4 of the RECIST guidelines for further guidance on progression in non-target disease (Eisenhauer et al, Eur J Cancer. 2009; 45:228-47).

### 5.8 Biological and Biochemical Functionality of the Heterodimeric Checkpoint Antibodies

Generally, the bispecific anti-CTLA4×anti-PD1 antibody (e.g., XmAb®20717) is administered to human subjects with certain solid cancerous tumors, and efficacy is assessed in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status along with “old fashioned” measurements such as tumor burden, size, invasiveness, LN involvement, metastasis, etc. can be done. Thus, any or all of the following can be evaluated: the inhibitory effects of the checkpoints on CD4+ T cell activation or proliferation, CD8+ T (CTL) cell activation or proliferation, CD8+ T cell-mediated cytotoxic activity and/or CTL mediated cell depletion, NK cell activity and NK mediated cell depletion, the potentiating effects of the checkpoints on Treg cell differentiation and proliferation and Treg- or myeloid derived suppressor cell (MDSC)-mediated immunosuppression or immune tolerance, and/or the effects of the checkpoints on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and 3H-Thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art. In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, 51Cr or 35S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

5.8.1. Assays to Measure Efficacy

In some embodiments, T cell activation is assessed using a Mixed Lymphocyte Reaction (MLR) assay as is known in the art. An increase in activity indicates immunostimulatory activity. Appropriate increases in activity are outlined herein.

In one embodiment, the signaling pathway assay measures increases or decreases in immune response as measured for example by phosphorylation or de-phosphorylation of different factors, or by measuring other post translational modifications. An increase in activity indicates immunostimulatory activity. Appropriate increases in activity are outlined herein.

In one embodiment, the signaling pathway assay measures increases or decreases in activation of αβ and/or γδ T cells as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in cytotoxic T cell activity as measured for example by direct killing of target cells like for example cancer cells or by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in NK and/or NKT cell activity as measured for example by direct killing of target cells like for example cancer cells or by cytokine secretion or by changes in expression of activation markers like for example CD107a, etc. An increase in activity indicates immunostimulatory activity. Appropriate increases in activity are outlined herein.

In one embodiment, the signaling pathway assay measures increases or decreases in αβ and/or γδ T-cell suppression, as measured for example by cytokine secretion or by proliferation or by changes in expression of activation markers like for example CD137, CD107a, PD1, etc. An increase in activity indicates immunostimulatory activity.

Appropriate increases in activity are outlined herein.

In one embodiment, the signaling pathway assay measures increases or decreases in pro-inflammatory cytokine secretion as measured, for example, by ELISA or by Luminex or by Multiplex bead based methods or by intracellular staining and FACS analysis or by Alispot, etc. An increase in activity indicates immunostimulatory activity. Appropriate increases in activity are outlined herein.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined herein.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined herein.

In one embodiment, the signaling pathway assay measures increases or decreases in Th1 response as measured for example by cytokine secretion or by changes in expression of activation markers. An increase in activity indicates immunostimulatory activity. Appropriate increases in activity are outlined herein.

In one embodiment, the signaling pathway assay measures increases or decreases in Th2 response as measured for example by cytokine secretion or by changes in expression of activation markers. An increase in activity indicates immunostimulatory activity. Appropriate increases in activity are outlined herein.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined herein.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined herein.

In one embodiment, the signaling pathway assay measures increases or decreases in M2 macrophage pro-tumorigenic activity, as measured for example by cytokine secretion or by changes in expression of activation markers. A decrease in response indicates immunostimulatory activity. Appropriate decreases are the same as for increases, outlined herein.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined herein.

In one embodiment, the signaling pathway assay measures increases or decreases in N2 neutrophils pro-tumorigenic activity, as measured for example by cytokine secretion or by changes in expression of activation markers. A decrease in response indicates immunostimulatory activity. Appropriate decreases are the same as for increases, outlined herein.

In one embodiment, the signaling pathway assay measures increases or decreases in inhibition of T cell activation, as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in inhibition of CTL activation as measured for example by direct killing of target cells like for example cancer cells or by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in αβ and/or γδ T cell exhaustion as measured for example by changes in expression of activation markers. A decrease in response indicates immunostimulatory activity.

Appropriate decreases are the same as for increases, outlined herein.

In one embodiment, the signaling pathway assay measures increases or decreases αβ and/or γδ T cell response as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in stimulation of antigen-specific memory responses as measured for example by cytokine secretion or by proliferation or by changes in expression of activation markers like for example CD45RA, CCR7 etc. An increase in activity indicates immunostimulatory activity. Appropriate increases in activity are outlined herein.

In one embodiment, the signaling pathway assay measures increases or decreases in apoptosis or lysis of cancer cells as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases in stimulation of cytotoxic or cytostatic effect on cancer cells. as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases direct killing of cancer cells as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases Th17 activity as measured for example by cytokine secretion or by proliferation or by changes in expression of activation markers. An increase in activity indicates immunostimulatory activity. Appropriate increases in activity are outlined herein.

In one embodiment, the signaling pathway assay measures increases or decreases in induction of complement dependent cytotoxicity and/or antibody dependent cell-mediated cytotoxicity, as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, T cell activation is measured for example by direct killing of target cells like for example cancer cells or by cytokine secretion or by proliferation or by changes in expression of activation markers like for example CD137, CD107a, PD1, etc. For T-cells, increases in proliferation, cell surface markers of activation (e.g., CD25, CD69, CD137, PD1), cytotoxicity (ability to kill target cells), and cytokine production (e.g., IL-2, IL-4, IL-6, IFNγ, TNF-α, IL-10, IL-17A) would be indicative of immune modulation that would be consistent with enhanced killing of cancer cells.

In one embodiment, NK cell activation is measured for example by direct killing of target cells like for example cancer cells or by cytokine secretion or by changes in expression of activation markers like for example CD107a, etc. For NK cells, increases in proliferation, cytotoxicity (ability to kill target cells and increases CD107a, granzyme, and perforin expression), cytokine production (e.g., IFNγ and TNF), and cell surface receptor expression (e.g., CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of 10%, 20%, 30%, 40%, 50%, 60%, 70%, 80%, 90%, 95% or 98 to 99% percent over the signal in either a reference sample or in control samples, for example test samples that do not contain an antibody provided herein. Similarly, increases of at least one-, two-, three-, four- or five-fold as compared to reference or control samples show efficacy.

5.8.2. Treatments

Once made, the compositions provided herein find use in a number of solid cancerous tumor applications, generally by inhibiting the suppression of T cell activation (e.g., T cells are no longer suppressed).

Accordingly, the bispecific anti-CTLA4×anti-PD1 antibody (e.g., XmAb®20717) finds use in the treatment of these cancers.

### 5.9 Dosage Regimen

A dose has a specific amount of antibody that is administered to a human subject over a defined time period. The amount of antibody administered to a human subject is also known as the dose amount. The time over which the dose amount is administered to a human subject is also known as the administration time. In some embodiments, the bispecific anti-CTLA4×anti-PD1 antibody (e.g., XmAb®20717) is administered to the human subject according to a dosage regimen described herein. Dosage regimen are adjusted to provide the optimum desired response (e.g., a therapeutic response). In some embodiments, the carboplatin, cabazitaxel, prednisone, and the bispecific anti-CTLA4×anti-PD1 antibody (e.g., XmAb®20717) are administered to the male human subject according to a dosage regimen described herein. In some embodiments, the carboplatin, docetaxel, prednisone, and the bispecific anti-CTLA4×anti-PD1 antibody (e.g., XmAb®20717) are administered to the male human subject according to a dosage regimen described herein. In some embodiments, the olaparib and the bispecific anti-CTLA4×anti-PD1 antibody (e.g., XmAb®20717) are administered to the male human subject according to a dosage regimen described herein. The efficient dosages and the dosage regimens for the bispecific anti-CTLA4×anti-PD1 antibody (e.g., XmAb®20717) depend on the disease or condition to be treated and may be determined by the persons skilled in the art. The efficient dosages and the dosage regimens for the bispecific anti-CTLA4×anti-PD1 antibody (e.g., XmAb®20717), carboplatin, cabazitaxel, docetaxel, and prednisone, depend on the disease or condition to be treated and may be determined by the persons skilled in the art. The efficient dosages and the dosage regimens for the bispecific anti-CTLA4×anti-PD1 antibody (e.g., XmAb®20717) and olaparib depend on the disease or condition to be treated and may be determined by the persons skilled in the art.

In one aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the prostate cancer is microsatellite instability-high (MSI-H) prostate cancer. In one embodiment, the prostate cancer is mismatch repair deficient (MMRD) prostate cancer. In one embodiment, the subject receives treatment about every 2 weeks (Q2W) for about two years.

In another aspect, provided herein is a method of treating an aggressive variant (anaplastic) adenocarcinoma of the prostate (AVPCa) in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the method further comprising orally administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating an aggressive variant (anaplastic) adenocarcinoma of the prostate (AVPCa) in a male human subject in need thereof, wherein the subject has not previously been administered docetaxel, and wherein the method comprises: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the method further comprises administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter.

In another aspect, provided herein is a method of treating a prostate cancer in a male human subject in need thereof, wherein the subject has not previously been administered docetaxel, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, wherein the subject has not previously been administered a PARP inhibitor, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In some embodiments, the method further comprising administering olaparib at a dose of about 300 mg. In some embodiments, the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

The dose amount may be determined or adjusted by measuring the amount of the bispecific anti-CTLA4×anti-PD1 antibody (e.g., XmAb®20717) in the blood upon administration, for instance taking out a biological sample and using anti-idiotypic antibodies which target the antigen binding region of the bispecific anti-CTLA4×anti-PD1 antibody (e.g., XmAb®20717). The dosage may be determined based upon the weight of the human subject, such as by multiplying the weight (in kg, for example) of the human subject by a dose amount (such as those described herein). Prior to the administering of the first cycle, such as the day before the first dose of the first cycle of administration, the weight of the human subject can be assessed. In some embodiments, the dosage is the same in at least two administrations, at least three, at least four, at least five, at least six, at least seven, at least eight, at least nine, at least ten, or more than at least ten administrations.

In some embodiments, provided herein are methods of treating an advanced gynecologic or genitourinary malignancy in a human subject in need thereof, comprising administering to the subject a flat dose of the bispecific anti-CTLA4×anti-PD1 antibody (e.g., XmAb®20717). In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments, the dosage of the bispecific anti-CTLA4×anti-PD1 antibody (e.g., XmAb®20717) is a flat dose based on the body weight. In some embodiments, the subject is administered about 1200 mg if the body weight of the subject is greater than or equal to 80 kg. In some embodiments, the subject is administered 1200 mg of the bispecific anti-CTLA4×anti-PD1 antibody (e.g., XmAb®20717) if the subject weighs greater or equal to 80 kg. In some embodiments, the subject is administered about 1000 mg if the body weight of the subject is less than 80 kg. In some embodiments, the subject is administered 1000 mg of the bispecific anti-CTLA4×anti-PD1 antibody (e.g., XmAb®20717) if the weight of the subject is less than 80 kg.

In some embodiments, the dose of the bispecific anti-CTLA4×anti-PD1 antibody (e.g., XmAb®20717) is between 500 to 2000 mg, 500 to 1500 mg, 600 to 1500 mg, 700 to 1500 mg, 800 to 1500 mg, 900 to 1500 mg, 950 to 1500 mg, or more than 950 to more than 1500 mg. In some embodiments, the dose of the bispecific anti-CTLA4×anti-PD1 antibody (e.g., XmAb®20717) is between 950 mg to 1000 mg, 950 mg to 1050 mg, 950 mg to 1100 mg, 950 mg to 1150 mg, 950 mg to 1200 mg, 950 mg to 1250 mg, 950 mg to 1300 mg, 950 mg to 1350 mg, 950 mg to 1400 mg, 950 mg to 1450 mg, 950 mg to 1500 mg, 950 mg to 2000 mg, or more than 950 mg to more than 2000 mg. In some embodiments, the dose of the bispecific anti-CTLA4×anti-PD1 antibody (e.g., XmAb®20717) is about, at least about, or at most about 500 mg, 600 mg, 700 mg, 800 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, or more than 2000 mg.

In one aspect, provided herein is a method of treating an advanced gynecologic or genitourinary malignancy in a human subject in need thereof, the method comprising administering to the subject a dose of a bispecific antibody according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle, wherein the bispecific antibody comprises a first monomer comprising SEQ ID NO:1, a second monomer comprising SEQ ID NO:2, and a light chain comprising SEQ ID NO:3. In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments of the methods provided herein, if the weight of the subject changes by more than 10% from baseline, the subject is optionally reassigned to a new dosing level and one or more subsequent doses are administered to the subject at the new dosing level. In some embodiments of the methods provided herein, if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

In some embodiments, the body weight of the subject is determined prior to Day 1 of a cycle. In some embodiments, the body weight of the subject is determined prior to Day 1 of a 21 day cycle. In some embodiments, the body weight of the subject is determined prior to Day 1 of each cycle. In some embodiments, the body weight of the subject is determined prior to Day 1 of each 21 day cycle. In some embodiments, the body weight of the subject is determined prior to Day 1 of the first cycle. In some embodiments, subjects who weigh less than 80 kilograms receive a specific dose of the bispecific anti-CTLA4×anti-PD1 antibody (e.g., XmAb®20717) (e.g., 1000 mg). In some embodiments, subjects who weight 80 kilograms or more receive a specific dose of the bispecific anti-CTLA4×anti-PD1 antibody (e.g., XmAb®20717) (e.g., 1200 mg). In some embodiments, the weight cutoff for dosage determination of the bispecific anti-CTLA4×anti-PD1 antibody (e.g., XmAb®20717) is 50 kg, 55 kg, 60 kg, 65 kg, 70 kg, 75 kg, 76 kg, 77 kg, 78 kg, 79 kg, 80 kg, 81 kg, 82 kg, 83 kg, 84 kg, 85 kg, 90 kg, 95 kg, 100 kg, 105 kg, 110 kg, or more than 110 kg. For example, if the subject weights 80 kg or more than 80 kg, the subject receives a specific dose of the bispecific anti-CTLA4×anti-PD1 antibody (e.g., XmAb®20717) (e.g., about 1200 mg) and if the subject weights less than 80 kg, the subject receives a specific dose of the bispecific anti-CTLA4×anti-PD1 antibody (e.g., XmAb®20717) (e.g., about 1000 mg). In some embodiments, if the subject weights 79 kg or more than 79 kg, the subject receives a specific dose of the bispecific anti-CTLA4×anti-PD1 antibody (e.g., XmAb®20717) (e.g., about 1200 mg) and if the subject weights less than 79 kg, the subject receives a specific dose of the bispecific anti-CTLA4×anti-PD1 antibody (e.g., XmAb®20717) (e.g., about 1000 mg). In some embodiments, a subject receives a dose of the bispecific anti-CTLA4×anti-PD1 antibody (e.g., XmAb®20717) on Day 1 of a cycle based on the body weight determined prior to Day 1 of the cycle. In some embodiments, the body weight of the subject affects the dosage of the bispecific anti-CTLA4×anti-PD1 antibody (e.g., XmAb®20717) to the subject and the dose is adjusted. For example, if at any time after treatment with the bispecific anti-CTLA4×anti-PD1 antibody (e.g., XmAb®20717) begins, the subject's weight changes (e.g., changes by 10% or more from baseline or from previous measurement) and the new weight requires reassignment to a different dose, subsequent doses can be provided at the new dose level until such a time that the body weight again requires assignment to the another dose level. Following the first dose of the first cycle administration, subsequent doses may be modified if the human subject's weight changes by a certain amount (for example, more than by about 5%, more than by about 10%, from the weight assessment prior to the first dose assessment or between any two assessments). In some embodiments, the weight of the subject may be recalculated for that infusion day or any subsequent infusion days using the current weight. In some embodiments, the bispecific anti-CTLA4×anti-PD1 antibody (e.g., XmAb®20717) is administered every 3 weeks. In some embodiments, the bispecific anti-CTLA4×anti-PD1 antibody (e.g., XmAb®20717) is administered every week, 2 weeks, 3 weeks, 4 weeks, 5 weeks, 6 weeks, 7 weeks, 8 weeks, 9 weeks, 10 weeks, or more than 10 weeks. In some embodiments, the bispecific anti-CTLA4×anti-PD1 antibody (e.g., XmAb®20717) is administered every 5 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer than 30 days. In some embodiments, the bispecific anti-CTLA4×anti-PD1 antibody (e.g., XmAb®20717) is administered at a dose of 1200 mg every 3 weeks. In some embodiments, the bispecific anti-CTLA4×anti-PD1 antibody (e.g., XmAb®20717) is administered at a dose of 1000 mg every 3 weeks. In some embodiments, the dose of the bispecific anti-CTLA4×anti-PD1 antibody (e.g., XmAb®20717) is determined based on Cₘₐₓ, AUC, and/or Cₗₐₛₜ. In some embodiments, two flat dose regimens (e.g., 1200 mg and 1000 mg) are used in the methods of the disclosure. In some embodiments, three, four, five, or more than five flat dose regimens are used in the methods of the disclosure. In some embodiments, each cycle is 21 days and the bispecific anti-CTLA4×anti-PD1 antibody (e.g., XmAb®20717) is administered on Day 1 of a cycle or of each cycle. In some embodiments, the bispecific anti-CTLA4×anti-PD1 antibody (e.g., XmAb®20717) is administered intravenously to a subject. In some embodiments, the bispecific anti-CTLA4×anti-PD1 antibody (e.g., XmAb®20717) is administered intravenously on Day 1 of a cycle to a subject. In some embodiments a cycle is 21 days. In some embodiments, a cycle is between 1 day and 30 days, 1 day and 28 days, 1 day and 25 days, 1 day and 21 days, and 1 day and 20 days. In some embodiments, a cycle is 15 days, 16 days, 17 days, 18 days, 19 days, 20 days, 21 days, 22 days, 23 days, 24 days, 25 days, 26 days, 27 days, 28 days, 29 days, and/or 30 days.

In some embodiments, the subject is a male human subject having a prostate cancer. In some embodiments, the subject is a male human subject having a microsatellite instability-high (MSI-H) prostate cancer. In some embodiments, the subject is a male human subject having a mismatch repair deficient (MMRD) prostate cancer. In some embodiments, the subject is a male human subject having an aggressive variant (anaplastic) adenocarcinoma of the prostate (AVPCa). In some embodiments, the subject is a male human subject having a prostate cancer that is an aggressive variant (anaplastic) adenocarcinoma of the prostate (AVPCa), wherein the prostate cancer has a mutation or other aberrancy in at least two genes independently selected from the group consisting of Rb1, TP53 and PTEN. In some embodiments, the subject is a male human subject having a prostate cancer that is an aggressive variant (anaplastic) adenocarcinoma of the prostate (AVPCa), wherein the subject has not previously been administered docetaxel, and wherein the prostate cancer has a mutation or other aberrancy in at least two genes independently selected from the group consisting of Rb1, TP53 and PTEN. n some embodiments, the subject is a male human subject having a prostate cancer, wherein the subject has not previously been administered docetaxel. In some embodiments, the subject is a male human subject having a prostate cancer, wherein the subject has received prior treatment with a polyadenosine diphosphate ribose polymerase (PARP) inhibitor. In some embodiments, the subject is a male human subject having a prostate cancer, wherein the cancer has a homologous recombination deficiency (HRD). In some embodiments, the subject is a male human subject having a prostate cancer, wherein the cancer has a biallelic loss of cyclin-dependent kinase 12 (CDK12). In some embodiments, the subject is a male human subject having a prostate cancer, wherein the cancer has a homologous recombination deficiency (HRD), and wherein the subject has not previously been administered docetaxel. In some embodiments, the subject is a male human subject having a prostate cancer, wherein the cancer has a biallelic loss of cyclin-dependent kinase 12 (CDK12), and wherein the subject has not previously been administered docetaxel. In some embodiments, the subject is a male human subject having a prostate cancer, wherein the cancer has a homologous recombination deficiency (HRD), and wherein the subject has received prior treatment with a polyadenosine diphosphate ribose polymerase (PARP) inhibitor. In some embodiments, the subject is a male human subject having a prostate cancer, wherein the cancer has a biallelic loss of cyclin-dependent kinase 12 (CDK12), and wherein the subject has received prior treatment with a polyadenosine diphosphate ribose polymerase (PARP) inhibitor. In some embodiments, the subject is a male human subject having a prostate cancer, wherein the cancer has a homologous recombination deficiency (HRD), wherein the subject has received prior treatment with a polyadenosine diphosphate ribose polymerase (PARP) inhibitor, and wherein the subject has not previously been administered docetaxel. In some embodiments, the subject is a male human subject having a prostate cancer, wherein the cancer has a biallelic loss of cyclin-dependent kinase 12 (CDK12), wherein the subject has received prior treatment with a polyadenosine diphosphate ribose polymerase (PARP) inhibitor, and wherein the subject has not previously been administered docetaxel. In some embodiments of the various methods provided herein, a dose of about 10 mg/kg of the bispecific anti-CTLA4×anti-PD1 antibody (e.g., XmAb®20717) is administered to a subject every two weeks. In a specific embodiment of the various methods provided herein, a dose of about 10 mg/kg of the bispecific anti-CTLA4×anti-PD1 antibody (e.g., XmAb®20717) is intravenously administered to a subject every two weeks.

In other embodiments of the various methods provided herein, from about 0.15 to about 15 mg/kg of the bispecific anti-CTLA4×anti-PD1 antibody (e.g., XmAb®20717) is administered to a subject every two weeks. In some embodiments, about 0.15 mg/kg of the bispecific anti-CTLA4×anti-PD1 antibody (e.g., XmAb®20717) is administered to a subject (e.g., every two weeks or every three weeks). In some embodiments, about 0.3 mg/kg of the bispecific anti-CTLA4×anti-PD1 antibody (e.g., XmAb®20717) is administered to a subject (e.g., every two weeks or every three weeks). In some embodiments, about 0.5 mg/kg of the bispecific anti-CTLA4×anti-PD1 antibody (e.g., XmAb®20717) is administered to a subject (e.g., every two weeks or every three weeks). In some embodiments, about 1 mg/kg of the bispecific anti-CTLA4×anti-PD1 antibody (e.g., XmAb®20717) is administered to a subject (e.g., every two weeks or every three weeks). In some embodiments, about 2 mg/kg of the bispecific anti-CTLA4×anti-PD1 antibody (e.g., XmAb®20717) is administered to a subject (e.g., every two weeks or every three weeks). In some embodiments, about 10 mg/kg of the bispecific anti-CTLA4×anti-PD1 antibody (e.g., XmAb®20717) is administered to a subject (e.g., every two weeks or every three weeks). In some embodiments, about 15 mg/kg of the bispecific anti-CTLA4×anti-PD1 antibody (e.g., XmAb®20717) is administered to a subject (e.g., every two weeks or every three weeks).

In other embodiments of the methods provided herein, the bispecific anti-CTLA4×anti-PD1 antibody (e.g., XmAb®20717) is administered to the subject on at a dose of about 10 mg/kg on day 1 of a first 28 day treatment cycle, and at a dose of about 10 mg/kg about every two weeks (Q2W) thereafter. In other embodiments of the methods provided herein, the bispecific anti-CTLA4×anti-PD1 antibody (e.g., XmAb®20717) is intravenously administered to the subject on at a dose of about 10 mg/kg on day 1 of a first 28 day treatment cycle, and at a dose of about 10 mg/kg about every two weeks (Q2W) thereafter. In certain embodiments of the methods provided herein, the bispecific anti-CTLA4×anti-PD1 antibody (e.g., XmAb®20717) is administered to the subject according to a 28 day treatment cycle. In certain embodiments of the methods provided herein, the bispecific anti-CTLA4×anti-PD1 antibody (e.g., XmAb®20717) is intravenously administered to the subject according to a 28 day treatment cycle. In certain embodiments of the methods provided herein, the bispecific anti-CTLA4×anti-PD1 antibody (e.g., XmAb®20717) is administered to the subject at a dose of about 10 mg/kg according to a 28 day treatment cycle. In certain embodiments of the methods provided herein, the bispecific anti-CTLA4×anti-PD1 antibody (e.g., XmAb®20717) is intravenously administered to the subject at a dose of about 10 mg/kg according to a 28 day treatment cycle. In other embodiments of the methods provided herein, the bispecific anti-CTLA4×anti-PD1 antibody (e.g., XmAb®20717) is administered to the subject on day 1 of a first 28 day treatment cycle, and about every two weeks (Q2W) thereafter. In other embodiments of the methods provided herein, the bispecific anti-CTLA4×anti-PD1 antibody (e.g., XmAb®20717) is intravenously administered to the subject on day 1 of a first 28 day treatment cycle, and about every two weeks (Q2W) thereafter. In other embodiments of the methods provided herein, the bispecific anti-CTLA4×anti-PD1 antibody (e.g., XmAb®20717) is administered to the subject according to a 28 day treatment cycle, and the bispecific anti-CTLA4×anti-PD1 antibody (e.g., XmAb®20717) is administered to the subject on day 1 of a first 28 day treatment cycle, and about every two weeks (Q2W) thereafter. In other embodiments of the methods provided herein, the bispecific anti-CTLA4×anti-PD1 antibody (e.g., XmAb®20717) is administered to the subject at a dose of about 10 mg/kg according to a 28 day treatment cycle, and the bispecific anti-CTLA4×anti-PD1 antibody (e.g., XmAb®20717) is administered to the subject on day 1 of a first 28 day treatment cycle, and about every two weeks (Q2W) thereafter. In some embodiments of the methods provided herein, the bispecific anti-CTLA4×anti-PD1 antibody (e.g., XmAb®20717) is intravenously administered to the subject according to a 28 day treatment cycle, and the bispecific anti-CTLA4×anti-PD1 antibody (e.g., XmAb®20717) is intravenously administered to the subject on day 1 of a first 28 day treatment cycle, and about every two weeks (Q2W) thereafter. In some embodiments of the methods provided herein, the bispecific anti-CTLA4×anti-PD1 antibody (e.g., XmAb®20717) is intravenously administered to the subject at a dose of about 10 mg/kg according to a 28 day treatment cycle, and the bispecific anti-CTLA4×anti-PD1 antibody (e.g., XmAb®20717) is intravenously administered to the subject on day 1 of a first 28 day treatment cycle, and about every two weeks (Q2W) thereafter. In certain embodiments, the subject is a male human subject having prostate cancer. In some embodiments, the prostate cancer is a microsatellite instability-high (MSI-H) prostate cancer. In some embodiments, the prostate cancer is a mismatch repair deficient (MMRD) prostate cancer. In some embodiments, the prostate cancer is an aggressive variant (anaplastic) adenocarcinoma of the prostate (AVPCa). In some embodiments, the AVPCa has a mutation or other aberrancy in at least two genes independently selected from the group consisting of Rb1, TP53 and PTEN.

In an exemplary embodiment, the intravenous dose of the bispecific anti-CTLA4×anti-PD1 antibody (e.g., XmAb®20717) is administered once between about 18 and about 25 days. In an exemplary embodiment, the intravenous dose of the bispecific anti-CTLA4×anti-PD1 antibody (e.g., XmAb®20717) is administered once between about 20 and about 22 days. In an exemplary embodiment, the intravenous dose of the bispecific anti-CTLA4×anti-PD1 antibody (e.g., XmAb®20717) is administered once between about 18 and about 23 days. In an exemplary embodiment, the intravenous dose of the bispecific anti-CTLA4×anti-PD1 antibody (e.g., XmAb®20717) is administered once between about 17 and about 22 days. In an exemplary embodiment, the intravenous dose of the bispecific anti-CTLA4×anti-PD1 antibody (e.g., XmAb®20717) is administered once between about 12 and about 17 days. In an exemplary embodiment, the intravenous dose of the bispecific anti-CTLA4×anti-PD1 antibody (e.g., XmAb®20717) is administered once between about 13 and about 15 days. In an exemplary embodiment, the intravenous dose of the bispecific anti-CTLA4×anti-PD1 antibody (e.g., XmAb®20717) is administered once every 13-15 days. In an exemplary embodiment, the intravenous dose of the bispecific anti-CTLA4×anti-PD1 antibody (e.g., XmAb®20717) is administered once between about 12 and about 16 days. In an exemplary embodiment, the intravenous dose of the bispecific anti-CTLA4×anti-PD1 antibody (e.g., XmAb®20717) is administered once every 12-16 days. In an exemplary embodiment, the intravenous dose of the bispecific anti-CTLA4×anti-PD1 antibody (e.g., XmAb®20717) is administered once every 14-16 days. In an exemplary embodiment, the intravenous dose of XmAb®20717 is administered once about every 14 days. In an exemplary embodiment, the intravenous dose of the bispecific anti-CTLA4×anti-PD1 antibody (e.g., XmAb®20717) is administered once every 14 days. In an exemplary embodiment, the intravenous dose of XmAb®20717 is administered once about every two weeks. In an exemplary embodiment, the intravenous dose of the bispecific anti-CTLA4×anti-PD1 antibody (e.g., XmAb®20717) is administered once every two weeks. In an exemplary embodiment, the intravenous dose of XmAb®20717 is administered once between about 13 and about 17 days. In an exemplary embodiment, the intravenous dose of the bispecific anti-CTLA4×anti-PD1 antibody (e.g., XmAb®20717) is administered once every 13-17 days. In an exemplary embodiment, the intravenous dose of XmAb®20717 is administered once about every 15 days. In an exemplary embodiment, the intravenous dose of the bispecific anti-CTLA4×anti-PD1 antibody (e.g., XmAb®20717) is administered once every 15 days. In an exemplary embodiment, the intravenous dose of XmAb®20717 is administered once about every 21 days. In an exemplary embodiment, the intravenous dose of the bispecific anti-CTLA4×anti-PD1 antibody (e.g., XmAb®20717) is administered once every 21 days.

In an exemplary embodiment, the administering of the intravenous dose to the human subject lasts between about 45 minutes and about 75 minutes. In an exemplary embodiment, the administering of the intravenous dose to the human subject lasts between 45 minutes and 75 minutes. In an exemplary embodiment, the administering of the intravenous dose to the human subject lasts about one hour. In an exemplary embodiment, the administering of the intravenous dose to the human subject lasts one hour.

In an exemplary embodiment, the bispecific anti-CTLA4×anti-PD1 antibody (e.g., XmAb®20717) is administered for a time period sufficient to treat the solid cancerous tumor. In an exemplary embodiment, the bispecific anti-CTLA4×anti-PD1 antibody (e.g., XmAb®20717) is administered for a time period sufficient to maintain the treatment of the solid cancerous tumor. In an exemplary embodiment, the time period is between about 1 and about 9 weeks. In an exemplary embodiment, the time period is between about 2 and about 7 weeks. In an exemplary embodiment, the time period is between about 3 and about 9 weeks. In an exemplary embodiment, the time period is between about 1 and about 8 weeks. In an exemplary embodiment, the time period is between about 3 and about 5 weeks. In an exemplary embodiment, the time period is about 4 weeks. In an exemplary embodiment, the time period is 4 weeks. In an exemplary embodiment, the time period is between about 7 and about 9 weeks. In an exemplary embodiment, the time period is about 8 weeks. In an exemplary embodiment, the time period is 8 weeks. In an exemplary embodiment, the time period is from about 1 week to about 10 years. In an exemplary embodiment, the time period is from about 1 week to about 9.5 years. In an exemplary embodiment, the time period is from about 1 week to about 9 years. In an exemplary embodiment, the time period is from about 1 week to about 8.5 years. In an exemplary embodiment, the time period is from about 1 week to about 8 years. In an exemplary embodiment, the time period is from about 1 week to about 7.5 years. In an exemplary embodiment, the time period is from about 1 week to about 7 years. In an exemplary embodiment, the time period is from about 1 week to about 6.5 years. In an exemplary embodiment, the time period is from about 1 week to about 6 years. In an exemplary embodiment, the time period is from about 1 week to about 5.5 years. In an exemplary embodiment, the time period is from about 1 week to about 5 years. In an exemplary embodiment, the time period is from about 1 week to about 4.5 years. In an exemplary embodiment, the time period is from about 1 week to about 4 years. In an exemplary embodiment, the time period is from about 1 week to about 3.5 years. In an exemplary embodiment, the time period is from about 1 week to about 3 years. In an exemplary embodiment, the time period is from about 1 week to about 2.5 years. In an exemplary embodiment, the time period is from about 1 week to about 2 years. In an exemplary embodiment, the time period is from about 1 week to about 1.5 years. In an exemplary embodiment, the time period is from about 1 week to about 1 year. In an exemplary embodiment, the time period is from about 1 week to about 3 months, or about 4 months, or about 5 months, or about 6 months, or about 7 months, or about 8 months, or about 9 months, or about 10 months or about 11 months. In an exemplary embodiment, the time period is until a positive therapeutic response is achieved. In an exemplary embodiment, the time period is as long as a positive therapeutic response is maintained. In an exemplary embodiment, the time period is until a complete response is achieved.

In an exemplary embodiment, the bispecific anti-CTLA4×anti-PD1 antibody (e.g., XmAb®20717) is administered once every 13-15 days (e.g., 14 days) for a time period lasting between about 1 and about 9 weeks. In an exemplary embodiment, the bispecific anti-CTLA4×anti-PD1 antibody (e.g., XmAb®20717) is administered once every 13-15 days (e.g., 14 days) for a time period lasting between about 2 and about 7 weeks. In an exemplary embodiment, the bispecific anti-CTLA4×anti-PD1 antibody (e.g., XmAb®20717) is administered once every 13-15 (e.g., 14 days) days for a time period lasting between about 3 and about 9 weeks. In an exemplary embodiment, the bispecific anti-CTLA4×anti-PD1 antibody (e.g., XmAb®20717) is administered once every 13-15 days (e.g., 14 days) for a time period lasting between about 1 and about 8 weeks. In an exemplary embodiment, the bispecific anti-CTLA4×anti-PD1 antibody (e.g., XmAb®20717) is administered once every 13-15 days (e.g., 14 days) for a time period lasting between about 3 and about 5 weeks. In an exemplary embodiment, the bispecific anti-CTLA4×anti-PD1 antibody (e.g., XmAb®20717) is administered once every 13-15 days (e.g., 14 days) for a time period lasting about 4 weeks. In an exemplary embodiment, the bispecific anti-CTLA4×anti-PD1 antibody (e.g., XmAb®20717) is administered once every 13-15 days (e.g., 14 days) for a time period lasting 4 weeks. In an exemplary embodiment, the bispecific anti-CTLA4×anti-PD1 antibody (e.g., XmAb®20717) is administered once every 13-15 days (e.g., 14 days) for a time period lasting between about 7 and about 9 weeks. In an exemplary embodiment, the bispecific anti-CTLA4×anti-PD1 antibody (e.g., XmAb®20717) is administered once every 13-15 days (e.g., 14 days) for a time period lasting about 8 weeks. In an exemplary embodiment, the bispecific anti-CTLA4×anti-PD1 antibody (e.g., XmAb®20717) is administered once every 13-15 days (e.g., 14 days) for a time period lasting 8 weeks. In an exemplary embodiment, the bispecific anti-CTLA4×anti-PD1 antibody (e.g., XmAb®20717) is administered once every 13-15 days (e.g., 14 days) for a time period lasting until a positive therapeutic response is achieved. In an exemplary embodiment, the bispecific anti-CTLA4×anti-PD1 antibody (e.g., XmAb®20717) is administered once every 13-15 days (e.g., 14 days) for a time period sufficient to treat the solid cancerous tumor. In an exemplary embodiment, the bispecific anti-CTLA4×anti-PD1 antibody (e.g., XmAb®20717) is administered once every 13-15 days (e.g., 14 days) for a time period sufficient to maintain the treatment of the solid cancerous tumor. In specific embodiments, the bispecific anti-CTLA4×anti-PD1 antibody (e.g., XmAb®20717) is administered to the subject about every two weeks (Q2W). In specific embodiments, the bispecific anti-CTLA4×anti-PD1 antibody (e.g., XmAb®20717) is administered to the subject every two weeks (Q2W). In certain embodiments, the bispecific anti-CTLA4×anti-PD1 antibody (e.g., XmAb®20717) is administered once every about 14 days. In certain embodiments, the bispecific anti-CTLA4×anti-PD1 antibody (e.g., XmAb®20717) is administered once every 14 days.

The dosage may be determined or adjusted by measuring the amount of bispecific anti-CTLA4×anti-PD1 antibody (e.g., XmAb®20717) provided herein in the blood upon administration using techniques known in the art, for instance taking out a biological sample and using anti-idiotypic antibodies which target the antigen binding region of the bispecific anti-CTLA4×anti-PD1 antibody (e.g., XmAb®20717).

In a specific embodiment, the intravenous dose of the bispecific anti-CTLA4×anti-PD1 antibody is 10 mg/kg. In certain embodiments, the intravenous dose bispecific anti-CTLA4×anti-PD1 antibody is 10 mg/kg, wherein the bispecific anti-CTLA4×anti-PD1 antibody comprises a first monomer comprising SEQ ID NO:1, a second monomer comprising SEQ ID NO:2, and a light chain comprising SEQ ID NO:3. In certain embodiments, the intravenous dose bispecific anti-CTLA4×anti-PD1 antibody is 10 mg/kg, wherein the bispecific anti-CTLA4×anti-PD1 antibody is XmAb®20717. In specific embodiment, the intravenous dose of 10 mg/kg of the bispecific anti-CTLA4×anti-PD1 antibody is administered to the subject once about every two weeks (Q2W). In certain embodiments, the subject is a male human subject having prostate cancer. In some embodiments, the prostate cancer is a microsatellite instability-high (MSI-H) prostate cancer. In some embodiments, the prostate cancer is a mismatch repair deficient (MMRD) prostate cancer. In some embodiments, the prostate cancer is an aggressive variant (anaplastic) adenocarcinoma of the prostate (AVPCa). In some embodiments, the AVPCa has a mutation or other aberrancy in at least two genes independently selected from the group consisting of Rb1, TP53 and PTEN.

### 5.10 Additional Exemplary Embodiments for the Treatment of Prostate Cancer in Male Human Subjects

PARAGRAPH A: In an exemplary embodiment, the dosing regimen lasts between about 12 and about 17 days. In an exemplary embodiment, the dosing regimen lasts between about 13 and about 15 days. In an exemplary embodiment, the dosing regimen lasts between about 12 and about 16 days. In an exemplary embodiment, the dosing regimen lasts between about 14 and about 16 days. In an exemplary embodiment, the dosing regimen lasts about 14 days. In an exemplary embodiment, the dosing regimen lasts 14 days. In an exemplary embodiment, the dosing regimen lasts about 15 days. In an exemplary embodiment, the dosing regimen lasts 15 days. In an exemplary embodiment, the dosing regimen lasts between about 80 and about 88 days. In an exemplary embodiment, the dosing regimen lasts between about 82 and about 86 days. In an exemplary embodiment, the dosing regimen lasts between about 83 and about 85 days. In an exemplary embodiment, the dosing regimen lasts about 84 days. In an exemplary embodiment, the dosing regimen lasts 84 days. In an exemplary embodiment, the dosing regimen lasts about 85 days. In an exemplary embodiment, the dosing regimen lasts 85 days.

PARAGRAPH B: In an exemplary embodiment, the dosing regimen occurs one time. In an exemplary embodiment, the dosing regimen occurs two times. In an exemplary embodiment, the dosing regimen occurs three times. In an exemplary embodiment, the dosing regimen occurs four times. In an exemplary embodiment, the dosing regimen occurs five times. In an exemplary embodiment, the dosing regimen occurs six times. In an exemplary embodiment, the dosing regimen occurs seven times. In an exemplary embodiment, the dosing regimen occurs eight times. In an exemplary embodiment, the dosing regimen occurs nine times. In an exemplary embodiment, the dosing regimen occurs ten times. In an exemplary embodiment, the dosing regimen repeats until a positive therapeutic response is achieved. In an exemplary embodiment, the dosing regimen repeats for as long as there is the positive therapeutic response. In an exemplary embodiment, the dosing regimen is administered to a male human subject for the treatment of a prostate cancer for as long as there is the positive therapeutic response.

PARAGRAPH C: In an exemplary embodiment, the intravenous dose of the bispecific anti-CTLA4×anti-PD1 antibody (e.g., XmAb®20717) is administered once between about 12 and about 17 days. In an exemplary embodiment, the intravenous dose of the bispecific anti-CTLA4×anti-PD1 antibody (e.g., XmAb®20717) is administered once between about 13 and about 15 days. In an exemplary embodiment, the intravenous dose of the bispecific anti-CTLA4×anti-PD1 antibody (e.g., XmAb®20717) is administered once every 13-15 days (e.g., 14 days). In an exemplary embodiment, the intravenous dose of the bispecific anti-CTLA4×anti-PD1 antibody (e.g., XmAb®20717) is administered once between about 12 and about 16 days. In an exemplary embodiment, the intravenous dose of the bispecific anti-CTLA4×anti-PD1 antibody (e.g., XmAb®20717) is administered once every 12-16 days. In an exemplary embodiment, the intravenous dose of XmAb®20717 is administered once every 14-16 days. In an exemplary embodiment, the intravenous dose of the bispecific anti-CTLA4×anti-PD1 antibody (e.g., XmAb®20717) is administered once about every 14 days. In an exemplary embodiment, the intravenous dose of the bispecific anti-CTLA4×anti-PD1 antibody (e.g., XmAb®20717) is administered once every 14 days. In an exemplary embodiment, the intravenous dose of the bispecific anti-CTLA4×anti-PD1 antibody (e.g., XmAb®20717) is administered once about every two weeks. In an exemplary embodiment, the intravenous dose of the bispecific anti-CTLA4×anti-PD1 antibody (e.g., XmAb®20717) is administered once every two weeks. In an exemplary embodiment, the intravenous dose of XmAb®20717 is administered once between about 13 and about 17 days. In an exemplary embodiment, the intravenous dose of the bispecific anti-CTLA4×anti-PD1 antibody (e.g., XmAb®20717) is administered once every 13-17 days. In an exemplary embodiment, the intravenous dose of the bispecific anti-CTLA4×anti-PD1 antibody (e.g., XmAb®20717) is administered once about every 15 days. In an exemplary embodiment, the intravenous dose of the bispecific anti-CTLA4×anti-PD1 antibody (e.g., XmAb®20717) is administered once every 15 days.

PARAGRAPH D: In an exemplary embodiment, the administering of the intravenous dose of the bispecific anti-CTLA4×anti-PD1 antibody (e.g., XmAb®20717) to the male human subject lasts between about 45 minutes and about 75 minutes. In an exemplary embodiment, the administering of the intravenous dose of the bispecific anti-CTLA4×anti-PD1 antibody (e.g., XmAb®20717) to the male human subject lasts between 45 minutes and 75 minutes. In an exemplary embodiment, the administering of the intravenous dose of the bispecific anti-CTLA4×anti-PD1 antibody (e.g., XmAb®20717) to the male human subject lasts between about 50 minutes and about 70 minutes. In an exemplary embodiment, the administering of the intravenous dose of the bispecific anti-CTLA4×anti-PD1 antibody (e.g., XmAb®20717) to the male human subject lasts between 50 minutes and 70 minutes. In an exemplary embodiment, the administering of the intravenous dose of the bispecific anti-CTLA4×anti-PD1 antibody (e.g., XmAb®20717) to the male human subject lasts about 60 minutes. In an exemplary embodiment, the administering of the intravenous dose of the bispecific anti-CTLA4×anti-PD1 antibody (e.g., XmAb®20717) to the male human subject lasts 60 minutes.

PARAGRAPH E: In an exemplary embodiment, the intravenous dose of XmAb®20717 is between about 9.0 mg/kg and about 11.0 mg/kg. In an exemplary embodiment, the intravenous dose of the bispecific anti-CTLA4×anti-PD1 antibody (e.g., XmAb®20717) is between about 9.5 mg/kg and about 10.5 mg/kg. In an exemplary embodiment, the intravenous dose of the bispecific anti-CTLA4×anti-PD1 antibody (e.g., XmAb®20717) is between about 9.8 mg/kg and about 10.2 mg/kg. In an exemplary embodiment, the intravenous dose of the bispecific anti-CTLA4×anti-PD1 antibody (e.g., XmAb®20717) is about 10 mg/kg. In an exemplary embodiment, the intravenous dose of the bispecific anti-CTLA4×anti-PD1 antibody (e.g., XmAb®20717) is 10 mg/kg. The dosage may be determined or adjusted by measuring the amount of the bispecific anti-CTLA4×anti-PD1 antibody (e.g., XmAb®20717) provided herein in the blood upon administration using techniques known in the art, for instance taking out a biological sample and using anti-idiotypic antibodies which target the antigen binding region of the bispecific anti-CTLA4×anti-PD1 antibody (e.g., XmAb®20717). The dosage may be determined based upon the weight of the male human subject, such as by multiplying the weight (in kg, for example) of the male human subject by a dose amount (such as those described herein). Prior to the administering of the first intravenous dose, such as the day before the first intravenous dose, the weight of the male human subject can be assessed. For male human subjects whose weight exceeds 100 kg, the intravenous dose can be calculated based on a weight of 100 kg rather than being calculated based upon the male human subject's actual body weight. Following the first intravenous dose, subsequent doses may be modified if the male human subject's weight changes by a certain amount (for example, more than by about 5%, more than by about 10%, from the weight assessment made prior to the first occurrence of the dosing regimen). At such a point, the weight may be recalculated for that infusion day using the current weight.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

PARAGRAPH F: In an exemplary embodiment, the intravenous dose of the carboplatin, with a target area under the serum concentration-time curve of between about 3 (AUC 3) and about 5 (AUC 5). In an exemplary embodiment, the intravenous dose of the carboplatin, with a target area under the serum concentration-time curve of about 4 (AUC 4). In an exemplary embodiment, the intravenous dose of the carboplatin, with a target area under the serum concentration-time curve of 4 (AUC 4). In an exemplary embodiment, the oral dose of olaparib is between about 270 mg and about 330 mg. In an exemplary embodiment, the oral dose of olaparib is between about 290 mg and about 310 mg. In an exemplary embodiment, the oral dose of olaparib is about 300 mg. In an exemplary embodiment, the oral dose of olaparib is 300 mg.

PARAGRAPH G: In an exemplary embodiment, the intravenous dose is administered once between every about 18 and about 24 days. In an exemplary embodiment, the intravenous dose is administered once between every about 19 and about 23 days. In an exemplary embodiment, the intravenous dose is administered once between every about 20 and about 22 days. In an exemplary embodiment, the intravenous dose is administered once about every 21 days. In an exemplary embodiment, the intravenous dose is administered once every 21 days. In an exemplary embodiment, the intravenous dose is administered once about every three weeks. In an exemplary embodiment, the intravenous dose is administered once every three weeks.

PARAGRAPH H: In an exemplary embodiment, the intravenous dose of cabazitaxel is between about 18 mg/m² and about 25 mg/m². In an exemplary embodiment, the intravenous dose of cabazitaxel is about 20 mg/m². In an exemplary embodiment, the intravenous dose of cabazitaxel is 20 mg/m².

PARAGRAPH I: In an exemplary embodiment, the intravenous dose of docetaxel is between about 55 mg/m² and about 75 mg/m². In an exemplary embodiment, the intravenous dose of docetaxel is about 60 mg/m². In an exemplary embodiment, the intravenous dose of docetaxel is 60 mg/m².

PARAGRAPH J: In an exemplary embodiment, the administering of the intravenous dose of carboplatin to the male human subject lasts between about 20 minutes and about 40 minutes. In an exemplary embodiment, the administering of the intravenous dose of carboplatin to the male human subject lasts between about 25 minutes and about 35 minutes. In an exemplary embodiment, the administering of the intravenous dose of carboplatin to the male human subject lasts about 30 minutes. In an exemplary embodiment, the administering of the intravenous dose of carboplatin to the male human subject lasts 30 minutes.

PARAGRAPH K: In an exemplary embodiment, the administering of the intravenous dose of cabazitaxel or docetaxel to the male human subject lasts between about 45 minutes and about 75 minutes. In an exemplary embodiment, the administering of the intravenous dose of cabazitaxel or docetaxel to the male human subject lasts between 45 minutes and 75 minutes. In an exemplary embodiment, the administering of the intravenous dose of cabazitaxel or docetaxel to the male human subject lasts between about 50 minutes and about 70 minutes. In an exemplary embodiment, the administering of the intravenous dose of cabazitaxel or docetaxel to the male human subject lasts between 50 minutes and 70 minutes. In an exemplary embodiment, the administering of the intravenous dose of cabazitaxel or docetaxel to the male human subject lasts about 60 minutes. In an exemplary embodiment, the administering of the intravenous dose of cabazitaxel or docetaxel to the male human subject lasts 60 minutes.

PARAGRAPH L: In an exemplary embodiment, the oral dose of prednisone is between about 3 mg and about 7 mg. In an exemplary embodiment, the oral dose of prednisone is about 5 mg. In an exemplary embodiment, the oral dose of prednisone is 5 mg.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

In some embodiments, the bispecific anti-CTLA4×anti-PD1 antibody (e.g., XmAb®20717) is administered intravenously. In some embodiments, the XmAb®20717 is administered once-every-two-weeks until disease progression, unacceptable toxicity, or individual choice.

In some embodiments, the bispecific anti-CTLA4×anti-PD1 antibody (e.g., XmAb®20717) is a front line therapy, second line therapy, third line therapy, fourth line therapy, fifth line therapy, or sixth line therapy.

In some embodiments, the bispecific anti-CTLA4×anti-PD1 antibody (e.g., XmAb®20717) treats a refractory solid cancerous tumor. In some embodiments, the bispecific anti-CTLA4×anti-PD1 antibody (e.g., XmAb®20717) is a maintenance therapy.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

Specific dosing regimens are also contemplated and encompassed by embodiments of the various methods provided herein for the treatment of a prostate cancer in a human male subject.

The specification for the dosage unit forms provided herein are dictated by and directly dependent on (a) the unique characteristics of the active compound and the particular therapeutic effect to be achieved, and (b) the limitations inherent in the art of compounding such an active compound for the treatment of sensitivity in individuals.

In one embodiment, each dosage regimen comprises at least one dose of the bispecific anti-CTLA4×anti-PD1 antibody (e.g., XmAb®20717) provided to the human subject (per week, per two weeks, per three weeks, or per month/over a set period of day(s) or week(s)). Dosage regimens are adjusted to provide the optimum desired response (e.g., a positive therapeutic response). The efficient dosages and the dosage regimens for the bispecific anti-CTLA4×anti-PD1 antibody (e.g., XmAb®20717) used in the present methods depend on the disease or condition to be treated. In a specific embodiment, the bispecific anti-CTLA4×anti-PD1 antibody is XmAb®20717. In an exemplary embodiment, provided is a method of achieving a positive therapeutic response against prostate cancer, comprising administering a bispecific antibody to a male human subject in need of treatment thereof with a dosing regimen comprising: an intravenous dose of the bispecific antibody as described in Paragraph E, administered on the first day of the dosing regimen, and administered over a time period described in Paragraph D; wherein the bispecific antibody comprises a first monomer comprising SEQ ID NO: 1, a second monomer comprising SEQ ID NO: 2, and a light chain comprising SEQ ID NO: 3, wherein the dosing regimen is as described in Paragraph A and wherein the dosing regimen occurs as described in Paragraph B. In an exemplary embodiment, the invention is according to an embodiment of this paragraph, wherein the prostate cancer is mCRPC. In an exemplary embodiment, the invention is according to an embodiment of this paragraph, wherein the prostate cancer is mCRPC, and the mCRPC is microsatellite instability-high [MSI-H]. In an exemplary embodiment, the invention is according to an embodiment of this paragraph, wherein the prostate cancer is mCRPC, and the mCRPC is mismatch repair deficient [MMRD]. In an exemplary embodiment, the invention is according to an embodiment of this paragraph, wherein prior to the first occurrence of the dosage regimen, the male human subject has been administered at least one previous prostate cancer treatments, which was a checkpoint inhibitor. In an exemplary embodiment, the invention is according to an embodiment of this paragraph, wherein prior to the first occurrence of the dosage regimen, the male human subject has been administered at least two previous prostate cancer treatments, one of which was a checkpoint inhibitor, and the other was not the bispecific antibody. In an exemplary embodiment, the invention is according to an embodiment of this paragraph, wherein prior to the first occurrence of the dosage regimen, an androgen suppression treatment had been administered to the male human subject. In an exemplary embodiment, the invention is according to an embodiment of this paragraph, wherein during an occurrence of the dosage regimen, an androgen suppression treatment is administered to the male human subject. In an exemplary embodiment, the invention is according to an embodiment of this paragraph, wherein the first monomer consists of SEQ ID NO: 1, the second monomer consists of SEQ ID NO: 2, and the light chain consists of SEQ ID NO: 3. In an exemplary embodiment, the invention is according to an embodiment of this paragraph, using the bispecific anti-CTLA4×anti-PD1 antibody (e.g., XmAb®20717) as disclosed in Section “III. Antibodies” of the disclosure. In an exemplary embodiment, the invention is according to an embodiment of this paragraph, further comprising, prior to the administering, assessing the weight of the male human subject.

In one aspect, the disclosure provides a method of achieving a positive therapeutic response against prostate cancer, administering carboplatin, cabazitaxel, prednisone, and a bispecific antibody to a male human subject in need of treatment thereof with a dosing regimen comprising: a) an intravenous dose of the carboplatin as described in Paragraph F, and administered as described in Paragraphs G & J; b) an intravenous dose of the cabazitaxel as described in Paragraph H, and administered as described in Paragraphs G & K; c) an oral dose of the prednisone, as described in Paragraph L, is administered twice a day; d) an intravenous dose of the bispecific antibody as described in Paragraph E, administered on the first day of the dosing regimen, and administered over a time period described in Paragraphs C and D; wherein the bispecific antibody comprises a first monomer comprising SEQ ID NO: 1, a second monomer comprising SEQ ID NO: 2, and a light chain comprising SEQ ID NO: 3, wherein the dosing regimen lasts as described in Paragraph A and wherein the dosing regimen repeats until the positive therapeutic response is achieved.

In one aspect, provided herein is a method of achieving a positive therapeutic response against prostate cancer, administering carboplatin, cabazitaxel, prednisone, and a bispecific antibody to a male human subject in need of treatment thereof with a dosing regimen comprising: a) an intravenous dose of the carboplatin as described in Paragraph F, and administered as described in Paragraphs G & J; b) an intravenous dose of the docetaxel as described in Paragraph I, and administered as described in Paragraphs G & K; c) an oral dose of the prednisone, as described in Paragraph L, is administered twice a day; d) an intravenous dose of the bispecific antibody as described in Paragraph E, administered on the first day of the dosing regimen, and administered over a time period described in Paragraphs C and D; wherein the bispecific antibody comprises a first monomer comprising SEQ ID NO: 1, a second monomer comprising SEQ ID NO: 2, and a light chain comprising SEQ ID NO: 3, wherein the dosing regimen is as described in Paragraph A and wherein the dosing regimen occurs as described in Paragraph B. In an exemplary embodiment, the invention is according to an embodiment of this paragraph, wherein the prostate cancer is mCRPC. In an exemplary embodiment, the invention is according to an embodiment of this paragraph, wherein the prostate cancer is mCRPC, and the mCRPC is positive for aberrancy for at least two of Rb1, TP53, and PTENb. In an exemplary embodiment, the invention is according to an embodiment of this paragraph, wherein the prostate cancer is mCRPC, and the mCRPC is a HRD/CDK12 mutation positive cancer, wherein, prior to the dosing regimen, a PARP inhibitor had been administered to the male human subject. In an exemplary embodiment, the invention is according to an embodiment of this paragraph, wherein a PARP inhibitor had been administered to the male human subject, and the male human subject had progressed after treatment. In an exemplary embodiment, the invention is according to an embodiment of this paragraph, wherein the mCRPC is a HRD/CDK12 mutation positive cancer, wherein, prior to the first occurrence of the dosing regimen, a PARP inhibitor had been administered to the male human subject. In an exemplary embodiment, the invention is according to an embodiment of this paragraph, wherein the prostate cancer is HRD positive in at least one gene selected from the group consisting of BRCA1, BRCA2, ATM, PALB2, CHEK2, and FANCA. In an exemplary embodiment, the invention is according to an embodiment of this paragraph, wherein the prostate cancer is positive for biallelic loss of CDK12. In an exemplary embodiment, the invention is according to an embodiment of this paragraph, wherein the prostate cancer is not positive for aberrancy for at least two of Rb1, TP53, and PTENb; not positive for HRD in one or more of the following genes: BRCA1, BRCA2, ATM, PALB2, CHEK2, FANCA; not positive for biallelic loss of CDK12; and not positive for microsatellite instability-high [MSI-H] or mismatch repair deficient [MMRD]. In an exemplary embodiment, the mCRPC is not positive for aberrancy for at least two of Rb1, TP53, and PTENb; not positive for HRD in one or more of the following genes: BRCA1, BRCA2, ATM, PALB2, CHEK2, FANCA; not positive for biallelic loss of CDK12; and not positive for microsatellite instability-high [MSI-H] or mismatch repair deficient [MMRD]. In an exemplary embodiment, the invention is according to an embodiment of this paragraph, wherein prior to the first occurrence of the dosage regimen, the male human subject has been administered at least two previous prostate cancer treatments, which are not the carboplatin, the cabazitaxel, the prednisone, or the bispecific antibody. In an exemplary embodiment, the invention is according to an embodiment of this paragraph, wherein prior to the first occurrence of the dosage regimen, an androgen suppression treatment had been administered to the male human subject. In an exemplary embodiment, the invention is according to an embodiment of this paragraph, wherein during an occurrence of the dosage regimen, an androgen suppression treatment is administered to the male human subject. In an exemplary embodiment, the invention is according to an embodiment of this paragraph, wherein the first monomer consists of SEQ ID NO: 1, the second monomer consists of SEQ ID NO: 2, and the light chain consists of SEQ ID NO: 3. In an exemplary embodiment, the invention is according to an embodiment of this paragraph, further comprising, prior to the administering, assessing the weight of the male human subject.

In an exemplary embodiment, provided herein is a method of achieving a positive therapeutic response against prostate cancer, comprising administering olaparib and a bispecific antibody to a male human subject in need of treatment thereof with a dosing regimen comprising: an intravenous dose of the bispecific antibody as described in Paragraph E, administered on the first day of the dosing regimen, and administered over a time period described in Paragraph D; and an oral dose of olaparib as described in Paragraph F, administered twice a day; wherein the bispecific antibody comprises a first monomer comprising SEQ ID NO: 1, a second monomer comprising SEQ ID NO: 2, and a light chain comprising SEQ ID NO: 3, wherein the dosing regimen is as described in Paragraph A and wherein the dosing regimen occurs as described in Paragraph B. In an exemplary embodiment, the invention is according to an embodiment of this paragraph, wherein the prostate cancer is mCRPC. In an exemplary embodiment, the invention is according to an embodiment of this paragraph, wherein the prostate cancer is mCRPC, and the mCRPC is a HRD/CDK12 mutation positive cancer, wherein, prior to the dosing regimen, a PARP inhibitor had not been administered to the male human subject. In an exemplary embodiment, the invention is according to an embodiment of this paragraph, wherein the mCRPC is a HRD/CDK12 mutation positive cancer, wherein, prior to the first occurrence of the dosing regimen, a PARP inhibitor had not been administered to the male human subject. In an exemplary embodiment, the invention is according to an embodiment of this paragraph, wherein the prostate cancer is HRD positive in at least one gene selected from the group consisting of BRCA1, BRCA2, ATM, PALB2, CHEK2, and FANCA. In an exemplary embodiment, the invention is according to an embodiment of this paragraph, wherein the prostate cancer is positive for biallelic loss of CDK12. In an exemplary embodiment, the invention is according to an embodiment of this paragraph, wherein prior to the first occurrence of the dosage regimen, the male human subject has been administered at least two previous prostate cancer treatments, neither of which are the olaparib or the bispecific antibody. In an exemplary embodiment, the invention is according to an embodiment of this paragraph, wherein prior to the first occurrence of the dosage regimen, an androgen suppression treatment had been administered to the male human subject. In an exemplary embodiment, the invention is according to an embodiment of this paragraph, wherein during an occurrence of the dosage regimen, an androgen suppression treatment is administered to the male human subject. In an exemplary embodiment, the invention is according to an embodiment of this paragraph, wherein the first monomer consists of SEQ ID NO: 1, the second monomer consists of SEQ ID NO: 2, and the light chain consists of SEQ ID NO: 3. In an exemplary embodiment, the invention is according to an embodiment of this paragraph, using the bispecific anti-CTLA4×anti-PD1 as provided herein (e.g., XmAb®717). In an exemplary embodiment, the invention is according to an embodiment of this paragraph, further comprising, prior to the administering, assessing the weight of the male human subject.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the prostate cancer is microsatellite instability-high (MSI-H) prostate cancer. In one embodiment, the prostate cancer is mismatch repair deficient (MMRD) prostate cancer. In one embodiment, the subject receives treatment about every 2 weeks (Q2W) for about two years.

In another aspect, provided herein is a method of treating an aggressive variant (anaplastic) adenocarcinoma of the prostate (AVPCa) in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the method further comprising orally administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating an aggressive variant (anaplastic) adenocarcinoma of the prostate (AVPCa) in a male human subject in need thereof, wherein the subject has not previously been administered docetaxel, and wherein the method comprises: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the method further comprises administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter.

In another aspect, provided herein is a method of treating a prostate cancer in a male human subject in need thereof, wherein the subject has not previously been administered docetaxel, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3; and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, wherein the subject has not previously been administered a PARP inhibitor, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the bispecific antibody comprises a first monomer comprising SEQ ID NO:1, a second monomer comprising SEQ ID NO:2, and a light chain comprising SEQ ID NO:3. In some embodiments, the method further comprising administering olaparib at a dose of about 300 mg. In some embodiments, the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In one embodiment, the prostate cancer is microsatellite instability-high (MSI-H) prostate cancer. In one embodiment, the prostate cancer is mismatch repair deficient (MMRD) prostate cancer. In one embodiment, the subject receives treatment about every 2 weeks (Q2W) for about two years.

In another aspect, provided herein is a method of treating an aggressive variant (anaplastic) adenocarcinoma of the prostate (AVPCa) in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the method further comprising orally administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating an aggressive variant (anaplastic) adenocarcinoma of the prostate (AVPCa) in a male human subject in need thereof, wherein the subject has not previously been administered docetaxel, and wherein the method comprises: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In one embodiment, the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In some embodiments, the method further comprises administering a steroid to the subject. In one embodiment, the steroid is prednisone administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a method of treating a prostate cancer in a male human subject in need thereof, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering cabazitaxel at a dose of about 20 mg/m², wherein the dose of the cabazitaxel is intravenously administered to the subject on day 1 of the first treatment cycle and about every three weeks (Q3W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter.

In another aspect, provided herein is a method of treating a prostate cancer in a male human subject in need thereof, wherein the subject has not previously been administered docetaxel, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter, and wherein the method further comprises: (a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) administering docetaxel at a dose of about 60 mg/m2 wherein the dose of the docetaxel is intravenously administered to the subject on day 1 of the first treatment cycle and about every three weeks (Q3W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In some embodiments, the subject is further administered a steroid. In some embodiments, the steroid is prednisone administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a method of treating a prostate cancer in a male human subject in need thereof, wherein the subject has not previously been administered a PARP inhibitor, the method comprising: administering to the subject according to a 28 day treatment cycle, a bispecific antibody at a dose of about 10 mg/kg, wherein the dose of the bispecific antibody is intravenously administered to the subject on day 1 of a first 28 day treatment cycle and about every two weeks (Q2W) thereafter. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In some embodiments, the method further comprising administering olaparib at a dose of about 300 mg. In some embodiments, the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

### 5.11 Additional Exemplary Embodiments for the Treatment of an Advanced Gynecologic or Genitourinary Malignancy in a Human Subject

In certain embodiments, provided is a method of treating an advanced solid cancerous tumor in a human subject, wherein the subject is administered and intravenous dose of a bispecific anti-CTLA4×anti-PD1 as provided herein (e.g., XmAb®717). In some embodiments, provided is a method of treating an advanced gynecologic malignancy in a human subject, wherein the subject is administered and intravenous dose of a bispecific anti-CTLA4×anti-PD1 as provided herein (e.g., XmAb®717). In some embodiments, provided is a method of treating an advanced genitourinary malignancy in a human subject, wherein the subject is administered and intravenous dose of a bispecific anti-CTLA4×anti-PD1 as provided herein (e.g., XmAb®717).

In certain embodiments, such as those provided below, the dose of the antibody is based upon the weight of the subject (body weight-based dosing). In other embodiments provided elsewhere herein, the dose of the antibody is a fixed dose.

In one exemplary embodiment, the intravenous dose is between about 0.05 mg/kg and about 12 mg/kg. In an exemplary embodiment, the intravenous dose is between about 0.15 mg/kg and about 10.0 mg/kg.

In an exemplary embodiment, the intravenous dose is between about 0.05 mg/kg and about 0.25 mg/kg. In an exemplary embodiment, the intravenous dose is between about 0.07 mg/kg and about 0.23 mg/kg. In an exemplary embodiment, the intravenous dose is between about 0.09 mg/kg and about 0.21 mg/kg. In an exemplary embodiment, the intravenous dose is between about 0.11 mg/kg and about 0.19 mg/kg. In an exemplary embodiment, the intravenous dose is between about 0.13 mg/kg and about 0.17 mg/kg. In an exemplary embodiment, the intravenous dose is about 0.15 mg/kg. In an exemplary embodiment, the intravenous dose is 0.15 mg/kg.

In an exemplary embodiment, the intravenous dose is between about 0.2 mg/kg and about 0.4 mg/kg. In an exemplary embodiment, the intravenous dose is between about 0.22 mg/kg and about 0.38 mg/kg. In an exemplary embodiment, the intravenous dose is between about 0.24 mg/kg and about 0.36 mg/kg. In an exemplary embodiment, the intravenous dose is between about 0.26 mg/kg and about 0.34 mg/kg. In an exemplary embodiment, the intravenous dose is between about 0.28 mg/kg and about 0.32 mg/kg. In an exemplary embodiment, the intravenous dose is about 0.3 mg/kg. In an exemplary embodiment, the intravenous dose is 0.3 mg/kg.

In an exemplary embodiment, the intravenous dose is between about 0.5 mg/kg and about 1.5 mg/kg. In an exemplary embodiment, the intravenous dose is between about 0.6 mg/kg and about 1.4 mg/kg. In an exemplary embodiment, the intravenous dose is between about 0.7 mg/kg and about 1.3 mg/kg. In an exemplary embodiment, the intravenous dose is between about 0.8 mg/kg and about 1.2 mg/kg. In an exemplary embodiment, the intravenous dose is between about 0.9 mg/kg and about 1.1 mg/kg. In an exemplary embodiment, the intravenous dose is about 1.0 mg/kg. In an exemplary embodiment, the intravenous dose is 1.0 mg/kg.

In an exemplary embodiment, the intravenous dose is between about 1.0 mg/kg and about 5.0 mg/kg. In an exemplary embodiment, the intravenous dose is between about 1.5 mg/kg and about 4.5 mg/kg. In an exemplary embodiment, the intravenous dose is between about 2.0 mg/kg and about 4.0 mg/kg. In an exemplary embodiment, the intravenous dose is between about 1.0 mg/kg and about 3.0 mg/kg. In an exemplary embodiment, the intravenous dose is between about 3.0 mg/kg and about 5.0 mg/kg. In an exemplary embodiment, the intravenous dose is between about 2.2 mg/kg and about 3.8 mg/kg. In an exemplary embodiment, the intravenous dose is between about 2.4 mg/kg and about 3.6 mg/kg. In an exemplary embodiment, the intravenous dose is between about 2.6 mg/kg and about 3.4 mg/kg. In an exemplary embodiment, the intravenous dose is between about 2.8 mg/kg and about 3.2 mg/kg. In an exemplary embodiment, the intravenous dose is between about 2.9 mg/kg and about 3.1 mg/kg. In an exemplary embodiment, the intravenous dose is about 3.0 mg/kg. In an exemplary embodiment, the intravenous dose is 3.0 mg/kg.

In an exemplary embodiment, the intravenous dose is between about 3.0 mg/kg and about 8.5 mg/kg. In an exemplary embodiment, the intravenous dose is between about 3.5 mg/kg and about 8.0 mg/kg. In an exemplary embodiment, the intravenous dose is between about 4.0 mg/kg and about 7.5 mg/kg. In an exemplary embodiment, the intravenous dose is between about 4.5 mg/kg and about 7.0 mg/kg. In an exemplary embodiment, the intravenous dose is between about 3.0 mg/kg and about 6.0 mg/kg. In an exemplary embodiment, the intravenous dose is between about 5.0 mg/kg and about 8.5 mg/kg. In an exemplary embodiment, the intravenous dose is between about 4.0 mg/kg and about 8.0 mg/kg. In an exemplary embodiment, the intravenous dose is between about 4.2 mg/kg and about 7.8 mg/kg. In an exemplary embodiment, the intravenous dose is between about 4.4 mg/kg and about 7.6 mg/kg. In an exemplary embodiment, the intravenous dose is between about 4.6 mg/kg and about 7.4 mg/kg. In an exemplary embodiment, the intravenous dose is between about 4.8 mg/kg and about 7.2 mg/kg. In an exemplary embodiment, the intravenous dose is between about 5.0 mg/kg and about 7.0 mg/kg. In an exemplary embodiment, the intravenous dose is between about 5.2 mg/kg and about 6.8 mg/kg. In an exemplary embodiment, the intravenous dose is between about 5.4 mg/kg and about 6.6 mg/kg. In an exemplary embodiment, the intravenous dose is between about 5.6 mg/kg and about 6.4 mg/kg. In an exemplary embodiment, the intravenous dose is between about 5.8 mg/kg and about 6.2 mg/kg. In an exemplary embodiment, the intravenous dose is about 6.0 mg/kg. In an exemplary embodiment, the intravenous dose is 6.0 mg/kg.

In an exemplary embodiment, the intravenous dose is between about 8.0 mg/kg and about 12.0 mg/kg. In an exemplary embodiment, the intravenous dose is between about 8.3 mg/kg and about 11.7 mg/kg. In an exemplary embodiment, the intravenous dose is between about 8.6 mg/kg and about 11.4 mg/kg. In an exemplary embodiment, the intravenous dose is between about 8.9 mg/kg and about 11.1 mg/kg. In an exemplary embodiment, the intravenous dose is between about 9.2 mg/kg and about 10.8 mg/kg. In an exemplary embodiment, the intravenous dose is between about 9.5 mg/kg and about 10.5 mg/kg. In an exemplary embodiment, the intravenous dose is between about 9.8 mg/kg and about 10.2 mg/kg. In an exemplary embodiment, the intravenous dose is about 10.0 mg/kg. In an exemplary embodiment, the intravenous dose is 10.0 mg/kg.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms provided herein are dictated by and directly dependent on (a) the unique characteristics of the active compound and the particular therapeutic effect to be achieved, and (b) the limitations inherent in the art of compounding such an active compound for the treatment of sensitivity in individuals.

In another aspect, provided herein is a method of treating an advanced gynecologic or genitourinary malignancy in a human subject in need thereof, the method comprising administering to the subject a dose of a bispecific antibody according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle. In one embodiment, the bispecific antibody is an anti-CTLA4×anti-PD1 bispecific antibody that comprises a first monomer comprising SEQ ID NO:1, a second monomer comprising SEQ ID NO:2, and a light chain comprising SEQ ID NO:3. In one embodiment, the bispecific antibody is the anti-CTLA4×anti-PD1 antibody, XmAb®717 (vudalimab). In one embodiment, the bispecific antibody is a biosimilar of XmAb®717 (vudalimab). In one embodiment, the bispecific antibody is a biobetter of XmAb®717 (vudalimab). In one embodiment, the bispecific antibody is a bioequivalent of XmAb®717 (vudalimab). In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments, the subject is intravenously administered the dose of the bispecific antibody at a constant infusion rate over the course of one hour. In some embodiments of the methods provided herein, if the weight of the subject changes by more than 10% from baseline, the subject is optionally reassigned to a new dosing level and one or more subsequent doses are administered to the subject at the new dosing level. In some embodiments of the methods provided herein, if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

### 5.12 Selection of Human Subjects

In various embodiments of the methods provided herein, human subjects can be selected based on criteria described herein.

5.12.1. Exemplary Selection of Human Subjects Having a Prostate Cancer

Human subjects can be selected based on criteria described herein. In an exemplary embodiment, the human subject is male. In an exemplary embodiment, the human subject is 18 years or older. In an exemplary embodiment, the human subject is between the ages of 18 years and 100 years. In an exemplary embodiment, the human subject is between the ages of 18 years and 80 years. In an exemplary embodiment, the human subject is between the ages of 45 years and 90 years. In an exemplary embodiment, the human subject is between the ages of 45 years and 80 years. In an exemplary embodiment, the human subject is between the ages of 50 years and 90 years. In an exemplary embodiment, the human subject is between the ages of 55 years and 85 years. In an exemplary embodiment, the human subject is between the ages of 60 years and 80 years. In an exemplary embodiment, the human subject is between the ages of 60 years and 70 years.

In an exemplary embodiment, the male human subject has progressive metastatic castration-resistant prostate cancer. In an exemplary embodiment, the male human subject has experienced PSA progression. In an exemplary embodiment, the male human subject is able to provide written informed consent. In an exemplary embodiment, the male human subject is 18 years or older.

In an exemplary embodiment, the male human subject has histologically confirmed diagnosis of carcinoma of the prostate. In an exemplary embodiment, the male human subject has progressive mCRPC based on PSA progression, defined as at least 2 rises in PSA with a minimum of a one week interval. In an exemplary embodiment, the male human subject has progressive mCRPC based on PSA progression, defined as at least 2 rises in PSA with a minimum of a 1 week interval, wherein 1.0 ng/mL is the minimal starting value if confirmed rise is the only indication of progression. In an exemplary embodiment, the human subject has progressive mCRPC based on soft-tissue progression per RECIST 1.1. In an exemplary embodiment, the male human subject has progressive mCRPC based on progression of bone disease (evaluable disease) or 2 or more new bone lesions by bone scan.

In an exemplary embodiment, prior to the first occurrence of the dosing regimen, a checkpoint inhibitor had been administered to the human subject. In an exemplary embodiment, the human subject has received treatment with any cytotoxic T-lymphocyte-associated protein (CTLA4), PD1, PDL1, or programmed cell death ligand 2 (PDL2) directed immunotherapy. In an exemplary embodiment, the human subject has been administered at least one previous prostate cancer treatment, which was a checkpoint inhibitor. In an exemplary embodiment, the human subject has been administered one previous prostate cancer treatment, which was a checkpoint inhibitor. In an exemplary embodiment, the human subject has been administered at least two previous prostate cancer treatments, one of which was a checkpoint inhibitor, and the other is not the anti-CTLA4×anti-PD1 bispecific antibody described herein. In an exemplary embodiment, the human subject has been administered two previous prostate cancer treatments, one of which was a checkpoint inhibitor, and the other is not the anti-CTLA4×anti-PD1 bispecific antibody described herein. In an exemplary embodiment, the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody. In an exemplary embodiment, the human subject has progressive mCRPC based on progression after treatment with at least 2 prior lines of anticancer therapy approved for treatment of metastatic prostate cancer.

In an exemplary embodiment, prior to the first occurrence of the dosing regimen, a PARP inhibitor has been administered to the human subject. In an exemplary embodiment, prior to the first occurrence of the dosing regimen, a PARP inhibitor has been administered to the human subject, and the human subject has progressed. In an exemplary embodiment, the human subject has been administered at least one previous prostate cancer treatment, which is not the carboplatin, the cabazitaxel, the prednisone, or the anti-CTLA4×anti-PD1 bispecific antibody described herein. In an exemplary embodiment, the male human subject has been administered one previous prostate cancer treatment, which is neither the carboplatin, the cabazitaxel, the prednisone, or the anti-CTLA4×anti-PD1 bispecific antibody described herein. In an exemplary embodiment, the male human subject has been administered at least two previous prostate cancer treatments, neither of which are the carboplatin, the cabazitaxel, the prednisone, or the anti-CTLA4×anti-PD1 bispecific antibody described herein. In an exemplary embodiment, the male human subject has been administered two previous prostate cancer treatments, neither of which are the carboplatin, the cabazitaxel, the prednisone, or the anti-CTLA4×anti-PD1 bispecific antibody described herein. In an exemplary embodiment, the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody. In an exemplary embodiment, the male human subject has progressive mCRPC based on progression after treatment with at least 2 prior lines of anticancer therapy approved for treatment of metastatic prostate cancer.

In an exemplary embodiment, prior to the first occurrence of the dosing regimen, a PARP inhibitor had not been administered to the male human subject. In an exemplary embodiment, the male human subject has been administered at least one previous prostate cancer treatment, which is neither the olaparib nor the anti-CTLA4×anti-PD1 bispecific antibody described herein. In an exemplary embodiment, the male human subject has been administered one previous prostate cancer treatment, which is neither the olaparib nor the anti-CTLA4×anti-PD1 bispecific antibody described herein. In an exemplary embodiment, the male human subject has been administered at least two previous prostate cancer treatments, neither of which are the olaparib or the anti-CTLA4×anti-PD1 bispecific antibody described herein. In an exemplary embodiment, the male human subject has been administered two previous prostate cancer treatments, neither of which are the olaparib or the anti-CTLA4×anti-PD1 bispecific antibody described herein. In an exemplary embodiment, the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody. In an exemplary embodiment, the male human subject has progressive mCRPC based on progression after treatment with at least 2 prior lines of anticancer therapy approved for treatment of metastatic prostate cancer.

In an exemplary embodiment, the previous prostate cancer treatment is a luteinizing hormone-releasing hormone analog which is a luteinizing hormone-releasing hormone agonist. In an exemplary embodiment, the luteinizing hormone-releasing hormone agonist is a peptide. In an exemplary embodiment, the luteinizing hormone-releasing hormone agonist is azagly-nafarelin, buserelin, deslorelin, fertirelin, GnRH, gonadorelin, goserelin, histrelin, lecirelin, leuprorelin, nafarelin, peforelin, or triptorelin. In an exemplary embodiment, the luteinizing hormone-releasing hormone agonist is buserelin, goserelin, histrelin, leuprorelin, or triptorelin.

In an exemplary embodiment, the previous prostate cancer treatment is a luteinizing hormone-releasing hormone analog which is a luteinizing hormone-releasing hormone antagonist. In an exemplary embodiment, the luteinizing hormone-releasing hormone antagonist is a peptide. In an exemplary embodiment, the luteinizing hormone-releasing hormone antagonist is abarelix, cetrorelix, degarelix, ganirelix, or ozarelix. In an exemplary embodiment, the luteinizing hormone-releasing hormone antagonist is degarelix. In an exemplary embodiment, the luteinizing hormone-releasing hormone antagonist is not a peptide. In an exemplary embodiment, the luteinizing hormone-releasing hormone antagonist is elagolix, linzagolix, opigolix, relugolix, or sufugolix.

In an exemplary embodiment, a previous prostate cancer treatment is a platinum chemotherapeutic. In an exemplary embodiment, the platinum chemotherapeutic is carboplatin, cisplatin, or oxaliplatin.

In an exemplary embodiment, a previous prostate cancer treatment is an androgen receptor signaling inhibitor (ARSI). In an exemplary embodiment, the ARSI is a first generation ARSI. In an exemplary embodiment, the first generation ARSI is bicalutamide or flutamide. In an exemplary embodiment, the ARSI is abiraterone, enzalutamide, apalutamide or darolutamide.

In an exemplary embodiment, a previous prostate cancer treatment is a bone-targeting radionuclide. In an exemplary embodiment, the bone-targeting radionuclide is radium-223. In an exemplary embodiment, the bone-targeting radionuclide is radium-223 chloride.

In an exemplary embodiment, a previous prostate cancer treatment is sipuleucel-T.

In an exemplary embodiment, a previous prostate cancer treatment is a checkpoint inhibitor antibody. In an exemplary embodiment, the checkpoint inhibitor antibody is an anti-PD1 antibody, an anti-CTLA4 antibody, or an anti-LAG3 antibody.

In an exemplary embodiment, the human subject has recovered from any toxicity related to previous anticancer treatment to ≤Grade 2. In an exemplary embodiment, the human subject is on androgen suppression treatment (e.g., luteinizing hormone-releasing hormone agonist) with castrate level of testosterone (≤50 ng/dL). In an exemplary embodiment, the human subject is on androgen suppression treatment (e.g., luteinizing hormone-releasing hormone agonist) with castrate level of testosterone (≤50 ng/dL) and be willing to continue such treatment. In an exemplary embodiment, the human subject has had a surgical orchiectomy. In an exemplary embodiment, the male human subject has had a surgical orchiectomy, and is not on androgen suppression treatment (e.g., luteinizing hormone-releasing hormone agonist). In an exemplary embodiment, the human subject has not had a surgical orchiectomy. In an exemplary embodiment, the human subject has not had a surgical orchiectomy and is on androgen suppression treatment (e.g., luteinizing hormone-releasing hormone agonist). In an exemplary embodiment, the human subject has a previously treated brain metastases which is radiologically stable (i.e., are without evidence of progression for at least 4 weeks by repeat imaging (note that the repeat imaging should be performed during study screening), are clinically stable, and are without requirement of steroid treatment for at least 14 days prior to first dose of study treatment.

In an exemplary embodiment, the human subject has had a histologically confirmed diagnosis of a prostate cancer described herein, methods of screening for which are known in the art. In an exemplary embodiment, the human subject has been diagnosed with a MSI-H and/or MMRD positive cancer, methods of screening for which are known in the art. In an exemplary embodiment, the human subject has been diagnosed with a HRD/CDK12 mutation positive prostate cancer, methods of screening for which are known in the art. In an exemplary embodiment, the human subject has been diagnosed with a prostate cancer which is positive for biallelic loss of CDK12, methods of screening for which are known in the art. In an exemplary embodiment, the human subject has been diagnosed with a prostate cancer which is HRD positive in at least one gene selected from the group consisting of BRCA1, BRCA2, ATM, PALB2, CHEK2, and FANCA, methods of screening for which are known in the art.

In an exemplary embodiment, the human subject has evaluable disease according to Prostate Cancer Working Group (PCWG3) Guidelines. In an exemplary embodiment, the human subject has an Eastern Cooperative Oncology Group (ECOG) performance status of 0 or 1, wherein 0=Normal activity—Fully active, able to carry on all predisease performance without restriction; 1=Symptoms, but ambulatory, restricted in physically strenuous activity, but ambulatory and able to carry out work of a light or sedentary nature (e.g., light housework, office work); 2=In bed<50% of the time, ambulatory and capable of all self-care, but unable to carry out any work activities, up and about more than 50% of waking hours; 3=In bed>50% of the time, capable of only limited self-care, confined to bed or chair more than 50% of waking hours; 4=100% bedridden, completely disabled, cannot carry on any self-care, totally confined to bed or chair; 5=Dead. In an exemplary embodiment, the human subject has adequate archival metastatic tumor tissue. In an exemplary embodiment, the human subject agrees to undergo a biopsy of at least 1 metastatic site. In an exemplary embodiment, the human subject agrees to undergo a fresh biopsy of primary prostate. In an exemplary embodiment, the human subject agrees to undergo a fresh biopsy of primary prostate, however, such a fresh biopsy is possible if there is clear local disease and no other measurable disease site or biopsiable bone lesion. In an exemplary embodiment, the human subject is taking prednisone from a previous prostate cancer therapy. In an exemplary embodiment, the human subject is hepatitis C virus [HCV] antibody positive but HCV RNA negative due to documented, curative prior antiviral treatment or natural resolution. In an exemplary embodiment, the human subject whose HBsAg is negative and HBcAb is positive may be treated if a hepatitis B virus [HBV] DNA test is negative and the human subject is retested for HBsAg and HBV DNA every 2 months.

In an exemplary embodiment, the human subject is not currently receiving anticancer therapies other than androgen deprivation therapy. In an exemplary embodiment, the human subject has not received treatment with any other anticancer therapy (i.e., other immunotherapy, chemotherapy, radiation therapy, etc.) within 2 weeks of the first administration of the first occurrence of the dosing regimen. In an exemplary embodiment, the human subject has not received treatment with any cytotoxic T-lymphocyte-associated protein (CTLA4), PD1, PDL1, or programmed cell death ligand 2 (PDL2) directed immunotherapy. In an exemplary embodiment, the human subject does not have known active central nervous system metastases and/or carcinomatous meningitis. In an exemplary embodiment, the human subject does not have a Platelet count<100×10⁹/L. In an exemplary embodiment, the human subject does not have a Hemoglobin level≤9.0 g/dL. In an exemplary embodiment, the human subject does not have an absolute neutrophil count≤1.7×10⁹ for subjects who will receive cabazitaxel; <1.0×10⁹/L for all others. In an exemplary embodiment, the human subject does not have an aspartate aminotransferase at screening>3×upper limit of normal (ULN) for subjects without known liver involvement by tumor or >5×ULN for subjects with known liver involvement by tumor. In an exemplary embodiment, the human subject does not have an alanine aminotransferase at screening>3×ULN for subjects without known liver involvement by tumor or >5×ULN for subjects with known liver involvement by tumor. In an exemplary embodiment, the human subject does not have bilirubin≥1.5×ULN (unless prior diagnosis and documentation of ongoing hemolysis or Gilbert's syndrome has been made). In an exemplary embodiment, the human subject does not have an estimated creatinine clearance<50 mL/minute calculated by the Cockcroft Gault or Modification of Diet in Renal Disease formulas. In an exemplary embodiment, the human subject does not have active known or suspected autoimmune disease (except vitiligo; type 1 diabetes mellitus or residual hypothyroidism due to an autoimmune condition that is treatable with hormone replacement therapy only; psoriasis, atopic dermatitis, or another autoimmune skin condition that is managed without systemic therapy; or arthritis that is managed without systemic therapy beyond oral acetaminophen and nonsteroidal anti-inflammatory drugs). In an exemplary embodiment, the human subject does not have a condition requiring systemic treatment with corticosteroids, prednisone equivalents, or other immunosuppressive medications within 14 days prior to first dose of the anti-CTLA4×anti-PD1 bispecific antibody described herein (except inhaled or topical corticosteroids or brief courses of corticosteroids given for prophylaxis of contrast dye allergic response). In an exemplary embodiment, the human subject does not have a condition requiring systemic treatment with corticosteroids, prednisone equivalents, or other immunosuppressive medications within 14 days prior to first dose of the carboplatin, the cabazitaxel, the prednisone, or the anti-CTLA4×anti-PD1 bispecific antibody described herein (except inhaled or topical corticosteroids or brief courses of corticosteroids given for prophylaxis of contrast dye allergic response). In an exemplary embodiment, the human subject does not have a condition requiring systemic treatment with corticosteroids, prednisone equivalents, or other immunosuppressive medications within 14 days prior to first dose of olaparib or the anti-CTLA4×anti-PD1 bispecific antibody described herein (except inhaled or topical corticosteroids or brief courses of corticosteroids given for prophylaxis of contrast dye allergic response). In an exemplary embodiment, the human subject has not received an organ allograft. In an exemplary embodiment, the human subject does not have known history of left ventricular ejection fraction≤40%. In an exemplary embodiment, the human subject does not have a history or evidence of any other clinically unstable/uncontrolled disorder, condition, or disease other than their primary malignancy that would pose a risk to patient safety or interfere with study evaluations, procedures, or completion. In an exemplary embodiment, the human subject does not have evidence of any serious bacterial, viral, parasitic, or systemic fungal infections within the 30 days prior to the first dose of the anti-CTLA4×anti-PD1 bispecific antibody described herein. In an exemplary embodiment, the human subject has not received a live-virus vaccine within 30 days prior to the first dose of first dose of the anti-CTLA4×anti-PD1 bispecific antibody described herein, with the exception of seasonal flu vaccines that do not contain live virus. In an exemplary embodiment, the human subject does not have evidence of any serious bacterial, viral, parasitic, or systemic fungal infections within the 30 days prior to the first dose of the carboplatin, the cabazitaxel, the prednisone, or the anti-CTLA4×anti-PD1 bispecific antibody described herein. In an exemplary embodiment, the human subject has not received a live-virus vaccine within 30 days prior to the first dose of first dose of the carboplatin, the cabazitaxel, the prednisone, or the anti-CTLA4×anti-PD1 bispecific antibody described herein, with the exception of seasonal flu vaccines that do not contain live virus. In an exemplary embodiment, the human subject does not have evidence of any serious bacterial, viral, parasitic, or systemic fungal infections within the 30 days prior to the first dose of olaparib or the anti-CTLA4×anti-PD1 bispecific antibody described herein. In an exemplary embodiment, the human subject has not received a live-virus vaccine within 30 days prior to the first dose of first dose of olaparib or the anti-CTLA4×anti-PD1 bispecific antibody described herein, with the exception of seasonal flu vaccines that do not contain live virus. In an exemplary embodiment, the human subject is not human immunodeficiency virus (HIV) positive subject with CD4+ T-cell (CD4+) counts<350 cells/μL. In an exemplary embodiment, the human subject does not have an HIV viral load greater than 400 copies/mL. In an exemplary embodiment, the human subject does not have a history of an AIDS (acquired immunodeficiency syndrome)-defining opportunistic infection within the past 12 months. In an exemplary embodiment, the human subject has not been on established antiretroviral therapy (ART) for at least 4 weeks prior to initiation of study drug dosing. (Effective ART is defined as a drug, dosage, and schedule associated with reduction and control of the viral load). In an exemplary embodiment, the human subject has not had a positive test for hepatitis C RNA. In an exemplary embodiment, the human subject has not had a positive test for hepatitis B surface antigen (HBsAg) or hepatitis B core antibody (HBcAb).

Blood samples can be collected from a human subject using any method known in the art, e.g., by venipuncture or fingerstick. Particular types of blood cells can be isolated, expanded, frozen, and used at a later time. Tissue samples can be obtained from a human subject using any method known in the art, e.g., by biopsy or surgery. CT imaging, ultrasound, or an endoscope can be used to guide this type of procedure. The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

5.12.2. Exemplary Selection of Human Subjects Having an Advanced Gynecologic or Genitourinary Malignancies

Human subjects can be selected based on criteria described herein. In an exemplary embodiment, the human subject is male. In an exemplary embodiment, the human subject is female. In an exemplary embodiment, the human subject is 18 years or older. In an exemplary embodiment, the human subject is between the ages of 18 years and 100 years. In an exemplary embodiment, the human subject is between the ages of 18 years and 80 years. In an exemplary embodiment, the human subject is between the ages of 45 years and 90 years. In an exemplary embodiment, the human subject is between the ages of 45 years and 80 years. In an exemplary embodiment, the human subject is between the ages of 50 years and 90 years. In an exemplary embodiment, the human subject is between the ages of 55 years and 85 years. In an exemplary embodiment, the human subject is between the ages of 60 years and 80 years. In an exemplary embodiment, the human subject is between the ages of 60 years and 70 years.

In one embodiment, the subject has platinum-resistant high-grade serous ovarian cancer (HGSOC).

In one embodiment, the subject has chemotherapy relapsed or refractory clear cell ovarian, endometrial, or peritoneal cancer. In one embodiment, the subject has immune-checkpoint-inhibitor-refractory microsatellite stable (MSS) endometrial cancer (EC). In one embodiment, the subject has previously treated recurrent or metastatic cervical cancer. In one embodiment, the subject has high-risk metastatic castration-resistant prostate cancer (mCRPC)

In certain embodiments, subjects receive XmAb®20717 on Day 1 of each 21-day cycle. In one embodiment, subjects who weigh 80 kilograms or more receive 1200 mg of XmAb®20717 intravenously (IV) every 3 weeks (Q3W) and subjects who weigh less than 80 kilograms receive 1000 mg XmAb®20717 IV Q3W. In certain embodiments, if, at any time after study drug administration has begun, the subject's weight changes by more than 10% from baseline (excluding weight attributable to ascites and/or peripheral edema), and the new weight requires reassignment to a different dosing tier, subsequent doses are received at the new dose level until such time as fulfillment of these rules may again require assignment to the other dose level.

In some embodiments, the subject must be able to provide written informed consent. In some embodiments, the cancer must have progressed after treatment with all approved and medically appropriate therapies or have no appropriate available therapies. In some embodiments, the subject has a histologically confirmed diagnosis of one of the following tumor types, along with clinical/pathologic confirmation of the additional requirements for each indication, as appropriate: (i) persistent or recurrent high-grade serous carcinoma of the ovary, fallopian tube, or peritoneum after treatment with platinum-based systemic chemotherapy (except subjects with a diagnosis of carcinosarcoma), (ii) persistent or recurrent clear cell carcinoma of the ovary, peritoneum, or endometrium after treatment with platinum-based systemic chemotherapy; (iii) advanced endometrial carcinoma that is not microsatellite instability-high (MSI-H) or deficient mismatch repair (dMMR) in patients who are not candidates for curative surgery or radiation, and that has progressed following treatment with no more than one prior line of systemic therapy and prior treatment with FDA-approved combination therapy consisting of a checkpoint inhibitor and a targeted agent, (iv) recurrent or metastatic cervical carcinoma previously treated with standard-of-care systemic chemotherapy and FDA-approved immunotherapy, if eligible, or (v) high-risk metastatic castration-resistant prostate cancer. In some embodiments, castration resistance is defined as progressive disease after surgical castration, or progression in the setting of medical androgen ablation with a castrate level of testosterone (<50 ng/dL). In some embodiments, high-risk disease is any visceral, soft tissue, or lymph node metastasis(es) with/without bone metastases.

In some embodiments, the subject has measurable disease by Response Evaluation Criteria in Solid Tumors (RECIST 1.1). In some embodiments, the subject has Eastern Cooperative Oncology Group performance status of 0 or 1.

In some embodiments, the subject does not currently receive other anticancer therapies, except that subjects with prostate cancer may continue to receive luteinizing hormone-releasing hormone (LHRH) analogue therapy.

In some embodiments, the subject has not had more than 2 prior chemotherapy. regimens for subjects in the cervical cancer, CCC, HGSOC, or prostate cancer cohorts.

In some embodiments, the subject has not had prior treatment with a CTLA4-targeted agent.

In some embodiments, the subject has not had prior treatment with nivolumab, pembrolizumab, or any other PD1-, PDL1- or programmed cell death ligand 2- (PDL2)-directed therapy. In certain embodiments, subjects with MSS EC may have received anti-PD1 therapy as part of an FDA-approved regimen in the approved disease setting. In some embodiments, subjects with cervical cancer may have received anti-PD1 therapy as an FDA-approved agent in the approved disease setting.

In some embodiments, the subject has not had treatment with any other anticancer therapy within 2 weeks of the start of study drug (i.e., other immunotherapy, chemotherapy, radiation therapy, etc.).

In some embodiments, the subject has not had a life-threatening (Grade 4) immune-mediated adverse event (AE) associated with prior administration of an immunotherapy agent.

In some embodiments, the subject has not failed to recover from any immunotherapy-related toxicity from prior cancer therapy to ≤Grade 1, except that subjects are eligible if a previous immunotherapy-related endocrinopathy is medically managed with hormone replacement therapy only.

In some embodiments, the subject has not failed to recover from any other cancer therapy-related toxicity (other than immune-related toxicity) related to previous anticancer treatment to ≤Grade 2.

In some embodiments, the subject does not have an active central nervous system metastases and/or carcinomatous meningitis.

In some embodiments, the subject does not have a platelet count<100×109/L. In some embodiments, the subject does not have a hemoglobin level≤9.0 g/dL. In some embodiments, the subject does not have an absolute neutrophil count<1.5×109/L. In some embodiments, the subject does not have an aspartate aminotransferase (AST) at screening>3×upper limit of normal (ULN) for subjects without known liver involvement by tumor; or >5×ULN for subjects with known liver involvement by tumor. In some embodiments, the subject does not have an alanine aminotransferase (ALT) at screening>3×ULN for subjects without known liver involvement by tumor; or >5×ULN for subjects with known liver involvement by tumor. In some embodiments, the subject does not have a bilirubin≥1.5×ULN (unless prior diagnosis and documentation of ongoing hemolysis or Gilbert's syndrome has been made). In some embodiments, the subject does not have an estimated creatinine clearance<50 mL/minute calculated by the Cockcroft Gault or Modification of Diet in Renal Disease formulas.

In some embodiments, the subject does not have an active known or suspected autoimmune disease (except that subjects are permitted to enroll if they have vitiligo; type 1 diabetes mellitus or residual hypothyroidism due to an autoimmune condition that is treatable with hormone replacement therapy only; autoimmune adrenal insufficiency that is managed with low-dose corticosteroids; psoriasis, atopic dermatitis, or another autoimmune skin condition that is managed without systemic therapy; or arthritis that is managed without systemic therapy beyond oral acetaminophen and nonsteroidal anti-inflammatory drugs).

In some embodiments, the subject does not have any condition requiring systemic treatment with corticosteroids, prednisone equivalents, or other immunosuppressive medications within 14 days prior to first dose of study drug (except that inhaled or topical corticosteroids or brief courses of corticosteroids given for prophylaxis of contrast dye allergic response are permitted).

In some embodiments, the subject has not had receipt of an organ allograft.

In some embodiments, the subject does not have a history of small or large bowel obstruction within 3 months of enrollment, including subjects with palliative gastric drainage catheters. Subjects with palliative diverting ileostomy or colostomy can be allowed if they have been symptom-free for more than 3 months.

In some embodiments, the subject does not have an ongoing bowel perforation or presence of bowel fistula or intra-abdominal abscess.

In some embodiments, the subject does not have refractory ascites, for example, ascites needing drainage catheter or therapeutic paracentesis more often than every 4 weeks.

In some embodiments, the subject does not have a histologic diagnosis of carcinosarcoma of the ovary.

In some embodiments, the subject does not have a symptomatic cord compression, or clinical or radiologic findings indicative of impending cord compression.

In some embodiments, the subject does not have a history or evidence of any other clinically unstable/uncontrolled disorder, condition, or disease (including, but not limited to, cardiopulmonary, renal, metabolic, hematologic, or psychiatric) other than their primary malignancy.

In some embodiments, the subject does not have evidence of any serious bacterial, viral, parasitic, or systemic fungal infections within the 30 days prior to the first dose of study drug.

In some embodiments, the subject has not had receipt of a live-virus vaccine within 30 days prior to first dose of study drug (seasonal flu and COVID-19 vaccines are permitted, as long as they do not contain live virus and are not administered within 24 hours of planned administration of XmAb®20717).

In some embodiments, the subject is not an HIV-positive subject with CD4+ T-cell (CD4+) counts<350 cells/μL, or an HIV viral load greater than 400 copies/mL, or a history of an acquired immunodeficiency syndrome (AIDS)-defining opportunistic infection within the past 12 months, or who has not been on established antiretroviral therapy (ART) for at least 4 weeks prior to initiation of study drug dosing.

In some embodiments, the subject does not have a positive test for hepatitis C RNA.

In some embodiments, the subject does not have a positive test for hepatitis B surface antigen (HBsAg) or hepatitis B core antibody (HBcAb).

In some embodiments, the subject is not pregnant or breastfeeding. In some embodiments, the subject does not have a positive urine pregnancy test (i.e., urine human chorionic gonadotropin).

### 5.13 Kits

In another aspect, provided herein is a kit comprising a bispecific anti-PD1×CTLA4 antibody, and instructions for administering said bispecific antibody to a subject.

In another aspect, provided herein is a kit comprising a bispecific anti-PD1×CTLA4 antibody, and instructions for use of said bispecific antibody in the treatment of a prostate cancer in a male human subject. In some embodiments, the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In a specific embodiment, the bispecific antibody comprises a first monomer comprising SEQ ID NO:1, a second monomer comprising SEQ ID NO:2, and a light chain comprising SEQ ID NO:3. In one embodiment, the prostate cancer is microsatellite instability-high (MSI-H) prostate cancer. In one embodiment, the prostate cancer is mismatch repair deficient (MMRD) prostate cancer. In one embodiment, the instructions provide that the subject receives treatment about every 2 weeks (Q2W) for about two years.

In another aspect, provided herein is a kit comprising a bispecific anti-PD1×CTLA4 antibody, and instructions for use of said bispecific antibody in the treatment of an aggressive variant (anaplastic) adenocarcinoma of the prostate (AVPCa) in a male human subject. In some embodiments, the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In some embodiments, the bispecific antibody comprises a first monomer comprising SEQ ID NO:1, a second monomer comprising SEQ ID NO:2, and a light chain comprising SEQ ID NO:3. In one embodiment, the instructions further provide that: (a) carboplatin is administered at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) cabazitaxel is administered at a dose of about 20 mg/m², wherein the dose of the cabazitaxel is intravenously administered to the subject on day 1 of the first treatment cycle and about every three weeks (Q3W) thereafter. In one embodiment, the instructions further provide that a steroid is orally administered to the subject. In one embodiment, the steroid is prednisone, and the instructions provide that the steroid is administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a kit comprising a bispecific anti-PD1×CTLA4 antibody, and instructions for use of said bispecific antibody in the treatment of an aggressive variant (anaplastic) adenocarcinoma of the prostate (AVPCa) in a male human subject, wherein the subject has not previously been administered docetaxel, and wherein the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In some embodiments, the bispecific antibody comprises a first monomer comprising SEQ ID NO:1, a second monomer comprising SEQ ID NO:2, and a light chain comprising SEQ ID NO:3. In one embodiment, the instructions further provide that: (a) carboplatin is administered at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) docetaxel is administered at a dose of about 60 mg/m2 wherein the dose of the docetaxel is intravenously administered to the subject on day 1 of the first treatment cycle and about every three weeks (Q3W) thereafter. In some embodiments, the instructions further provide that a steroid is administered to the subject. In one embodiment, the steroid is prednisone, and the instructions provide that the steroid is administered at a dose of about 5 mg twice per day (b.i.d.) on day 1 of the first treatment cycle, and about twice per day (b.i.d.) thereafter. In some embodiments, the cancer has a mutation or other aberrancy in at least two genes independently selected from the group consisting of Rb1, TP53 and PTEN. In some embodiments, the subject receives more than one 28 day treatment cycle. In some embodiments, the subject receives up to twenty-four 28 day treatment cycles.

In another aspect, provided herein is a kit comprising a bispecific anti-PD1×CTLA4 antibody, and instructions for use of said bispecific antibody in the treatment of a prostate cancer in a male human subject in need thereof, wherein the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In some embodiments, the bispecific antibody comprises a first monomer comprising SEQ ID NO:1, a second monomer comprising SEQ ID NO:2, and a light chain comprising SEQ ID NO:3. In certain embodiments, the instructions further provide that: (a) carboplatin is administered at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) cabazitaxel is administered at a dose of about 20 mg/m², wherein the dose of the cabazitaxel is intravenously administered to the subject on day 1 of the first treatment cycle and about every three weeks (Q3W) thereafter. In some embodiments, the instructions further provide that a steroid is administered to the subject. In one embodiment, the steroid is prednisone, and the instructions provide that the steroid is administered at a dose of about 5 mg twice per day (b.i.d.) on day 1 of the first treatment cycle, and about twice per day (b.i.d.) thereafter.

In another aspect, provided herein is a kit comprising a bispecific anti-PD1×CTLA4 antibody, and instructions for use of said bispecific antibody in the treatment of a prostate cancer in a male human subject in need thereof, wherein the subject has not previously been administered docetaxel, and wherein the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In some embodiments, the bispecific antibody comprises a first monomer comprising SEQ ID NO:1, a second monomer comprising SEQ ID NO:2, and a light chain comprising SEQ ID NO:3. In certain embodiments, the instructions further provide that: (a) carboplatin is administered at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and (b) docetaxel is administered at a dose of about 60 mg/m² wherein the dose of the docetaxel is intravenously administered to the subject on day 1 of the first treatment cycle and about every three weeks (Q3W) thereafter. In some embodiments, the instructions further provide that a steroid is administered to the subject. In one embodiment, the steroid is prednisone, and the instructions provide that the steroid is administered at a dose of about 5 mg twice per day (b.i.d.) on day 1 of the first treatment cycle, and about twice per day (b.i.d.) thereafter. In some embodiments, the subject has received prior treatment with a polyadenosine diphosphate ribose polymerase (PARP) inhibitor. In some embodiments, the cancer has a homologous recombination deficiency (HRD). In some embodiments, the cancer has a biallelic loss of cyclin-dependent kinase 12 (CDK12).

In another aspect, provided herein is a kit comprising a bispecific anti-PD1×CTLA4 antibody, and instructions for use of said bispecific antibody in the treatment of a prostate cancer in a male human subject in need thereof, wherein the subject has not previously been administered a PARP inhibitor, and wherein the instructions provide that the bispecific antibody is administered to the subject according to a 28 day treatment cycle at a dose of about 10 mg/kg, wherein the dose of the bispecific antibody is intravenously administered to the subject on day 1 of a first 28 day treatment cycle and about every two weeks (Q2W) thereafter. In certain embodiments, the bispecific antibody comprises a first monomer comprising SEQ ID NO:1, a second monomer comprising SEQ ID NO:2, and a light chain comprising SEQ ID NO:3. In some embodiments, the instructions further provide that olaparib is administered at a dose of about 300 mg. In some embodiments, the instructions provide that the dose of the olaparib is orally administered twice per day (b.i.d.) to the subject on day 1 of the first treatment cycle, and about twice per day (b.i.d.) thereafter. In some embodiments, the cancer has a homologous recombination deficiency (HRD). In some embodiments, the cancer has a biallelic loss of cyclin-dependent kinase 12 (CDK12).

In another aspect, provided herein is a kit comprising a bispecific anti-PD1×CTLA4 antibody, and instructions for use of said bispecific antibody in the treatment of an advanced gynecologic or genitourinary malignancy in a human subject in need thereof, and wherein the instructions provide that the bispecific antibody is administered to the subject according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle. In certain embodiments, the bispecific antibody comprises a first monomer comprising SEQ ID NO:1, a second monomer comprising SEQ ID NO:2, and a light chain comprising SEQ ID NO:3. In some embodiments, the malignancy is a platinum-resistant high-grade serous ovarian cancer (HGSOC). In some embodiments, the malignancy is a platinum-resistant high-grade fallopian tube cancer. In some embodiments, the malignancy is a platinum-resistant high-grade peritoneum cancer. In some embodiments, the malignancy is a chemotherapy relapsed or refractory clear cell ovarian cancer. In some embodiments, the malignancy is a chemotherapy relapsed or refractory clear cell endometrial cancer. In some embodiments, the malignancy is a chemotherapy relapsed or refractory clear cell peritoneal cancer. In some embodiments, the malignancy is an immune-checkpoint-inhibitor-refractory microsatellite stable (MSS) endometrial cancer. In some embodiments, the malignancy is a previously treated recurrent cervical cancer. In some embodiments, the malignancy is a previously treated metastatic cervical cancer. In some embodiments, the malignancy is a high-risk metastatic castration-resistant prostate cancer (mCRPC). In some embodiments, the malignancy is an advanced endometrial carcinoma that is not microsatellite instability-high (MSI-H) or deficient mismatch repair (dMMR). In some embodiments of the kits herein, the instructions provide that if the weight of the subject changes by more than 10% from baseline, the subject is optionally reassigned to a new dosing level and one or more subsequent doses are administered to the subject at the new dosing level. In some embodiments of the kits herein, the instructions provide that if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

### 5.14 Additional Embodiments

In one set of embodiments, provided herein is:

A1. A method of achieving a positive therapeutic response against prostate cancer, comprising:
administering a bispecific antibody to a male human subject in need of treatment thereof with a dosing regimen comprising:
an intravenous dose of the bispecific antibody, of between about 9.5 mg/kg and about 10.5 mg/kg, is administered on the first day of the dosing regimen; and
wherein the bispecific antibody comprises a first monomer comprising SEQ ID NO: 1, a second monomer comprising SEQ ID NO: 2, and a light chain comprising SEQ ID NO: 3
wherein the dosing regimen lasts between about 13 and about 15 days and wherein the dosing regimen repeats until the positive therapeutic response is achieved.

A2. The method of embodiment A1, wherein the male human subject is administered the dosing regimen for as long as there is the positive therapeutic response.

A3. The method of embodiment A1, wherein the dosing regimen occurs one or two or three or four times.

A4. The method of any one of embodiments A1 to A3, wherein the method further comprises:
prior to the administration of the dosing regimen, assessing an oncologic marker of the male human subject,
wherein the oncologic marker is selected from the group consisting of prostate-specific antigen (PSA) level, bone lesions, visceral lesions, and malignant lymph nodes,
thereby obtaining a baseline assessment, and
after the first administration of the bispecific antibody, assessing the oncologic marker of the male human subject, thereby obtaining a response assessment,
wherein the oncologic marker selected for the response assessment is the same as selected for the baseline assessment,
comparing the baseline assessment and the response assessment, and
determining the positive therapeutic response is achieved when the response assessment is an improvement over the baseline assessment.

A5. The method of embodiment A4, wherein there is more than one response assessment.

A6. The method of embodiment A4, wherein the oncologic marker is PSA level, and the response assessment is taken between about 21 days and about 28 days after the intravenous dose of the bispecific antibody, and the positive therapeutic response is when the PSA level in the response assessment is one or more levels below the PSA level in the baseline assessment.

A7. The method of embodiment A4, wherein the oncologic marker is bone lesions, and the positive therapeutic response is when there are fewer bone lesions in the response assessment than in the baseline assessment.

A8. The method of embodiment A4, wherein the oncologic marker is visceral lesions, and the positive therapeutic response is when there is a lower number of the visceral lesions, or a smaller size of the visceral lesions, in the response assessment than in the baseline assessment.

A9. The method of embodiment A4, wherein the oncologic marker is malignant lymph nodes, and the positive therapeutic response is when there is a lower number of the malignant lymph nodes, or a smaller size of the malignant lymph nodes, in the response assessment than in the baseline assessment.

A10. The method of any one of embodiments A1 to A9, wherein the prostate cancer is metastatic castration-resistant prostate cancer (mCRPC).

A11. The method of embodiment A10, wherein the mCRPC is microsatellite instability-high (MSI-H) and/or mismatch repair deficient (MMRD).

A12. The method of embodiment A11, wherein, prior to the dosing regimen, a checkpoint inhibitor had been administered to the male human subject.

A13. The method of any one of embodiments A1 to A12, wherein the male human subject has been administered at least one previous prostate cancer treatment, which is not the bispecific antibody.

A14. The method of any one of embodiments A1 to A13, wherein the male human subject has been administered at least two previous prostate cancer treatments, neither of which is the bispecific antibody.

A15. The method of embodiment A13 or A14, wherein the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody.

A16. The method of any one of embodiments A1 to A15, further comprising administering an androgen suppression treatment to the male human subject.

A17. The method of embodiment A16, wherein the androgen suppression treatment is a luteinizing hormone-releasing hormone analog.

A18. The method of any of embodiments A1 to A15, wherein the male human subject has received a surgical orchiectomy.

A19. The method of any one of embodiments A1 to A16, wherein the male human subject has an Eastern Cooperative Oncology Group (ECOG) performance status of 0 or 1.

A20. The method of any one of embodiments A1 to A19, wherein the intravenous dose of the bispecific antibody is about 10 mg/kg.

A21. The method of any one of embodiments A1 to A20, wherein the bispecific antibody is administered over between about 50 minutes and about 70 minutes.

A22. The method of any one of embodiments A1 to A21, wherein the first monomer consists of SEQ ID NO: 1, the second monomer consists of SEQ ID NO: 2, and the light chain consists of SEQ ID NO: 3.

A23. The method of any one of embodiments A1 to A22, further comprising, prior to the administering, assessing the weight of the male human subject.

In a second set of embodiments, provided herein is:

B 1. A method of achieving a positive therapeutic response against prostate cancer, comprising:
administering carboplatin, cabazitaxel, prednisone, and a bispecific antibody to a male human subject in need of treatment thereof with a dosing regimen comprising:
a) an intravenous dose of the carboplatin, with a target area under the serum concentration-time curve of between about 3 (AUC 3) and about 5 (AUC 5), is administered on the first day of the dosing regimen, and once about 20 and about 22 days thereafter;
b) an intravenous dose of the cabazitaxel, of between about 18 mg/m² and about 25 mg/m², is administered on the first day of the dosing regimen, and once about 20 and about 22 days thereafter;
c) an oral dose of the prednisone, of between about 3 mg and about 7 mg, is administered twice a day;
d) an intravenous dose of the bispecific antibody, of between about 9.5 mg/kg and about 10.5 mg/kg, is administered on the first day of the dosing regimen, and once about 13 and about 15 days thereafter;
wherein the bispecific antibody comprises a first monomer comprising SEQ ID NO: 1, a second monomer comprising SEQ ID NO: 2, and a light chain comprising SEQ ID NO: 3
wherein the dosing regimen repeats until the positive therapeutic response is achieved.

B2. The method of embodiment B1, wherein the male human subject is administered the dosing regimen for as long as there is the positive therapeutic response.

B3. The method of embodiment B 1, wherein the dosing regimen occurs one or two times.

B4. The method of any one of embodiments B1 to B3, wherein the method further comprises:
prior to the administration of the dosing regimen, assessing an oncologic marker of the male human subject,
wherein the oncologic marker is selected from the group consisting of prostate-specific antigen (PSA) level, bone lesions, visceral lesions, and malignant lymph nodes,
thereby obtaining a baseline assessment, and
after the first administration of the bispecific antibody, assessing the oncologic marker of the male human subject, thereby obtaining a response assessment,
wherein the oncologic marker selected for the response assessment is the same as selected for the baseline assessment,
comparing the baseline assessment and the response assessment, and
determining the positive therapeutic response is achieved when the response assessment is an improvement over the baseline assessment.

B5. The method of embodiment B4, wherein there is more than one response assessment.

B6. The method of embodiment B4, wherein the oncologic marker is PSA level, and the response assessment is taken between about 21 days and about 28 days after the intravenous dose of the bispecific antibody, and the positive therapeutic response is when the PSA level in the response assessment is one or more levels below the PSA level in the baseline assessment.

B7. The method of embodiment B4, wherein the oncologic marker is bone lesions, and the positive therapeutic response is when there are fewer bone lesions in the response assessment than in the baseline assessment.

B8. The method of embodiment B4, wherein the oncologic marker is visceral lesions, and the positive therapeutic response is when there is a lower number of the visceral lesions, or a smaller size of the visceral lesions, in the response assessment than in the baseline assessment.

B9. The method of embodiment B4, wherein the oncologic marker is malignant lymph nodes, and the positive therapeutic response is when there is a lower number of the malignant lymph nodes, or a smaller size of the malignant lymph nodes, in the response assessment than in the baseline assessment.

B10. The method of any one of embodiments B1 to B11, wherein the prostate cancer is metastatic castration-resistant prostate cancer (mCRPC).

B11. The method of embodiment B10, wherein the mCRPC is aggressive variant (anaplastic) adenocarcinoma of the prostate (AVPCa).

B12. The method of embodiment B11, wherein the AVPCa is positive for aberrancy in at least two genes selected from the group consisting of Rb1, TP53, and PTENb.

B13. The method of embodiment B10, wherein the mCRPC is a homologous recombination deficient (HRD)/cyclin-dependent kinase 12 (CDK12) mutation positive tumor.

B14. The method of embodiment B13, wherein, prior to the first administration of the dosing regimen, a poly-adenosine diphosphate ribose polymerase (PARP) inhibitor had been administered to the male human subject.

B15. The method of embodiment B14, wherein the prostate cancer of the male human subject progressed after the PARP inhibitor administration.

B16. The method of embodiment B13, wherein the HRD/CDK12 mutation positive tumor is HRD positive in at least one gene selected from the group consisting of BRCA1, BRCA2, ATM, PALB2, CHEK2, and FANCA.

B17. The method of embodiment B13, wherein the HRD/CDK12 mutation positive tumor is positive for biallelic loss of CDK12.

B18. The method of any one of embodiments B1 to B17, wherein the prostate cancer is metastatic castration-resistant prostate cancer (mCRPC) which does not have a targetable mutation.

B19. The method of any one of embodiments B1 to B18, wherein the male human subject has been administered at least one previous prostate cancer treatment, which is not the carboplatin, the cabazitaxel, the prednisone, or the bispecific antibody.

B20. The method of any one of embodiments B1 to B19, wherein the male human subject has been administered at least two previous prostate cancer treatments, neither of which are the carboplatin, the cabazitaxel, the prednisone, or the bispecific antibody.

B21. The method of embodiment B19 or B20, wherein the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody.

B22. The method of any one of embodiments B1 to B21, further comprising administering an androgen suppression treatment to the male human subject.

B23. The method of embodiment B22, wherein the androgen suppression treatment is a luteinizing hormone-releasing hormone analog.

B24. The method of any of embodiments B1 to B21, wherein the male human subject has received a surgical orchiectomy.

B25. The method of any one of embodiments B1 to B24, wherein the male human subject has an Eastern Cooperative Oncology Group (ECOG) performance status of 0 or 1.

B26. The method of any one of embodiments B1 to B25, wherein the intravenous dose of the carboplatin is with a target area under the serum concentration-time curve of about 4 (AUC 4).

B27. The method of any one of embodiments B1 to B26, wherein the carboplatin is administered over between about 20 minutes and about 40 minutes.

B28. The method of any one of embodiments B1 to B27, wherein the intravenous dose of cabazitaxel is about 20 mg/m².

B29. The method of any one of embodiments B1 to B28, wherein the cabazitaxel is administered over between about 50 minutes and about 70 minutes.

B30. The method of any one of embodiments B1 to B29, wherein the oral dose of the prednisone is about 5 mg.

B31. The method of any one of embodiments B1 to B30, wherein the intravenous dose of the bispecific antibody is about 10 mg/kg.

B32. The method of any one of embodiments B1 to B31, wherein the bispecific antibody is administered over between about 50 minutes and about 70 minutes.

B33. The method of any one of embodiments B1 to B32, wherein the first monomer consists of SEQ ID NO: 1, the second monomer consists of SEQ ID NO: 2, and the light chain consists of SEQ ID NO: 3.

B34. The method of any one of embodiments B1 to B33, further comprising, prior to the administering, assessing the weight of the male human subject.

B35. A method of achieving a positive therapeutic response against prostate cancer, comprising:
administering carboplatin, docetaxel, prednisone, and a bispecific antibody to a male human subject in need of treatment thereof with a dosing regimen comprising:
a) an intravenous dose of the carboplatin, with a target area under the serum concentration-time curve of between about 3 (AUC 3) and about 5 (AUC 5), is administered on the first day of the dosing regimen, and once about 20 and about 22 days thereafter;
b) an intravenous dose of the doctaxel, of between about 55 mg/m² and about 75 mg/m², is administered on the first day of the dosing regimen, and once about 20 and about 22 days thereafter;
c) an oral dose of the prednisone, of between about 3 mg and about 7 mg, is administered twice a day;
d) an intravenous dose of the bispecific antibody, of between about 9.5 mg/kg and about 10.5 mg/kg, is administered on the first day of the dosing regimen, and once about 13 and about 15 days thereafter;
wherein the bispecific antibody comprises a first monomer comprising SEQ ID NO: 1, a second monomer comprising SEQ ID NO: 2, and a light chain comprising SEQ ID NO: 3
wherein the dosing regimen repeats until the positive therapeutic response is achieved.

B36. The method of embodiment 35, wherein the male human subject has been administered at least one previous prostate cancer treatment, which is not the carboplatin, the docetaxel, the prednisone, or the bispecific antibody.

B37. The method of embodiment B35, wherein the male human subject has been administered at least two previous prostate cancer treatments, neither of which are the carboplatin, the docetaxel, the prednisone, or the bispecific antibody.

B38. The method of embodiment B35, wherein the intravenous dose of docetaxel is about 60 mg/m².

B39. The method of embodiment B35, wherein the docetaxel is administered over between about 50 minutes and about 70 minutes.

In a third set of embodiments, provided herein is:

C1. A method of achieving a positive therapeutic response against prostate cancer, comprising:

administering olaparib and a bispecific antibody to a male human subject in need of treatment thereof with a dosing regimen comprising:
a) an intravenous dose of the bispecific antibody, of between about 9.5 mg/kg and about 10.5 mg/kg, administered on the first day of the dosing regimen; and
b) an oral dose of olaparib, of between about 270 mg and about 330 mg, administered twice a day;
wherein the bispecific antibody comprises a first monomer comprising SEQ ID NO: 1, a second monomer comprising SEQ ID NO: 2, and a light chain comprising SEQ ID NO: 3
wherein the dosing regimen lasts between about 13 and about 15 days and wherein the dosing regimen repeats until the positive therapeutic response is achieved.

C2. The method of embodiment C1, wherein the male human subject is administered the dosing regimen for as long as there is the positive therapeutic response.

C3. The method of embodiment C1, wherein the dosing regimen occurs one or two or three or four times.

C4. The method of any one of embodiments C1 to C3, wherein the method further comprises:
prior to the administration of the dosing regimen, assessing an oncologic marker of the male human subject,
wherein the oncologic marker is selected from the group consisting of prostate-specific antigen (PSA) level, bone lesions, visceral lesions, and malignant lymph nodes,
thereby obtaining a baseline assessment, and
after the first administration of the bispecific antibody, assessing the oncologic marker of the male human subject, thereby obtaining a response assessment,
wherein the oncologic marker selected for the response assessment is the same as selected for the baseline assessment,
comparing the baseline assessment and the response assessment, and
determining the positive therapeutic response is achieved when the response assessment is an improvement over the baseline assessment.

C5. The method of embodiment C4, wherein there is more than one response assessment.

C6. The method of embodiment C4, wherein the oncologic marker is PSA level, and the response assessment is taken between about 21 days and about 28 days after the intravenous dose of the bispecific antibody, and the positive therapeutic response is when the PSA level in the response assessment is one or more levels below the PSA level in the baseline assessment.

C7. The method of embodiment C4, wherein the oncologic marker is bone lesions, and the positive therapeutic response is when there are fewer bone lesions in the response assessment than in the baseline assessment.

C8. The method of embodiment C4, wherein the oncologic marker is visceral lesions, and the positive therapeutic response is when there is a lower number of the visceral lesions, or a smaller size of the visceral lesions, in the response assessment than in the baseline assessment.

C9. The method of embodiment C4, wherein the oncologic marker is malignant lymph nodes, and the positive therapeutic response is when there is a lower number of the malignant lymph nodes, or a smaller size of the malignant lymph nodes, in the response assessment than in the baseline assessment.

C10. The method of any one of embodiments C1 to C9, wherein the prostate cancer is metastatic castration-resistant prostate cancer (mCRPC).

C11. The method of embodiment C10, wherein the mCRPC is a homologous recombination deficient (HRD)/cyclin-dependent kinase 12 (CDK12) mutation positive cancer, wherein, prior to the dosing regimen, a poly-adenosine diphosphate ribose polymerase (PARP) inhibitor had not been administered to the male human subject.

C12. The method of embodiment C11, wherein the HRD/CDK12 mutation positive cancer is HRD positive in at least one gene selected from the group consisting of BRCA1, BRCA2, ATM, PALB2, CHEK2, and FANCA.

C13. The method of embodiment C11, wherein the HRD/CDK12 mutation positive cancer is positive for biallelic loss of CDK12.

C14. The method of any one of embodiments C1 to C13, wherein the male human subject has been administered at least one previous prostate cancer treatment, which is neither the olaparib nor the bispecific antibody.

C15. The method of any one of embodiments C1 to C14, wherein the male human subject has been administered at least two previous prostate cancer treatments, neither of which are the olaparib or the bispecific antibody.

C16. The method of embodiment C14 or C15, wherein the previous prostate cancer treatment is selected from the group consisting of a luteinizing hormone-releasing hormone analog, a taxane, a platinum chemotherapeutic, an androgen receptor signaling inhibitor, a bone-targeting radionuclide, sipuleucel-T, and a checkpoint inhibitor antibody.

C17. The method of any one of embodiments C1 to C16, further comprising administering an androgen suppression treatment to the male human subject.

C18. The method of embodiment C17, wherein the androgen suppression treatment is a luteinizing hormone-releasing hormone analog.

C19. The method of any of embodiments C1 to C16, wherein the male human subject has received a surgical orchiectomy.

C20. The method of any one of embodiments C1 to C19, wherein the male human subject has an Eastern Cooperative Oncology Group (ECOG) performance status of 0 or 1.

C21. The method of any one of embodiments C1 to C20, wherein the oral dose of the olaparib is about 300 mg.

C22. The method of any one of embodiments C1 to C21, wherein the intravenous dose of the bispecific antibody is about 10 mg/kg.

C23. The method of any one of embodiments C1 to C22, wherein the bispecific antibody is administered over a time period between about 50 minutes and about 70 minutes.

C24. The method of any one of embodiments C1 to C23, wherein the first monomer consists of SEQ ID NO: 1, the second monomer consists of SEQ ID NO: 2, and the light chain consists of SEQ ID NO: 3.

C25. The method of any one of embodiments C1 to C24, further comprising, prior to the administering, assessing the weight of the male human subject.

In a fourth set of embodiments, provided herein is:

D1. A method for treating a gynecologic or genitourinary cancer in a human subject, comprising: administering to the human subject having the solid cancerous tumor an intravenous dose, once every 13-15 days, of between about 0.05 mg/kg and about 12 mg/kg of a bispecific antibody comprising a first monomer comprising SEQ ID NO: 1, a second monomer comprising SEQ ID NO: 2, and a light chain comprising SEQ ID NO: 3 for a time period sufficient to treat the gynecologic or genitourinary cancer.

D2. The method of embodiment D1, wherein the is an ovarian cancer, a clear cell carcinoma, an endometrial cancer, a cervical cancer, or a prostate cancer.

D3. The method of embodiment D1, wherein the cancer is a clear cell carcinoma.

D4. The method of embodiment D3, wherein the clear cell carcinoma is a clear cell ovarian, endometrial, or peritoneal carcinoma.

D5. The method of embodiment D3 or D4, wherein the clear cell carcinoma is a persistent or recurrent clear cell carcinoma.

D6. The method of any one of embodiments D3 to D5, wherein the clear cell carcinoma was previously treated with a platinum-based systemic chemotherapy.

D7. The method of embodiment D1, wherein the cancer is a platinum-resistant high-grade serous ovarian cancer (HGSOC).

D8. The method of embodiment D1, wherein the cancer is an endometrial cancer.

D9. The method of embodiment D8, wherein the endometrial cancer is not microsatellite instability-high (MSI-H) or deficient mismatch repair (dMMR).

D10. The method of embodiment D8, wherein the endometrial cancer is an immune-checkpoint-inhibitor-refractory microsatellite stable (MSS) endometrial cancer (EC).

D11. The method of embodiment D1, wherein the cancer is a cervical cancer.

D12. The method of embodiment D11, wherein the cervical cancer is a previous treated recurrent or metastatic cervical cancer.

D13. The method of embodiment D12, wherein the cervical cancer was previously treated with chemotherapy or an immunotherapy.

D14. The method of embodiment D13, wherein the chemotherapy is a standard-of-care systemic chemotherapy.

D15. The method of embodiment D1, wherein the cancer is a prostate cancer.

D16. The method of embodiment D15, wherein the prostate cancer is a high-risk metastatic castration-resistant prostate cancer (mCRPC).

D17. The method of any one of embodiments D1 to D16, wherein the intravenous dose is: between about 0.05 mg/kg and about 0.25 mg/kg; or between about 0.2 mg/kg and about 0.4 mg/kg; or between about 0.5 mg/kg and about 1.5 mg/kg; or between about 2.0 mg/kg and about 4.0 mg/kg; or between about 8.0 mg/kg and about 12.0 mg/kg.

D18. The method of any one of embodiments D1 to D17, wherein the intravenous dose is between about 0.15 mg/kg and about 10.0 mg/kg.

D19. The method of any one of embodiments D1 to D18, wherein the administering of the intravenous dose to the human subject is between about 45 minutes and about 75 minutes.

D20. The method of any one of embodiments D1 to D19, wherein the time period sufficient to treat the cancer is between about 3 weeks and about 9 weeks.

D21. The method of any one of embodiments D1 to D20, further comprising, prior to the administering, assessing the weight of the human subject.

In a fifth set of embodiments, provided herein is:

E1. A method of treating a prostate cancer in a male human subject in need thereof, the method comprising:
administering to the subject according to a 28 day treatment cycle, a bispecific antibody at a dose of about 10 mg/kg,
wherein the dose of the bispecific antibody is intravenously administered to the subject on day 1 of a first 28 day treatment cycle and about every two weeks (Q2W) thereafter, and
wherein the bispecific antibody comprises a first monomer comprising SEQ ID NO:1, a second monomer comprising SEQ ID NO:2, and a light chain comprising SEQ ID NO:3.

E2. A method according to embodiment E1, wherein the prostate cancer is microsatellite instability-high (MSI-H) prostate cancer.

E3. A method according to embodiment E1, wherein the prostate cancer is mismatch repair deficient (MMRD) prostate cancer.

E4. A method according to any one of embodiments E1 to E3, wherein the subject receives treatment about every 2 weeks (Q2W) for about two years.

E5. A method according to embodiment E1, wherein the prostate cancer is an aggressive variant (anaplastic) adenocarcinoma of the prostate (AVPCa), and wherein the method further comprises:
(a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and
(b) administering cabazitaxel at a dose of about 20 mg/m2, wherein the dose of the cabazitaxel is intravenously administered to the subject on day 1 of the first treatment cycle and about every three weeks (Q3W) thereafter.

E6. A method according to embodiment E5, further comprising orally administering a steroid to the subject.

E7. A method according to embodiment E6, wherein the steroid is prednisone administered at a dose of about 5 mg twice per day (b.i.d.) on day 1 of the first treatment cycle, and about twice per day (b.i.d.) thereafter.

E8. A method according to embodiment E1, wherein the prostate cancer is an aggressive variant (anaplastic) adenocarcinoma of the prostate (AVPCa), and wherein the subject has not previously been administered docetaxel, wherein the method further comprises:
(a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and
(b) administering docetaxel at a dose of about 60 mg/m2 wherein the dose of the docetaxel is intravenously administered to the subject on day 1 of the first treatment cycle and about every three weeks (Q3W) thereafter.

E9. A method according to embodiment E8, further comprising administering a steroid to the subject.

E10. A method according to embodiment E9, wherein the steroid is prednisone administered at a dose of about 5 mg twice per day (b.i.d.) on day 1 of the first treatment cycle, and about twice per day (b.i.d.) thereafter.

E11. A method according to any of embodiments E5 to E10, wherein the cancer has a mutation or other aberrancy in at least two genes independently selected from the group consisting of Rb1, TP53 and PTEN.

E12. The method of any one of embodiments E5 to E11, wherein the subject receives more than one 28 day treatment cycle.

E13. The method of any of embodiments E5 to E12, wherein the subject receives up to twenty-four 28 day treatment cycles.

E14. A method according to embodiment E1, wherein the method further comprises:
(a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and
(b) administering cabazitaxel at a dose of about 20 mg/m2, wherein the dose of the cabazitaxel is intravenously administered to the subject on day 1 of the first treatment cycle and about every three weeks (Q3W) thereafter.

E15. A method according to embodiment E14, wherein the subject is further administered a steroid.

E16. A method according to embodiment E15, wherein the steroid is prednisone administered at a dose of about 5 mg twice per day (b.i.d.) on day 1 of the first treatment cycle, and about twice per day (b.i.d.) thereafter.

E17. A method according to embodiment E1, wherein the subject has not previously been administered docetaxel, and wherein the method further comprises:
(a) administering carboplatin at a therapeutically effective dose that results in a target area under the serum concentration-time curve of 4 (AUC4) in the subject, wherein the dose of the carboplatin is intravenously administered to the subject on day 1 of the first treatment cycle and about every three weeks (Q3W) thereafter; and
(b) administering docetaxel at a dose of about 60 mg/m2 wherein the dose of the docetaxel is intravenously administered to the subject on day 1 of the first treatment cycle and about every three weeks (Q3W) thereafter.

E18. A method according to embodiment E17, wherein the subject is administered a steroid.

E19. A method according to embodiment E18, wherein the steroid is prednisone administered at a dose of about 5 mg twice per day (b.i.d.) on day 1 of the first treatment cycle, and about twice per day (b.i.d.) thereafter.

E20. A method according to any of embodiments E14 to E19, wherein the subject has received prior treatment with a polyadenosine diphosphate ribose polymerase (PARP) inhibitor.

E21. A method according to any of embodiments E14 to E20, wherein the cancer has a homologous recombination deficiency (HRD).

E22. A method according to any of embodiments E14 to E21, wherein the cancer has a biallelic loss of cyclin-dependent kinase 12 (CDK12).

E23. A method according to embodiment E1, wherein the subject has not previously been administered a PARP inhibitor, and wherein the method further comprising administering olaparib at a dose of about 300 mg.

E24. A method according to embodiment E23, wherein the dose of the olaparib is orally administered twice per day (b.i.d.) to the subject on day 1 of the first treatment cycle, and about twice per day (b.i.d.) thereafter.

E25. A method according to embodiment E23 or E24, wherein the cancer has a homologous recombination deficiency (HRD).

E26. A method according to any of embodiments E23 to E25, wherein the cancer has a biallelic loss of cyclin-dependent kinase 12 (CDK12).

E27. A method of treating an advanced gynecologic or genitourinary malignancy in a human subject in need thereof, the method comprising administering to the subject a dose of a bispecific antibody according to a 21 day treatment cycle, wherein the dose of the bispecific antibody is about 1200 mg if the subject weighs 80 kg or more, or wherein the dose of the bispecific antibody is about 1000 mg if the subject weighs less than 80 kg, wherein the dose of the bispecific antibody is intravenously administered to the subject on day 1 of each 21 day treatment cycle, wherein the bispecific antibody comprises a first monomer comprising SEQ ID NO:1, a second monomer comprising SEQ ID NO:2, and a light chain comprising SEQ ID NO:3.

E28. The method of embodiment E27, wherein the malignancy is a platinum-resistant high-grade serous ovarian cancer (HGSOC).

E29. The method of embodiment E27, wherein the malignancy is a platinum-resistant high-grade fallopian tube cancer.

E30. The method of embodiment E27, wherein the malignancy is a platinum-resistant high-grade peritoneum cancer.

E31. The method of embodiment E27, wherein the malignancy is a chemotherapy relapsed or refractory clear cell ovarian cancer.

E32. The method of embodiment E27, wherein the malignancy is a chemotherapy relapsed or refractory clear cell endometrial cancer.

E33. The method of embodiment E27, wherein the malignancy is a chemotherapy relapsed or refractory clear cell peritoneal cancer.

E34. The method of embodiment E27, wherein the malignancy is an immune-checkpoint-inhibitor-refractory microsatellite stable (MSS) endometrial cancer.

E35. The method of embodiment E27, wherein the malignancy is a previously treated recurrent cervical cancer.

E36. The method of embodiment E27, wherein the malignancy is a previously treated metastatic cervical cancer.

E37. The method of embodiment E27, wherein the malignancy is a high-risk metastatic castration-resistant prostate cancer (mCRPC).

E38. The method of embodiment E27, wherein the malignancy is an advanced endometrial carcinoma that is not microsatellite instability-high (MSI-H) or deficient mismatch repair (dMMR).

E39. The method of any one of embodiments E22 to E33, wherein if the weight of the subject changes by more than 10% from baseline, the subject is optionally reassigned to a new dosing level and one or more subsequent doses are administered to the subject at the new dosing level.

E40. The method of any one of embodiment E27 to E39, wherein if the subject initially receives three cycles of the 1000 mg dose of the bispecific antibody without experiencing a ≥Grade 2 immune-related adverse event (irAE), then the subject receives 1200 mg of the bispecific antibody beginning with the fourth cycle and all subsequent cycles.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification controls.

### 6. Examples

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, and 2014/0288275, as well as PCT Publication WO2014/145806, as well as U.S. Applications 62/085,027, 14/952,714, and 15/141,350, all of which are expressly incorporated by reference in their entirety and particularly for the techniques outlined therein.

### 6.1 Example 1: Treatment of Solid Cancerous Tumors (Phase 1)

### XmAb®20717 Treatment Plan

This is a two-part Phase 1, multiple-dose, ascending-dose escalation study and expansion designed to define a Maximum Tolerated Dose and/or Recommended Dose (MTD/RD) and regimen, as well as preliminarily assessing potential anti-tumor activity of XmAb®20717 in subjects with selected advanced solid cancerous tumors. All eligible subjects have relapsed or refractory disease after standard therapy.

XmAb®20717 is a humanized bsAb that binds both PD1 and CTLA4. The XmAb®20717 pharmaceutical composition is a sterile liquid supplied in single-use glass vials. Each 10 mL vial is filled with 10.5 mL of pharmaceutical composition containing 10.0±1.0 mg/mL of XmAb®20717, in 20 mM histidine, 250 mM sorbitol, and 0.01% (w/v) polysorbate-80 at pH 6.2. Each product vial is intended to deliver 10.0 mL of drug solution.

Prior to administration, XmAb®20717 is diluted to the final required concentration in an ethylene/polypropylene copolymer infusion bag containing 0.9% Sodium Chloride Injection, USP. After dilution, the bag containing XmAb®20717 should be gently inverted 2 to 3 times to mix the solution. The bag should not be shaken.

XmAb®20717 administration should begin as soon as possible after the dosing solution is made. If there is a delay in administration, the dosing solution may be stored at 2-8° C. for no more than 24 hours or at room temperature for no more than 4 hours prior to infusion. The full-calculated dose is administered based on the subject's actual baseline weight measurement in kilograms. Following the first dose, subsequent doses are modified only if the subject's weight changes by more than 10% from the Day −1 weight, at which point it is recalculated using the current weight. In Part A of the study, subjects are enrolled into escalating dose cohorts to establish an MTD/RD(s) for a dosing regimen consisting of infusions on Days 1 and 15 of each 28-day cycle.

The decision to escalate to higher-dose cohorts proceeds according to the Dose Escalation Plan, and the final decision to escalate to a higher dose level is based on review of the aggregate safety data for all subjects through Cycle 1 Day 28. For each escalation cohort, the first subject in the cohort is dosed and observed for a minimum of 24 hours before study drug is administered to the remainder of the cohort. All subjects are assessed for the development of dose-limiting toxicity (DLT) during treatment with XmAb®20717. The assessment period is defined as: Cycle 1, Days 1 to 28. Part B of the study begins once the MTD/RD(s) and dosing regimen are established. In Part B, additional subjects with advanced melanoma (excluding uveal), renal cell carcinoma (clear cell predominant type), and non-small cell lung carcinoma are enrolled into disease-specific expansion cohorts of up to 20 subjects each.

### Dosing Schedule

Each subject was administered XmAb®20717 IV at a constant infusion rate over 1 hour. The initial treatment period for each subject in this study was 2 cycles. Each cycle was 28 days long and consisted of 2 doses of XmAb®20717, on Days 1 and 15. Six dose levels were planned for the dose-escalation phase of the study (Part A). The dose levels are: 0.15, 0.3, 1.0, 3.0, 6.0, and 10.0 mg/kg. A subject's first dose was based on the Day −1 baseline weight in kilograms. Subsequent doses were modified only if the subject's weight changed by more than 10% from the Day −1 weight, at which point the dose was recalculated using the subject's current weight. That dose was continued for the remainder of the trial, unless there was a subsequent 10% weight change.

TABLE 3
Dose Level Scheme

| | | |
|---|---|---|
| Cohort | Planned Dose | Human Subjects |

| | | | |
|---|---|---|---|
| 1 | 0.15 | mg/kg | 3 (+3) |
| 2 | 0.3 | mg/kg | 3 (+3) |
| 3 | 1.0 | mg/kg | 3 (+3) |
| 4 | 3.0 | mg/kg | 3 (+3) |
| 5 | 6.0 | mg/kg | 3 (+3) |
| 6 | 10.0 | mg/kg | 3 (+3) |

A minimum of 3 subjects were enrolled in each dose-escalation cohort. No 2 subjects within a cohort started treatment with XmAb®20717 on the same day; the first subject was dosed and observed for a minimum of 24 hours before study drug was administered to the remainder of the cohort. All subjects were assessed for the development of dose-limiting toxicities (DLT) during treatment with XmAb®20717. If none of the first 3 subjects experienced a DLT during the period, escalation to the next dosing level occurred. If any of the first 3 subjects in a dosing cohort experienced a DLT during the period, the cohort was expanded to a total of 6 human subjects or until a second subject in the cohort experienced a DLT. If there were no additional DLTs, escalation to the next dose level occurred. If there is 1 or more additional DLTs, the MTD has been exceeded, and the next lower dose level is expanded to 6 subjects. If no more than 1 subject experiences a DLT at the deescalated dose level, it is the MTD.

TABLE 4
Dose Escalation Scheme
Standard Dose Escalation Phase

| | Number of Subjects | |
|---|---|---|
| | Enrolled and As- | |
| Number of Human | sessable for Safety | |
| Subjects with | Following 2 Doses | |
| at Least One DLT | of XmAb ®20717 | Escalation Decision |
| 0 | 3 | Escalate to the next higher |
| | | dose |
| 1 | 3 | Enroll 3 additional human |
| | | subjects at the same |
| | | dose level |
| 1 | 6 | Escalate to the next higher |
| | | dose level |
| 2 | Up to 3 or 6 | No dose escalation may |
| | | occur; The dose must be de- |
| | | escalated. |

DLT = dose-limiting toxicity

Results: As of Feb. 5, 2020, 34 patients were treated in cohorts 1-6 at fixed doses of 0.15 to 10 mg/kg. Patients had a median age of 57 years (range 32-81), a median time since initial diagnosis of 42 months (range 3-313) and a median of 4 prior systemic therapies (range 0-9). 68% of patients had a TNM stage of III/IV and 68% had been exposed to checkpoint therapy.

XmAb®20717 treatment was generally well-tolerated through the highest dose cohort tested. Overall rates of Gr3/4 immune-related AEs occurred in 8 (24%) patients including elevations of transaminases 3 (9%), rash 2 (6%), lipase and amylase 1 (3%, without clinical symptoms or radiographic evidence of pancreatitis), lipase (alone) 1 (3%), pruritus 1 (3%), hyperglycemia 1 (3%), arthritis 1 (3%) and colitis 1 (3%), all reversible.

Responses were evaluated based on RECIST 1.1 criteria and there was 1 complete response reported (melanoma, progressed on prior pembrolizumab) at 10 mg/kg (highest dose level). Dose-dependent pharmacodynamic activity consistent with dual PD1/CTLA4 blockade was noted, namely a proliferative burst of both CD8 and CD4 T cells and induction of IFN-inducible chemokines (Table 5).

TABLE 5
Maximum change from baseline in the first two cycles

| | | | | IP-10 | MIG |
|---|---|---|---|---|---|
| | Dose Cohort | CD8+Ki67+% | CD4+Ki67+% | Geometric mean of fold | |
| Cohort | (mg/kg) | Mean change | | change | |
| 1 | 0.15 | 5.6 (n = 2) | 1.8 (n = 2) | 1.3 (n = 3) | 1.8 (n = 3) |
| 2 | 0.3 | 1.7 (n = 3) | 1.6 (n = 3) | 1.0 (n = 3) | 1.2 (n = 3) |
| 3 | 1 | 8.9 (n = 5) | 5.2 (n = 5) | 1.4 (n = 6) | 2.0 (n = 6) |
| 4 | 3 | 16.0 (n = 5) | 9.1 (n = 5) | 2.6 (n = 6) | 2.8 (n = 6) |
| 5 | 6 | 13.6 (n = 7) | 6.8 (n = 7) | 3.5 (n = 8) | 3.2 (n = 8) |
| 6 | 10 | 21.4 (n = 7) | 11.5 (n = 7) | 2.7 (n = 7) | 3.8 (n = 7) |

Conclusions: XmAb®20717 was generally safe and demonstrated PD activity in heavily pretreated patients with selected advanced solid tumors.

### 6.2 Example 2: Treatment of Solid Cancerous Tumors (Prostate Cancer, Phase 2)

### XmAb®20717 Treatment Plan

Vudalimab (XmAb®20717) is a humanized bispecific monoclonal antibody that simultaneously targets PD-1 and CTLA4, and binds preferentially to PD1/CTLA4 dual-positive cells.

Preliminary data from a Phase 1 study in heavily pretreated patients with advanced solid tumors showed that monotherapy treatment with 10 mg/kg of XmAb®20717 every two weeks (Q2W) was generally well tolerated. The most common irAEs were rash, pruritus, and increased transaminases. Treatment with XmAb®20717 was associated with complete and partial responses in tumor types typically responsive to single-agent immune checkpoint inhibitor (ICI) therapy (melanoma, RCC, NSCLC), as well as those that are not (mCRPC, ovarian cancer). Durability of responses in the mCRPC patients (PRs in 2 of 4 mCRPC patients with measurable disease) was 41.3 and 27 weeks, and both patients were without progression on bone scans, and had confirmed PSA decreases≥50% following treatment.

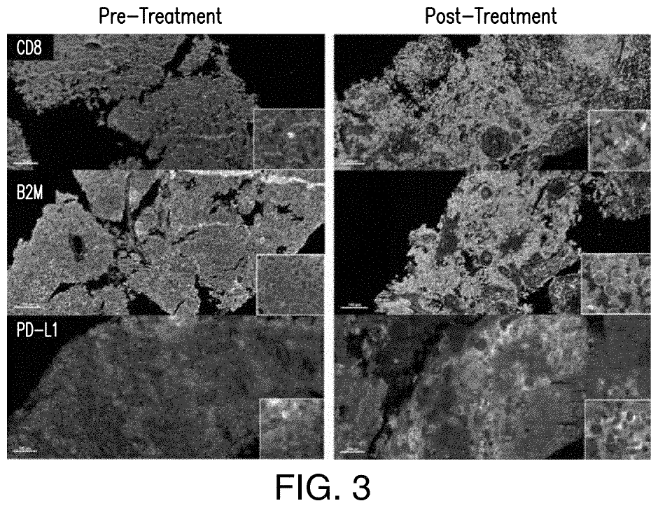

As shown in FIG. 3, potent antitumor immune response was observed in an mCRPC patient with partial response following treatment with XmAb®20717 10 mg/kg Q2W. Multiplex immunofluorescence staining of paired pre-treatment and posttreatment biopsies demonstrated increases in cellular immunity (CD8+ T cells), marked induction of MHC I antigen presenting machinery (B2M), and IFN pathway response (upregulation of PD-L1).

Results from the Phase 1 study, showed that XmAb®20717 (vudalimab) monotherapy was well-tolerated and associated with complete and partial responses in patients with multiple tumor types, including mCRPC. Although prostate adenocarcinoma generally is considered to be an immunotherapy-resistant cancer, patient selection could be focused on those with tumors with molecular characteristics that have shown increased sensitivity to immune checkpoint inhibitors (ICIs), such as those associated with aggressive variant disease, CDK12 inactivation, and microsatellite instability-high (MSI-H) or mismatch repair-deficient (MMRD) status. In addition, altering the tumor microenvironment to promote antitumor immunity by combining ICIs with chemotherapy or targeted agents also has the potential to increase clinical benefit.

The potential for dual PD1/CTLA4 blockade to improve outcomes in patients with mCRPC relative to those observed for agents directed at individual checkpoints has been demonstrated previously with the combination of nivolumab and ipilimumab (Sharma P, et al. Cancer Cell. 2020; 38:489-499). Identifying additional strategies to optimize response to ICI therapy in this population would address a continued high unmet need. Although prostate adenocarcinoma generally is considered to be an immunotherapy-resistant cancer, patient selection could be focused on those with tumors with molecular characteristics that have shown increased sensitivity to ICIs, such as those associated with aggressive variant disease (Lee L, et al. JCO Precis Oncol. 2018:1-8), CDK12 inactivation (Antonarakis E S, et al. JCO Precis Oncol. 2020; 4:370-381; Wu Y M, et al. Cell. 2018; 173:1770-1782), and microsatellite instability-high (MSI-H) or mismatch repair-deficient (MMRD) status (Abida W, et al. JAMA Oncol. 2019; 5:471-478). In addition, altering the tumor microenvironment to promote antitumor immunity by combining ICIs with chemotherapy or targeted agents also has the potential to increase clinical benefit (Picardo S L, Hansen A R. Ann Transl Med. 2019; 7:346).

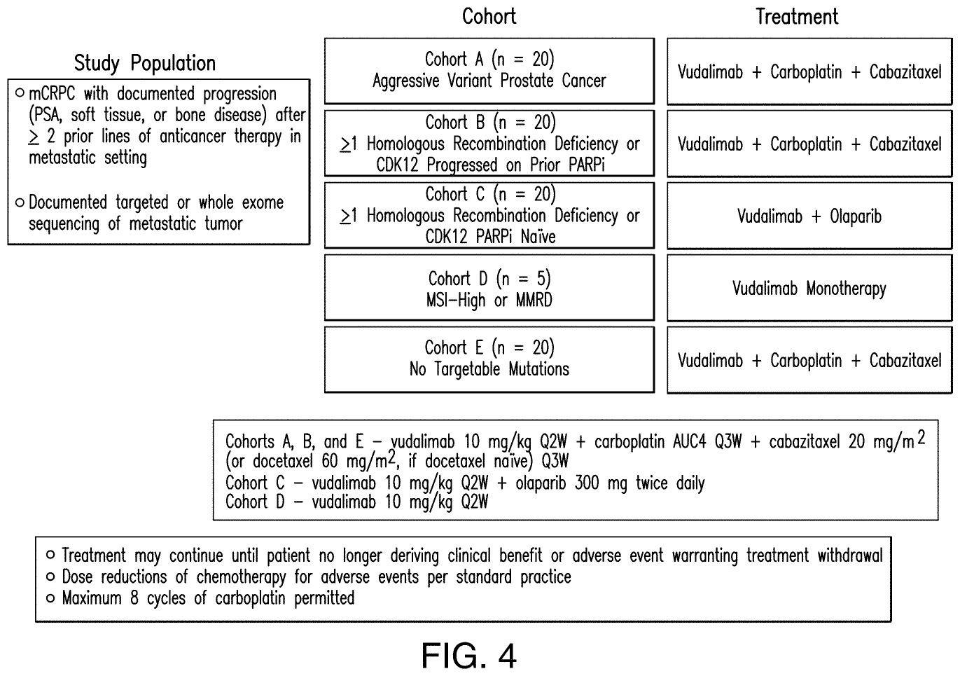

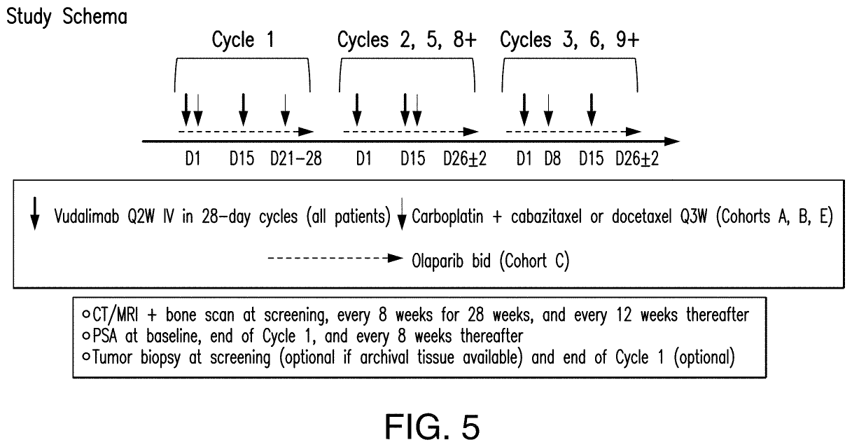

Therefore, the Phase 2, multicenter, parallel-group, open-label study (NCT05005728) was designed to evaluate the safety and antitumor activity of vudalimab (XmAb®20717), a bispecific anti-PD1×anti-CTLA4 antibody, in combination with other anticancer agents or alone in subgroups of mCRPC patients with and without specific tumor molecular subtypes (FIG. 4 and FIG. 5).

### XmAb®20717 Treatment Plan

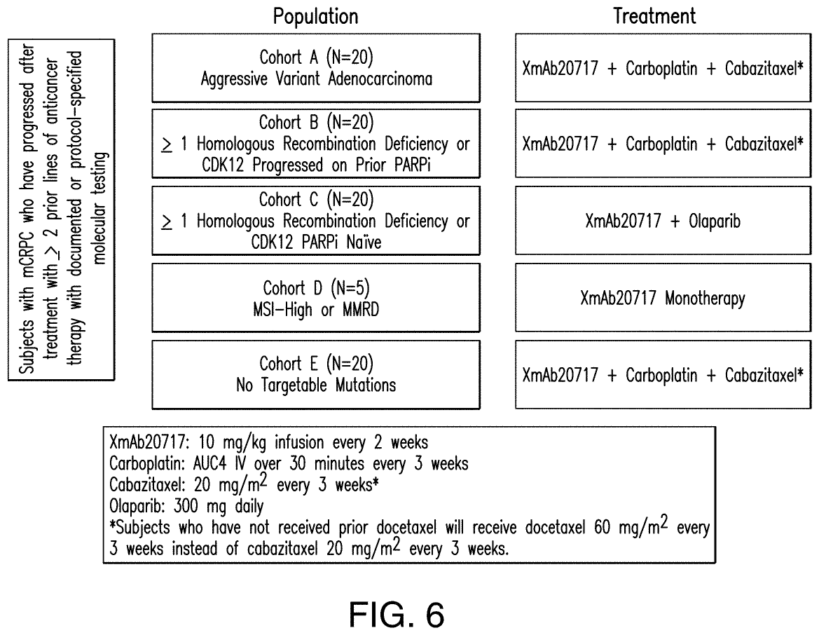

This is a Phase 2, multiple-dose, multiple-arm, parallel assignment study to evaluate the safety and antitumor activity of XmAb®20717 in subjects with metastatic castration-resistant prostate cancer (mCRPC) who have progressed after treatment with at least 2 prior lines of anticancer therapy. The study enrolls subjects in five cohorts (i.e., Cohorts A-E), with up to 20 subjects enrolled in Cohorts A, B, C, and E, and up to 5 subjects enrolled in Cohort D (FIG. 4 and FIG. 6). Cohorts are molecularly defined as follows based on the results of acceptable, documented prior diagnostic.

Cohort A (AVPCa): Subjects with aggressive variant (anaplastic) adenocarcinoma of the prostate (AVPCa) receive XmAb®20717 (10 mg/kg infusion every 2 weeks) plus carboplatin target area under the serum concentration-time curve of 4 (AUC 4) intravenously (IV) over 30 minutes every 3 weeks and cabazitaxel 20 mg/m² IV every 3 weeks.

Cohort B (homologous recombination deficiency [HRD]/cyclin-dependent kinase 12[CDK12] poly-adenosine diphosphate ribose polymerase [PARP] Progressors): Subjects who have progressed on prior PARP inhibitors with at least one of the specified homologous recombination deficiencies receive XmAb®20717 (10 mg/kg infusion every 2 weeks) plus carboplatin AUC 4 IV over 30 minutes every 3 weeks and cabazitaxel 20 mg/m² IV every 3 weeks.

Cohort C (HRD/CDK12 PARP Naïve): Subjects with at least one of the specified molecular abnormalities and who have not yet received a PARP inhibitor receive olaparib 300 mg orally twice daily and XmAb®20717 (10 mg/kg infusion every 2 weeks).

Cohort D (MSI-H or MMRD): Subjects whose tumors have been shown to be microsatellite instability-high [MSI-H] or mismatch repair deficient [MMRD] receive XmAb®20717 alone (10 mg/kg infusion every 2 weeks).

Cohort E (No Targetable Mutations): Subjects receive XmAb®20717 (10 mg/kg infusion every 2 weeks) plus carboplatin AUC 4 IV over 30 minutes every 3 weeks and cabazitaxel 20 mg/m² IV every 3 weeks.

Subjects with a positive molecular test for one of the following molecular abnormalities are enrolled and receive treatment in Cohort A: Positive for aberrancy in at least 2 of the following genes: Rb1, TP53, PTEN.

Subjects with a positive molecular test for one of the following molecular abnormalities are enrolled and receive treatment in Cohort B or C: Positive for HRD in one or more of the following genes: BRCA1, BRCA2, ATM, PALB2, CHEK2, FANCA; and/or positive for biallelic loss of CDK12.

Subjects with a targeted whole exome sequencing or next generation sequencing analysis of a metastatic lesion which is negative for Cohort A and B molecular abnormalities, as well as negative for microsatellite instability-high [MSI-H] or mismatch repair deficient [MMRD], are enrolled and receive treatment in Cohort E.

All patients will receive vudalimab 10 mg/kg intravenously every 2 weeks. Cohorts A, B, and E (n=20 each) also will receive carboplatin AUC 4+cabazitaxel 20 mg/m2 (or docetaxel 60 mg/m2, if not received prior) every 3 weeks; Cohort C (n=20) also will receive olaparib 300 mg 2×/day; and Cohort D (n=5) will receive vudalimab monotherapy. The primary objective is to evaluate the safety/tolerability of treatment based on adverse events. Secondary objectives include evaluating objective response RECIST 1.1, as modified by PCWG3), radiographic progression-free survival, and PSA response. Exploratory objectives include assessing pharmacodynamic activity in peripheral blood and tumor, and correlations of response with cohort-specific molecular tumor characteristics. Enrollment has been initiated.

Male human subjects with a positive MSI-H/MMRD status will be enrolled and receive treatment as described herein: the male human subjects will be administered XmAb®20717 at an intravenous dose of 10 mg/kg on Days 1 and 15 of each 28-day cycle.

XmAb®20717 is a humanized bsAb that binds both CTLA4 and PD1. The XmAb®20717 pharmaceutical composition is a sterile liquid supplied in single-use glass vials. Each 10 mL vial is filled with 10.5 mL of pharmaceutical composition containing 10.0±1.0 mg/mL of XmAb®20717, in 20 mM histidine, 250 mM sorbitol, and 0.01% (w/v) polysorbate-80 at pH 6.2. Each product vial is intended to deliver 10.0 mL of drug solution.

Prior to administration, XmAb®20717 will be diluted to the final required concentration in an ethylene/polypropylene copolymer infusion bag containing 0.9% Sodium Chloride Injection, USP. After dilution, the bag containing XmAb®20717 should be gently inverted 2 to 3 times to mix the solution. The bag should not be shaken.

XmAb®20717 administration should begin as soon as possible after the dosing solution is made. If there is a delay in administration, the dosing solution may be stored at 2-8° C. for no more than 24 hours or at room temperature for no more than 4 hours prior to infusion. The full-calculated dose will be administered based on the subject's actual baseline weight measurement in kilograms. Following the first dose, subsequent doses will be modified only if the subject's weight changes by more than 10% from the Day −1 weight, at which point it will be recalculated using the current weight.

Subjects will undergo biochemical and tumor assessments according to PCWG3 criteria described herein.

Recruitment

Subjects will be recruited through the clinics at each participating institution. Each subject will be assigned a subject screening number at the time of consent.

Screening

Subjects will be consented before undergoing any screening procedures. After consent, subjects must undergo all the assessments listed for the screening period on the Schedule of Assessments to determine preliminary eligibility. The staff at each clinical site will arrange the subject's study visit schedule.

Determination of Eligibility

After preliminary eligibility is established, site staff will submit an enrollment package to Xencor as detailed in the Subject Registration Guidelines. The Xencor Medical Monitor (or designee) will review the enrollment documents provided and confirm the subject's eligibility. If eligibility is confirmed, at this time, the subject will be assigned a subject enrollment number and a cohort. Study treatment cannot begin until the subject's eligibility has been confirmed and a subject number and treatment assignment have been provided to the site.

Subjects who sign a consent form but do not initiate protocol treatment for any reason (e.g., subjects who are screen failures) will be replaced.

Treatment Assignment

Subject Screening Number: Subjects who consent for the study will be assigned a Subject Screening Number. Subject Enrollment Number: Subjects eligible to receive study treatment will be assigned a Subject Enrollment Number.

Dosing Schedule

XmAb®20717 will be administered at a dose of 10 mg/kg on Days 1 and 15 of each 28-day cycle. Subjects are enrolled as follows:

Cohort A (AVPCa): Subjects with AVPCa receive XmAb®20717 (10 mg/kg infusion every 2 weeks) plus carboplatin target AUC of 4 (AUC 4) IV over 30 minutes every 3 weeks and cabazitaxel 20 mg/m2 IV every 3 weeks.

Cohort B (HRD/CDK12 poly-adenosine diphosphate ribose polymerase [PARP] Progressors): Subjects who have progressed on prior PARP inhibitors with at least one of the specified homologous recombination deficiencies receive XmAb®20717 (10 mg/kg infusion every 2 weeks) plus carboplatin AUC 4 IV over 30 minutes every 3 weeks and cabazitaxel 20 mg/m2 IV every 3 weeks.

Cohort C (HRD/CDK12 PARP Naïve): Subjects with at least one of the specified homologous recombination deficiencies and who have not yet received a PARP inhibitor receive olaparib 300 mg orally twice daily and XmAb®20717 (10 mg/kg infusion every 2 weeks).

Cohort D (MSI-H or MMRD): Subjects whose tumors have been shown to be MSI-H or MMRD receive XmAb®20717 alone (10 mg/kg infusion every 2 weeks).

Cohort E (No Targetable Mutations): Subjects receive XmAb®20717 (10 mg/kg infusion every 2 weeks) plus carboplatin AUC 4 IV over 30 minutes every 3 weeks and cabazitaxel 20 mg/m2 IV every 3 weeks.

Subjects in Cohorts A, B, or E may receive up to 8 cycles of carboplatin. Additional cycles of carboplatin may be considered if the Investigator and Medical Monitor agree that a subject is deriving clinical benefit and does not have significant toxicity.

Subjects in Cohorts A, B, or E who have not received prior docetaxel receive docetaxel 60 mg/m2 IV every 3 weeks instead of cabazitaxel 20 mg/m2 every 3 weeks.

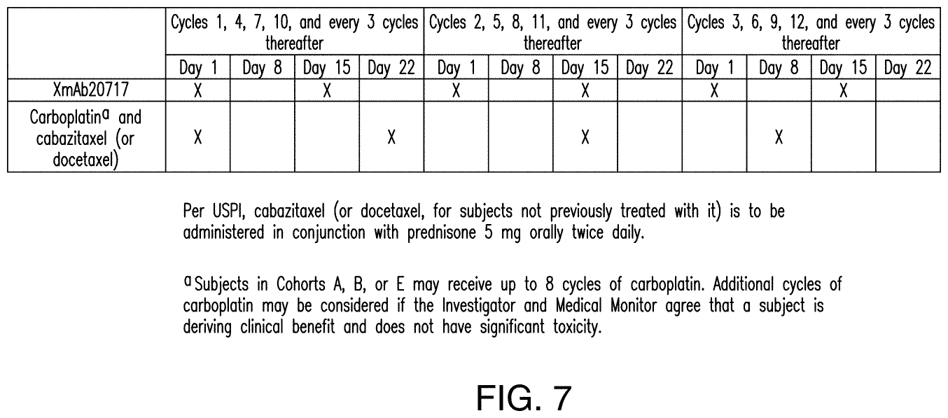

FIG. 7 shows XmAb®20717, carboplatin, and cabazitaxel administration days in each cycle.

Discontinuation of Therapy

In the absence of treatment delays because of AEs, protocol-mandated treatment will continue until a criterion, including, but not limited to, the following applies: Withdrawal by subject: subject decides to withdraw from the study; •Holding of XmAb®20717 for >12 weeks if due to a treatment-related AE, unless continued treatment is approved by the Medical Monitor; •Holding of XmAb®20717 for a continuous time>4 weeks if caused for reason other than treatment-related AE; •Holding of concurrent chemotherapy for >21 days because of persistent cytopenia; •Subject failure to follow study requirements; •Progressive disease and/or no clinical benefit as determined by the treating physician per PCWG3 guidelines (Scher, 2016); •Intercurrent illness that prevents further administration of treatment; •AE: unacceptable AE(s) that may or may not be directly related to treatment but that, in the judgment of the treating physician, makes it dangerous for the subject to be retreated; •Physician decision: general or specific changes in the subject's condition that render the subject unacceptable for further treatment in the judgment of the Investigator; •Study termination by sponsor.

Subjects who show signs of progression may continue treatment on study past initial progression if, as determined by the Investigator, they could still clinically benefit from continued treatment, as outlined in the PCWG3 recommendations. If a subject voluntarily withdraws from the study, attempts should be made to contact the subject to determine and record the reason(s) for discontinuation. All subjects who discontinue the study secondary to an AE must be followed until resolution or stabilization of the AE.

Dose Adjustment Criteria

General Safety Considerations

The following should be taken into consideration in decisions regarding management of treatment-related AEs: •The study drug and commercial drugs administered in this study have class-specific safety profiles based on their mechanisms of action, but may also cause AEs that overlap; •As a general approach, all AEs should be managed with supportive care at the earliest signs of toxicity, including both pharmacological and nonpharmacological treatments, according to consensus management guidelines; •Study treatments may be continued in the event of Grade 1 AEs if appropriate supportive care has been initiated to ameliorate symptoms. Should this be ineffective, or if toxicities become unacceptable, dose modifications of study treatment should be considered to prevent worsening of toxicity; •Grade 2 and 3 AEs usually require dose modification, including dose reductions and/or interruptions. Dose interruptions of any study drug for AEs may occur at any time and independently at the discretion of the Investigator; •Dose modifications as specified in the United States package insert (USPI) should be followed for each commercial drug administered as part of the combination arms on this study; •If XmAb®20717 is interrupted for more than 12 weeks, treatment should be discontinued unless continuation is approved by the Medical Monitor; •Reductions in the XmAb®20717 dose require approval of the Medical Monitor.

Criteria for Adjustment or Stopping Doses

Dose Modification and Toxicity Management for Immune-Related Adverse Events

IrAEs may involve every organ or tissue (Michot, et al. Eur J Cancer. 2016; 54:139-48). Most irAEs occur within the first 12 weeks of exposure to an immune-checkpoint inhibitor, but some of them may appear with a delayed onset. Diagnosis of irAEs should be based on exposure to XmAb®20717 and a reasonable immune-based mechanism of the observed AE. Whenever possible, histologic examination or other immune-based diagnostic evaluations should be used to support the diagnosis. Other etiologic causes, including AEs due to tumor progression, should be ruled out.

The spectrum of irAEs is wide and can be general or organ-specific. Examples of general irAEs in subjects treated with immune-checkpoint inhibitors are fatigue, fever, and chills. Organ-specific irAEs consist of pneumonitis, colitis, hepatitis, nephritis and renal dysfunction, skin adverse reactions, encephalitis, myocarditis, and endocrinopathies.

Early recognition and management of irAEs associated with immune-oncology agents may mitigate severe toxicity. Medical management of irAEs should focus on suppressing the immune response with nonsteroidal and steroidal anti-inflammatory medication. Management algorithms, which provide guidelines on holding, rechallenging, and discontinuation of treatment, have been developed by the National Comprehensive Cancer Network (NCCN) and should be followed for subjects with suspected irAEs. Reductions in the XmAb®20717 dose require approval of the Medical Monitor.

Dose Modifications for Myelosuppression

Subjects receiving chemotherapy may develop treatment-related neutropenia and thrombocytopenia, despite prophylactic growth factor support. For management of chemotherapy-related myelosuppression, refer to the USPI for each specific agent. Provided there are no other signs, symptoms, or laboratory abnormalities suggestive of immunotoxicity, dosing of XmAb®20717 can be continued, despite myelosuppression, while the chemotherapy is held and/or reduced. However, if myelosuppression (platelets<50,000 cells/mm³, absolute neutrophil (ANC)<1500 cells/mm³, or hemoglobin (Hgb)<8.0 g/dL despite transfusion) persists beyond 14 days after a prior cycle of chemotherapy, XmAb®20717 will be held to enable work-up of chemotherapy-unrelated causes, including immune-related thrombocytopenia, neutropenia, and/or anemia. If chemotherapy cannot be resumed within 21 days due to persistent cytopenia, the subject should be discontinued from all study treatment.

Criteria for Study Termination

If the Investigator or the Sponsor becomes aware of conditions or events that suggest a possible hazard to subjects if the clinical study continues, the study may be terminated after appropriate consultation among the involved parties.

Conditions that will prompt such a consultation and may warrant termination of the clinical study include, but are not limited to, the following: •The discovery of an unexpected or unacceptable risk to the subjects enrolled in the clinical study; •Failure to enroll subjects at the required rate; •A decision by the Sponsor to suspend or discontinue development of XmAb®20717; •Sponsor decision. The clinical study may also be terminated at the Sponsor's discretion for these or any other reasons. Should the study be terminated, and/or a site closed for whatever reason, all documentation pertaining to the study and investigational medicinal product (IMP) must be returned to the Sponsor. Any actions required for assessing or maintaining subject safety will continue as required, despite termination of the study or site.

Study Assessments

The Schedule of Assessments is shown herein.

Pretreatment Evaluations

Only those subjects who meet all inclusion and no exclusion criteria may be enrolled into this study. Prior to the initiation of any on-study testing, including screening testing, the subject must have signed the informed consent form (ICF) and received a Subject Screening Number. The pretreatment period lasts for up to 28 days and includes screening evaluations and baseline assessments (defined as the assessment immediately prior to the first dose).

Prior to administration of XmAb®20717, each subject must have completed all screening activities and received approval for enrollment and a cohort assignment from Xencor.

Screening Period (Day −28 to Day −2)

Subjects will undergo a Screening Visit(s) 2 to 28 days prior to the planned first day of study treatment. Please see the footnotes in the Schedule of Assessments and below for acceptable time windows for specific assessments. Screening procedures and assessments are as follows: • Obtain signed informed consent, • Obtain documentation of targeted sequencing of metastatic prostate cancer to determine mutational status of MSI-H or MMRD; • Review inclusion and exclusion criteria; • Collect tissue from formalin-fixed paraffin embedded (FFPE) archival tumor tissue (20 slides, if available, or from fresh pretreatment biopsy); • Collect a blood sample for assessment of ctDNA; • Demographics; • Medical history; • Disease-specific history, including any available genomic information on the primary or metastatic tumor and sequencing data; • Physical examination (PE); • PSA testing within 14 days of the first dose of drug; • Computed tomography (CT) or magnetic resonance imaging (MRI) within 28 days prior to the first dose of study drug for baseline tumor assessment per RECIST 1.1 described herein, as modified by PCWG3 described herein; • Bone scan, if not previously performed within 56 days of the first dose of study drug; • Eastern Cooperative Oncology Group (ECOG) performance status; • Vital signs; • Electrocardiogram (ECG), standard 12-lead; • Complete blood count (CBC) with differential; • Coagulation panel; • Chemistry panel, including amylase and lipase; • Urinalysis; • Testosterone; • Screening test for hepatitis B surface antigen (HBsAg) and hepatitis B core antibody (HBcAb); a subject whose HBsAg is negative and HBcAb is positive may be enrolled if a hepatitis B virus [HBV]DNA test is negative and either the subject is treated with potent antiviral therapy or is retested for HBV DNA every month); • Screening tests for human immunodeficiency virus (HIV) and hepatitis C virus (i.e., HCVRNA); • Record prior and ongoing concomitant medications; • Record AEs (using the National Cancer Institute's Common Terminology Criteria for Adverse Events [NCI-CTCAE] for grading).

Subject Enrollment

Subjects who have completed screening and baseline activities, and whose eligibility checklists have been approved by the Medical Monitor, will be enrolled onto the trial.

Baseline Assessments (Day-1 or Day 1 Predose)

If a procedure or assessment is performed on both Day −1 and Day 1, the one performed closest to the time of dosing is considered the baseline. Please see the footnotes in the Schedule of Assessments and below for acceptable time windows for specific assessments. Baseline procedures and assessments are as follows: • ECOG performance status; • Vital signs; • PE, including height and weight; • ECG, standard 12-lead; • CBC with differential; • Coagulation panel; • Chemistry panel, including amylase and lipase; • Thyroid function tests: thyroid-stimulating hormone (TSH), triiodothyronine (T3), free thyroxine (fT4); • Adrenocorticotropic hormone (ACTH), cortisol (8 AM); • HgBA1C; • Urinalysis; • Blood for cell surface markers by flow cytometry and similar bioanalytical methods (See Laboratory Manual for detailed instructions); • Blood for peripheral cytokine levels (See Laboratory Manual for detailed instructions); • Blood sample stored for potential RNA/transcriptomic profiling (PAXgene); • Serum sample for pembrolizumab or nivolumab levels (subjects in Cohort D who have received pembrolizumab or nivolumab within 18 weeks prior to first dose of study drug); • PSA; • Record AEs; • Record prior and ongoing concomitant medications.

Treatment Cycle Assessments

The schedule of required procedures and clinical site study visit days for each cycle is detailed in the Schedule of Assessments. The day prior to Cycle 1 Day 1 is Day-1.

Clinical Assessments

Predose PEs and clinical evaluations may be performed up to 24 hours prior to a scheduled infusion of XmAb®20717, except where indicated otherwise: • Abbreviated, symptom-directed PE, including weight; • Vital signs (predose assessments must be performed immediately prior to infusion); • ECOG performance status; • Record AEs; • Record prior and ongoing concomitant medications.

Laboratory Assessments

Details for laboratory assessments are provided; details for PK, pharmacodynamics, and flow cytometry are provided in the Laboratory Manuals. Procedures during treatment cycles and for follow-up after End of Treatment (EOT) are detailed herein. Required procedures are also specified in the Schedule of Assessments.

Laboratory assessments include: • CBC; • Coagulation panel; • Chemistry panel, including amylase and lipase; • Thyroid function tests: TSH, T3, fT4; • ACTH, cortisol (8 AM); • HgB A1C; • Urinalysis; Blood for cell surface markers by flow cytometry and similar bioanalytical methods (See Laboratory Manual for detailed instructions); • PK (See Laboratory Manual for detailed instructions); • Blood for peripheral cytokine levels (See Laboratory Manual for detailed instructions); • ADA blood samples (See Laboratory Manual for detailed instructions); • Blood sample stored for potential RNA/transcriptomic profiling (PAXgene); • ECG, standard 12-lead; Tumor assessment by CT/MRI (RECIST 1.1; Eisenhauer et al, Eur J Cancer. 2009; 45:228-47, as modified by PCWG3 (Scher et al. J Clin Oncol. 2016; 34(12):1402-18); PSA measurement (except for Cohort A); • Bone scans per PCWG3 (Scher et al. J Clin Oncol. 2016; 34(12):1402-18); • Post-treatment fresh tumor biopsies should be obtained but are optional and require specific subject consent; • Blood for assessment of ctDNA.

Pharmacokinetic/Pharmacodynamic Assessments

Serum Sampling for Pharmacokinetic Analyses, Anti-Drug Antibodies, and Cytokines/Inflammatory Factors

The Sponsor or designee will provide cryotubes, labels, and requisition forms. Serum cryotubes will be labeled at the clinical site with the subject enrollment number, date, visit day and time point, and time of sampling. Samples will be stored on site at −70° C. until the Sponsor notifies the sites to ship the samples on dry ice to the designated lab for sample analysis. Detailed instructions for processing and shipping serum samples are provided in the Laboratory Manual. Venous blood samples for serum analyses of ADA and the cytokine panel will be obtained according to the Schedule of Assessments. ADA samples may also be analyzed for soluble factors (including but not limited to sCTLA4, sPD1, and sPDL1) that may interfere with the assay. Blood samples for analyses of PK will be obtained according to the Sampling for PK table.

Pharmacodynamics

Detailed instructions for processing and shipping of peripheral blood and tumor biopsy tissue for pharmacodynamic assessments are provided in the Laboratory Manual. For the sampling schedules, see the Schedule of Assessments. The Sponsor/designated vendor will provide blood sampling tubes, labels, requisition forms, and shipping containers.

In peripheral venous blood, baseline and serial assessment of B-cell, natural killer cell, and T-cell numbers, as well as markers of T-cell activation, will be assessed. A blood sample will also be collected at baseline and on treatment and stored for potential RNA/transcriptomic profiling. Archival tissue and/or pre- and post-XmAb®20717 fresh tumor biopsies, if available, will be examined by immunohistochemical and fluorescent immunohistochemical assays for immune cell density, intratumoral and juxta-tumoral immune and tumor cell expression of PDL1, PD1, and other immune checkpoint markers, transcriptomic analysis of the tumor and tumor microenvironment, and next generation sequencing analysis of mutations and tumor mutational burden, for correlation with clinical responses. A blood sample will also be collected for ctDNA mutation profiling.

End of Treatment

Subjects will have their EOT assessments at the end of their last cycle of treatment. If a subject terminates before the end of a cycle, the EOT assessments will be performed on the day of study termination. Subjects with a positive ADA at study termination will be followed every 28 days (±3 days) until ADA is negative or until administration of another anticancer therapy. Follow-up will cease once all scheduled samples have been collected or when the subject begins another anticancer treatment regimen. Subjects will be followed for AEs for 70 days after EOT or until the first administration of another anticancer therapy, whichever comes first.

Post-Treatment Follow-up Period (14 Days, 28 Days, and 70 Days after EOT)

Subjects will have visits 14 days post-EOT, 28 days post-EOT, and 70 days post-EOT. Scans for subjects who discontinued treatment for reasons other than disease progression should be collected until disease progression. Once these visits and assessments have been completed, the subject will be considered as having completed participation in the clinical trial. If a subject enrolls into another trial or requires a change in anticancer treatment before either of these scheduled visits, the visits may be performed earlier. If a subject is unable/unwilling to return for any scheduled visits after the end of treatment, he/she will be considered as having completed study requirements at the end of treatment.

Long-Term Follow-up Period (6 and 12 Months After EOT)

Subjects will be contacted by site staff by telephone, email, or mail at 6 and 12 months after their active participation in the study ends to gather information about disease progression and survival. Scans for subjects who discontinued treatment for reasons other than disease progression should be collected until disease progression. Completion of the long-term follow-up period is not required for a subject to be considered as having completed the clinical trial.

Screening Period

Subjects will undergo a Screening Visit(s) 2 to 28 days prior to the planned first day of study treatment. Screening procedures and assessments are as follows: Obtain signed informed consent; Obtain documentation of targeted sequencing of metastatic prostate cancer to determine mutational status of MSI-H or MMRD; Review inclusion and exclusion criteria; Collect tissue from formalin-fixed paraffin embedded (FFPE) archival tumor tissue (20 slides, if available, or from fresh pretreatment biopsy); Collect a blood sample for assessment of ctDNA; Demographics; Medical history; Disease-specific history, including any available genomic information on the primary or metastatic tumor and sequencing data; Physical Examination (PE); PSA testing within 14 days of the first dose of drug (except subjects in Cohort A); Computed tomography (CT) or magnetic resonance imaging (MM) within 28 days prior to the first dose of study drug for baseline tumor assessment per RECIST 1.1 (Eisenhauer et al. Eur J Cancer. 2009; 45:228-47), as modified by PCWG3 (Scher et al. J Clin Oncol. 2016; 34(12):1402-18); Bone scan, if not previously performed within 56 days of the first dose of study drug; Eastern Cooperative Oncology Group (ECOG) performance status; Vital signs; Electrocardiogram (ECG), standard 12-lead; Complete blood count (CBC) with differential; Coagulation panel; Chemistry panel, including amylase and lipase; Urinalysis; Testosterone; Screening test for hepatitis B surface antigen (HBsAg) and hepatitis B core antibody (HBcAb); a subject whose HBsAg is negative and HBcAb is positive may be enrolled if a hepatitis B virus [HBV] DNA test is negative and either the subject is treated with potent antiviral therapy or is retested for HBV DNA every month); Screening tests for human immunodeficiency virus (HIV) and hepatitis C virus (i.e., HCV-RNA); Record prior and ongoing concomitant medications; Record Adverse Events (using the National Cancer Institute's Common Terminology Criteria for Adverse Events [NCI-CTCAE] for grading)

Subjects who have completed screening and baseline activities, and whose eligibility checklists have been approved by the Medical Monitor, will be enrolled onto the trial.

Please see the footnotes in the Schedule of Assessments and below for acceptable time windows for specific assessments. Baseline procedures and assessments are as follows: ECOG performance status; Vital signs; Physical Examination, including height and weight; ECG, standard 12-lead; CBC with differential; Coagulation panel; Chemistry panel, including amylase and lipase; Thyroid function tests: thyroid-stimulating hormone (TSH), triiodothyronine (T3), freethyroxine (fT4); Adrenocorticotropic hormone (ACTH), cortisol (8 AM)•HgBA1C; Urinalysis; Blood for cell surface markers by flow cytometry and similar bioanalytical methods; Blood for peripheral cytokine levels; Blood sample stored for potential RNA/transcriptomic profiling (PAXgene); PSA (except subjects in Cohort A); Record AEs; Record prior and ongoing concomitant medications.

Predose PEs and clinical evaluations may be performed up to 24 hours prior to a scheduled infusion of XmAb®20717, except where indicated otherwise: Abbreviated, symptom-directed PE, including weight; Vital signs (predose assessments should be performed immediately prior to infusion); ECOG performance status; Record AEs; Record prior and ongoing concomitant medications.

Laboratory assessments include: CBC; Coagulation panel; Chemistry panel, including amylase and lipase; Thyroid function tests: TSH, T3, fT4; ACTH, cortisol (8 AM); HgB A1C; Urinalysis; Blood for cell surface markers by flow cytometry and similar bioanalytical methods; PK; Blood for peripheral cytokine levels; ADA blood samples; Blood sample stored for potential RNA/transcriptomic profiling; ECG, standard 12-lead; Tumor assessment by CT/MRI (RECIST 1.1; Eisenhauer et al, Eur J Cancer. 2009; 45:228-47, as modified by PCWG3 (Scher et al. J Clin Oncol. 2016; 34(12):1402-18); PSA measurement (except Cohort A); Bone scans per PCWG3 (Scher et al. J Clin Oncol. 2016; 34(12):1402-18); Post-treatment fresh tumor biopsies should be obtained but are optional and require specific subject consent; Blood for assessment of ctDNA.

TABLE 6
Schedule of Assessments

| | | | | | | | | | | | | Post-treatment | | | | |
|---|---|---|---|---|---|---|---|---|---|---|---|---|---|---|---|---|
| | | | | | | | | | | | | | | | Safety | |
| | | | | | | | | | | | | | | | Follow- | |
| | Pre- | | | | | | | | | | | | | | up | Follow- |
| | treatment | | Cycle 1 | | | | | | | | | | 14 d | 28 d | 70 d | up |
| | Screen | | | | | | Cycle 2 and higher^{a} | | | | | | post- | post | post- | (6 and |
| Evaluation or | (Days | | | | 15 | | 1 | 8 | 15 | 22 | Day | | EOT | EOT | EOT | 12 mo |
| Procedure | −28 | Day | | | (±2 | 21- | (±2 | (±2 | (±2 | (±2 | 26 ± | | (±2 | (±2 | (+5 | after |
| Day | to −2) | −1 | 1 | 2 | days) | 28 | days) | days) | days) | days) | 2 | EOT | days) | days) | days) | EOT) |
| Informed consent | X | | | | | | | | | | | | | | | |
| Collection of | X | | | | | | | | | | | | | | | |
| archival FFPE tumor | | | | | | | | | | | | | | | | |
| material | | | | | | | | | | | | | | | | |
| Review of inclusion/ | X | | | | | | | | | | | | | | | |
| exclusion criteria | | | | | | | | | | | | | | | | |
| Biopsy of metastatic | X | | | | | X | | | | | | | | | | |
| disease or primary | | | | | | | | | | | | | | | | |
| disease site ^{b} | | | | | | | | | | | | | | | | |
| Medical history ^{c} | X | | | | | | | | | | | | | | | |
| Disease-specific history ^{c} | X | | | | | | | | | | | | | | | |
| Demographics | X | | | | | | | | | | | | | | | |
| Physical examination c | X | X | X | X | X | | X | | X | | | X | X | X | X | |
| Vital signs ^{a, d} | X | X | X | X | X | X | X | X | X | X | | X | X | X | X | |
| Height | | X | | | | | | | | | | | | | | |
| Weight ^{e} | | X | | | X | | X | | X | | | X | X | X | | |
| ECOG performance status ^{f} | X | X | X | | X | | X | | X | | | X | X | X | X | |
| 12-lead ECG ^{g} | X | | X | | X | | X | | X | | | | | X | | |
| CBC w/differential | X | X | X | X | X | | X | | X | | | X | X | X | X | |
| (absolute and percentage), | | | | | | | | | | | | | | | | |
| platelet count ^{f, h} | | | | | | | | | | | | | | | | |
| Chemistry panel ^{f, h} | X | X | X | X | X | | X | | X | | | X | X | X | X | |
| Coagulation panel ^{f, h} | X | X | X | X | X | | X | | | | | X | X | X | X | |
| Urinalysis ^{f, h} | X | X | X | | X | | X | | | | | X | X | X | X | |
| Testosterone | X | | | | | | | | | | | | | | | |
| HBsAg, HBcAb, | X | | | | | | | | | | | | X | | | |
| HCV, HIV ⁱ | | | | | | | | | | | | | | | | |
| ACTH, cortisol ^{j, k} | | | X | | | | X | | | | | X | | X | | |
| TSH, T3, fT4 ^{k} | | | X | | | | X | | | | | X | | X | | |
| Hgb A1C ^{k} | | | X | | | | | | | | | X | | | | |
| Flow cytometry (blood) ^{k} | | | X | X | X | | X | | X | | | X | X | X | | |
| Blood for peripheral | | | X | X | X | | X | | | | | X | | X | | |
| cytokine levels ^{k} | | | | | | | | | | | | | | | | |
| PAXgene RNA sample | | | X | | X | | | | | | | | | | | |
| (blood) ^{k,l} | | | | | | | | | | | | | | | | |
| ctDNA (blood) | X | | | | | X | | | | | | | | | | |
| XmAb ®20717 | | | X | | X | | X | | X | | | | | | | |
| administration ^{m} | | | | | | | | | | | | | | | | |
| PK blood sampling ⁿ | | | X | | X | | X | | X | | | X | X | | | |
| Immunogenicity (ADA) | | | X | | X | | X | | X | | | X | | X | | |
| blood sampling ^{k, o} | | | | | | | | | | | | | | | | |
| Serum sample for | | | X | | | | | | | | | | | | | |
| pembrolizumab or | | | | | | | | | | | | | | | | |
| nivolumab levels ^{p} | | | | | | | | | | | | | | | | |
| CT scan/MRI for RECIST | X | | | | | | | | | | X | X | | | | |
| assessment ^{q, r} | | | | | | | | | | | | | | | | |
| PSA ₛ | | | | | | | | | | | | | | | | |
| Bone scan _{q, r, t} | X | | | | | | | | | | X | X | | | | |
| Monitor/record adverse | X | X | X | X | X | X | X | X | X | X | X | X | X | X | X | |
| events | | | | | | | | | | | | | | | | |
| Record prior and | X | X | X | X | X | X | X | X | X | X | X | X | X | X | X | |
| ongoing concomitant | | | | | | | | | | | | | | | | |
| medication | | | | | | | | | | | | | | | | |
| Phone/email/mail contact | | | | | | | | | | | | | | | | X |
| for progression/survival | | | | | | | | | | | | | | | | |

ACTH = adrenocorticotropic hormone;
ADA = anti-drug antibody;
CBC = complete blood count;
CT = computed tomography;
ctDNA = circulating tumor DNA;
d = day;
ECG = electrocardiogram;
ECOG = Eastern Cooperative Oncology Group;
EOT = end of treatment;
FFPE = formalin-fixed paraffinized embedded;
fT4 = free thyroxine;
HBcAb = hepatitis B core antibody;
HBsAg = hepatitis B surface antigen;
HCV = hepatitis C virus;
Hgb A1C = hemoglobin A1C;
HIV = human immunodeficiency virus;
HRD = homologous recombination deficiency;
IV = intravenous;
MMRD = mismatch repair deficient;
mo = month;
MRI = magnetic resonance imaging;
MSI-H = microsatellite instability-high;
PE = physical examination;
PK = pharmacokinetic;
PSA = prostate-specific antigen;
RECIST = Response Evaluation Criteria in Solid Tumors;
T3 = triiodothyronine;
TSH = thyroid-stimulating hormone.
^{a} The Day 8 and Day 22 assessments can be performed if the subject has chemotherapy infusions on those days.
^{b} For subjects undergoing biopsy of accessible metastatic tumor, the procedure can be performed after the subject has consented and registered to the study. Fresh prostate biopsy is acceptable if there is clear local disease and no other measurable disease site or biopsiable bone lesion.
^{c} Complete medical history and PE at screening. For all the other time points an abbreviated, symptom-directed PE is to be performed. A thorough disease-specific history can be recorded at screening, including prior surgery, radiotherapy, and systemic therapy (including duration of treatment, reason for discontinuation, and best overall response), Gleason score at diagnosis, metastatic disease status at diagnosis, PSA levels for the prior 6 months, and any available genomic information on the primary or metastatic tumor and sequencing data.
^{d} Blood pressure and pulse rate, body temperature, respiratory rate. On days of XmAb ®20717 infusions, vital signs should be taken predose and 30 minutes (±5 minutes) after start of infusion, immediately before end of infusion, and 30 (±5) and 60 (±5) minutes after end of infusion. For subjects in Cohorts A, B, and E, on days where carboplatin and cabazitaxel (or docetaxel) are the only infusions, vital signs should be taken predose. On nondosing days, vital signs should be measured before blood sampling. Vital signs should be taken with subject in the same position. For subjects in Cohorts A, B, and E, on days where carboplatin and cabazitaxel (or docetaxel) are the only infusions, vital signs should be taken predose.
^{e} Baseline body weight to be collected on either Day −1 or Day 1.
f If the assessment is performed or the sample is collected within 3 days prior to dosing on Day 1, it does not need to be repeated on Day 1.
^{g} On dosing days in the first 2 cycles of therapy, ECGs should be performed prior to infusion, 2 to 4 hours after end of infusion, and if clinically indicated. On dosing days after the first 2 cycles of therapy, ECGs should be performed prior to infusion. ECGs should be performed after the subject has been resting supine or semirecumbent for ≥5 minutes. All ECGs should be taken with the subject in the same position.
^{h} May be performed up to 24 hours prior to infusion. These safety laboratories can be performed by both the central laboratory and local clinical site laboratories to allow review of the results by clinical site personnel before XmAb ®20717 infusion.
ⁱ May be performed up to 4 weeks prior to screening.
^{j} Cortisol should be drawn as close to 8 AM as possible.
^{k} Preinfusion on day of dose.
^{l} A blood sample can be collected at baseline and on treatment and stored for potential RNA/transcriptomic profiling.
^{m} XmAb ®20717 can be administered as a 1-hour IV infusion (±10 minutes unless there is approval from the Medical Monitor for a different infusion rate and duration). Subjects will be observed for at least 1 hour following the end of infusion. For subjects in Cohort C, olaparib are administered orally twice daily. See dosing schedules for chemotherapy for subjects in Cohorts A, B, and E. For subjects in Cohort C, olaparib are administered orally twice daily. See dosing schedules for chemotherapy for subjects in Cohorts A, B, and E. For subjects in Cohort C, olaparib are administered orally twice daily.
ⁿ [HOLD]
^{o} For Cycles 3 and higher, ADA sampling will be performed prior to infusion on Day 15. Subjects with a positive ADA at termination can be followed every 28 days (±3 days) until ADA reverts to baseline or until the next intervening treatment.
^{p} This sampling should be performed predose on Day 1 for those subjects in Cohort D (MSI-H/MMRD) who have received pembrolizumab or nivolumab within 18 weeks prior to the first dose of study drug.
^{q} Tumor assessment per RECIST 1.1, as modified by PCWG3. Radiographic evaluations and tumor measurements can be undertaken every 9 weeks (±1 week) for 28 weeks, then every 12 weeks (±1 week) for assessment of both response and disease progression. During post-treatment follow-up, scans for subjects who discontinued treatment for reasons other than disease progression can be collected until disease progression. Radiologic documentation can be provided for subjects removed from study for progressive disease. The date of radiographic evaluations can be calculated from the date of first treatment. Images can be evaluated locally for response and progression. EOT assessment can be performed only if such an assessment has not been performed in the past 60 days.
^{r} For CT scans, MRI, and bone scans, imaging for the Cycle 2 Day 26 time point may be performed within 7 days rather than within 2 days.
ₛ Pretreatment PSA can be performed within 14 days prior to the first dose of drug. Post-treatment PSA can be performed at the end of Cycle 1 and repeated every 2 cycles (Cycle 3, Cycle 5, Cycle 7, etc.) thereafter in subjects except those in Cohort A.
ₜ Historical bone scan, if performed within 56 days of Cycle 1 Day 1, is acceptable for pretreatment.

Inclusion Criteria for Subjects of the Study include:
Able to provide written informed consent;
Adult (age≥18 years);
Sex: Male;
Histologically confirmed diagnosis of carcinoma of the prostate;
Documented progressive mCRPC based on at least one of the following criteria:
PSA progression, defined as at least 2 rises in PSA with a minimum of a 1 week interval (1.0 ng/mL is the minimal starting value if confirmed rise is the only indication of progression)
Soft-tissue progression per RECIST 1.1
Progression of bone disease (evaluable disease) or 2 or more new bone lesions by bone scan;
Prostate cancer must have progressed after treatment with at least 2 prior lines of anticancer therapy approved for treatment of metastatic prostate cancer; prior treatment of subjects in Cohort D (MSI-H or MMRD) should include a checkpoint inhibitor approved by FDA for that indication;
Subjects who did not have a surgical orchiectomy should be on androgen suppression treatment (e.g., luteinizing hormone-releasing hormone agonist) with castrate level of testosterone (≤50 ng/dL) and be willing to continue the treatment throughout the study;
Documentation of the following genetic features, based on prior appropriately validated metastatic tissue analysis (targeted or whole exome sequencing panel performed by Clinical Laboratory Improvement Amendments-certified laboratory):
   Cohort A (AVPCa), subjects should be positive for aberrancy of at least 2 of the following: Rb1, TP53, PTEN;
   Cohort B (HRD), subjects must meet one of the following criteria: Positive for HRD in one or more of the following genes: BRCA1, BRCA2, ATM, PALB2, CHEK2, FANCA; Positive for biallelic loss of CDK12;
   Cohort C (HRD)—At least one of the following criteria: Positive for HRD in one or more of the following genes: BRCA1, BRCA2, ATM, PALB2, CHEK2, FANCA; Positive for biallelic loss of CDK12;
   Cohort D, subjects must have positive MSI-H/MMRD status;
   Cohort E, subjects must have had a targeted whole exome sequencing or next generation sequencing analysis of a metastatic lesion that rules out eligibility for other cohorts;
Evaluable disease according to PCWG3 criteria;
Adequate archival metastatic tumor tissue or agree to undergo a biopsy of at least 1 metastatic site (fresh biopsy of primary prostate is only allowed if there is clear local disease and no other measurable disease site or biopsiable bone lesion);
ECOG performance status of 0 or 1;
Able and willing to complete the study according to the study schedule.

Exclusion Criteria for Subjects of the Study include:
Currently receiving anticancer therapies other than androgen deprivation therapy (ADT);
Treatment with any other anticancer therapy within 2 weeks of the start of study drug (i.e., other immunotherapy, chemotherapy, radiation therapy, etc.);
Prior treatment with any CTLA4, PD1, PDL1, or PDL2-directed immunotherapy, except subjects in Cohort D, who will have had prior FDA-approved checkpoint inhibitor therapy;
Grade 4 immune-mediated AE related to prior immunotherapy (applicable to subjects eligible for Cohort D);
Failure to recover from any toxicity related to previous anticancer treatment to ≤Grade 2;
Have known active central nervous system metastases and/or carcinomatous meningitis. Subjects with previously treated brain metastases may participate provided they are radiologically stable, i.e., are without evidence of progression for at least 4 weeks by repeat imaging (note that the repeat imaging should be performed during study screening), are clinically stable, and are without requirement of steroid treatment for at least 14 days prior to first dose of study treatment;
Platelet count<100×109/L;
Hemoglobin level≤9.0 g/dL;
Absolute neutrophil count≤1.7×109 for subjects who will receive cabazitaxel; <1.0×109/L for all others;
Aspartate aminotransferase at screening>3×upper limit of normal (ULN) for subjects without known liver involvement by tumor or >5×ULN for subjects with known liver involvement by tumor;
Alanine aminotransferase at screening>3×ULN for subjects without known liver involvement by tumor or >5×ULN for subjects with known liver involvement by tumor;
Bilirubin≥1.5×ULN (unless prior diagnosis and documentation of ongoing hemolysis or Gilbert's syndrome has been made);
Estimated creatinine clearance<50 mL/minute calculated by the Cockcroft Gault or Modification of Diet in Renal Disease formulas;
Active known or suspected autoimmune disease (except vitiligo; type 1 diabetes mellitus or residual hypothyroidism due to an autoimmune condition that is treatable with hormone replacement therapy only; psoriasis, atopic dermatitis, or another autoimmune skin condition that is managed without systemic therapy; or arthritis that is managed without systemic therapy beyond oral acetaminophen and nonsteroidal anti-inflammatory drugs);
Have any condition requiring systemic treatment with corticosteroids, prednisone equivalents, or other immunosuppressive medications within 14 days prior to first dose of study drug (except inhaled or topical corticosteroids or brief courses of corticosteroids given for prophylaxis of contrast dye allergic response). Subjects who are currently taking prednisone from a previous prostate cancer therapy will be permitted to enroll in the study;
Receipt of an organ allograft;
Known history of left ventricular ejection fraction≤40%;
History or evidence of any other clinically unstable/uncontrolled disorder, condition, or disease other than their primary malignancy that, in the opinion of the Investigator, would pose a risk to patient safety or interfere with study evaluations, procedures, or completion;
Evidence of any serious bacterial, viral, parasitic, or systemic fungal infections within the 30 days prior to the first dose of study drug;
Receipt of a live-virus vaccine within 30 days prior to the first dose of study drug (seasonal flu vaccines that do not contain live virus are permitted);
A human immunodeficiency virus (HIV) positive subject with CD4+ T-cell (CD4+) counts<350 cells/μL, or an HIV viral load greater than 400 copies/mL, or a history of an AIDS (acquired immunodeficiency syndrome)-defining opportunistic infection within the past 12 months, or who has not been on established antiretroviral therapy (ART) for at least 4 weeks prior to initiation of study drug dosing. (Effective ART is defined as a drug, dosage, and schedule associated with reduction and control of the viral load);
Positive test for hepatitis C RNA (a subject who is hepatitis C virus [HCV] antibody positive but HCV RNA negative due to documented, curative prior antiviral treatment or natural resolution is eligible);

Positive test for hepatitis B surface antigen (HBsAg) or hepatitis B core antibody (HBcAb; a subject whose HBsAg is negative and HBcAb is positive may be enrolled if a hepatitis B virus [HBV] DNA test is negative and the subject is retested for HBsAg and HBV DNA every 2 months

### 6.3 Example 3: Evaluation of XmAb®20717 Activity

The activity of XmAb®20717 was determined in an in vitro assay measuring IL-2 secretion from human lymphocytes stimulated with SEB, a method of assessing in vitro activity that has been used for other checkpoint inhibitors, including nivolumab. SEB-stimulated PBMC were treated with XmAb®20717 or comparators/controls for 24 hours, and lymphocyte function was determined by measuring by ELISA the amount of IL-2 in culture supernatants.

When compared to an anti-RSV isotype-control bivalent antibody (XENP15074), XmAb®20717 promoted a 4.1-fold increase in IL-2 secretion while XENP16432, a benchmark anti-PD1 bivalent antibody derived from the Fv of nivolumab with substitutions in the Fc domain similar to those in the XmAb®20717 Fc domain, promoted a 2.6-fold increase in IL-2 secretion versus control. When compared to PD1 blockade by a bivalent PD1 antibody, XmAb®20717 promoted a 1.5-fold increase in IL-2 secretion.

To determine if the avidity arising from XmAb®20717's simultaneous binding to PD1 and CTLA4 contributed to IL-2 secretion, the intact bispecific antibody was compared to a mixture of its monovalent and monospecific component antibodies. Compared to a mixture of XENP20111 (a monovalent anti-PD1 scFv-Fc component antibody of XmAb®20717) and XENP20059 (a monovalent anti-CTLA4 Fab-Fc component antibody of XmAb®20717), XmAb®20717 promoted a 1.6-fold increase in IL-2 secretion, suggesting that the increased avidity of XmAb®20717 for dual-positive PD1 and CTLA4-expressing T cells contributes to its observed in vitro.

### 6.4 Example 4: A Study of XmAb®20717 in Patients With Selected Advanced Gynecologic and Genitourinary Malignancies

This example describes a study of XmAb®20717 in patients with selected advanced gynecologic and genitourinary malignancies. In particular, this study describes a Phase 2 study of XmAb®20717 in patients with selected gynecological malignancies and high-risk metastatic castration-resistant prostate cancer.

Study Description:

Brief Summary:

This is a Phase 2, multicenter, two-stage, open-label, parallel-group study designed to evaluate the efficacy and safety of XmAb®20717 in patients with selected advanced gynecologic and genitourinary malignancies.

Detailed Description:

This is a Phase 2, multicenter, two-stage, open-label, parallel-group study designed to evaluate the efficacy and safety of XmAb®20717 in patients with selected advanced gynecologic and genitourinary malignancies and to identify tumor types for further evaluation.

In Stage 1, subjects will be enrolled into 1 of 5 tumor-specific, parallel cohorts (n=10 each):
Platinum-resistant high-grade serous ovarian cancer (HGSOC)
Chemotherapy relapsed or refractory clear cell ovarian, endometrial, or peritoneal cancer
Immune-checkpoint-inhibitor-refractory microsatellite stable (MSS) endometrial cancer (EC)
Previously treated recurrent or metastatic cervical cancer
High-risk metastatic castration-resistant prostate cancer (mCRPC)

Within each tumor-specific cohort in Stage 1, a primary endpoint of ORR at 12 weeks, based on investigator review, will be used to determine cohort expansion into Stage 2.

Each Stage 1 cohort that achieves an ORR of ≥20% (at least 2 out of 10 subjects with an objective response) will enroll up to an additional 20 subjects in Stage 2. Cohorts with an ORR of less than 20% will discontinue enrollment. However, additional factors will be considered in determining an expansion into Stage 2 (e.g., enrollment rate, complete versus partial response, and DOR).

### Conditions:

Conditions:
Ovarian Cancer Clear Cell Carcinoma Endometrial Cancer Cervical Carcinoma
Metastatic Castration-Resistant Prostate Cancer (mCRPC)

Keywords:
Endometrial Cancer Ovarian Cancer
Cervical Cancer
Prostate Cancer
Gynecological Cancer
Fallopian Tube Cancer
Peritoneal Cancer
Clear Cell Carcinoma
XmAb®20717

### Study Design:

Study Type: Interventional

Primary Purpose: Treatment

Study Phase: Phase 2

Interventional Study Model: Sequential Assignment

Number of Arms: 1

Masking: None (Open Label)

Allocation: N/A

Enrollment: 150 [Anticipated]

### Arms and Interventions:

Arms—Experimental: XmAb®20717.

Assigned Interventions—Biological/Vaccine: XmAb®20717; Monoclonal bispecific antibody

### Outcome Measures:

Primary Outcome Measure: ORR as assessed by RECIST 1.1 criteria (efficacy)

To determine the Objective Response Rate as assessed by Response Evaluation Criteria in Solid Tumors (RECIST) version 1.1

Time Frame: 12 weeks

### Eligibility:

Minimum Age: 18 Years

Maximum Age: N/A

Sex: All

Gender Based: No

Accepts Healthy Volunteers: No

Inclusion Criteria:

### Inclusion Criteria:

Able to provide written informed consent
Adult (age≥18 years)
Cancer must have progressed after treatment with all approved and medically appropriate therapies or have no appropriate available therapies
Histologically confirmed diagnosis of one of the following tumor types, along with clinical/pathologic confirmation of the additional requirements for each indication, as appropriate:
a. Persistent or recurrent clear cell carcinoma of the ovary, peritoneum, or endometrium after treatment with platinum-based systemic chemotherapy
b. Persistent or recurrent high-grade serous carcinoma of the ovary, fallopian tube, or peritoneum after treatment with platinum-based systemic chemotherapy (except subjects with a diagnosis of carcinosarcoma)
c. Recurrent or metastatic cervical carcinoma previously treated with standard-of-care systemic chemotherapy and FDA-approved immunotherapy, if eligible
d. Advanced endometrial carcinoma that is not microsatellite instability-high (MSI-H) or deficient mismatch repair (dMMR) in patients who are not candidates for curative surgery or radiation, and that has progressed following treatment with no more than one prior line of systemic therapy and prior treatment with FDA-approved combination therapy consisting of a checkpoint inhibitor and a targeted agent
e. High-risk metastatic castration-resistant prostate cancer:
   Castration resistance defined as progressive disease after surgical castration, or progression in the setting of medical androgen ablation with a castrate level of testosterone (<50 ng/dL)
   High-risk disease is any visceral, soft tissue, or lymph node metastasis(es) with/without bone metastases
Measurable disease by Response Evaluation Criteria in Solid Tumors (RECIST 1.1)
Adequate available archival formalin-fixed paraffin-embedded block(s)/slides containing tumor and/or adequate predose fresh tumor biopsy tissue
Eastern Cooperative Oncology Group performance status of 0 or 1
Female subjects of childbearing potential must agree to use a highly effective method of birth control during and for 4 weeks after completion of study. Women are considered to be of childbearing potential unless it is documented that they are over the age of 60 OR postmenopausal by history with no menses for 1 year and confirmed by follicle-stimulating hormone (using local reference ranges) OR have a history of hysterectomy and/or bilateral oophorectomy OR have a history of bilateral tubal ligation. Highly effective methods of birth control include hormonal birth control (oral, intravaginal, or transdermal), or progestogen-only hormonal contraception associated with inhibition of ovulation (oral, injectable, or intrauterine), intrauterine devices (IUDs), intrauterine hormone-releasing system, bilateral tubal occlusion, vasectomized partner (provided partner is the sole sexual partner and there has been a medical assessment of surgical success), or sexual abstinence
Fertile male subjects must be willing to
practice a highly effective method of birth control during and for 4 weeks after completion of study
Male subjects must agree not to donate sperm from screening through 4 weeks after completion of study
Able and willing to complete the entire study according to the study schedule

### Exclusion Criteria:

Subjects currently receiving other anticancer therapies, except that subjects with prostate cancer may continue to receive luteinizing hormone-releasing hormone (LHRH) analogue therapy
More than 2 prior chemotherapy regimens for subjects in the cervical cancer, CCC,

HGSOC, or prostate cancer cohorts
Prior treatment with a CTLA4-targeted agent
Prior treatment with nivolumab, pembrolizumab, or any other PD1-, PDL1- or programmed cell death ligand 2- (PDL2)-directed therapy, except that:
   Subjects with MSS EC may have received anti-PD1 therapy as part of an FDA-approved regimen in the approved disease setting
   Subjects with cervical cancer may have received anti-PD1 therapy as an FDA-approved agent in the approved disease setting
Treatment with any other anticancer therapy within 2 weeks of the start of study drug (i.e., other immunotherapy, chemotherapy, radiation therapy, etc.)
A life-threatening (Grade 4) immune-mediated adverse event (AE) associated with prior administration of an immunotherapy agent
Failure to recover from any immunotherapy-related toxicity from prior cancer therapy to ≤Grade 1, except that subjects are eligible if a previous immunotherapy-related endocrinopathy is medically managed with hormone replacement therapy only
Failure to recover from any other cancer therapy-related toxicity (other than immune-related toxicity) related to previous anticancer treatment to ≤Grade 2
Have known active central nervous system metastases and/or carcinomatous meningitis
Platelet count<100×109/L
Hemoglobin level≤9.0 g/dL
Absolute neutrophil count<1.5×109/L
Aspartate aminotransferase (AST) at screening>3×upper limit of normal (ULN) for subjects without known liver involvement by tumor; or >5×ULN for subjects with known liver involvement by tumor
Alanine aminotransferase (ALT) at screening
>3×ULN for subjects without known liver involvement by tumor; or >5×ULN for subjects with known liver involvement by tumor
Bilirubin≥1.5×ULN (unless prior diagnosis and documentation of ongoing hemolysis or Gilbert's syndrome has been made)
Estimated creatinine clearance<50 mL/minute calculated by the Cockcroft Gault or Modification of Diet in Renal Disease formulas
Active known or suspected autoimmune disease (except that subjects are permitted to enroll if they have vitiligo; type 1 diabetes mellitus or residual hypothyroidism due to an autoimmune condition that is treatable with hormone replacement therapy only; autoimmune adrenal insufficiency that is managed with low-dose corticosteroids; psoriasis, atopic dermatitis, or another autoimmune skin condition that is managed without systemic therapy; or arthritis that is managed without systemic therapy beyond oral acetaminophen and nonsteroidal anti-inflammatory drugs)
Has any condition requiring systemic treatment with corticosteroids, prednisone equivalents, or other immunosuppressive medications within 14 days prior to first dose of study drug (except that inhaled or topical corticosteroids or brief courses of corticosteroids given for prophylaxis of contrast dye allergic response are permitted)
Receipt of an organ allograft
History of small or large bowel obstruction within 3 months of enrollment, including subjects with palliative gastric drainage catheters. Subjects with palliative diverting ileostomy or colostomy are allowed if they have been symptom-free for more than 3 months.
Ongoing bowel perforation or presence of bowel fistula or intra-abdominal abscess
Subjects with refractory ascites, for example, ascites needing drainage catheter or therapeutic paracentesis more often than every 4 weeks
Histologic diagnosis of carcinosarcoma of the ovary
Symptomatic cord compression, or clinical or radiologic findings indicative of impending cord compression
History or evidence of any other clinically unstable/uncontrolled disorder, condition, or disease (including, but not limited to, cardiopulmonary, renal, metabolic, hematologic, or psychiatric) other than their primary malignancy, that, in the opinion of the Investigator, would pose a risk to patient safety or interfere with study evaluations, procedures, or completion
Evidence of any serious bacterial, viral, parasitic, or systemic fungal infections within the 30 days prior to the first dose of study drug
Receipt of a live-virus vaccine within 30 days prior to first dose of study drug (seasonal flu and COVID-19 vaccines are permitted, as long as they do not contain live virus and are not administered within 24 hours of planned administration of XmAb®20717)
An HIV-positive subject with CD4+ T-cell (CD4+) counts<350 cells/μL, or an HIV viral load greater than 400 copies/mL, or a history of an acquired immunodeficiency syndrome (AIDS)-defining opportunistic infection within the past 12 months, or who has not been on established antiretroviral therapy (ART) for at least 4 weeks prior to initiation of study drug dosing. (Effective ART is defined as a drug, dosage, and schedule associated with reduction and control of the viral load. HIV positive subjects who do not meet any of these exclusion criteria are eligible.)
Positive test for hepatitis C RNA (a subject who is hepatitis C virus [HCV] antibody positive but HCV RNA negative due to documented, curative prior antiviral treatment or natural resolution is eligible)
Positive test for hepatitis B surface antigen (HBsAg) or hepatitis B core antibody (HBcAb); a subject whose HBsAg is negative and HBcAb is positive may be enrolled if a hepatitis B virus (HBV) DNA test is negative and the subject is retested for HBsAg and HBV DNA every 2 months. (See the protocol for treatment requirements for subjects with HBV who become HBsAg and HBV DNA positive during the study.)
Subject is pregnant or breastfeeding or planning to become pregnant while enrolled in the study, up to the final end-of-treatment visit
Positive urine pregnancy test (i.e., urine human chorionic gonadotropin)

### 6.5 Example 5: A Phase 2 Study of XmAb®20717 in Patients With Selected Gynecological Malignancies and High-Risk Metastatic Castration-Resistant Prostate Cancer

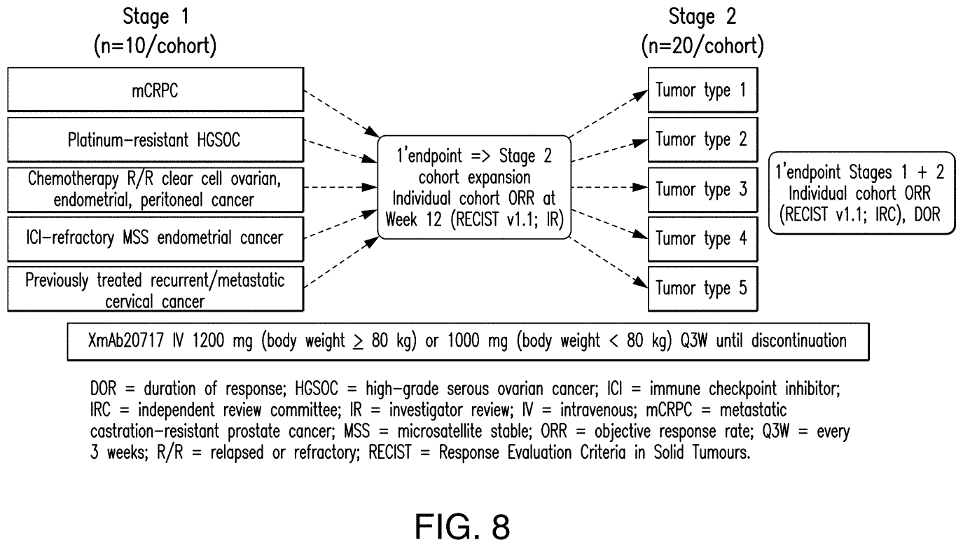

This example describes a Phase 2 Study of XmAb®20717 in patients with selected gynecological malignancies and high-risk metastatic castration-resistant prostate cancer. Up to 50 subjects are enrolled in Stage 1, and up to 100 subjects are enrolled in Stage 2 of the study (FIG. 8). XmAb®20717 is administered as an IV infusion at a constant rate over 1 hour (±10 minutes unless there is approval for a different infusion rate and duration) on Day 1 of each 21-day cycle. XmAb®20717 drug product (DP) is a sterile liquid supplied in single-use glass vials. XmAb®20717 DP is filled with 10.0 mL of DP containing 10.0 mg/mL of XmAb®20717 in 20 mM histidine, 250 mM sorbitol, and 0.01% (weight/volume) polysorbate-80 at pH 6.2. Each 100 mg DP vial is intended to deliver 10.0 mL of drug solution. Subjects who weigh 80 kilograms or more receive 1200 mg of XmAb®20717 IV every 3 weeks (Q3W) and subjects who weigh less than 80 kilograms receive 1000 mg XmAb®20717 IV Q3W.

### Objectives and Endpoints:

| | | |
|---|---|---|
| | | JUSTIFICATION |
| | | FOR |
| OBJECTIVES | ENDPOINTS | ENDPOINTS |

Primary

| | | |
|---|---|---|
| Efficacy: To assess the | overall response rate (ORR) | Typical endpoint for such a |
| antitumor activity of | | study |
| XmAb ®20717 | | |

Secondary

| | | |
|---|---|---|
| Efficacy: To assess the | best observed response | Typical endpoint for such a |
| antitumor activity of | (BOR), duration of response | study |
| XmAb ®20717 | (DOR), progression-free | |
| | survival (PFS), overall | |
| | survival (OS) | |
| | Biochemical response in | |
| | mCRPC subjects | |
| Pharmacokinetics (PK): | Cₘₐₓ, tₘₐₓ, t_{1/2}, AUC_{0-∞}, CL, | Typical endpoint for such a |
| To characterize the PK of | V_{d}, Vdₛₛ, AUC₀₋ₗₐₛₜ, AUCₜₐᵤ, | study |
| XmAb ®20717 | and kₑ | |
| Safety: To determine the | Incidence of treatment- | Typical endpoint for such a |
| safety and tolerability | emergent adverse events | study |
| profile of XmAb ®20717 | (TEAEs), incidence of | |
| | clinically significant changes | |
| | in safety laboratory tests, | |
| | physical exam findings, vital | |
| | signs, electrocardiograms | |
| | (ECGs) | |

Tertiary/Exploratory

| | | |
|---|---|---|
| Patient | Tumor markers, including, but | To identify candidate |
| Stratification/Predictive: | not limited to, programmed | biomarkers that may predict |
| To establish potential | cell death ligand 1 (PDL1), | enhanced clinical response in |
| biomarkers associated with | tumor mutational burden | the tumor indications tested, |
| clinical response in | (TMB), levels of programmed | for future study as patient |
| mandatory baseline biopsies | cell death protein 1 (PD1), | selection tools |
| and blood | cytotoxic T lymphocyte | |
| | associated protein 4 (CTLA4), | |
| | and activation markers in | |
| | intratumoral T cells, | |
| | transcriptional profiles, and | |
| | genomic alterations | |
| | Genomic alterations in blood | To identify potential |
| | as measured longitudinally in | biomarkers of response and |
| | circulating tumor (ct) DNA | resistance. To compare |
| | | ctDNA levels on treatment to |
| | | tumor assessments as an |
| | | exploratory measure of |
| | | clinical response |
| Pharmacodynamics: To | Levels of peripheral | To explore |
| explore pharmacodynamic | leukocytes and | pharmacodynamic effects |
| effects of XmAb ®20717 | markers of T-cell | and their association with |
| in blood and in baseline | activation, | clinical response |
| and optional on treatment | proliferation, and | |
| tumor biopsies | exhaustion | |
| | Intratumoral T cells | |
| | and activation marker | |
| | exnression pre- versus | |
| | post-treatment; | |
| | changes in | |
| | transcriptional profiles | |

AUC = area under the serum concentration-time curve; AUC_{0-∞} = AUC from time zero to infinity; AUC₀₋ₗₐₛₜ = AUC from time zero to time of the last measurable concentration; AUCₜₐᵤ = AUC for a dosing interval, tau; BOR = best observed response; CL = clearance; Cₘₐₓ, = maximum observed concentration; ctDNA = circulating tumor DNA; CTLA4 = cytotoxic T lymphocyte associated protein; DOR = duration of response; ECG = electrocardiogram; ke = terminal elimination rate constant; mCRPC = metastatic castration-resistant prostate cancer; ORR = objective response rate; OS = overall survival; PD1 = programmed cell death protein 1; PDL1 = programmed cell death ligand 1; PFS = progression-free survival; PK = pharmacokinetics; t_{1/2} = the terminal phase elimination half-life; TEAE = treatment- emergent adverse event; tmax, = time at which Cmax occurs; TMB = tumor mutational burden; V_{d} = volume of distribution; Vdₛₛ = volume of distribution at steady state.

Best Observed Response (BOR): The number and percentage of subjects achieving a best response of complete response (CR), partial response (PR), stable disease (SD), and progressive disease (PD) per RECIST 1.1 are presented based on the number of subjects with evaluable response.

Duration of Response (DOR): The duration of response (DOR) is calculated from the time of first documented response that is subsequently confirmed until the date of documented progression or death in the absence of disease progression. Relapse (recurrence after CR) or progression (after PR) are both considered as progression events. Progression-Free Survival (PFS): An analysis of PFS, measured relative to Day 1, are performed based on the Kaplan-Meier method if a sufficient number of events are observed. Median time and its 95% confidence intervals, as well as the 25th and 75th quartiles, are summarized. Progression is determined using the criteria described in RECIST 1.1 and PCWG3, for subjects in the prostate cancer cohort. The date of progression is defined as the date of progression or death. Overall Survival (OS): OS is defined as the time from the date of first dose until death due to any cause. Analysis of OS is performed based on the Kaplan-Meier method if a sufficient number of events are observed.

Biochemical Response (mCRPC Subjects): The number and percentage of mCRPC subjects with PSA response, defined as a ≥50% decrease from baseline or an observed value of <0.2 ng/mL, are presented with the exact 95% confidence intervals of the response rate. Time to PSA response and time to PSA progression may also be evaluated based on the Kaplan-Meier method.

Methodology and Treatment Plan: This Phase 2 study is a multicenter, two-stage, open-label, parallel-group using a two-stage design to evaluate the efficacy and safety of XmAb®20717 in patients with selected advanced gynecologic and genitourinary malignancies and to identify tumor types for further evaluation. A control group is not planned for this study. The two-stage design allows for early stopping if the response rate is unacceptably low during Stage 1 for a tumor-specific cohort. Cohorts meeting the response rate criteria in Stage 1 enrolls 20 additional subjects in Stage 2. Those that advance to Stage 2 are evaluated for ORR and DOR endpoints. The cumulative data from Stages 1 and 2 (n=30 per cohort) informs the design of future studies.

In Stage 1, subjects are enrolled into 1 of 5 tumor-specific, parallel cohorts (n=10 each):
Platinum-resistant high-grade serous ovarian cancer (HGSOC)
Chemotherapy relapsed or refractory clear cell ovarian, endometrial, or peritoneal cancer
Immune-checkpoint-inhibitor-refractory microsatellite stable (MSS) endometrial cancer (EC)
Previously treated recurrent or metastatic cervical cancer
High-risk metastatic castration-resistant prostate cancer (mCRPC)

Within each tumor-specific cohort in Stage 1, a primary endpoint of ORR at 12 weeks, based on investigator review, is used to determine cohort expansion into Stage 2. Each Stage 1 cohort that achieves an ORR of ≥20% (at least 2 out of 10 subjects with an objective response) enroll up to an additional 20 subjects in Stage 2. Cohorts with an ORR of less than 20% discontinue enrollment. However, additional factors are considered in determining an expansion into Stage 2 (e.g., enrollment rate, complete versus partial response, and DOR).

For each tumor-specific cohort that advances into Stage 2, the primary endpoint of ORR, based on independent central review, and the secondary endpoint of DOR is based on the total number of subjects enrolled into Stages 1 and 2 (i.e., n=30/cohort).

Subjects receive XmAb®20717 on Day 1 of each 21-day cycle. Subjects who weigh 80 kilograms or more receive 1200 mg of XmAb®20717 intravenously (IV) every 3 weeks (Q3W) and subjects who weigh less than 80 kilograms receive 1000 mg XmAb®20717 IV Q3W. However, if, at any time after study drug administration has begun, the subject's weight changes by more than 10% from baseline (excluding weight attributable to ascites and/or peripheral edema), and the new weight requires reassignment to a different dosing tier, subsequent doses are received at the new dose level until such time as fulfillment of these rules may again require assignment to the other dose level.

Diagnosis and Criteria for Inclusion/Exclusion: Each subject must meet all of the following inclusion criteria to be enrolled in the study:
Able to provide written informed consent
Adult (age≥18 years)
Cancer must have progressed after treatment with all approved and medically appropriate therapies or have no appropriate available therapies
Histologically confirmed diagnosis of one of the following tumor types, along with clinical/pathologic confirmation of the additional requirements for each indication, as appropriate:
a. Persistent or recurrent high-grade serous carcinoma of the ovary, fallopian tube, or peritoneum after treatment with platinum-based systemic chemotherapy (except subjects with a diagnosis of carcinosarcoma)
b. Persistent or recurrent clear cell carcinoma of the ovary, peritoneum, or endometrium after treatment with platinum-based systemic chemotherapy
c. Advanced endometrial carcinoma that is not microsatellite instability-high (MSI-H) or deficient mismatch repair (dMMR) in patients who are not candidates for curative surgery or radiation, and that has progressed following treatment with no more than one prior line of systemic therapy and prior treatment with FDA-approved combination therapy consisting of a checkpoint inhibitor and a targeted agent
d. Recurrent or metastatic cervical carcinoma previously treated with standard-of-care systemic chemotherapy and FDA-approved immunotherapy, if eligible
e. High-risk metastatic castration-resistant prostate cancer:
   Castration resistance defined as progressive disease after surgical castration, or progression in the setting of medical androgen ablation with a castrate level of testosterone (<50 ng/dL)
   High-risk disease is any visceral, soft tissue, or lymph node metastasis(es) with/without bone metastases
Measurable disease by Response Evaluation Criteria in Solid Tumors (RECIST 1.1)
Adequate available archival formalin-fixed paraffin-embedded block(s)/slides containing tumor and/or adequate predose fresh tumor biopsy tissue
Eastern Cooperative Oncology Group performance status of 0 or 1
Female subjects of childbearing potential must agree to use a highly effective method of birth control during and for 4 weeks after completion of study. Women are considered to be of childbearing potential unless it is documented that they are over the age of 60 or postmenopausal by history with no menses for 1 year and confirmed by follicle-stimulating hormone (using local reference ranges) or have a history of hysterectomy and/or bilateral oophorectomy or have a history of bilateral tubal ligation. Highly effective methods of birth control include hormonal birth control (oral, intravaginal, or transdermal), or progestogen-only hormonal contraception associated with inhibition of ovulation (oral, injectable, or intrauterine), intrauterine devices (IUDs), intrauterine hormone-releasing system, bilateral tubal occlusion, vasectomized partner (provided partner is the sole sexual partner and there has been a medical assessment of surgical success), or sexual abstinence
Fertile male subjects must be willing to practice a highly effective method of birth control during and for 4 weeks after completion of study
Male subjects must agree not to donate sperm from screening through 4 weeks after completion of study
Able and willing to complete the entire study according to the study schedule

Subjects who meet any of the following criteria are excluded from the study:
Subjects currently receiving other anticancer therapies, except that subjects with prostate cancer may continue to receive luteinizing hormone-releasing hormone (LHRH) analogue therapy
More than 2 prior chemotherapy regimens for subjects in the cervical cancer, CCC, HGSOC, or prostate cancer cohorts
Prior treatment with a CTLA4-targeted agent
Prior treatment with nivolumab, pembrolizumab, or any other PD1-, PDL1- or programmed cell death ligand 2- (PDL2)-directed therapy, except that:
a. Subjects with MSS EC may have received anti-PD1 therapy as part of an FDA-approved regimen in the approved disease setting
b. Subjects with cervical cancer may have received anti-PD1 therapy as an FDA-approved agent in the approved disease setting
Treatment with any other anticancer therapy within 2 weeks of the start of study drug (i.e., other immunotherapy, chemotherapy, radiation therapy, etc.)
A life-threatening (Grade 4) immune-mediated adverse event (AE) associated with prior administration of an immunotherapy agent
Failure to recover from any immunotherapy-related toxicity from prior cancer therapy to ≤Grade 1, except that subjects are eligible if a previous immunotherapy-related endocrinopathy is medically managed with hormone replacement therapy only
Failure to recover from any other cancer therapy-related toxicity (other than immune-related toxicity) related to previous anticancer treatment to ≤Grade 2
Have known active central nervous system metastases and/or carcinomatous meningitis
Platelet count<100×10⁹/L
Hemoglobin level≤9.0 g/dL
Absolute neutrophil count<1.5×10⁹/L
Aspartate aminotransferase (AST) at screening>3×upper limit of normal (ULN) for subjects without known liver involvement by tumor; or >5×ULN for subjects with known liver involvement by tumor
Alanine aminotransferase (ALT) at screening>3×ULN for subjects without known liver involvement by tumor; or >5×ULN for subjects with known liver involvement by tumor
Bilirubin≥1.5×ULN (unless prior diagnosis and documentation of ongoing hemolysis or Gilbert's syndrome has been made)
Estimated creatinine clearance<50 mL/minute calculated by the Cockcroft Gault or

Modification of Diet in Renal Disease formulas
Active known or suspected autoimmune disease (except that subjects are permitted to enroll if they have vitiligo; type 1 diabetes mellitus or residual hypothyroidism due to an autoimmune condition that is treatable with hormone replacement therapy only; autoimmune adrenal insufficiency that is managed with low-dose corticosteroids; psoriasis, atopic dermatitis, or another autoimmune skin condition that is managed without systemic therapy; or arthritis that is managed without systemic therapy beyond oral acetaminophen and nonsteroidal anti-inflammatory drugs)
Has any condition requiring systemic treatment with corticosteroids, prednisone equivalents, or other immunosuppressive medications within 14 days prior to first dose of study drug (except that inhaled or topical corticosteroids or brief courses of corticosteroids given for prophylaxis of contrast dye allergic response are permitted)
Receipt of an organ allograft
History of small or large bowel obstruction within 3 months of enrollment, including subjects with palliative gastric drainage catheters. Subjects with palliative diverting ileostomy or colostomy are allowed if they have been symptom-free for more than 3 months.
Ongoing bowel perforation or presence of bowel fistula or intra-abdominal abscess
Subjects with refractory ascites, for example, ascites needing drainage catheter or therapeutic paracentesis more often than every 4 weeks
Histologic diagnosis of carcinosarcoma of the ovary
Symptomatic cord compression, or clinical or radiologic findings indicative of impending cord compression
History or evidence of any other clinically unstable/uncontrolled disorder, condition, or disease (including, but not limited to, cardiopulmonary, renal, metabolic, hematologic, or psychiatric) other than their primary malignancy, that, in the opinion of the Investigator, would pose a risk to patient safety or interfere with study evaluations, procedures, or completion
Evidence of any serious bacterial, viral, parasitic, or systemic fungal infections within the 30 days prior to the first dose of study drug
Receipt of a live-virus vaccine within 30 days prior to first dose of study drug (seasonal flu and COVID-19 vaccines are permitted, as long as they do not contain live virus and are not administered within 24 hours of planned administration of XmAb®20717)
An HIV-positive subject with CD4+ T-cell (CD4+) counts<350 cells/μL, or an HIV viral load greater than 400 copies/mL, or a history of an acquired immunodeficiency syndrome (AIDS)-defining opportunistic infection within the past 12 months, or who has not been on established antiretroviral therapy (ART) for at least 4 weeks prior to initiation of study drug dosing. (Effective ART is defined as a drug, dosage, and schedule associated with reduction and control of the viral load. HIV positive subjects who do not meet any of these exclusion criteria are eligible.)
Positive test for hepatitis C RNA (a subject who is hepatitis C virus [HCV] antibody positive but HCV RNA negative due to documented, curative prior antiviral treatment or natural resolution is eligible)
Positive test for hepatitis B surface antigen (HBsAg) or hepatitis B core antibody (HBcAb); a subject whose HBsAg is negative and HBcAb is positive may be enrolled if a hepatitis B virus (HBV) DNA test is negative and the subject is retested for HBsAg and HBV DNA every 2 months. (See the protocol for treatment requirements for subjects with HBV who become HBsAg and HBV DNA positive during the study.)
Subject is pregnant or breastfeeding or planning to become pregnant while enrolled in the study, up to the final end-of-treatment visit
Positive urine pregnancy test (i.e., urine human chorionic gonadotropin)

### Criteria for Evaluation:

Efficacy: Antitumor activity of XmAb®20717 as assessed by ORR (RECIST 1.1), BOR (RECIST 1.1), as well as DOR, PFS, and OS. Prostate cancer subjects also undergo prostate-specific antigen (PSA) measurement and appropriate imaging scans to allow tumor evaluation per Prostate Cancer Working Group 3 (PCWG3).

Safety: as assessed by: incidence of treatment-emergent adverse events (TEAEs) and incidence of clinically significant changes in safety laboratory tests, physical exams findings, vital signs, and/or ECGs.

Pharmacokinetics: Serum XmAb®20717 concentration is measured and is used to compute PK parameters (e.g., maximum observed concentration [Cₘₐₓ], time at which Cₘₐₓ occurs [tₘₐₓ], area under the serum concentration-time curve [AUC] from time zero to infinity [AUC_{0-∞}], AUC from time zero to time of the last measurable concentration [AUG)] AUC for a dosing interval, tau [AUCₜₐᵤ], the terminal phase elimination half-life [t_{1/2}], clearance of drug from the body [CL], volume of distribution [V_{d}], volume of distribution at steady state [Vdₛₛ], and terminal elimination rate constant [ke]).

Pharmacodynamics: Changes in T-cell subsets and expression of activation/exhaustion markers as measured by flow cytometry assessment and similar bioanalytical methods, and changes in expression profiles of fresh tumor samples (if available) by immunohistochemistry and fluorescent immunohistochemistry.

Continuous variables are summarized with N (sample size), mean, median, standard deviation, minimum, and maximum, and discrete variables are summarized with frequencies and percentages. Safety and efficacy summaries are presented for each tumor-specific cohort or dose level separately and, for safety, also for the combined subject total.

Immunogenicity Assays: The incidence and titer of anti-XmAb®20717 antibodies are reported by cohort and sample collection time point.

Analysis Sets: The analysis sets defined below are used for analysis:
Safety Analysis Set: All subjects who receive at least 1 infusion of XmAb®20717. Safety variables include AEs, vital signs, PE findings, clinical laboratory safety assessments, and ECG parameters. Treatment-emergent AEs (TEAEs) are defined as events with onset dates on or after the start of study treatment or events that are present before the first infusion of XmAb®20717 and subsequently worsen in severity. AE-related endpoints include: TEAEs; Treatment-emergent serious adverse event (TE-SAEs); Treatment-related TEAEs; Treatment-related TE-SAEs; TEAEs by severity that are defined by National Cancer Institute's Common Terminology Criteria for Adverse Events (NCI-CTCAE, Version 5.0); TEAEs resulting in the permanent discontinuation of XmAb®20717; and irAEs (immunotherapy-related adverse event).
Efficacy Analysis Set: All subjects who receive at least 1 infusion of XmAb®20717. This analysis set is used for efficacy analyses.
Pharmacodynamic Analysis Set: All subjects who receive at least 1 infusion of XmAb®20717 and have at least 1 preinfusion and 1 postinfusion set of biomarker data that is considered as sufficient, interpretable, and available for analysis. This analysis set is used for all pharmacodynamic data analyses.
Pharmacokinetic Analysis Set: All subjects who receive at least 1 infusion of XmAb®20717 and have at least 1 set of postinfusion PK data that is considered as sufficient, interpretable, and available for analysis. This analysis set is used for all PK data analyses.

Sample Size: Subjects are enrolled into parallel, disease-specific cohorts in 2 stages. The total sample size of each cohort across the 2 stages depends on the ORR in Stage 1. In Stage 1, approximately 10 subjects are enrolled into each cohort. If there are at least 2 responders (ORR of at least 20%), approximately an additional 20 subjects are enrolled in Stage 2. For any given cohort in Stage 1, if the Stage 1 ORR is less than 20%, additional enrollment may not be initiated in Stage 2; however, consideration may be given to additional factors in making the final determination (e.g., enrollment rate, complete versus partial response, and DOR). The maximum sample size of each cohort is based on demonstrating if the true ORR in each cohort exceeds 10%. Assuming a true response rate of 33%, a sample size of 30 subjects provides 84% power to rule out an inactivity rate of 10% with a one-sided Type I error rate of 5%. The minimum sample size across all cohorts is approximately 50, and the maximum sample size is approximately 150. Subjects who discontinue treatment are replaced.

Objective Response Rate (ORR): ORR is defined as the proportion of subjects with measurable disease at baseline, with at least one visit response of CR or PR based on RECIST 1.1. Two-sided exact 95% confidence intervals for the ORR are determined using the binomial distribution. Investigator assessment of ORR at Week 12 are used to determine cohort expansion into Stage 2. However, the final efficacy analysis are based on the ORR by IRC using RECIST 1.1 criteria and supported by the Investigator determined ORR. An IRC conducts a blinded review of radiology scans performed for final evaluation of study efficacy. The review is conducted by an imaging laboratory to document response (CR, PR, SD, or PD) to therapy on all subjects with at least one post-baseline RECIST 1.1 disease assessment.

Schedule of activities: schedule of activities for stages 1 and 2 are summarized in Table 7 below.

TABLE 7
Schedule of Activities for Stages 1 and 2

| | | | | | | | | | | | | | | | | Post-Treatment | | | | |
|---|---|---|---|---|---|---|---|---|---|---|---|---|---|---|---|---|---|---|---|---|
| | | | | | | | | | | | | | | | | | | | | Follow- |
| | Screen | | | | | | | | | | | | | | | | | | Safety | up |
| | Screen | | | | | | | | | | | | Cycles 9 | | | | | | Follow- | (6 and |
| Evaluation or | (Days | Cycle 1 | | | | Cycle 2 | | | Cycles 3-8 | | | | and higher | | | | 14 d | 28 d | up 70 d | 12 mo |
| Procedure | −28 | | 3- | 8- | 14- | | 8- | 14- | | 3- | 8- | 14- | | 8- | 14- | | post- | post- | post- | after |
| Cycles/Days | to −1) | 1 | 5 ^{a} | 12 ^{b} | 20 | 1 | 12 ^{b} | 20 | 1 | 5 ^{a} | 12 ^{b} | 20 | 1 | 12 ^{b} | 20 | EOT | EOT | EOT | EOT | EOT) |
| Informed consent | X | | | | | | | | | | | | | | | | | | | |
| Collection of | X | | | | | | | | | | | | | | | | | | | |
| archival FFPE | | | | | | | | | | | | | | | | | | | | |
| tumor material ^{c} | | | | | | | | | | | | | | | | | | | | |
| Review of | X | | | | | | | | | | | | | | | | | | | |
| inclusion/ | | | | | | | | | | | | | | | | | | | | |
| exclusion criteria | | | | | | | | | | | | | | | | | | | | |
| Tumor biopsy ^{d} | X | | | | | X | | | | | | | | | | | | | | |
| Medical history ^{e} | X | X | | | | | | | | | | | | | | | | | | |
| Demographics | X | | | | | | | | | | | | | | | | | | | |
| PE ^{e} | X | X | | | | X | | | X | | | | X | | | X | X | X | X | |
| Vital signs^{f} | X | X | | X | | X | X | | X | | X | | X | X | | X | X | X | X | |
| Assessment of | | | | X | | | X | | | | X | | | X | | | | | | |
| Clinical Symptoms ^{g} | | | | | | | | | | | | | | | | | | | | |
| Height | | X | | | | | | | | | | | | | | | | | | |
| Weight | | X | | | | X | | | X | | | | X | | | | X | | | |
| ECOG performance | X | X | | | | X | | | X | | | | X | | | X | X | X | X | |
| status | | | | | | | | | | | | | | | | | | | | |
| 12-lead ECG^{h} | X | X | | | | X | | | | | | | | | | X | | X | | |
| CBC w/differential | X | X | | X | | X | X | | X | | X | | X | X | | X | X | X | | |
| (absolute and | | | | | | | | | | | | | | | | | | | | |
| percentage), platelet | | | | | | | | | | | | | | | | | | | | |
| count ⁱ | | | | | | | | | | | | | | | | | | | | |
| Chemistry panel ⁱ | X | X | | X | | X | X | | X | | X | | X | X | | X | X | X | | |
| Coagulation panelⁱ | X | X | | | | X | | | X | | | | X | | | X | X | X | | |
| Urinalysisⁱ | X | | | | | X | | | X | | | | X | | | X | | | | |
| Urine β-hCG | X | X | | | | X | | | X | | | | X | | | X | | X | | |
| (females of | | | | | | | | | | | | | | | | | | | | |
| childbearing | | | | | | | | | | | | | | | | | | | | |
| potential) ⁱ | | | | | | | | | | | | | | | | | | | | |
| Serum FSH | X | | | | | | | | | | | | | | | | | | | |
| (postmenopausal | | | | | | | | | | | | | | | | | | | | |
| women only) ^{j} | | | | | | | | | | | | | | | | | | | | |
| Testosterone ^{k} | X | | | | | | | | | | | | | | | | | | | |
| HBV, HCV, HIV | X | | | | | | | | | | | | | | | X | | | | |
| testing ^{l} | | | | | | | | | | | | | | | | | | | | |
| ACTH, cortisol ^{m, n} | | X | | | | X | | | | | | | | | | X | | | | |
| TSH, T3, fT4 ⁿ | | X | | | | X | | | | | | | | | | X | | | | |
| Blood for flow | | X | | X | | X | X | | X | | X | | | | | X | | | | |
| cytometry ⁿ | | | | | | | | | | | | | | | | | | | | |
| Blood for peripheral | X | X | | X | | X | X | | X | | X | | | | | X | | | | |
| cytokine levels ⁿ | | | | | | | | | | | | | | | | | | | | |
| Blood for ctDNA | | X | | | | X | | | X | | | | X | | | X | | | | |
| assessment ^{o} | | | | | | | | | | | | | | | | | | | | |
| PAXgene RNA | | X | | | | X | | | | | | | | | | X | | | | |
| Study drug | | X | | | | X | | | X | | | | X | | | | | | | |
| administration ^{p} | | | | | | | | | | | | | | | | | | | | |
| PK blood sampling ^{q} | | X | X | X | | X | | | X | X | X | | X | | | X | X | X | | |
| Immunogenicity | | X | | X | | X | | | X | | | | X | | | X | | X | | |
| (ADA) blood | | | | | | | | | | | | | | | | | | | | |
| sampling ^{r} | | | | | | | | | | | | | | | | | | | | |
| Serum sample for | | X | | | | | | | | | | | | | | | | | | |
| pembrolizumab and | | | | | | | | | | | | | | | | | | | | |
| nivolumab levels ^{s} | | | | | | | | | | | | | | | | | | | | |
| RECIST 1.1 tumor | X | | | | | | | X | | | | X | | | X | X | | | | |
| assessments ^{t} | | | | | | | | | | | | | | | | | | | | |
| PSA ^{u} | X | | | | X | | | | | | | X | | | X | X | | | | |
| Bone Scan ^{v} | X | | | | | | | X | | | | X | | | X | X | | | | |
| Monitor/record AEs | X | X | | X | | X | X | | X | | X | | X | X | | X | X | X | X | |
| Record concomitant | X | X | | X | | X | X | | X | | X | | X | X | | X | X | X | X | |
| medication | | | | | | | | | | | | | | | | | | | | |
| Phone/email/mail | | | | | | | | | | | | | | | | | | | | X |
| contact for | | | | | | | | | | | | | | | | | | | | |
| progression/survival | | | | | | | | | | | | | | | | | | | | |

Abbreviations:
ACTH = adrenocorticotropic hormone;
ADA = anti-drug antibody;
AE = adverse event;
β-hCG = beta unit of human chorionic gonadotrophin;
C#D# = Cycle # Day #;
CBC = complete blood count;
ctDNA = circulating tumor DNA;
d = day;
ECG = electrocardiogram;
ECOG = Eastern Cooperative Oncology Group;
eCRF = electronic case report form;
EOI = end of infusion;
EOT = end of treatment;
FFPE = formalin-fixed paraffin embedded;
FSH = follicle-stimulating hormone;
fT4 = free thyroxine;
HBcAb = hepatitis B core antibody;
HBsAg = hepatitis B surface antigen;
HBV = hepatitis B virus;
HCV = hepatitis C virus;
HIV = human immunodeficiency virus;
IV = intravenous;
mo = month;
PE = physical examination;
PK = pharmacokinetics;
PSA = prostate-specific antigen;
RECIST = Response Evaluation Criteria in Solid Tumours;
T3 = triiodothyronine;
TSH = thyroid- stimulating hormone.
^{a} For each assessment during the Day 3 to 5 period, one assessment should be performed at any time. For Cycles 3 to 8, the Day 3 to 5 PK assessment should be performed at Cycle 5.
^{b} For each assessment during the Day 8 to 12 period, one assessment should be performed at any time. Blood draws and vital signs may be performed either by clinical site personnel or by home health care.
^{c} If archival FFPE is not available, a fresh tumor biopsy may be taken instead.
^{d} Fresh tumor biopsies require specific subject consent. Fresh tumor biopsies are mandatory at screening for all patients with accessible lesions. The on-treatment biopsies at C2D1 are optional and can be collected between C1D18 and C2D8.
^{e} Complete medical history and PE at screening. For all the other time points, an abbreviated medical history and an abbreviated, symptom-directed PE is to be performed and any findings recorded on the AE eCRF.
^{f} Blood pressure, heart rate, body temperature, respiratory rate, measured while subject is in same position for each instance, if possible. On infusion days, vital signs should be taken predose, immediately before the end of the infusion, and 30 (±5) minutes after end of infusion; on non-dosing days, vitals should be measured before any blood sampling.
^{g} Open-ended questioning about symptoms and/or AEs. This assessment will be performed by clinical site personnel at either an in-person or a telemedicine visit.
^{h} On C1D1 an ECG should be performed prior to infusion and 1 to 2 horns after EOT ECG should be performed prior to infusion on C2D1, and it should be collected at EOT. ECG should also be performed at any time thereafter during the subject’s participation in the study if clinically indicated.
ⁱ Safety laboratories performed prior to a scheduled dose may be drawn up to 24 hours prior to infusion. These safety laboratories will be performed by both the central laboratory and local clinical site laboratories to allow review of the results by clinical site personnel before proceeding with the planned XmAb ®20717 infusion. Scheduled safety laboratories performed on other study days may be performed ±24 hours of the scheduled time/day. Safety laboratories may also be drawn at any time as clinically indicated.
^{j} Once postmenopausal status has been established by FSH, this test need not be repeated during the clinical trial.
^{k} Required for prostate cancer patients only.
^{l} May be performed up to 4 weeks prior to screening. A subject whose HBsAg is negative and HBcAb is positive may be enrolled if an HBV DNA test is negative and the subject is retested for HBsAg and HBV DNA every 2 months. Subjects who have positive screening tests HIV or HBV but who are eligible and allowed to emoll in the trial need not repeat these screening tests at EOT.
^{m} Cortisol should be drawn as close to 8 AM as possible.
ⁿ Preinfusion on day of dose. Blood for flow cytometry and cytokines should be collected through Cycle 3 Day 8-12. An EOT sample should also be collected.
^{o} Preinfusion on day of dose. Beginning with C3D1, ctDNA should be collected every other cycle at C3D1, C5D1, C7D1 and C9D1, and then at EOT.
^{p} XmAb ®20717 is administered as a 1-hour (±10 minutes) IV infusion unless there is approval from the Medical Monitor for a different infusion rate and duration. After their first infusion of study drug, subjects will be observed for an hour. For subsequent doses of study drug, subjects with no infusion reaction related to the first dose of study drug may be observed for 30 minutes. For subjects who experience an infusion reaction with any infusion of XmAb ®20717, plans for prophylaxis and observation should be agreed upon by the site’s treating physician and the Medical Monitor.
^{q} In Cycle 1 and Cycle 5, PK samples will be drawn on Day 1 as shown in Table 10. During all other cycles, PK samples will be drawn on Day 1 at predose and at 5 minutes post-EOI. PK samples will also be drawn at EOT, 14 days after EOT, and 28 days after EOT.
^{r} ADA sampling will be performed predose on Day 1 of each cycle, Cycle 1 Day 8 to 12, at EOT, and 28 days post-EOT.
^{s} Nivolumab and/or pembrolizumab sampling will be performed predose on Day 1 only for those subjects who have previously received these antibody treatments within 6 months of the first dose of study drug.
^{t} Imaging and tumor assessment per RECIST 1.1 at screening, at the end of Cycle 2, and at the end of every second cycle of treatment (every 6 weeks) through Cycle 8. Thereafter, imaging and RECIST assessments should be performed at the end of every 3 cycles (every 9 weeks). All scans for baseline tumor assessments should be performed within 28 days prior to the first dose of dmg. EOT assessment should be performed only if such an assessment has not been performed in the past 60 days. Subjects who discontinue XmAb ®20717 due to an AE will continue tumor response assessments according to this schedule until death or disease progression requiring therapy, whichever comes first. For subjects who discontinue treatment for reasons other than disease progression, assessment of response based on standard-of-care imaging will be collected until death or disease progression requiring therapy, whichever comes first.
^{u} PSA (performed by the central laboratory) in prostate cancer subjects only. PSA to be measured during screening, at the end of Cycle 1, and repeated every 2 cycles (Cycle 1, Cycle 3, Cycle 5, etc.) thereafter. Subjects who experience a PSA decrease of ≥50% will have an additional PSA sample drawn 3 to 4 weeks after the response is identified.
^{v} Bone scan is only required for those in the prostate cancer cohort. Historical bone scan, if performed within 56 days of C1D1, is acceptable for baseline. Bone scan is to be performed at the end of Cycle 2, then every 3 cycles (9 weeks) for 28 weeks (Cycles 5, 8, and 11), and then every 4 cycles (12 weeks) thereafter. An EOT bone scan assessment will be performed unless a bone scan has been performed within the past 60 days.

Data From Clinical and Nonclinical Studies of XmAb®20717: In nonclinical studies, XmAb®20717 was shown to bind to human and cynomolgus monkey T cells and to simultaneously occupy the PD1 and CTLA4 binding sites on the surface of activated T cells, blocking programmed cell death ligands 1 and 2 (PDL1 and PDL2) from binding to these cells. XmAb®20717 was also shown to promote interleukin 2 secretion, demonstrating superior activity when bound to super-antigen-stimulated human lymphocytes than simultaneous blockade of CTLA4 and PD1 by the 2 monovalent antibodies that are components of XmAb®20717. These results suggest that the increased avidity of XmAb®20717 for dual-positive PD1 and CTLA4-expressing T cells contributes to increased activity. In the first-in-human (FIH) clinical study, XmAb®20717-01, a dose of 10 mg/kg XmAb®20717 every 2 weeks (Q2W) has been associated with confirmed complete and partial responses to therapy in a variety of tumor types, including melanoma, renal cell carcinoma, castration-resistant prostate cancer, and ovarian cancer.

Justification for Dose: As of Mar. 28, 2021, XmAb®20717 was administered to 145 subjects at dose levels ranging from 0.15 to 15 mg/kg administered Q2W in the FIH Phase 1 clinical trial, XmAb®20717-01. The starting dose level for the FIH study was based on both nonclinical and toxicology studies. Studies of XmAb®20717 binding to staphylococcal enterotoxin B-stimulated human T cells showed that the primary binding of the molecule is to PD1, and, further, that the binding affinity of the PD1 monovalent component arm of XmAb®20717 is comparable to that of the FDA-approved anti-PD1 monoclonal antibody nivolumab. Based on pharmacokinetic (PK) modelling using toxicokinetic data from a good laboratory practice toxicology study in cynomolgus monkeys, it was expected that the serum concentration achieved by administration of 0.3 mg/kg XmAb®20717 would exceed that required for biological activity during the entire 14-day period between doses. Allowing for an additional safety factor and noting that this dose is approximately 15% of the lowest approved dosing level for nivolumab (1 mg/kg Q2W), 0.15 mg/kg was chosen as the starting dose for the FIE study.

In the Phase 1 study, 42 subjects with advanced solid tumors were treated with XmAb®20717 at doses ranging from 0.15 to 15 mg/kg in the dose-escalation phase of the study. Although a maximum tolerated dose was not identified, and all administered dose levels were considered tolerable by the Dose Escalation Review Committee, 10 mg/kg Q2W was selected as the dose to be administered in 5 expansion cohorts into which 103 subjects were subsequently enrolled: advanced melanoma, non-small cell lung cancer, renal cell carcinoma, mCRPC, and a cohort that enrolled subjects with tumor types for which there was no extant checkpoint inhibitor (CI) approval but for which there was published evidence of response to CI treatment.

During the study, multiple PK samples were taken from subjects, assayed, and analyzed. The population PK analysis of these data showed that the PK of XmAb®20717 is characterized by two-phase distribution and elimination and a half-life of approximately 12.3 days. Further analysis and modeling of the PK data for subjects treated at the 10 mg/kg dose level showed that while some factors that influence variability in drug exposure among individuals who received the same weight-based dose level may remain unknown, body weight is the most influential factor. Exposure was increased in subjects with higher body weight. In addition, analysis of exposure in conjunction with the clinical response and safety data showed that higher drug exposure was correlated with a higher probability of achieving a clinical response and a higher probability of experiencing Grade 3 or higher irAEs.

As the clinical development program for XmAb®20717 advances, a simpler, more convenient dosing regimen would be advantageous. Therefore, in order to facilitate a regimen based on flat dosing and less frequent dose administration, PK modeling was conducted. The goal of the exercise was identifying a flat dosing schema that results in mean maximum observed concentration (Cₘₐₓ), area under the serum concentration-time curve (AUC), and last measurable concentration (Cₗₐₛₜ) values approximately equivalent to those obtained with the acceptable weight-based doses administered in the clinical trial. Specifically, the goal was to identify a flat dosing regimen that allowed administration of study drug every three weeks, while maintaining the approximate AUC and Cₗₐₛₜ parameters of the 10 mg/kg Q2W dose and not exceeding the Cₘₐₓ of the highest dose that was administered and found tolerable in the clinical trial—that is, 15 mg/kg Q2W.

Therefore, the PK profiles of all subjects treated in the FIH clinical trial were analyzed and modeled to derive flat doses equivalent to weight-based doses of 10 and 15 mg/kg, and then to project exposure over a 3-week dosing interval. The flat dose of 1200 mg was found to meet the defined conditions for Cₘₐₓ, AUC, and Cₗₐₛₜ. Because flat dosing results in the potential for overdosing of lower body weight subjects, further analysis and modeling were conducted with all subjects in the trial, who represented a wide range of body weights. This allowed the definition of a body weight cutoff and construction of a two-tiered flat dosing regimen, with subjects who weigh 80 kilograms or more receiving 1200 mg of XmAb®20717 IV Q3W and subjects who weigh less than 80 kilograms receiving 1000 mg XmAb®20717 IV Q3W.

TABLE 8
XmAb ®20717 dosage form and route of administration

| | Product |
|---|---|
| Product Name | XmAb ®20717 |
| Dosage Form | 10 mg XmAb ®20717 in 10 mL of 250 mM |
| | sorbitol and 0.01% (w/v) polysorbate 80 |
| | in 20 mM histidine buffered solution |
| | in a 10 mL vial |
| Unit Dose | 10 mg vial |
| Route of | Intravenous administration over 1 |
| Administration | hour (±10 minutes unless there is |
| | approval from the Medical Monitor for |
| | a different infusion rate and duration) |
| Physical | Clear to slightly opalescent, colorless |
| Description | to yellow liquid; practically free of |
| | visible particulates |

Dosing and Administration: Subjects are administered XmAb®20717 IV at a constant infusion rate over the course of 1 hour on the first day of each 21-day cycle. The subject's dose level will be assigned according to his/her baseline weight. Subject weight categories and the corresponding dose levels are detailed in Table 9 below. A subject who is initially assigned to the lower (1000 mg of XmAb®20717 Q3W) dose level by body weight and who receives 3 cycles (3 doses) of XmAb®20717 without experiencing a ≥Grade 2 irAE, may, according to investigator discretion, receive the higher dose level (1200 mg of XmAb®20717 Q3W) beginning with the fourth dose and for all doses thereafter until discontinuation of study drug.

TABLE 9
XmAb ®20717 Dosing

| | |
|---|---|
| Subject Weight (in kilograms) | Dose Level of XmAb ®20717 |

| | | |
|---|---|---|
| ≥80 | kg | 1200 mg Q3W |
| <80 | kg | 1000 mg Q3W |

Q3W = every 3 weeks.

However, if, at any time after study drug administration has begun, the subject's weight changes by more than 10% from baseline (excluding weight attributable to ascites and/or peripheral edema), and the new weight requires reassignment to a different dosing tier (Table 9), subsequent doses are received at the new dose level until such time as fulfillment of these rules may again require assignment to the other dose level.

In addition, a subject's dose level may be modified if he/she experiences an irAE(s) that allows continued dosing with study drug. After sufficient resolution of the AE(s), the subject's dose of XmAb®20717 may be reduced upon reinstitution of study drug dosing in certain circumstances.

Continuation of Therapy: Subjects who appear to benefit from XmAb®20717 treatment may continue to receive drug until unacceptable toxicity develops, disease progression occurs, or the Investigator believes there is no longer a benefit to the subject. To continue to receive study drug, a subject must also satisfy the following criteria:
Does not meet any criteria for stopping dosing.
Has recovered sufficiently from any immune-related toxicity so that he/she is eligible for continuation/re-initiation of XmAb®20717 dosing.
Has recovered from any other toxicity to ≤Grade 2 or baseline
Has clinical benefit, as assessed by the Investigator

Subjects who have PD per RECIST 1.1 or per Prostate Cancer Clinical Trials Working Group 3 (PCWG3) criteria (prostate cancer subjects only) may continue to receive XmAb®20717 if they:
Meet all the criteria for continuation of dosing listed above
Have not experienced a decline in ECOG performance status since the baseline assessment
Do not have signs or symptoms of unequivocal PD or PD in sensitive anatomical sites

For subjects who continue to receive XmAb®20717, tumor assessments are repeated as described above and in the SoA (Table 7). Subjects who discontinue XmAb®20717 due to an AE continue tumor response and safety assessments as described in the SoA (Table 7) until death or disease progression requiring therapy, whichever comes first. Subjects who discontinue treatment with XmAb®20717 for reasons other than an AE are examined for safety follow-up 10 weeks after XmAb®20717 discontinuation, unless initiation of other anticancer therapy has occurred due to disease progression. For subjects who discontinue treatment for reasons other than an AE or disease progression, information regarding disease status, including assessment of response based on standard-of-care imaging, are collected until death or disease progression requiring therapy, whichever comes first.

Concomitant Therapy: Subjects may not concurrently receive other therapeutics for treatment of their malignancy, except as described below. Subjects should also not receive live prophylactic vaccinations while enrolled in the study. For subjects with HBV who were eligible for the study (i.e., those who had a negative HBsAg, a positive HBcAb, and a negative HBV DNA test result at screening, and who must be retested for HBsAg and HBV DNA every 2 months during the study), if, during the course of participation in the study, the subject's HBsAg and HBV DNA tests become positive, suppressive antiviral therapy must be initiated before the subject resumes dosing with study drug.

Permitted Therapy: Subjects who experience infusion reactions can receive intervention and supportive measures. These may include, but are not limited to, epinephrine, antihistamines, corticosteroids, IV fluids, vasopressors, oxygen, bronchodilators, and acetaminophen.
Subjects may also receive the following additional therapy during the study:
   Treatment for AEs
   Antiemetics, antidiarrheals, anticholinergics, antispasmodics, antipyretics, antihistamines, analgesics, antibiotics, antihyperglycemics, and other medications intended to treat symptoms related to cancer, cancer therapy, or concurrent conditions
   Concomitant palliative localized radiation of lesions is permitted, as long as the irradiated lesion is not evaluated as a target lesion for the purposes of RECIST assessment
   Antiviral treatments for subjects with a positive HbcAb (and negative HBV DNA test) at screening or who test positive for HBV DNA during the study
   Transfusions of blood products such as red blood cells (RBCs) and platelets, if medically indicated
   Histamine receptors, type 2 antagonists, or proton-pump inhibitors for treatment of indigestion, nausea, and/or vomiting
   Multivitamins
   Killed inactivated prophylactic vaccines, such as the influenza vaccine, provided they are not given within 24 hours prior to dosing with XmAb®20717
   Inactivated or other non-live prophylactic vaccines for COVID-19, provided they are not given within 24 hours prior to dosing with XmAb®20717
   LHRH analogues in subjects with prostate cancer
   Bisphosphonates or denosumab in subjects with prostate cancer. Subjects taking bisphosphonates or denosumab should not have their dosing regimen altered during the study unless medically warranted

If a subject experiences Grade 3 elevation of AST and/or ALT without elevation of total bilirubin above the baseline level, and if the liver function test (LFT) elevation is identified as a possible irAE, rechallenge with XmAb®20717 may be considered after all of the following have occurred: a) the aspartate aminotransferase (AST) and/or alanine aminotransferase (ALT) value(s) has/have returned to Grade 0 or baseline, if the subject's baseline level was higher than Grade 0; b) all treatment for the presumed irAE has been completed, including all tapering dosing of corticosteroids; and c) the Medical Monitor has given approval for rechallenge.

Tumor Assessments: During the screening period before the first dose of study drug, at the completion of Cycles 2, 4, 6, and 8, and at the completion of every third cycle of treatment thereafter, all subjects undergo tumor assessment of response/progression per RECIST 1.1. In addition to RECIST 1.1 tumor assessment by imaging, subjects in the prostate cohort of this study have tumor/disease assessments via bone scintigraphy and measurement of serum prostate-specific antigen (PSA) levels, according to the guidelines for timing of these assessments contained in PCWG3 (Scher H I, Morris M J, Stadler W M, et al. Trial design and objectives for castration-resistant prostate cancer: updated recommendations from the Prostate Cancer Clinical Trials Working Group 3. J Clin Oncol. 2016; 34(12):1402-18) as described in the SoA (Table 7). Subjects who have a RECIST 1.1 assessment of PD may continue to receive study drug until PD is confirmed by a subsequent RECIST 1.1 assessment.

Pharmacokinetic/Pharmacodynamic Assessments: Venous blood samples for serum analyses of ADA are obtained according to the SoA (Table 7). A serum sample for measurement of pembrolizumab and nivolumab levels are collected on Day 1 only for those subjects who have received pembrolizumab or nivolumab within 6 months prior to the first dose of study drug. Blood samples for analyses of PK are obtained according to Table 10. Archival tissue and/or pre- and post-XmAb®20717 fresh tumor biopsies are examined by immunohistochemical and fluorescent immunohistochemical assays for immune cell density, intratumoral and juxtatumoral immune and tumor cell expression of PDL1, PD1, and other immune checkpoint markers, transcriptomic analysis of the tumor and tumor microenvironment, and next generation sequencing analysis of mutations and TMB, for correlation with clinical responses. A blood sample is collected to measure circulating tumor DNA (ctDNA) at the time points indicated to explore associations with response and acquired resistance and to explore associations of somatic tumor genomic alterations in blood with tumor assessments. Germline genomic DNA is sequenced but used for the purpose of confirming somatic origin of intratumoral and ctDNA mutations. In peripheral venous blood, baseline and serial assessment of B-cell, NK cell, and T-cell numbers, soluble factors including cytokines, as well as markers of T-cell activation, proliferation, and exhaustion are assessed. A PAXgene RNA blood sample is collected at baseline and on treatment to explore mRNA/transcriptomic changes in response to XmAb®20717 treatment.

TABLE 10
Sampling for Pharmacokinetics (Stages 1 and 2)

| | | | | | | | | Cycle | Post- |
|---|---|---|---|---|---|---|---|---|---|
| | | | | Cycles | | | | 6 and | Treatment |
| | Cycle 1 | | | 2-4 | Cycle 5 | | | Higher | (not timed) |
| Study Day: | 1 | 3-5 | 8-12 | 1 | 1 | 3-5 | 8-12 | 1 | EOT PK |
| Predose | PK | | | PK | PK | | | PK | 14 days post- |
| 5 minutes post-EOI | PK | | | PK | PK | | | PK | EOT PK |
| 1 hour post-EOI | PK | | | | PK | | | | 28 days post- |
| 2 hours post-EOI | PK | | | | PK | | | | EOT PK |
| Anytime ^{a} | | PK | PK | | | PK | PK | | |

EOI = end of infusion;
EOT = end of treatment;
PK = pharmacokinetics.
^{a} One PK sample should be drawn at any time during the listed period.

Safety and Other Assessments: Safety and tolerability assessments include the following: AEs; clinical safety assessments; laboratory assessments; vital signs; physical examination (PE) findings; and electrocardiogram (ECG) parameters.

Screening Period (Day −28 to −1): Subjects undergo a screening visit(s) 2 to 28 days prior to the planned first day of study treatment. Screening assessments are as follows:
Collect formalin-fixed paraffin embedded (FFPE) archival and/or fresh tumor sample or slides
Pretreatment fresh tumor biopsies are mandatory for all patients with accessible lesions and require consent
Review inclusion and exclusion criteria
Medical history, including any available genomic information on the primary or metastatic tumor and sequencing data from prior biopsy or surgery
Demographics
PE
Perform tumor assessment per RECIST 1.1 within 28 days prior to the first dose of drug
Bone scan, if not previously performed within 56 days of the first dose of study drug (prostate cancer subjects only)
PSA testing within 14 days of the first dose of study drug (prostate cancer subjects only)
Testosterone (prostate cancer subjects only)
ECOG performance status
Vital signs
ECG standard 12-lead, supine or semirecumbent position
Complete blood count with differential (CBC)
Chemistry panel, including amylase and lipase
Coagulation panel
Screening test for HbsAg and HbcAb
Screening tests for HIV and HCV
β-hCG pregnancy test for women of childbearing potential or FSH for postmenopausal women
Urinalysis
Blood for peripheral cytokine levels
Record concomitant medications
Record AEs

Baseline Assessments (Day 1 Predose): If a test or assessment is performed both during the Screening period and on Cycle 1 Day 1, the assessment closest to the time of dosing is considered the baseline:
Abbreviated medical history
Abbreviated PE, including height and weight
ECOG performance status
Vital signs
ECG, standard 12-lead, supine or semirecumbent position
CBC with differential
Chemistry panel, including amylase and lipase
Coagulation panel
Thyroid function tests: thyroid-stimulating hormone (TSH), triiodothyronine (T3), free thyroxine (fT4)
adrenocorticotropic hormone (ACTH), cortisol (8 AM)
β-hCG pregnancy test for women of childbearing potential
Blood for peripheral cytokine levels
Immunogenicity (anti-drug antibody [ADA]) blood sample(s)
Blood for PK
Serum for pembrolizumab and nivolumab levels predose on Day 1 only for those subjects who have previously received pembrolizumab or nivolumab within 6 months of the first dose of XmAb®20717
Blood for flow cytometry, PAXgene RNA, and ctDNA
Record concomitant medications and AEs

Treatment Cycle Assessments (Day 1 to End of Last Cycle): The schedule of required procedures and clinical site study days for each cycle is detailed in the SoA (Table 7). The day prior to Cycle 1 Day 1 is Day −1.

Clinical Assessments: PEs and clinical evaluations may be performed up to 24 hours prior to a scheduled infusion of XmAb®20717: abbreviated, symptom-directed PE, including weight; vital signs; ECOG performance status; record concomitant medications; and record AEs.

Laboratory Assessments: Clinically significant abnormal laboratory results that are not caused by the underlying disease or are not consistent with the subject's medications are recorded as AEs. The following will not be considered as AE: a not clinically significant, out-of-range value/finding; a not clinically significant, out-of-range value/finding explainable by anticipated or known effect of study drug or concomitant drugs; and/or a clinically significant value/finding that is consistent with the subject's underlying disease. A clinically significant, out-of-range value/finding should be reported as an AE.

The following laboratory variables are determined as outlined below:
Hematology: RBC, hemoglobin, hematocrit, erythrocyte mean corpuscular volume, RBC distribution width, white blood cell count with differential (percent and absolute; neutrophils, lymphocytes, monocytes, eosinophils, basophils), and platelet count
Coagulation panel: prothrombin time/international normalized ratio and activated partial thromboplastin time
Clinical chemistry: chemistry panel (sodium, chloride, potassium, bicarbonate, blood urea nitrogen, creatinine, glucose, uric acid, calcium, phosphorous, total bilirubin, ALT, AST, total protein, albumin, alkaline phosphatase, lactate dehydrogenase), serum gamma-glutamyltranspeptidase, amylase, and lipase
Thyroid function tests: TSH, T3, fT4
Measures of adrenal function: ACTH, cortisol (8 AM)
Urinalysis: pH, specific gravity, ketones, leukocytes, bilirubin, blood, protein, urobilinogen, nitrite, microscopic (reflex), clarity, color, and glucose
Tests for viral hepatitis and HIV: HBsAg, HBcAb, HCV, HIV
β-hCG pregnancy test for women of childbearing potential
PSA (subjects with prostate cancer, only)
Blood for cell surface markers by flow cytometry and similar bioanalytical methods
PK
Blood for peripheral cytokine levels
ADA blood samples
ECG, standard 12-lead
RECIST 1.1 tumor assessment; tumor assessment by computed tomography (CT)/magnetic resonance imaging (MM), measurement, and bone scans
Post-treatment fresh tumor biopsies (optional)

Vital Signs: On infusion days, vital signs are taken predose, immediately before end of infusion (EOI), and 30 (±5) minutes after EOI. On nondosing days, vital signs are measured prior to blood sampling. The following vital signs will be measured: blood pressure (systolic and diastolic [mmHg]); heart rate (beats per minute); respiratory rate (breaths per minute); and/or body temperature (° C.). Blood pressure and heart rate recordings are made after the subject has been at rest≥5 minutes.

Physical Examination (PE): Complete PE includes an assessment of general appearance and a review of systems (dermatologic, head, eyes, ears, nose, mouth, throat, neck, thyroid, lymph nodes, respiratory, cardiovascular, gastrointestinal, genitourinary, extremities, musculoskeletal, neurologic, and psychiatric). An abbreviated PE is symptom-directed and need not include detailed examination of all systems. Baseline body weight is used to determine the dose of XmAb®20717 to be administered.

Electrocardiogram: The 12-lead ECGs are performed after the subject has been resting supine or semirecumbent for ≥5 minutes. The ECG includes all 12 standard leads and a Lead II rhythm strip on the bottom of the tracing.

End of Study: The end of the study is defined as completion of the last visit or procedure shown in the schedule of activities (SoA) in the trial globally. Completion of the long-term follow up period is not required for a subject to be considered as having completed the study.

Post-Treatment Follow-Up Period (14, 28, and 70 Days After End of Treatment (EOT): Subjects who discontinue treatment for reasons other than an AE have visits 14 days post-EOT, 28 days post-EOT, and 70 days post-EOT. Assessment of response based on standard-of-care imaging are collected for subjects who discontinue treatment for reasons other than an AE or disease progression until death or disease progression requiring therapy, whichever comes first. Subjects are followed for AEs for 70 days after EOT or until the first administration of another anticancer therapy, whichever comes first.

Long-Term Follow-up Period (6 and 12 Months after EOT): Subjects are contacted at 6 and 12 months after their participation in the study for information on disease progression and survival.

Adverse Events: An adverse event or AE is any untoward medical occurrence in a study-subject administered XmAb®20717. An AE can be any unfavorable and unintended sign (including a clinically significant abnormal laboratory finding), symptom, or disease temporally associated with the use of XmAb®20717. AEs may include the onset of a new illness and the exacerbation of preexisting conditions. A Serious Adverse Event (SAE) is an AE occurring during the study period that fulfills one or more of the following:
1. Results in death
2. Is life-threatening; this means that the subject was at immediate risk of death at the time of the event. It does not mean that the event hypothetically might have caused death if it were more severe
3. Requires inpatient hospitalization or prolongation in existing hospitalization (more than 24 hours as an inpatient, or prolongation of planned hospitalization by 24 hours due to an AE)
4. Results in persistent or significant incapacity or substantial disruption of the ability to conduct normal life functions
5. Is a congenital anomaly or birth defect
6. Is an important medical event. Important medical events are events that may not be immediately life-threatening but are clearly of major clinical significance. Important medical events may jeopardize the subject and may require intervention to prevent one of the other serious outcomes listed above. Examples of such medical events include allergic bronchospasm requiring intensive treatment in an emergency room or in a physician's office, blood dyscrasias or seizures that do not result in subject hospitalization, and the development of drug dependency or drug abuse

All AEs are assessed for severity, also known as intensity or grade, utilizing the NCI-CTCAE (National Cancer Institute's Common Terminology Criteria for Adverse Events) grading scale (Version 5.0). AEs not contained within NCI-CTCAE, Version 5.0, are rated on a 5-point scale as described in Table 11. The following shall not be reported as an AE/SAE: signs/symptoms and conditions related to disease progression of the primary condition under the investigation (e.g., tumor-related pain, metastasis, new lesions, etc.) and hospitalization or death due to disease progression.

TABLE 11
Severity Grading Scale

| | |
|---|---|
| Mild (Grade 1) | Mild events are those which are easily |
| | tolerated without disruption of normal |
| | daily activity. |
| Moderate (Grade 2) | Moderate events are those which cause |
| | sufficient discomfort and interfere with |
| | daily activity. |
| Severe (Grade 3) | Severe events are those which incapacitate |
| | and prevent usual activity. |
| Life-threatening | An AE that has life-threatening consequences, |
| consequences | for which urgent intervention is indicated, |
| (Grade 4) | that puts the subject at risk of death at the time |
| | of the event if immediate intervention is not |
| | undertaken, or that causes blindness or deafness. |
| Fatal (Grade 5) | The termination of life as a result of an AE. |

Assessment of Potential Risks and Benefits: XmAb®20717 has been characterized through several in vitro binding experiments which demonstrate that the drug binds both PD1 and CTLA4. The number of double-positive cells within the tumor microenvironment has been shown to correlate with clinical responses to an anti-PD1 antibody, and these cells are therefore thought to be important in initiating, generating, and establishing an antitumor immune response after CI therapy. The immunological and antitumor effects of XmAb®20717 have been studied through experiments in mice that demonstrate the ability of the antibody to activate the immune system and promote the killing of tumor cells. Studies in cynomolgus monkeys, the only relevant species for this antibody, showed that dosing of XmAb®20717 at levels in excess of the human equivalent levels to be administered in this clinical trial resulted in no identifiable toxicity.

Current understanding of the risks associated with XmAb®20717 in humans is based on preliminary data from Study XmAb®20717-01. AEs observed following treatment with XmAb®20717 in this study were predominantly immunotherapy-related AEs (irAEs) that are consistent with the types of irAEs reported in clinical trials and post-approval treatment data for other PD1- and CTLA4-directed CIs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof are encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

1-40. (canceled)

41. A method of treating a prostate cancer in a male human subject in need thereof, the method comprising:
administering to the male human subject according to a 28 day treatment cycle, a bispecific antibody at a dose of about 10 mg/kg,
wherein the dose of the bispecific antibody is intravenously administered to the male human subject on day 1 of a first 28 day treatment cycle and about every two weeks (Q2W) thereafter, and
wherein the bispecific antibody comprises a first monomer comprising SEQ ID NO:1, a second monomer comprising SEQ ID NO:2, and a light chain comprising SEQ ID NO:3.

42. The method according to claim 41, wherein the prostate cancer is microsatellite instability-high (MSI-H) prostate cancer.

43. The method according to claim 41, wherein the prostate cancer is mismatch repair deficient (MMRD) prostate cancer.

44. The method according to claim 41, wherein the method further comprises:
(a) administering carboplatin at a therapeutically effective dose that results in a target area under the serum concentration-time curve of 4 (AUC4) in the male human subject, wherein the dose of the carboplatin is intravenously administered to the male human subject on day 1 of the first 28 day treatment cycle and about every three weeks (Q3W) thereafter; and
(b) administering cabazitaxel at a dose of about 20 mg/m², wherein the dose of the cabazitaxel is intravenously administered to the male human subject on day 1 of the first 28 day treatment cycle and about every three weeks (Q3W) thereafter.

45. The method according to claim 41, wherein the male human subject has not previously been administered docetaxel, and wherein the method further comprises:
(a) administering carboplatin at a therapeutically effective dose that results in a target area under the serum concentration-time curve of 4 (AUC4) in the male human subject, wherein the dose of the carboplatin is intravenously administered to the male human subject on day 1 of the first 28 day treatment cycle and about every three weeks (Q3W) thereafter; and
(b) administering docetaxel at a dose of about 60 mg/m² wherein the dose of the docetaxel is intravenously administered to the male human subject on day 1 of the first 28 day treatment cycle and about every three weeks (Q3W) thereafter.

46. The method according to claim 41, wherein the prostate cancer is an aggressive variant (anaplastic) adenocarcinoma of the prostate (AVPCa).

47. The method according to claim 46, wherein the prostate cancer has a mutation or other aberrancy in at least two genes independently selected from the group consisting of Rb1, TP53 and PTEN.

48. The method of claim 41, wherein the male human subject receives more than one 28 day treatment cycle.

49. The method according to claim 41, wherein the male human subject has received prior treatment with a polyadenosine diphosphate ribose polymerase (PARP) inhibitor.

50. The method according to claim 41, wherein the prostate cancer has a homologous recombination deficiency (HRD).

51. The method according to claim 41, wherein the prostate cancer has a biallelic loss of cyclin-dependent kinase 12 (CDK12).

52. The method according to claim 41, wherein the male human subject has not previously been administered a PARP inhibitor, and wherein the method further comprises administering olaparib to the male human subject at a dose of about 600 mg per day.

53. The method according to claim 52, wherein the prostate cancer has a HRD.

54. The method according to claim 52, wherein the prostate cancer has a biallelic loss of CDK12.

55. The method according to claim 41, further comprising orally administering a steroid to the male human subject.

56. The method according to claim 55, wherein the steroid is prednisone administered at a dose of about 5 mg twice per day (b.i.d.) on day 1 of the first 28 day treatment cycle, and about twice per day (b.i.d.) thereafter.

57. A method of treating an advanced gynecologic or genitourinary malignancy in a human subject in need thereof, the method comprising administering to the human subject a dose of a bispecific antibody according to a 21 day treatment cycle, wherein the dose of the bispecific antibody is about 1200 mg if the human subject weighs 80 kg or more, or wherein the dose of the bispecific antibody is about 1000 mg if the human subject weighs less than 80 kg, wherein the dose of the bispecific antibody is intravenously administered to the human subject on day 1 of each 21 day treatment cycle, wherein the bispecific antibody comprises a first monomer comprising SEQ ID NO:1, a second monomer comprising SEQ ID NO:2, and a light chain comprising SEQ ID NO:3.

58. The method of claim 57, wherein the malignancy is a platinum-resistant high-grade serous ovarian cancer (HGSOC), a platinum-resistant high-grade fallopian tube cancer, a platinum-resistant high-grade peritoneum cancer, a chemotherapy relapsed or refractory clear cell ovarian cancer, a chemotherapy relapsed or refractory clear cell endometrial cancer, a chemotherapy relapsed or refractory clear cell peritoneal cancer, an immune-checkpoint-inhibitor-refractory microsatellite stable (MSS) endometrial cancer, a previously treated recurrent cervical cancer, a previously treated metastatic cervical cancer, a high-risk metastatic castration-resistant prostate cancer (mCRPC), an advanced endometrial carcinoma that is not MSI-H or deficient mismatch repair (dMMR).

59. The method of claim 57, wherein if the weight of the human subject changes by more than 10% from baseline, the human subject is reassigned to a new dosing level and one or more subsequent doses are administered to the human subject at the new dosing level.

60. The method of claim 57, wherein if the human subject initially receives three 21 day treatment cycles of the 1000 mg dose of the bispecific antibody without experiencing a ≥Grade 2 immune-related adverse event (irAE), then the human subject receives 1200 mg of the bispecific antibody beginning with a fourth 21 day treatment cycle and all subsequent cycles.
